(19) United States
(12) Patent Application Publication
(10) Pub. No.: US 2012/0021920 A1
(43) Pub. Date: Jan. 26, 2012

(54) RADIATION THERAPY BIOMARKERS

(75) Inventors: Liewei Wang, Rochester, MN (US); Richard M. Weinshilboum, Rochester, MN (US); Krishna Kalari, Jacksonville, FL (US); Brooke L. Fridley, Rochester, MN (US); Yu Xin Qin, Rochester, MN (US)

(73) Assignee: Mayo Foundation for Medical Education and Research, Rochester, MN (US)

(21) Appl. No.: 13/259,928

(22) PCT Filed: Apr. 1, 2010
(86) PCT No.: PCT/US2010/029592

§ 371 Date: Sep. 27, 2011

Related U.S. Application Data
(60) Provisional application No. 61/166,493, filed on Apr. 3, 2009.

(51) Int. Cl. C40B 20/00; C12Q 1/68; C40B 30/00

(52) U.S. Cl. 506/2; 506/7; 435/6.11

(57) ABSTRACT

Materials and methods related to using biomarkers for prediction of response to radiation therapy.

### CROSS-REFERENCE TO RELATED APPLICATIONS

This application claims benefit of priority from U.S. Provisional Application Ser. No. 61/166,493, filed on Apr. 3, 2009.

### STATEMENT AS TO FEDERALLY SPONSORED RESEARCH

This invention was made with government support under grant no. GM061388, awarded by the National Institute of General Medical Sciences. The government has certain rights in the invention.

### TECHNICAL FIELD

This document relates to biomarkers for prediction of response to therapy, and more particularly to biomarkers that can be used to predict whether a subject will respond to radiation therapy.

### BACKGROUND

Radiation is widely used to treat a variety of cancers. For some tumor types, radiation can be the most important element in therapy. Response to radiation varies widely, however. In addition, severe side effects (e.g., skin lesions, pulmonary fibrosis, or esophagitis, depending on the organ(s) being treated) can result from radiation treatment.

### SUMMARY

This document is based in part on the discovery that single nucleotide polymorphisms (SNPs) may be useful as biomarkers to predict a subject's response to radiation therapy. Many factors can contribute to variation in response to radiation therapy, including genetic variation. A subject's response to radiation may be at least partially inherited. If a subject is determined to have biomarkers indicating that radiation treatment is likely to be effective, the treatment may be used. Conversely, if a subject's biomarker analysis indicates that radiation treatment is not likely to be effective, the subject can be spared the potentially adverse side effects of radiation, and other avenues of treatment can be attempted.

In one aspect, this document features an in vitro method for predicting the effectiveness of radiation therapy in a subject, comprising (a) providing a biological sample from the subject; (b) assaying the biological sample to determine whether it comprises a radiation therapy signature; and (c) classifying the subject as having a greater likelihood of responding to radiation therapy if the signature is present in the biological sample, and classifying the subject as having a lesser likelihood of responding to radiation therapy if the signature is not present in the biological sample. The method can further comprise communicating to a medical professional information regarding whether or not the signature is present in the biological sample. The method can further comprise communicating to a medical professional information indicating that the presence of the signature correlates with effectiveness of radiation therapy. The radiation therapy signature can comprise one or more markers selected from the group consisting of rs7000734, rs16885294, rs1561715, rs1610110, rs2859631, rs12569163, rs16889440, rs7554126, rs4742269, rs7591064, rs11250464, rs1853665, rs4392868, rs17598306, rs1471356, and rs4554799. The radiation therapy signature can comprise one or more markers selected from the group consisting of rs4392868, rs297550, rs1946944, rs12548426, rs1610110, rs1561715, rs1561714, rs4734298, rs7000734, and rs212551. The radiation therapy signature can comprise one or more markers selected from the group consisting of rs1610110, rs1561715, rs1561714, rs4392868, rs7000734, and rs212551. The radiation therapy signature can comprise one or more markers selected from the group consisting of rs6863920, rs6449478, rs2409791, rs4326096, rs206789, and rs286158. The radiation therapy signature can comprise one or more markers selected from the group consisting of rs4463400, rs6578080, rs4397386, rs7387053, rs4487737, rs13275618, rs6578083, rs7814976, rs7841539, rs6578084, and rs9324519. The radiation therapy signature can comprise one or more markers selected from the group consisting of rs12553351 and rs4742269. The radiation therapy signature can comprise rs1610110, rs1561715, or rs7000734.

In another aspect, this document features an in vitro method for determining a dose of radiation for administration to a subject, comprising (a) providing a biological sample from the subject; (b) assaying the biological sample to determine whether it comprises a radiation therapy signature; and (c) determining that the dose is lower if the signature is present in the biological sample than if the signature is not present in the biological sample, and determining that the dose is higher if the signature is not present in the biological sample than if the signature is present in the biological sample. The method can comprise communicating to a medical professional information regarding whether or not the signature is present in the biological sample. The method can further comprise communicating to a medical professional information indicating that the presence of the signature correlates with a lower dose of radiation therapy. The radiation therapy signature can comprise one or more markers selected from the group consisting of rs7000734, rs16885294, rs1561715, rs1610110, rs2859631, rs12569163, rs16889440, rs7554126, rs4742269, rs7591064, rs11250464, rs1853665, rs4392868, rs17598306, rs1471356, and rs4554799. The radiation therapy signature can comprise one or more markers selected from the group consisting of rs4392868, rs297550, rs1946944, rs12548426, rs1610110, rs1561715, rs1561714, rs4734298, rs7000734, and rs212551.

The radiation therapy signature can comprise one or more markers selected from the group consisting of rs1610110, rs1561715, rs1561714, rs4392868, rs7000734, and rs212551. The radiation therapy signature can comprise one or more markers selected from the group consisting of rs6863920, rs6449478, rs2409791, rs4326096, rs206789, and rs286158. The radiation therapy signature can comprise one or more markers selected from the group consisting of rs4463400, rs6578080, rs4397386, rs7387053, rs4487737, rs13275618, rs6578083, rs7814976, rs7841539, rs6578084, and rs9324519. The radiation therapy signature can comprise one or more markers selected from the group consisting of rs12553351 and rs4742269. The radiation therapy signature can comprise rs1610110, rs1561715, or rs7000734.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of the human variation cell line model system described herein.

FIG. 2 is a plot of area under the curve (AUC) vs. SNPs based on p-value and chromosomal position. The y-axis gives the −log₁₀(p-value) for the association, while the x-axis gives the relative chromosomal position for each SNP. Each dot represents an association identified. The analysis was conducted using a panel of 300 human lymphoblastoid cell lines (100 Caucasian American, 100 African American, and 100 Han-Chinese American cell lines), adjusted for ethnicity.

FIG. 3 is a plot of AUC vs. chromosome 8 SNPs based on p-value and chromosomal position. The y-axis gives the −log₁₀(p-value) for the association, while the x-axis gives the relative position on chromosome 8 for each SNP. Each dot represents an association identified. The analysis was conducted using a panel of 100 human (Caucasian American) lymphoblastoid cell lines.

FIG. 4A is a graph plotting representative radiation cytotoxicity dose-response curves. Two cell lines from each of the three ethnic groups studied were selected to illustrate a range of radiation cytotoxicity “dose response” curves. Squares indicate African-Americans (AA), triangles Caucasian-Americans (CA) and circles Han Chinese-Americans (HCA). The x-axis indicates log 10 (radiation dose) and the y-axis indicates the proportion of cell surviving after radiation exposure. FIG. 4B is a graph plotting the relationship of ethnic group to radiation AUC. FIG. 4C is a graph plotting the gender effect on radiation AUC. FIG. 4D is a graph plotting a frequency distribution histogram of In AUC values for 277 cell lines.

FIG. 5A is a graph plotting genome-wide association of basal expression with radiation AUC for 277 cell lines. The y-axis represents the −log 10(p value) for the association of individual expression array probe sets. Expression probe sets are plotted on the x-axis based on the chromosomal locations of their genes. If genes had more than one probe set, the one with the lowest p value was plotted. The horizontal line indicates a p value of 10⁻³. FIG. 5B is a graph plotting genome-wide SNP association with radiation AUC for 277 cell lines. The y-axis represents −log 10(p value) for the association of each SNP with radiation AUC. SNPs are plotted on the x-axis based on their chromosomal locations. The horizontal line indicates a p value of 10⁻⁴. FIG. 5C is a graph plotting the most significant “locus” on chromosome 8 that was associated with radiation AUC. Diamonds indicate SNPs observed by genotyping, while triangles indicate imputed SNPs. The y-axis represents −log 10(p value) for the association of each SNP with radiation AUC, and the x-axis represents the location of the SNP on chromosome 8.

FIG. 6 is a diagram depicting linkage disequilibrium analysis for 6 top SNPs on chromosome 8, locus “8C,” in AA and CA subjects.

FIG. 7 is a diagram of the strategy used to select genes for functional validation. Genome-wide association studies for radiation AUC were performed with 1.3 million SNPs or 54,000 expression probe sets. A SNP locus-Expression-AUC “integrated analysis” was performed using “loci” that contained at least 2 SNPs associated with radiation AUC with p values<10⁻⁴, 54,000 expression probe sets and radiation AUC associations were used to identify SNPs associated with radiation AUC through their influence on gene expression (SNP-Expression p value<10⁻⁴, Expression-AUC p value<10⁻³). Finally, 23 candidate genes, including 1 from a SNP locus-AUC analysis, 12 from Expression-AUC analyses and 10 from SNP locus-Expression-AUC “integrated analyses”, were selected for functional validation studies performed with multiple cancer cell lines.

FIG. 8 presents data from siRNA screening of candidate genes by MTS assay in multiple cancer cell lines. Data are shown for five of the top 23 candidate genes, which were selected on the basis of “integrated analyses” and their potential biological implications, and were studied functionally in HupT3, MIA-PaCa2 and HeLa cancer cell lines by MTS assay after siRNA knockdown performed with two “unvalidated” or one “validated” siRNA when available. Dark gray bars and squares indicate data for non-transfected cells; white bars and triangles indicate negative control siRNA; and striped bars, gray bars, and circles indicate data for specific siRNAs. If two siRNAs were available, “significance” was defined as a gene with a significant change in apparent AUC for both in comparison with control siRNA. FIG. 8A, Candidate gene symbols. FIG. 8B, QRT-PCR. The y-axis indicates relative gene expression after siRNA knockdown when compared with “all star negative” siRNA. FIG. 8C, MTS assays. The x-axis indicates the log 10 (radiation dose) and the y-axis indicates the proportion of cells surviving after exposure to radiation.

FIG. 9 presents data from siRNA screening of candidate genes by colony-forming assay performed with multiple cancer cell lines. Data for five of the 23 top candidate genes were selected for colony-forming assays using HupT3, MIA-PaCa2, HeLa and A549 cancer cell lines. QRT-PCR also was performed to determine knockdown efficiency. Dark gray bars and squares indicate data for non-transfected cells; white bars and triangles indicate negative control siRNA; and gray bars and circles indicate data for specific siRNAs. FIG. 9A, Candidate gene symbols. FIG. 9B, QRT-PCR to assess expression levels for each candidate gene after knockdown in each cell line. FIG. 9C, Colony-forming assays performed with HupT3, MIA-PaCa2, HeLa and A549 cancer cell lines. The x-axis indicates the log 10 (radiation dose), and the y-axis indicates colony-forming ability relative to control.

FIG. 10A is a pair of graphs plotting association of genotypes for two SNPs downstream of DEPDC1B with expression levels of DEPDC1B (left panel) and BORA (right panel) in 277 cell lines. FIG. 10B is a graph plotting the correlation of DEPDC1B and BORA log 2 expression in the 277 LCLs studied using microarray. The data are expressed as relative standard deviates (Z values). R represents the Pearson correlation coefficient. FIG. 10C is a pair of graphs plotting real time QRT-PCR to assess BORA expression levels after knockdown of DEPDC1B in HeLa (left panel) or HupT3 (right panel) cell lines. Cells were transfected with either negative control siRNA (grey) or specific siRNA for DEPDC1B (black). Controls (white) were untransfected cells. RNA was isolated 24 or 48 hours later to perform RT-PCR to determine DEPDC1B and BORA mRNA levels. Expression is expressed relative to levels after negative siRNA.

FIGS. 11A, 11B, and 11C are diagrams of the results of Ingenuity Pathway analyses. A total of 240 candidate genes, including 211 genes from either expression vs. AUC correlations (p value<10⁻³) or from “integrated analyses” (SNP vs. Expression p value<10⁻⁴, and Expression vs. AUC p value<10⁻³), and 29 genes from 27 “loci” that were associated with radiation AUC, with each locus containing at least 2 SNPs within 50 kb with p values<10⁻⁴, were used in the network analyses.

### DETAILED DESCRIPTION

### Genotype-Phenotype Association Studies

This document relates to results obtained using a panel of immortalized human lymphoblastoid cell lines obtained from healthy individuals of varying ethnicities. The panel can allow one to query genomic variation across the entire genome for the effects of inheritance on variation in response to drug or radiation therapy. Such a panel can be used for preclinical testing for common, functionally significant gene sequence variation that influences treatment phenotypes. For example, a cell line panel can be used to test individual responses to therapies such as radiation treatment, drugs (e.g., anti-cancer drugs and immunosuppressants), and drug metabolites. Further, pharmaceutical companies can test drugs on a cell line panel prior to clinical trials, and medical researchers can use such a cell line panel to determine genetic reasons for adverse reactions to therapies, or failure of a therapy to be efficacious.

Therapeutic response phenotypes can vary from life-threatening adverse reactions at one end of the spectrum to lack of the desired therapeutic efficacy at the other. Thus, a cell line panel such as that described herein can be used to define, prior to patient exposure, the possible effect of common DNA sequence variation on response to a particular therapy. For example, in depth resequencing data can be obtained in the cell lines for genes encoding proteins in known pathways for radiation effects (e.g., DNA damage, DNA repair, and cell cycle pathways), as well as drug metabolism, drug transport, and drug effects. In addition, genome-wide single nucleotide polymorphisms (SNPs) across the entire genome can be obtained for the individual cell lines for use in genome-wide association studies. Genotype-phenotype correlation analyses using SNPs and intragene haplotype (the combination of SNPs on a given allele) resulting from gene resequencing and genome-wide SNPs can be performed to identify “therapogenomic” and pharmacogenomic candidate genes, both within traditional pharmacokinetic (PK) and pharmacodynamic (PD) pathways, as well as across the entire genome. Expression array data for every gene in the human genome encoding a protein, as well as exon array data and genome-wide gene copy number information also can be obtained for the cell lines.

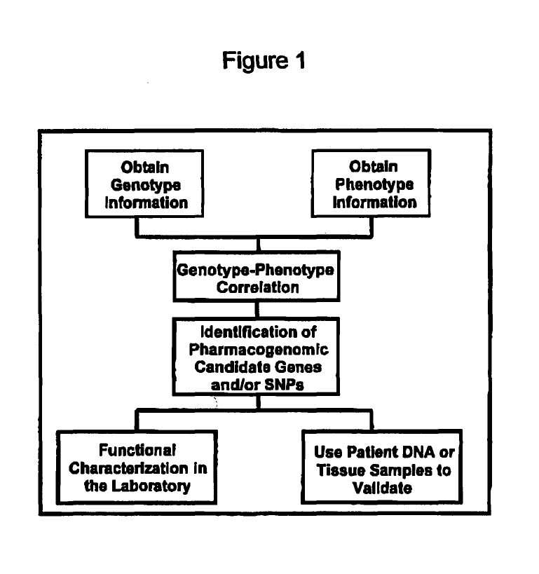

Further, as future techniques for defining DNA sequence variation are developed, culminating in complete genomic sequence for each cell line, those techniques can be added to accumulate a dense array of information—in effect, a “data warehouse”—with respect to differences in DNA sequence and structure that can be correlated with variation in drug-related phenotypes. Those phenotypes may include variation in gene expression, variation in cytotoxicity, variation in apoptosis, variation in nucleic acid methylation, and variation in metabolites in response to varying levels of radiation or varying concentrations of drug, for example. All of this information can be used to perform both “pathway-based” and “genome-wide” genotype-phenotype correlations to identify genetic polymorphisms and/or haplotypes that can be used to develop hypotheses with the cell lines, which then can be tested functionally in the laboratory and also in the clinic, using patient DNA or tissue samples (see FIG. 1). Therefore, the panel of cell lines described herein can be used to identify and characterize the effect of common variation in DNA sequence and structure in human populations on therapy response phenotypes that might be responsible for individual differences in adverse reactions to radiation therapy or in the efficacy of radiation therapy. It is noted that in addition to sequence information, data related to levels of metabolites, polypeptides, and mRNAs can be obtained from the panel of cell lines and correlated to individual variation in therapeutic effects.

Cells used in the model system described herein can be obtained commercially, for example, from the non-profit Coriell Institute for Medical Research (online at cimr.umdnj.edu). For example, the Human Variation Panel cell lines available from Coriell can be used. The Human Variation Panel includes immortalized lymphoblastoid cell lines collected from 100 African American (AA), 100 Caucasian American (CA), 100 Han-Chinese American (HCA) subjects and 23 CEPH (Utah family) cell lines. The panel used in the methods described herein can include any suitable number of individual cell lines from any ethnic group. For example, the panel can include from 50 to 100 individual AA cell lines, from 50 to 100 CA cell lines, and/or from 50 to 100 HCA cell lines. DNA from the cell lines can be used for in-depth resequencing of genes of interest, and also to obtain genome-wide SNP data for use during genome-wide association studies. The advantage of this system is that the cells are “renewable” and broadly accessible to the general scientific community. In addition, these cell lines represent ethnically diverse population groups.

Modern genomic tools (e.g., genome-wide SNPs and in depth resequencing of functionally important genes) can be used with the cell line panel to identify genes that might be associated with therapeutic response phenotypes. Phenotypes correlated with this genetic variation can include, for example, expression array and metabolomic data, therapy-induced cytotoxicity, methylation status, copy number, and cell cycle effects. SNPs or genes showing significant association with these phenotypes then can be tested functionally and, eventually, clinically. In essence, each of the cell lines in the panel can be viewed as an individual “patient” with a unique genotype and a series of associated phenotypes that can be used for preclinical screening of candidate genes and SNPs. A tremendous advantage of this model system is the fact that high throughput genetic data for these cell lines can be added continuously. Therefore, unlike patient-based information, data for these cell lines can “accumulate” and be used for studies involving a variety of therapeutic response phenotypes and a virtually endless series of therapies or therapy candidates.

SNP and haplotype associations can be performed with cell-based phenotypes and/or with phenotypes related to the response to treatment of disease with particular therapies. Cell-based phenotypes include, for example, cytotoxicity, levels of intracellular metabolites, and gene expression before and after treatment in lymphoblastoid cells. Patient-related phenotypes include, for example, overall patient survival and/or time to progression after treatment, as well as therapy-related toxicity phenotypes, including neutrophil and platelet counts.

The association of each SNP with the quantitative phenotypes of metabolite concentration, cytotoxicity, and level of gene expression, as well as neutrophil and platelet counts can be evaluated with linear models in which genotypes for a SNP are evaluated with two indicators as covariates. This provides a 2 degree-of-freedom (df) test for each SNP. To assess single SNP genotype associations with patient survival time and time to progression, the Kaplan-Meier method can be used to estimate survival curves for the different genotypes. The curves can be compared using log-rank tests. Survival time as a function of genotype can be examined using the Cox proportional hazards model, and hazard ratios can be used to examine the survival rate by genotype (Cox (1972) Journal of the Royal Statistical Society Series B:187-220). Disease status, age at time of treatment, gender and duration of treatment can be included as covariates in the proportional hazards models.

In addition to the association of phenotypes with SNPs, their association with intragene haplotypes can be evaluated for candidate genes using a global test of association. Since haplotypes are not observed directly, unknown phase can be accounted for using the score statistics developed by Schaid et al. ((2002) Am J Hum Genet 70:425-34). To estimate the magnitude of effects from haplotypes found to be significant using the score statistics, haplotype regression methods can be used. See, e.g., Lake et al. (2003) Hum Hered 55:56-65. Intragene haplotypes can be associated with clinical response using survival time and time to progression as phenotypes. All possible pairs of haplotypes can be evaluated for each patient, and the posterior probability can be associated with each haplotype using the EM algorithm, as implemented in the Splus library Haplostat (Schaid et al., supra). These posterior probabilities can be used to create expected design matrices to evaluate the association of haplotypes with survival time via the Cox model.

As used herein, a “radiation therapy signature” refers to a SNP profile where one or more (e.g., one, two, three, four, five, six, seven, eight, nine, or ten) of the SNPs listed in the tables presented herein (e.g., Table 1, Table 6, Table 7, Table 8, or Table 9) are present in a mammal. In some cases, for example, a radiation therapy signature can be a profile where 10, 20, 30, 40, 50, 60, 70, 80, 90, or 100 percent of the SNPs listed in Table 1, Table 6, Table 7, Table 8, Table 9, or a combination thereof, are present in a mammal.

In addition to sequence information, data related to levels of one or more metabolites, polypeptides, and/or RNAs (e.g., mRNAs) can be obtained from cell lines and correlated to drug responses. Cell lines can be characterized for any number of SNPs, metabolites, polypeptides, and RNAs (e.g., at least 100, at least 1,000, at least 10,000, at least 20,000, at least 50,000, or at least 100,000 SNPs, metabolites, polypeptides, or RNAs). In some embodiments, a cell can be characterized for all known SNPs, and levels of all known metabolites, all known polypeptides, and/or all known mRNAs.

In some cases, information obtained for particular therapeutic agents can be extrapolated to other agents that have similar metabolic pathways. Further, information regarding the cellular response (e.g., apoptosis and metabolism) in various ethnic groups for various doses of particular agents can be obtained to determine whether higher or lower doses may be needed for efficacy and/or to avoid toxicity. For example, if it is determined that a particular ethnicity is likely to be more resistant to a therapeutic agent (e.g., radiation), a higher dose can be used, whereas if it is determined that a particular ethnicity is likely to be more responsive to the agent, a lower dose may be used. If it is determined that a particular ethnicity is more likely to experience adverse side effects in response to a therapeutic agent, a lower dose can be used, whereas if it is determined that a particular ethnicity is less likely to experience adverse side effects in response to the agent, a higher dose may be used.

### Methods

This document provides methods for assessing a subject's likelihood of responding to radiation therapy, and/or for determining radiation dose levels. The methods provided herein can include, for example, testing a biological sample obtained from a subject to determine whether the sample contains one or more biomarkers indicating that the subject is likely to respond to radiation therapy, or to experience adverse side effects from radiation therapy. Such methods also can be used to, for example, determine whether a subject should be treated with a lower rather than a higher dose of radiation (e.g., if a biological sample from the subject contains a nucleotide polymorphism associated with responsiveness to radiation therapy or with adverse side effects to radiation therapy, it can be an indication that the subject should be treated with a lower dose of radiation than if the subject did not contain the polymorphism).

Any suitable biological sample can be used. A biological sample can be, for example, blood, serum, plasma, urine, cerebrospinal fluid, pleural fluid, sputum, peritoneal fluid, bladder washings, oral washings, tissue samples, touch preps, or fine-needle aspirates.

In some embodiments, a biomarker can be a nucleotide sequence variant (e.g., rs4392868, rs297550, rs1946944, rs12548426, rs1610110, rs1561715, rs1561714, rs4734298, rs7000734, or rs212551). Nucleotide sequence variants can be detected, for example, by sequencing exons, introns, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection (Schafer et al. (1995) Nat. Biotechnol. 15:33-39), denaturing high performance liquid chromatography (DHPLC, Underhill et al. (1997) Genome Res. 7:996-1005), infrared matrix-assisted laser desorption/ionization (IR-MALDI) mass spectrometry (WO 99/57318), and combinations of such methods.

Genomic DNA generally is used in the analysis of nucleotide sequence variants, although mRNA also can be used. Genomic DNA is typically extracted from a biological sample such as a peripheral blood sample, but can be extracted from other biological samples, including tissues (e.g., mucosal scrapings of the lining of the mouth or from renal or hepatic tissue). Routine methods can be used to extract genomic DNA from a blood or tissue sample, including, for example, phenol extraction. Alternatively, genomic DNA can be extracted with kits such as the QIAAMP® Tissue Kit (Qiagen, Chatsworth, Calif.) and the WIZARD® Genomic DNA purification kit (Promega).

An amplification step typically is performed before proceeding with the detection method. For example, exons or introns of a gene can be amplified and then directly sequenced. Dye primer sequencing can be used to increase the accuracy of detecting heterozygous samples.

Allele specific hybridization is an example of a method that can be used to detect sequence variants, including complete haplotypes of a subject (e.g., a mammal such as a human). See, Stoneking et al. (1991) Am. J. Hum. Genet. 48:370-382; and Prince et al. (2001) Genome Res. 11:152-162. In practice, samples of DNA or RNA from one or more mammals can be amplified using pairs of primers and the resulting amplification products can be immobilized on a substrate (e.g., in discrete regions). Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. High stringency conditions can include the use of low ionic strength solutions and high temperatures for washing. For example, nucleic acid molecules can be hybridized at 42° C. in 2×SSC (0.3M NaCl/0.03 M sodium citrate/0.1% sodium dodecyl sulfate (SDS) and washed in 0.1×SSC (0.015M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For nucleotide sequence variants that introduce a restriction site, restriction digest with the particular restriction enzyme can differentiate the alleles. For sequence variants that do not alter a common restriction site, mutagenic primers can be designed that introduce a restriction site when the variant allele is present or when the wild type allele is present. A portion of a nucleic acid can be amplified using the mutagenic primer and a wild type primer, followed by digest with the appropriate restriction endonuclease.

Certain variants, such as insertions or deletions of one or more nucleotides, change the size of the DNA fragment encompassing the variant. The insertion or deletion of nucleotides can be assessed by amplifying the region encompassing the variant and determining the size of the amplified products in comparison with size standards. For example, a region of a gene can be amplified using a primer set from either side of the variant. One of the primers is typically labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type allele would have amplification products only in the reaction using the wild type primer. Similarly, patient samples containing solely the variant allele would have amplification products only in the reaction using the variant primer. Allele-specific PCR also can be performed using allele-specific primers that introduce priming sites for two universal energy-transfer-labeled primers (e.g., one primer labeled with a green dye such as fluorescein and one primer labeled with a red dye such as sulforhodamine). Amplification products can be analyzed for green and red fluorescence in a plate reader. See, Myakishev et al. (2001) Genome 11:163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

In some embodiments, a biomarker can be a variant polypeptide. Antibodies having specific binding affinity can be used to detect variant polypeptides. Variant polypeptides can be produced in various ways, including recombinantly, as known in the art. Host animals such as rabbits, chickens, mice, guinea pigs, and rats can be immunized by injection of a variant polypeptide. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Polyclonal antibodies are heterogeneous populations of antibody molecules that are contained in the sera of the immunized animals. Monoclonal antibodies, which are homogeneous populations of antibodies to a particular antigen, can be prepared using a variant polypeptide and standard hybridoma technology. In particular, monoclonal antibodies can be obtained by any technique that provides for the production of antibody molecules by continuous cell lines in culture such as described by Kohler et al. (1975) Nature 256:495, the human B-cell hybridoma technique (Kosbor et al. (1983) Immunology Today 4:72; Cote et al. (1983) Proc. Natl. Acad. Sci USA 80:2026), and the EBV-hybridoma technique (Cole et al., Monoclonal Antibodies and Cancer Therapy, Alan R. Liss, Inc., pp. 77-96 (1983). Such antibodies can be of any immunoglobulin class including IgG, IgM, IgE, IgA, IgD and any subclass thereof. A hybridoma producing monoclonal antibodies can be cultivated in vitro or in vivo.

Antibody fragments that have specific binding affinity for a variant polypeptide can be generated using known techniques. For example, such fragments include but are not limited to F(ab')2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al. (1989) Science 246:1275. Once produced, antibodies or fragments thereof are tested for antigen recognition by standard immunoassay methods including ELISA techniques, radioimmunoassays and Western blotting. See, Short Protocols in Molecular Biology, Chapter 11, Green Publishing Associates and John Wiley & Sons, edited by Ausubel et al., 1992.

In some embodiments, a biomarker can be a level of a nucleic acid (e.g., an RNA) polypeptide, or metabolite that is altered with respect to, for example, a control level of the nucleic acid, polypeptide, or metabolite. Levels of nucleic acids, polypeptides, and metabolites can be determined using any suitable methods, including those that are known in the art. These include, for example, antibody-based methods, reverse transcriptase PCR (RT-PCR) methods, and any other methods that can be used to measure the level of a nucleic acid, polypeptide, or metabolite in a biological sample.

The methods provided herein can be used to predict the effectiveness of radiation therapy or the likelihood of an adverse response to radiation therapy in a subject (e.g., a mammal such as a rat, a dog, or a human). For example, a method can include determining whether a biological sample from a subject comprises one or more SNPs (e.g., rs4392868, rs297550, rs1946944, rs12548426, rs1610110, rs1561715, rs1561714, rs4734298, rs7000734, or rs212551), and classifying the subject as having a greater likelihood of responding to radiation therapy if the variant allele is present in the biological sample, and classifying the subject as having a lesser likelihood of responding to radiation therapy if the wild type allele is present in the biological sample. It is to be noted that although resistance to radiation therapy may indicate less disease response, it also may indicate protection against radiation damage. In addition, it is to be noted that allele frequencies will vary among ethnic groups. Thus, the markers disclosed herein may be useful to “individualize” this type of medical therapy.

The methods provided herein also can be used to determine a dose of radiation for administration to a subject. For example, using a method as described herein, it can be determined that a dose for a particular subject should be lower if a variant allele (e.g., rs4392868, rs297550, rs1946944, rs12548426, rs1610110, rs1561715, rs1561714, rs4734298, rs7000734, or rs212551) is present in a biological sample from the subject, and determining that the dose should be higher if the corresponding wild type allele is present in the biological sample.

The methods described herein can be carried out using a computer programmed to receive data (e.g., data from a chip containing a panel of SNPs, indicating whether a subject contains SNPs associated with sensitivity or resistance to radiation therapy). The computer can output for display information related to a subject's biomarkers, and the likelihood that the subject will be sensitive or resistant to radiation therapy.

This document also provides methods and materials to assist medical or research professionals in determining whether or not a subject is likely to respond to or be adversely affected by radiation therapy. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether a subject has one or more biomarkers associated with sensitivity or resistance to radiation therapy (e.g., the SNPs listed in Table 1), and (2) communicating information about the biomarkers to that professional.

In some embodiments, a method for assessing the likelihood that radiation therapy will be effective in a subject can include receiving a biological sample obtained from the subject, assaying the sample to determine whether it contains one or more particular variant alleles (e.g., rs4392868, rs297550, rs1946944, rs12548426, rs1610110, rs1561715, rs1561714, rs4734298, rs7000734, or rs212551), communicating to a medical professional information about whether the wild type or variant allele(s) are present in the sample, and, in some cases, before or after the first step, communicating to a medical professional information indicating that the presence of the variant allele(s) correlates with responsiveness to radiation therapy. Similarly, a method for determining a dose of radiation for administration to a subject can include receiving a biological sample obtained from a subject, assaying the sample to determine whether it contains one or more particular variant alleles (e.g., rs4392868, rs297550, rs1946944, rs12548426, rs1610110, rs1561715, rs1561714, rs4734298, rs7000734, or rs212551), communicating to a medical professional information about whether the wild type or variant allele(s) are present in the sample, and, in some cases, before or after the first step, communicating to a medical professional information indicating that the presence of the variant allele(s) correlates with a lower suggested dose.

After information regarding a subject's biomarkers is reported, a medical professional can take one or more actions that can affect patient care. For example, a medical professional can record the information in a subject's medical record. In some cases, a medical professional can record that the subject is likely or not likely to respond to radiation therapy, or otherwise transform the patient's medical record, to reflect the patient's medical condition. In some cases, a medical professional can review and evaluate a patient's medical record, and can assess multiple treatment strategies for clinical intervention of a patient's condition.

A medical professional can communicate information regarding biomarker analysis to a subject or a subject's family. In some cases, a medical professional can provide a subject and/or a subject's family with information regarding radiation therapy, including treatment options and potential side effects. In some cases, a medical professional can provide a copy of a subject's medical records to communicate information regarding biomarker analysis and/or disease states to a specialist.

A research professional can apply information regarding a subject's biomarkers to advance research into radiation toxicity. For example, a researcher can compile data on the presence of particular biomarkers (e.g., SNPs) with information regarding the efficacy of radiation therapy, or side effects associated with radiation therapy. In some cases, a research professional can obtain a subject's biomarker information to evaluate the subject's enrollment, or continued participation in a research study or clinical trial. In some cases, a research professional can communicate a subject's biomarker information to a medical professional, or can refer a subject to a medical professional for clinical assessment and/or treatment.

Any appropriate method can be used to communicate information to another person (e.g., a professional), and information can be communicated directly or indirectly. For example, a laboratory technician can input biomarker information into a computer-based record. In some cases, information can be communicated by making an physical alteration to medical or research records. For example, a medical professional can make a permanent notation or flag a medical record for communicating information to other medical professionals reviewing the record. Any type of communication can be used (e.g., mail, e-mail, telephone, and face-to-face interactions). Information also can be communicated to a professional by making that information electronically available to the professional. For example, information can be placed on a computer database such that a medical professional can access the information. In addition, information can be communicated to a hospital, clinic, or research facility serving as an agent for the professional.

### Articles of Manufacture

This document also provides articles of manufacture that can include, for example, materials and reagents that can be used to determine whether a subject has a biomarker for predicting response to radiation therapy. An article of manufacture can include, for example, nucleic acids and/or polypeptides immobilized on a substrate (e.g., in discrete regions, with different populations of isolated nucleic acids or polypeptides immobilized in each discrete region). Suitable substrates can be of any shape or form and can be constructed from, for example, glass, silicon, metal, plastic, cellulose, or a composite. For example, a suitable substrate can include a multiwell plate or membrane, a glass slide, a chip, or polystyrene or magnetic beads. Nucleic acid molecules or polypeptides can be synthesized in situ, immobilized directly on the substrate, or immobilized via a linker, including by covalent, ionic, or physical linkage. Linkers for immobilizing nucleic acids and polypeptides, including reversible or cleavable linkers, are known in the art. See, for example, U.S. Pat. No. 5,451,683 and WO98/20019. Immobilized nucleic acid molecules are typically about 20 nucleotides in length, but can vary from about 10 nucleotides to about 1000 nucleotides in length.

In practice, to detect a particular allele of a nucleic acid, for example, a sample of DNA or RNA from a subject can be amplified, the amplification product hybridized to an article of manufacture containing populations of isolated nucleic acid molecules in discrete regions, and hybridization can be detected. Typically, the amplified product is labeled to facilitate detection of hybridization. See, for example, Hacia et al. (1996) Nature Genet. 14:441-447; and U.S. Pat. Nos. 5,770,722 and 5,733,729.

The invention will be further described in the following example, which does not limit the scope of the invention described in the claims.

### EXAMPLES

### Example 1

Using an in vitro system, 300 “Human Variation Panel” lymphoblastoid cell lines were used to identify SNPs associated with radiation sensitivity. These 300 cell lines were obtained from the Coriell Institute supported by National Institute of General Medical Science (NIGMS). These EBV transformed lymphoblastoid cell lines were derived from 100 Caucasian-American, 100 African-American, and 100 Han-Chinese American subjects. Therefore, the cell lines have diverse genetic backgrounds across three ethnic groups. In addition, 1.3 million SNPs were obtained using two different platforms—the Affymetrix 6.0 SNP chip and the Illumina 550K and 510S Genechip—for each cell line. Since expression array data are available for all 300 cell lines, this system was ideal for generating and testing pharmacogenomic hypothesis.

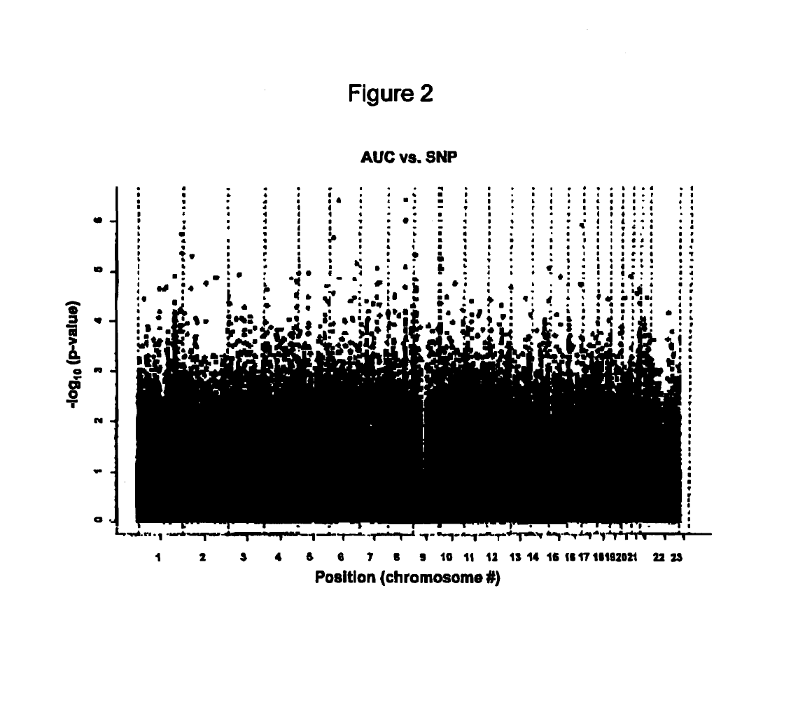

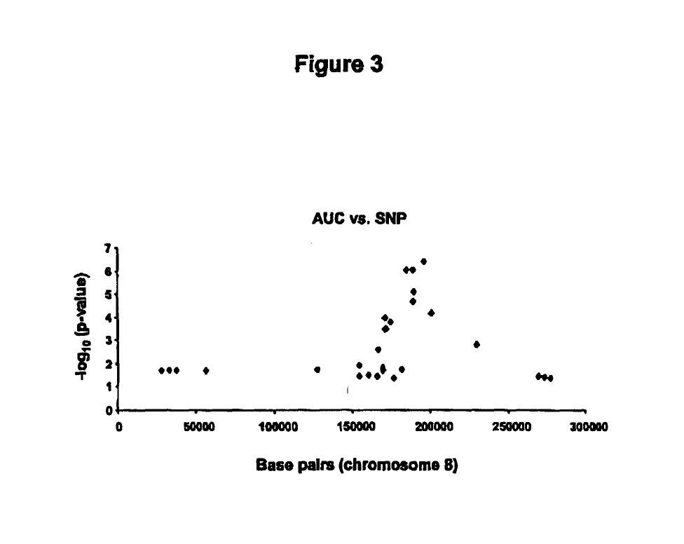

To identify SNPs and/or genes that might be associated with radiation sensitivity, radiation cytotoxicity studies were performed with the 300 cell lines. Radiation doses ranging from 2 to 10 Gray were used to treat each cell line for three days. Cell viability was assessed with a 96-well CellTiter-Blue Cell Viability Assay (Promega Corp.; Madison, Wis.), and the area under the curve (AUC) was calculated based on the dose-response curve for each cell line using the R package. The distribution of AUC across all three ethnicities was used as the radiation response phenotype shown in FIG. 2. This genome-wide SNP association identified ten SNPs (Table 1) on chromosome 8q22 that were in linkage disequilibrium and were significantly associated with radiation sensitivity. The distribution of AUC chromosome 8 SNPs in the CA cell lines is shown in FIG. 3. The “SNP signals” listed in Table 1 represent a “p value peak,” and SNPs with the lowest p values (i.e., the tops of the peaks in FIGS. 2 and 3) are particularly useful. The frequencies of these ten SNPs in each ethnicity are shown in Table 2.

The SNPs listed in Table 1 also were significantly associated with expression levels for 229 genes having p values less than 10⁻⁴. These genes, which are listed in Table 3, are involved in DNA damage, DNA repair, and cell cycle pathways, and are biologically relevant to radiation treatment. Therefore, the ten SNPs identified in these studies may be useful biomarkers for prediction of response to radiation therapy.

TABLE 1

| SNP | Polymorphism | P value |
|---|---|---|
| rs4392868 | G/A | 10⁻⁶ |
| rs297550 | G/A | 10⁻⁴ |
| rs1946944 | A/C | 10⁻⁴ |
| rs12548426 | A/G | 10⁻⁴ |
| rs1610110 | C/T | 10⁻⁷ |
| rs1561715 | A/G | 10⁻⁷ |
| rs1561714 | A/C | 10⁻⁵ |
| rs4734298 | G/A | 10⁻⁴ |
| rs7000734 | C/T | 10⁻⁷ |
| rs212551 | G/A | 10⁻⁵ |

TABLE 2

| | AA | HCA | CA |
|---|---|---|---|
| rs4392868 | | | |
| A | 0.91 | 0.02 | 0.21 |
| G | 0.08 | 0.98 | 0.79 |
| rs297550 | | | |
| G | 0.96 | 0.99 | 0.84 |
| A | 0.04 | 0.01 | 0.16 |
| rs1946944 | | | |
| A | 0.22 | 0.05 | 0.4 |
| C | 0.78 | 0.95 | 0.6 |
| rs12548426 | | | |
| A | 0.79 | 0.93 | 0.58 |
| G | 0.21 | 0.07 | 0.42 |
| rs1610110 | | | |
| C | 0.93 | 1 | 0.8 |
| T | 0.07 | 0 | 0.2 |
| rs1561715 | | | |
| A | 0.93 | 1 | 0.8 |
| G | 0.07 | 0 | 0.2 |
| rs1561714 | | | |
| A | 0.08 | 0 | 0.2 |
| C | 0.92 | 1 | 0.8 |
| rs4734298 | | | |
| G | 0.8 | 0.93 | 0.6 |
| A | 0.2 | 0.07 | 0.4 |
| rs7000734 | | | |
| C | 0.06 | 0 | 0.2 |
| T | 0.94 | 1 | 0.8 |
| rs212551 | | | |
| G | 0.84 | 0.98 | 0.75 |
| A | 0.16 | 0.02 | 0.25 |

TABLE 3
A kinase (PRKA) anchor protein 13
Abelson helper integration site
acidic (leucine-rich) nuclear phosphoprotein 32 family, member B
actin, gamma 1
Actinin, alpha 4
adenylate kinase 2
ADP-ribosylation factor related protein 2
ADP-ribosylhydrolase like 2
amyotrophic lateral sclerosis 4
ankyrin repeat domain 23
AP1 gamma subunit binding protein 1
arginase, type II
asparagine-linked glycosylation 12 homolog (yeast, alpha-1,6-
mannosyltransferase)
ATPase family, AAA domain containing 2
ATPase, Ca++ transporting, cardiac muscle, fast twitch 1
ATPase, H+ transporting, lysosomal V0 subunit a isoform 1
ATP-binding cassette, sub-family B (MDR/TAP), member 4
baculoviral IAP repeat-containing 5 (survivin)
baculoviral IAP repeat-containing 6 (apollon)
bolA-like 3 (E. coli)
Bromodomain containing 4
catenin (cadherin-associated protein), alpha-like 1
catenin (cadherin-associated protein), delta 1
CD44 antigen (homing function and Indian blood group system)
CDC28 protein kinase regulatory subunit 1B
CDC45 cell division cycle 45-like (S. cerevisiae)
CDNA clone IMAGE:30332316
CDNA FLJ31593 fis, clone NT2RI2002481
CDNA FLJ33139 fis, clone UTERU1000109
CDNA FLJ36209 fis, clone THYMU2000022
CDNA FLJ36837 fis, clone ASTRO2011422
CDNA FLJ38765 fis, clone KIDNE2014489
CDNA FLJ40647 fis, clone THYMU2017522
CDNA FLJ41675 fis, clone HCASM2002148
CDNA FLJ42561 fis, clone BRACE3006463
CDNA FLJ42786 fis, clone BRAWH3006761
CDNA FLJ46024 fis, clone SPLEN2022785
CDNA FLJ46713 fis, clone TRACH3016885
CDNA: FLJ23006 fis, clone LNG00414
cell division cycle 2, G1 to S and G2 to M
cell division cycle associated 2
cell division cycle associated 3
cell division cycle associated 7-like
CHK1 checkpoint homolog (S. pombe)
chondroitin sulfate glucuronyltransferase
chromodomain helicase DNA binding protein 9
chromosome 1 open reading frame 155
chromosome 1 open reading frame 183
chromosome 1 open reading frame 38
chromosome 10 open reading frame 84
Chromosome 14 open reading frame 32
chromosome 14 open reading frame 58
chromosome 16 open reading frame 51
chromosome 19 open reading frame 2
Chromosome 20 open reading frame 155
chromosome 20 open reading frame 30
chromosome 9 open reading frame 40
chymase 1, mast cell
Clone FLB8034 PRO2158
coenzyme Q3 homolog, methyltransferase (yeast); coenzyme Q3 homolog,
methyltransferase (yeast)
coenzyme Q4 homolog (yeast)
Coiled-coil domain containing 18
COP9 constitutive photomorphogenic homolog subunit 8 (Arabidopsis)
core-binding factor, beta subunit
Cullin 4A
cyclin A2
cyclin B2
cyclin D2
cyclin-dependent kinase inhibitor 1A (p21, Cip1)
DAZ associated protein 1
dihydrouridine synthase 4-like (S. cerevisiae)
dilute suppressor
dynamin 3
E3 ubiquitin protein ligase, HECT domain containing, 1
EH-domain containing 1
ephrin-A1
Epidermal growth factor receptor pathway substrate 15
erythropoietin receptor
exosome component 8
family with sequence similarity 54, member A
family with sequence similarity 72, member A
F-box protein 5
FLJ20105 protein
FLJ39739 protein
Forkhead box O3A
Formin binding protein 1
Full length insert cDNA clone ZD50H02
G protein-coupled receptor 64
G protein-coupled receptor kinase interactor 2
gelsolin (amyloidosis, Finnish type)
general transcription factor IIIA
GRIP1 associated protein 1
hairy/enhancer-of-split related with YRPW motif 2
haloacid dehalogenase-like hydrolase domain containing 1A
heterogeneous nuclear ribonucleoprotein D-like
high-mobility group nucleosomal binding domain 2
histone 1, H2ai
Histone 1, H2bd
histone 1, H2be
histone 1, H2bf
histone 1, H2bi
histone 1, H2bk
Hypothetical LOC344887
Hypothetical protein BC009732
hypothetical protein FLJ10211
hypothetical protein FLJ10241
Hypothetical protein FLJ14082
hypothetical protein FLJ20534
Hypothetical protein FLJ25715
hypothetical protein FLJ38725
hypothetical protein LOC137886
Hypothetical protein LOC149478
hypothetical protein LOC284356
hypothetical protein LOC285954
hypothetical protein LOC286149
hypothetical protein MGC15875
hypothetical protein MGC20235
hypothetical protein MGC20235
Hypothetical protein MGC24039
hypothetical protein MGC5576
hypothetical protein PRO2852
inhibitor of growth family, member 2
insulin-like growth factor 2 receptor
interleukin 1, beta
isocitrate dehydrogenase 1 (NADP+), soluble
jagged 1 (Alagille syndrome)
KIAA0101
KIAA0319-like
KIAA0372
KIAA0922 protein
KIAA1659 protein
kinesin family member 1C
kinesin family member 2C
kinesin family member 9
L-3-hydroxyacyl-Coenzyme A dehydrogenase, short chain
lamin A/C
lanosterol synthase (2,3-oxidosqualene-lanosterol cyclase)
Leucine rich repeat (in FLII) interacting protein 2
leucine zipper protein 5
ligand of numb-protein X
lymphoblastic leukemia derived sequence 1
lysocardiolipin acyltransferase
major vault protein
Malate dehydrogenase 2, NAD (mitochondrial)
Mdm2, transformed 3T3 cell double minute 2, p53 binding protein
(mouse)
membrane-spanning 4-domains, subfamily A, member 7;
membrane-spanning 4-domains, subfamily A, member 7
meningioma expressed antigen 5 (hyaluronidase)
mitogen-activated protein kinase kinase kinase 5
neurexin 3
nicastrin
nicotinamide nucleotide adenylyltransferase 2
N-myristoyltransferase 2
Nuclear receptor co-repressor 2
Nuclear receptor subfamily 3, group C, member 1
(glucocorticoid receptor)
nucleophosmin (nucleolar phosphoprotein B23, numatrin)
nucleoporin 37 kDa
NudC domain containing 2
PDZ binding kinase
peroxiredoxin 6
phosphoprotein enriched in astrocytes 15
pituitary tumor-transforming 3
pleckstrin homology-like domain, family A, member 3
polo-like kinase 4 (Drosophila)
primase, polypeptide 1, 49 kDa
proline rich 6
Protein phosphatase 1, regulatory (inhibitor) subunit 16B
purinergic receptor P2X, ligand-gated ion channel, 4
RAB3 GTPase activating protein subunit 1 (catalytic)
RAB5B, member RAS oncogene family
radixin
RAS p21 protein activator 3
replication factor C (activator 1) 3, 38 kDa
replication factor C (activator 1) 4, 37 kDa
replication factor C (activator 1) 5, 36.5 kDa
retinoblastoma-associated factor 600
Rho GTPase activating protein 19
ribonucleotide reductase M2 B (TP53 inducible)
ribosomal protein L10a
ribosomal protein S27-like
RIM binding protein 2
RNA binding motif protein 20
RNA methyltransferase like 1
serpin peptidase inhibitor, clade A (alpha-1 antiproteinase, antitrypsin),
member 9
SH3-domain GRB2-like pseudogene 2
Sideroflexin 1
Similar to Hypothetical protein KIAA0563
Similar to Microneme antigen
Small nuclear ribonucleoprotein polypeptide N
solute carrier family 25, member 37
sperm associated antigen 5
spermatogenesis associated 18 homolog (rat)
ST6 (alpha-N-acetyl-neuraminyl-2,3-beta-galactosyl-1,3)-N-
acetylgalactosaminide alpha-2,6-sialyltransferase 2
stathmin 1/oncoprotein 18
steroid sulfatase (microsomal), arylsulfatase C, isozyme S
syntaxin 16
syntaxin binding protein 4
TBC1 domain family, member 7
TBP-1 interacting protein
testes development-related NYD-SP21
thiamin pyrophosphokinase 1
timeless homolog (Drosophila)
timeless-interacting protein
TRAFs and NIK-associated protein
Transcribed locus, moderately similar to XP_517655.1 PREDICTED:
similar to KIAA0825 protein [Pan troglodytes]
Transcribed locus, moderately similar to XP_519290.1 PREDICTED:
reelin [Pan troglodytes]
Transcribed locus, weakly similar to NP_055301.1 neuronal thread
protein AD7c-NTP [Homo sapiens]
Transcribed locus, weakly similar to NP_997349.1 FLJ46489
protein [Homo sapiens]
transgelin
transmembrane protein 48
transmembrane protein 76
trophoblast-derived noncoding RNA
TTK protein kinase
tumor necrosis factor receptor superfamily, member 11b (osteoprotegerin)
tumor necrosis factor receptor superfamily, member 14 (herpesvirus entry
mediator)
tyrosyl-DNA phosphodiesterase 1
U2-associated SR140 protein
ubiquitin protein ligase E3B
ubiquitin specific peptidase 1
Ubiquitin specific peptidase 13 (isopeptidase T-3)
UDP-N-acetyl-alpha-D-galactosamine:polypeptide N-
acetylgalactosaminyltransferase 10 (GalNAc-T10)
UL16 binding protein 1
vaccinia related kinase 1
WD repeat domain 68
X Kell blood group precursor-related family, member 4
zinc binding alcohol dehydrogenase, domain containing 2
zinc finger protein 330
zinc finger protein 608
Zinc finger RNA binding protein
ZW10 interactor

### Example 2

Further genome-wide basal gene expression profiles and genome-wide SNPs for 277 human lymphoblastoid cell lines (LCLs) were used to identify and functionally validate SNPs/genes that might contribute to variation in radiation response. Basal gene expression data was obtained for all 277 cell lines using Affymetrix U133 plus 2.0 Gene Chips as well as genome-wide SNP data using Illumina HumanHap 550K and 510S BeadChips together with publicly available Affymetrix SNP Array 6.0 SNP data, resulting in a total of over 1.3 million SNPs per cell line. Radiation cytotoxicity assays were then performed with the same LCLs to obtain area under the radiation dose response curve (AUC) as an in vitro radiation response phenotype. A genome-wide association study was performed using the 1.3 million SNPs, basal gene expression array data and AUC as a radiation response phenotype. Twenty-seven “loci” were identified, defined as at least 2 SNPs within 50 kb having p values<10⁻⁴ that were associated with radiation AUC. Genes also were identified with expression levels that were associated with radiation AUC with p<10⁻³. By then performing an “integrated analysis” of SNPs, gene expression and radiation AUC, a total of 23 candidate genes were selected to perform siRNA knockdown with multiple tumor cell lines, followed by MTS and colony-forming functional validation assays to identify genes that influenced radiation exposure sensitivity.

The series of experiments described below represents the application of genome-wide expression and SNP data from a cell line-based model system to identify genes associated with radiation sensitivity. Those genes, selected on the basis of their association with SNPs, with expression array data or the results of an “integrated analysis” using both SNP and expression array data, were then validated functionally. The p values used during the initial selection process were purposely not stringent since all candidate genes were going to be functionally validated. The functional validation involved siRNA gene knockdown performed with cancer cell lines, followed by MTS cytotoxicity and colony-forming assays. These studies were conducted to identify and functionally validate biomarkers for radiation response, and to identify novel mechanisms that might contribute to radiation sensitivity.

### Materials and Methods

Cell lines: EBV-transformed LCLs from 93 African-American (AA), 89 Caucasian-American (CA), and 95 Han Chinese-American (HCA) unrelated healthy subjects (sample sets HD100AA, HD100CAU, HD100CHI) were purchased from the Coriell Cell Repository (Camden, N.J.). These samples had been collected and anonymized by NIGMS. Human pancreatic cancer MIA-PaCa2 and HupT3 cell lines were gifts from Dr. Daniel D. Billadeau, Mayo Clinic. Human cervical cancer HeLa and non-small cell lung cancer A549 cell lines were obtained from the ATCC (Manassas, Va.).

LCLs were cultured in RPMI 1640 medium (Mediatech, Manassas, Va.) supplemented with 15% heat-inactivated Fetal Bovine Serum (FBS) (Mediatech). HeLa and MIA-PaCa2 cell lines were cultured in DMEM medium containing 10% FBS. HupT3 and A549 cell lines were grown in RPMI 1640 medium with 10% FBS.

Cell proliferation assay: Cell proliferation assays were performed in triplicate at each radiation dose. Specifically, 100 μl of cells (5×10⁵ cells/ml) were plated into 96-well plates (Corning, Lowell, Mass.) Li et al. (2008) Cancer Res. 68:7050-7058, and were treated with ionizing radiation at 0, 0.156, 0.3125, 0.625, 1.25, 2.5, 5, 10 and 20 Gy using ¹³⁷Cesium gamma-rays (J. L. Shepherd and Associates Mark I Model 25 Irradiator, San Fernando, Calif.). After incubation for 3 days, 20 μl of CellTiter 96 AQueous Non-Radioactive Cell Proliferation Assay solution (Promega Corporation, Madison, Wis.) was added to each well. Plates were read in a Safire2 plate reader (Tecan AG, Switzerland). Nineteen LCLs were selected randomly, and the MTS assay was repeated approximately one year after the initial assay. Cytotoxicity for human tumor cell lines was determined in a similar fashion except the cells were incubated overnight before radiation treatment at 0, 0.25, 0.5, 1, 2.5, 5, 10 and 20 Gy, followed by MTS and colony-forming assays.

Expression array assays: Total RNA was extracted from each of the cell lines using Qiagen RNeasy Mini kits (QIAGEN, Inc.). RNA quality was tested using an Agilent 2100 Bioanalyzer, followed by hybridization to Affymetrix U133 Plus 2.0 Gene-Chips. A total of 54,613 probe sets were used in the analyses. Expression array data were obtained for all of the cell lines, 174 of which had been used in previous studies (Li et al. (2008), supra; and Li et al. (2009) PLoS One 4:e7765.

Genome-wide SNP analysis: DNA from all of the LCLs was genotyped using

Illumina HumanHap 550K and 510S BeadChips, which assayed 561,298 and 493,750 SNPs, respectively. Genotyping was performed in the Genotype Shared Resource (GSR) at the Mayo Clinic, Rochester, MN. Publicly available Affymetix SNP Array 6.0 Chip SNP data also was obtained for the same cell lines, which involved 643,600 SNPs unique to the Affymetrix SNP array. SNPs that deviated from Hardy-Weinberg Equilibrium (HWE, minimum exact test for HWE) (Wigginton et al. (2005) Am. J. Hum. Genet. 76:887-893) and the stratified test for HWE (Schaid et al. (2006) Am. J. Hum. Genet. 79:1071-1080) (p values<0.001); SNPs with call rates<95%; or SNPs with MAFs<5% were removed from the analysis.

Transient transfection and RNA interference: siRNAs for the candidate genes and “all star negative control” siRNA were purchased from QIAGEN (Hiden, Germany). The human HeLa cervical cancer cell line and the human pancreatic cancer cell lines, MIA-PaCa2 and HupT3, were used for the siRNA knockdown experiments. Reverse transfection was performed in 96-well plates. About 3000-4000 cells were mixed with 0.1 p1 of LIPOFECTAMINE™ RNAiMAX reagent (Invitrogen, Carlsbad, Calif.) and 10 nM siRNA for these experiments.

Colony forming assays: “All star negative control” and specific siRNAs were transfected into MIA-PaCa2, HupT3, HeLa and A459 cell lines. After 24 hours, about 400-1000 cells were plated in triplicate in 6-well plates. After exposure to radiation, cells were incubated for up to 7 days. Colonies in each well were fixed with methanol and stained with crystal violet. All colonies were counted visually or by the use of Quantity One (Bio-Rad, Hercules, Calif.).

Real-time quantitative reverse transcription-PCR: Total RNA was isolated from cultured cells transfected with control or specific siRNAs with the Qiagen RNeasy kit (QIAGEN Inc. Valencia, Calif.), followed by QRT-PCR performed with the 1-step, Brilliant SYBR Green QRT-PCR master mix kit (Stratagene, La Jolla, Calif.). Specifically, primers purchased from QIAGEN were used to perform QRT-PCR using the Stratagene Mx3005P™ Real-Time PCR detection system (Stratagene, La Jolla, CA). All experiments were performed in triplicate with β-actin as an internal control. Reverse transcribed Universal Human reference RNA (Stratagene) was used to generate a standard curve. Control reactions lacked RNA template.

Statistical methods: The radiation cytotoxicity phenotype, AUC, was calculated based on a logistic model. Three different logistic functions (four parameter, three free parameters with a fixed asymptote at 0%, and three free parameters with a fixed asymptote at 100%) were used to fit the data with the R package “drc” (available online at cran.r-project.org/doc/packages/drc.pdf). The best fit of the three logistic models with the lowest mean square error was used to determine the cytotoxicity AUC phenotype. AUC was determined by numerically computing the area under the estimated dose-response curve, from dose 0 to 20 Gy. AUC values were then log transformed. Expression array data were normalized on a log 2 scale using GCRMA (Wu et al. (2004) J. Amer. Stat. Assoc. 99:909-917). The normalized expression data and log transformed AUC values were then regressed on gender. Because LCLs from multiple races/ethnic groups were used, before completing the genetic association analysis, population stratification was assessed and was adjusted using the method developed by Price et al. ((2006) Nat. Genet. 38:904-909). This approach uses an eigen analysis for detecting and adjusting for population stratification, in which the eigen analysis was performed within each of the three racial groups. Using the top five eigenvectors within each race, the individual genotypes were adjusted using the model Gᵢⱼ=αⱼ+γₖⱼ+εᵢⱼ, with Gᵢⱼ representing the genotype for the ith cell line in racial group j (j=1, 2, 3), αⱼ the race effect for race j, and γₖⱼ the kth eigenvector, k=1, . . . , 5, effect for race j. Similarly, for genetic analysis of SNPs with AUC or expression, the log transformed AUC values and normalized log 2 expression values were also adjusted for race using the five eigenvectors, in addition to gender.

Association analyses of expression-AUC, expression-SNP and SNP-AUC were then completed using Pearson correlations and adjusted AUC, SNP and expression data. False Discovery q-values (Storey and Tibishirani (2003) Proc. Natl. Acad. Sci. USA 100:9440-9445) also were computed for each test. Pairwise LD was estimated using r-squared statistics and was displayed graphically using the Haploview software (Barrett et al. (2005) Bioinformatics 21:263-265). Genes and SNPs were annotated using NCBI Build 36.3. The pathway analysis was performed using Ingenuity Pathway Analysis (IPA; Ingenuity Systems, Redwood City, Calif.).

To integrate the genotype, expression, and drug cytotoxicity data, loci containing at least 2 SNPs associated with AUC with p values<10⁻⁴ within 50 kb of each other were first identified, followed by identification of all SNPs located within the specific loci with p values<0.001. Next, a determination was made as to which expression probe sets were associated with these SNPs (p values<10⁻⁴). Finally, to determine whether the expression probe sets associated with these SNPs were also associated with radiation AUC values, expression probe sets associated with radiation AUC with a p<0.001 were identified. A similar approach has been used successfully to detect novel candidate genes for functional follow-up (Li et al. (2009), supra).

For the most significant “locus” on chromosome 8, SNPs were imputed for a region 200 Kb in length on either side of the most significant SNP. Imputation was performed using MACH 1.0 (Li and Abecasis (2006) Am. J. Hum. Genet. S79:2290), with HapMap data as the reference panel. Specifically, AA SNPs were imputed with CEPH and YRI data, CA with CEU, and HCA with CHB and JPT.

### Results

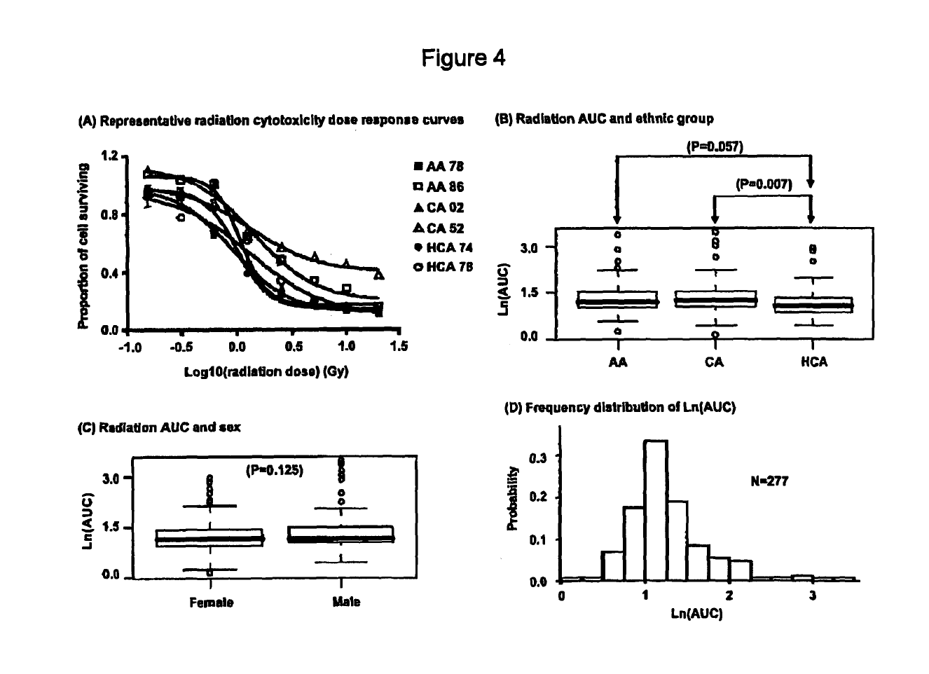

Radiation cytotoxicity: Radiation cytotoxicity studies were performed to determine the range of variation in radiation AUC among the individual cell lines studied. FIG. 4A shows representative radiation cytotoxicity data for a set of cell lines. AUC values differed significantly among the three racial groups studied, with cells from Han Chinese-American (HCA) subjects appearing to be more sensitive to radiation than were those of the cells from Caucasian-American (CA) subjects (p=0.007, FIG. 4B). Gender did not have a significant effect on AUC (p=0.125, FIG. 4C). The median AUC value for the 277 cell lines was 3.17, but the distribution was skewed. However, the distribution of values for the In transformed AUC was nearly “normal,” with a mean value of 1.27 and a range ±2 SD of the mean of 0.28 to 2.26, i.e., an 8-fold range (FIG. 4D). A biological replication study also was performed for the cytotoxicity data. Specifically, cytotoxicity assays were repeated one year after the initial studies for 19 randomly selected cell lines. The results obtained at the two different times were significantly correlated (Rp=0.51, p=0.026).

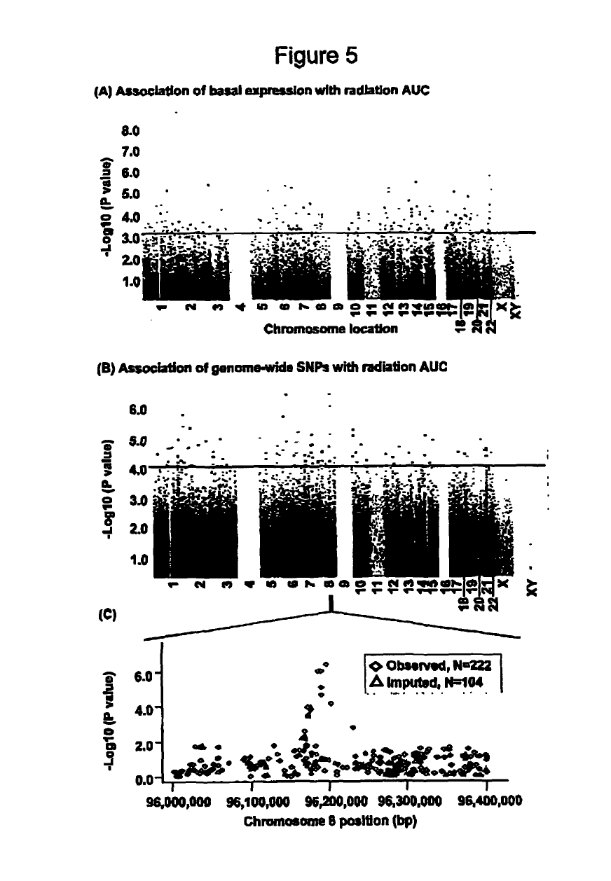

Correlation between expression and AUC: Correlation analyses were then performed for the association of expression array and radiation AUC data to identify genes with expression levels that might be associated with radiation AUC (FIG. 5A). The association analysis identified 50 expression probe sets that were associated with radiation AUC with p values<10⁻⁴ (q values<0.096), and 270 individual probe sets with p values<10⁻³ (q values<0.182). These 270 expression probe sets represented 211 annotated genes, but only one of the probe sets remained significant after Bonferroni correction (p<0.0002). The top 20 probe sets are listed in Table 4, and the entire list of 270 expression probe sets is found in Table 5.

TABLE 4
Top 20 expression probe sets associated with radiation cytotoxicity (AUC values).

| | | Bonferroni | | | | | |
|---|---|---|---|---|---|---|---|
| Probe ID | P value | p value | R value* | Q value | Chr | Gene Symbol | RefSeq ID |
| 243826_at | 1.97E−09 | 0.0001 | 0.351 | 9.68E−05 | 2 | — | — |
| 209605_at | 1.96E−06 | 0.1073 | −0.281 | 4.17E−02 | 22 | TST | NM_003312 |
| 1554768_a_at | 3.63E−06 | 0.1981 | −0.274 | 4.17E−02 | 14 | MAD2L1 | NM_002358 |
| 231149_s_at | 4.78E−06 | 0.2610 | −0.271 | 4.17E−02 | 3 | ULK4 | NM_017886 |
| 210639_s_at | 5.35E−06 | 0.2920 | −0.270 | 4.17E−02 | 6 | ATG5 | NM_004849 |
| 234863_x_at | 6.66E−06 | 0.3638 | −0.267 | 4.17E−02 | 6 | FBXO5 | NM_001142522 |
| 204004_at | 7.46E−06 | 0.4072 | −0.266 | 4.17E−02 | 12 | PAWR | NM_002583 |
| 238104_at | 8.01E−06 | 0.4376 | 0.265 | 4.17E−02 | 23 | — | — |
| 207238_s_at | 8.81E−06 | 0.4809 | −0.264 | 4.17E−02 | 1 | PTPRC | NM_002838 |
| 201345_s_at | 8.90E−06 | 0.4863 | −0.263 | 4.17E−02 | 5 | UBE2D2 | NM_003339 |
| 219869_s_at | 1.04E−05 | 0.5688 | −0.262 | 4.17E−02 | 4 | SLC39A8 | NM_001135146 |
| 242364_x_at | 1.07E−05 | 0.5848 | 0.261 | 4.17E−02 | 17 | LOC100131096 | XM_001720907 |
| 204005_s_at | 1.10E−05 | 0.6034 | −0.261 | 4.17E−02 | 12 | PAWR | NM_002583 |
| 204887_s_at | 1.57E−05 | 0.8572 | −0.256 | 5.51E−02 | 4 | PLK4 | NM_014264 |
| 214173_x_at | 1.83E−05 | 0.9986 | −0.254 | 5.56E−02 | 19 | C19orf2 | NM_003796 |
| 201014_s_at | 1.90E−05 | 1.0000 | −0.254 | 5.56E−02 | 4 | PAICS | NM_001079524 |
| 227620_at | 1.92E−05 | 1.0000 | −0.254 | 5.56E−02 | 9 | SLC44A1 | NM_080546 |
| 227806_at | 2.45E−05 | 1.0000 | −0.251 | 6.69E−02 | 16 | C16orf74 | NM_206967 |
| 212896_at | 2.60E−05 | 1.0000 | −0.250 | 6.71E−02 | 5 | SKIV2L2 | NM_015360 |
| 225562_at | 2.77E−05 | 1.0000 | −0.249 | 6.81E−02 | 13 | RASA3 | NM_007368 |

*R values represent correlation coefficients for the association.

TABLE 5
The top 270 expression probe sets that were associated with radiation AUC with p values < 10⁻³.

| | | Bonferroni | | | | | |
|---|---|---|---|---|---|---|---|
| Probe ID | P value | p value | R value* | Q value | Chr | Gene Symbol^{†} | RefSeq ID |
| 243826_at | 1.97E−09 | 0.0001 | 0.351 | 9.68E−05 | 2 | — | — |
| 209605_at | 1.96E−06 | 0.1073 | −0.281 | 4.17E−02 | 22 | TST | NM_003312 |
| 1554768_a_at | 3.63E−06 | 0.1981 | −0.274 | 4.17E−02 | 14 | 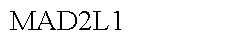 | NM_002358 |
| 231149_s_at | 4.78E−06 | 0.2610 | −0.271 | 4.17E−02 | 3 | ULK4 | NM_017886 |
| 210639_s_at | 5.35E−06 | 0.2920 | −0.270 | 4.17E−02 | 6 | ATG5 | NM_004849 |
| 234863_x_at | 6.66E−06 | 0.3638 | −0.267 | 4.17E−02 | 6 | FBXO5 | NM_001142522 |
| 204004_at | 7.46E−06 | 0.4072 | −0.266 | 4.17E−02 | 12 | 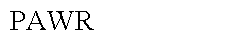 | NM_002583 |
| 238104_at | 8.01E−06 | 0.4376 | 0.265 | 4.17E−02 | 23 | — | — |
| 207238_s_at | 8.81E−06 | 0.4809 | −0.264 | 4.17E−02 | 1 | PTPRC | NM_002838 |
| 201345_s_at | 8.90E−06 | 0.4863 | −0.263 | 4.17E−02 | 5 | 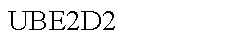 | NM_003339 |
| 219869_s_at | 1.04E−05 | 0.5688 | −0.262 | 4.17E−02 | 4 | SLC39A8 | NM_001135146 |
| 242364_x_at | 1.07E−05 | 0.5848 | 0.261 | 4.17E−02 | 17 | LOC100131096 | XM_001720907 |
| 204005_s_at | 1.10E−05 | 0.6034 | −0.261 | 4.17E−02 | 12 | 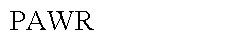 | NM_002583 |
| 204887_s_at | 1.57E−05 | 0.8572 | −0.256 | 5.51E−02 | 4 | 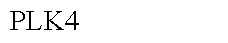 | NM_014264 |
| 214173_x_at | 1.83E−05 | 0.9986 | −0.254 | 5.56E−02 | 19 | 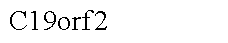 | NM_003796 |
| 201014_s_at | 1.90E−05 | 1.0000 | −0.254 | 5.56E−02 | 4 | PAICS | NM_001079524 |
| 227620_at | 1.92E−05 | 1.0000 | −0.254 | 5.56E−02 | 9 | SLC44A1 | NM_080546 |
| 227806_at | 2.45E−05 | 1.0000 | −0.251 | 6.69E−02 | 16 | C16orf74 | NM_206967 |
| 212896_at | 2.60E−05 | 1.0000 | −0.250 | 6.71E−02 | 5 | SKIV2L2 | NM_015360 |
| 225562_at | 2.77E−05 | 1.0000 | −0.249 | 6.81E−02 | 13 | RASA3 | NM_007368 |
| 226223_at | 2.91E−05 | 1.0000 | −0.248 | 6.82E−02 | 20 | — | — |
| 1556932_at | 3.77E−05 | 1.0000 | 0.245 | 8.30E−02 | 7 | — | — |
| 205071_x_at | 3.93E−05 | 1.0000 | −0.244 | 8.30E−02 | 5 | XRCC4 | NM_003401 |
| 202708_s_at | 4.06E−05 | 1.0000 | 0.244 | 8.30E−02 | 1 | HIST2H2BE | NM_003528 |
| 208382_s_at | 4.36E−05 | 1.0000 | −0.243 | 8.31E−02 | 22 | DMC1 | NM_007068 |
| 202511_s_at | 4.54E−05 | 1.0000 | −0.243 | 8.31E−02 | 6 | ATG5 | NM_004849 |
| 205240_at | 4.57E−05 | 1.0000 | −0.242 | 8.31E−02 | 1 | GPSM2 | NM_013296 |
| 212620_at | 5.13E−05 | 1.0000 | 0.241 | 8.61E−02 | 15 | ZNF609 | NM_015042 |
| 220953_s_at | 5.40E−05 | 1.0000 | −0.240 | 8.61E−02 | 5 | 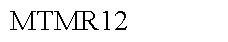 | NM_001040446 |
| 211363_s_at | 5.40E−05 | 1.0000 | −0.240 | 8.61E−02 | 9 | MTAP | NM_002451 |
| 216880_at | 5.43E−05 | 1.0000 | −0.240 | 8.61E−02 | 14 | RAD51L1 | NM_002877 |
| 1557984_s_at | 5.94E−05 | 1.0000 | −0.239 | 8.93E−02 | 12 | RPAP3 | NM_024604 |
| 205716_at | 6.01E−05 | 1.0000 | −0.239 | 8.93E−02 | 7 | SLC25A40 | NM_018843 |
| 210570_x_at | 6.25E−05 | 1.0000 | −0.238 | 8.93E−02 | 5 | MAPK9 | NM_001135044 |
| 206760_s_at | 6.79E−05 | 1.0000 | 0.237 | 8.93E−02 | 19 | FCER2 | NM_002002 |
| 212058_at | 6.92E−05 | 1.0000 | −0.237 | 8.93E−02 | 3 | 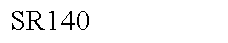 | NM_001080415 |
| 204128_s_at | 7.02E−05 | 1.0000 | −0.237 | 8.93E−02 | 13 | RFC3 | NM_002915 |
| 210972_x_at | 7.10E−05 | 1.0000 | −0.236 | 8.93E−02 | 14 | TRA@ /// TRAC | — |
| | | | | | | /// TRAJ17 /// | |
| | | | | | | TRAV20 | |
| 229053_at | 7.25E−05 | 1.0000 | 0.236 | 8.93E−02 | 16 | SYT17 | NM_016524 |
| 204772_s_at | 7.27E−05 | 1.0000 | −0.236 | 8.93E−02 | 9 | 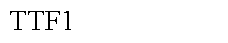 | NM_007344 |
| 232270_at | 8.19E−05 | 1.0000 | 0.234 | 9.55E−02 | 9 | C9orf3 | NM_032823 |
| 233655_s_at | 8.59E−05 | 1.0000 | −0.234 | 9.55E−02 | 9 | FAM29A | NM_017645 |
| 212588_at | 8.78E−05 | 1.0000 | −0.233 | 9.55E−02 | 1 | PTPRC | NM_002838 |
| 216274_s_at | 8.79E−05 | 1.0000 | −0.233 | 9.55E−02 | 15 | SEC11A | NM_014300 |
| 209980_s_at | 9.06E−05 | 1.0000 | −0.233 | 9.55E−02 | 17 | SHMT1 | NM_004169 |
| 216685_s_at | 9.19E−05 | 1.0000 | −0.233 | 9.55E−02 | 9 | MTAP | NM_002451 |
| 205159_at | 9.41E−05 | 1.0000 | −0.233 | 9.55E−02 | 22 | CSF2RB | NM_000395 |
| 228374_at | 9.73E−05 | 1.0000 | −0.232 | 9.55E−02 | 10 | C10orf28 | NM_014472 |
| 204633_s_at | 9.78E−05 | 1.0000 | −0.232 | 9.55E−02 | 14 | RPS6KA5 | NM_004755 |
| 1569607_s_at | 9.89E−05 | 1.0000 | 0.232 | 9.55E−02 | 1 | ANKRD20A1 /// | NM_001012419 |
| | | | | | | ANKRD20A2 /// | |
| | | | | | | ANKRD20A3 /// | |
| | | | | | | ANKRD20A4 /// | |
| | | | | | | C21orf81 /// | |
| | | | | | | LOC100132733 | |
| | | | | | | /// LOC284232 /// | |
| | | | | | | LOC643187 /// | |
| | | | | | | LOC647595 /// | |
| | | | | | | LOC653436 /// | |
| | | | | | | LOC727770 /// | |
| | | | | | | LOC728783 | |
| 203768_s_at | 1.01E−04 | 1.0000 | −0.232 | 9.55E−02 | 23 | 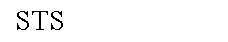 | NM_000351 |
| 214540_at | 1.01E−04 | 1.0000 | 0.232 | 9.55E−02 | 6 | HIST1H2BO | NM_003527 |
| 200806_s_at | 1.05E−04 | 1.0000 | −0.231 | 9.59E−02 | 12 | HSPD1 | NM_002156 |
| 205429_s_at | 1.06E−04 | 1.0000 | −0.231 | 9.59E−02 | 7 | MPP6 | NM_016447 |
| 211902_x_at | 1.08E−04 | 1.0000 | −0.231 | 9.59E−02 | 14 | TRA@ | — |
| 221504_s_at | 1.11E−04 | 1.0000 | 0.230 | 9.59E−02 | 8 | ATP6V1H | NM_015941 |
| 220721_at | 1.12E−04 | 1.0000 | −0.230 | 9.59E−02 | 19 | ZNF614 | NM_025040 |
| 204652_s_at | 1.13E−04 | 1.0000 | −0.230 | 9.59E−02 | 7 | 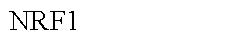 | NM_001040110 |
| 220390_at | 1.16E−04 | 1.0000 | 0.230 | 9.69E−02 | 11 | AGBL2 | NM_024783 |
| 205981_s_at | 1.20E−04 | 1.0000 | −0.229 | 9.78E−02 | 4 | ING2 | NM_001564 |
| 219715_s_at | 1.22E−04 | 1.0000 | −0.229 | 9.78E−02 | 14 | 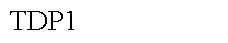 | NM_001008744 |
| 201688_s_at | 1.24E−04 | 1.0000 | −0.229 | 9.79E−02 | 8 | 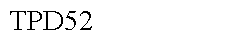 | NM_001025252 |
| 210455_at | 1.31E−04 | 1.0000 | −0.228 | 1.01E−01 | 10 | C10orf28 | NM_014472 |
| 209724_s_at | 1.34E−04 | 1.0000 | −0.228 | 1.01E−01 | 18 | ZFP161 | NM_001143823 |
| 206759_at | 1.34E−04 | 1.0000 | 0.228 | 1.01E−01 | 19 | FCER2 | NM_002002 |
| 226231_at | 1.36E−04 | 1.0000 | −0.227 | 1.01E−01 | 20 | — | — |
| 205124_at | 1.38E−04 | 1.0000 | 0.227 | 1.01E−01 | 19 | MEF2B | NM_001134794 |
| 1552634_a_at | 1.51E−04 | 1.0000 | −0.226 | 1.08E−01 | 19 | ZNF101 | NM_033204 |
| 209671_x_at | 1.52E−04 | 1.0000 | −0.226 | 1.08E−01 | 14 | TRA@ /// TRAC | — |
| 201689_s_at | 1.55E−04 | 1.0000 | −0.225 | 1.09E−01 | 8 | 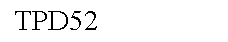 | NM_001025252 |
| 220088_at | 1.69E−04 | 1.0000 | 0.224 | 1.13E−01 | 19 | C5AR1 | NM_001736 |
| 1566001_at | 1.70E−04 | 1.0000 | 0.224 | 1.13E−01 | 16 | — | — |
| 201946_s_at | 1.72E−04 | 1.0000 | −0.224 | 1.13E−01 | 12 | CCT2 | NM_006431 |
| 209267_s_at | 1.72E−04 | 1.0000 | −0.224 | 1.13E−01 | 4 | SLC39A8 | NM_001135146 |
| 218970_s_at | 1.73E−04 | 1.0000 | −0.224 | 1.13E−01 | 10 | CUTC | NM_015960 |
| 1555852_at | 1.79E−04 | 1.0000 | −0.223 | 1.15E−01 | 6 | — | — |
| 203524_s_at | 1.81E−04 | 1.0000 | −0.223 | 1.15E−01 | 22 | MPST | NM_001013436 |
| 201079_at | 1.84E−04 | 1.0000 | 0.223 | 1.15E−01 | 15 | SYNGR2 | NM_004710 |
| 217640_x_at | 1.85E−04 | 1.0000 | −0.223 | 1.15E−01 | 18 | C18orf24 | NM_001039535 |
| 1554883_a_at | 1.87E−04 | 1.0000 | −0.223 | 1.15E−01 | 5 | ERCC8 | NM_000082 |
| 211088_s_at | 1.93E−04 | 1.0000 | −0.222 | 1.17E−01 | 4 | 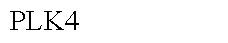 | NM_014264 |
| 203294_s_at | 1.96E−04 | 1.0000 | −0.222 | 1.17E−01 | 18 | LMAN1 | NM_005570 |
| 213231_at | 1.98E−04 | 1.0000 | 0.222 | 1.17E−01 | 19 | DMWD | NM_004943 |
| 223495_at | 2.09E−04 | 1.0000 | 0.221 | 1.19E−01 | 19 | CCDC8 | NM_032040 |
| 203218_at | 2.10E−04 | 1.0000 | −0.221 | 1.19E−01 | 5 | MAPK9 | NM_001135044 |
| 227261_at | 2.12E−04 | 1.0000 | −0.221 | 1.19E−01 | 13 | KLF12 | NM_007249 |
| 229285_at | 2.13E−04 | 1.0000 | −0.221 | 1.19E−01 | 1 | RNASEL | NM_021133 |
| 206667_s_at | 2.15E−04 | 1.0000 | −0.221 | 1.19E−01 | 5 | SCAMP1 | NM_004866 |
| 210813_s_at | 2.20E−04 | 1.0000 | −0.220 | 1.19E−01 | 5 | XRCC4 | NM_003401 |
| 206617_s_at | 2.21E−04 | 1.0000 | 0.220 | 1.19E−01 | 23 | RENBP | NM_002910 |
| 214335_at | 2.21E−04 | 1.0000 | 0.220 | 1.19E−01 | 19 | RPL18 | NM_000979 |
| 202468_s_at | 2.24E−04 | 1.0000 | −0.220 | 1.19E−01 | 9 | 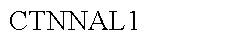 | NM_003798 |
| 242843_at | 2.29E−04 | 1.0000 | −0.220 | 1.21E−01 | 1 | BCAN | NM_021948 |
| 213498_at | 2.40E−04 | 1.0000 | 0.219 | 1.24E−01 | 11 | CREB3L1 | NM_052854 |
| 206668_s_at | 2.44E−04 | 1.0000 | −0.219 | 1.24E−01 | 5 | SCAMP1 | NM_004866 |
| 243399_at | 2.47E−04 | 1.0000 | 0.219 | 1.24E−01 | 2 | — | — |
| 223207_x_at | 2.47E−04 | 1.0000 | 0.219 | 1.24E−01 | 9 | PHPT1 | NM_001135861 |
| 202180_s_at | 2.47E−04 | 1.0000 | 0.219 | 1.24E−01 | 16 | MVP | NM_005115 |
| 233560_x_at | 2.54E−04 | 1.0000 | −0.218 | 1.26E−01 | 20 | MCM8 | NM_032485 |
| 219286_s_at | 2.57E−04 | 1.0000 | −0.218 | 1.26E−01 | 1 | RBM15 | NM_022768 |
| 219079_at | 2.62E−04 | 1.0000 | −0.218 | 1.26E−01 | 6 | CYB5R4 | NM_016230 |
| 202143_s_at | 2.62E−04 | 1.0000 | −0.218 | 1.26E−01 | 2 | COPS8 | NM_006710 |
| 226826_at | 2.64E−04 | 1.0000 | −0.218 | 1.26E−01 | 5 | — | — |
| 1554543_at | 2.79E−04 | 1.0000 | 0.217 | 1.32E−01 | 17 | SPAG9 | NM_001130528 |
| 209182_s_at | 2.89E−04 | 1.0000 | 0.216 | 1.33E−01 | 10 | C10orf10 | NM_007021 |
| 223171_at | 2.89E−04 | 1.0000 | −0.216 | 1.33E−01 | 18 | DYM | NM_017653 |
| 200650_s_at | 2.89E−04 | 1.0000 | −0.216 | 1.33E−01 | 1 | 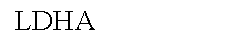 | NM_001135239 |
| 201344_at | 2.93E−04 | 1.0000 | −0.216 | 1.33E−01 | 5 | 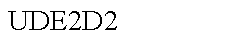 | NM_003339 |
| 238661_at | 2.98E−04 | 1.0000 | 0.216 | 1.33E−01 | 8 | LOC100130155 | XM_001720201 |
| 237158_s_at | 3.01E−04 | 1.0000 | −0.216 | 1.33E−01 | 12 | MPHOSPH9 | NM_022782 |
| 244398_x_at | 3.02E−04 | 1.0000 | −0.216 | 1.33E−01 | 1 | ZNF684 | NM_152373 |
| 230134_s_at | 3.05E−04 | 1.0000 | −0.215 | 1.33E−01 | 9 | RC3H2 | NM_001100588 |
| 221048_x_at | 3.08E−04 | 1.0000 | −0.215 | 1.33E−01 | 17 | C17orf80 | NM_001100621 |
| 201691_s_at | 3.11E−04 | 1.0000 | −0.215 | 1.33E−01 | 8 | 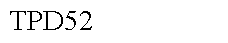 | NM_001025252 |
| 228485_s_at | 3.13E−04 | 1.0000 | −0.215 | 1.33E−01 | 9 | SLC44A1 | NM_080546 |
| 225193_at | 3.16E−04 | 1.0000 | −0.215 | 1.33E−01 | 22 | — | — |
| 201641_at | 3.19E−04 | 1.0000 | 0.215 | 1.33E−01 | 19 | BST2 | NM_004335 |
| 220794_at | 3.21E−04 | 1.0000 | 0.215 | 1.33E−01 | 1 | GREM2 | NM_022469 |
| 220073_s_at | 3.22E−04 | 1.0000 | 0.215 | 1.33E−01 | 12 | PLEKHG6 | NM_018173 |
| 211080_s_at | 3.26E−04 | 1.0000 | −0.214 | 1.33E−01 | 14 | NEK2 | NM_002497 |
| 218667_at | 3.28E−04 | 1.0000 | −0.214 | 1.33E−01 | 23 | PJA1 | NM_001032396 |
| 202089_s_at | 3.31E−04 | 1.0000 | −0.214 | 1.33E−01 | 18 | SLC39A6 | NM_001099406 |
| 238977_at | 3.39E−04 | 1.0000 | −0.214 | 1.35E−01 | 2 | MCM6 | NM_005915 |
| 205722_s_at | 3.42E−04 | 1.0000 | 0.214 | 1.35E−01 | 8 | GFRA2 | NM_001495 |
| 221397_at | 3.43E−04 | 1.0000 | 0.214 | 1.35E−01 | 12 | TAS2R10 | NM_023921 |
| 36888_at | 3.50E−04 | 1.0000 | −0.213 | 1.36E−01 | 19 | KIAA0841 | NM_015302 |
| 224076_s_at | 3.51E−04 | 1.0000 | −0.213 | 1.36E−01 | 8 | WHSC1L1 | NM_017778 |
| 207079_s_at | 3.60E−04 | 1.0000 | −0.213 | 1.38E−01 | 14 | MED6 | NM_005466 |
| 205891_at | 3.65E−04 | 1.0000 | 0.213 | 1.39E−01 | 17 | ADORA2B | NM_000676 |
| 238528_at | 3.68E−04 | 1.0000 | −0.213 | 1.39E−01 | 15 | UBR1 | NM_174916 |
| 223584_s_at | 3.74E−04 | 1.0000 | −0.212 | 1.39E−01 | 7 | 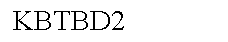 | NM_015483 |
| 1562505_at | 3.77E−04 | 1.0000 | 0.212 | 1.39E−01 | 20 | — | — |
| 225683_x_at | 3.81E−04 | 1.0000 | 0.212 | 1.39E−01 | 9 | MAMDC4 /// | NM_001135861 |
| | | | | | | PHPT1 | |
| 221195_at | 3.84E−04 | 1.0000 | −0.212 | 1.39E−01 | 17 | RNFT1 | NM_016125 |
| 1554474_a_at | 3.88E−04 | 1.0000 | 0.212 | 1.39E−01 | 6 | MOXD1 | NM_015529 |
| 1554397_s_at | 3.88E−04 | 1.0000 | −0.212 | 1.39E−01 | 11 | UEVLD | NM_001040697 |
| 219155_at | 3.96E−04 | 1.0000 | −0.211 | 1.39E−01 | 17 | PITPNC1 | NM_012417 |
| 1565269_s_at | 3.97E−04 | 1.0000 | −0.211 | 1.39E−01 | 12 | ATF1 | NM_005171 |
| 225000_at | 3.97E−04 | 1.0000 | −0.211 | 1.39E−01 | 3 | PRKAR2A | NM_004157 |
| 223585_x_at | 3.97E−04 | 1.0000 | −0.211 | 1.39E−01 | 7 | 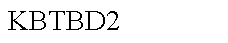 | NM_015483 |
| 222714_s_at | 4.07E−04 | 1.0000 | −0.211 | 1.39E−01 | 8 | LACTB2 | NM_016027 |
| 202352_s_at | 4.08E−04 | 1.0000 | −0.211 | 1.39E−01 | 17 | PSMD12 | NM_002816 |
| 220083_x_at | 4.10E−04 | 1.0000 | −0.211 | 1.39E−01 | 1 | UCHL5 | NM_015984 |
| 234023_s_at | 4.13E−04 | 1.0000 | −0.211 | 1.39E−01 | 13 | CENPJ | NM_018451 |
| 224596_at | 4.13E−04 | 1.0000 | −0.211 | 1.39E−01 | 9 | SLC44A1 | NM_080546 |
| 1554271_a_at | 4.14E−04 | 1.0000 | −0.211 | 1.39E−01 | 1 | 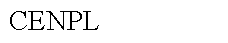 | NM_001127181 |
| 219501_at | 4.18E−04 | 1.0000 | 0.211 | 1.40E−01 | 13 | ENOX1 | NM_001127615 |
| 218622_at | 4.21E−04 | 1.0000 | −0.210 | 1.40E−01 | 12 | NUP37 | NM_024057 |
| 230070_at | 4.31E−04 | 1.0000 | 0.210 | 1.42E−01 | 11 | CNIH2 | NM_182553 |
| 218073_s_at | 4.33E−04 | 1.0000 | −0.210 | 1.42E−01 | 1 | 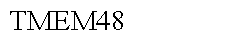 | NM_018087 |
| 209643_s_at | 4.42E−04 | 1.0000 | 0.210 | 1.43E−01 | 17 | PLD2 | NM_002663 |
| 215063_x_at | 4.42E−04 | 1.0000 | 0.210 | 1.43E−01 | 1 | LRRC40 | NM_017768 |
| 219030_at | 4.54E−04 | 1.0000 | −0.209 | 1.44E−01 | 2 | TPRKB | NM_016058 |
| 203984_s_at | 4.54E−04 | 1.0000 | −0.209 | 1.44E−01 | 1 | CASP9 | NM_001229 |
| 206935_at | 4.55E−04 | 1.0000 | 0.209 | 1.44E−01 | 13 | PCDH8 | NM_002590 |
| 224984_at | 4.66E−04 | 1.0000 | 0.209 | 1.47E−01 | 16 | NFAT5 | NM_001113178 |
| 231918_s_at | 4.80E−04 | 1.0000 | −0.208 | 1.50E−01 | 5 | GFM2 | NM_032380 |
| 219292_at | 4.85E−04 | 1.0000 | −0.208 | 1.51E−01 | 8 | THAP1 | NM_018105 |
| 217801_at | 4.92E−04 | 1.0000 | 0.208 | 1.52E−01 | 20 | ATP5E | NM_001001977 |
| 216526_x_at | 4.99E−04 | 1.0000 | 0.208 | 1.52E−01 | 6 | HLA-C | NM_002117 |
| 229173_at | 5.00E−04 | 1.0000 | −0.208 | 1.52E−01 | 2 | KIAA1715 | NM_030650 |
| 222387_s_at | 5.03E−04 | 1.0000 | −0.208 | 1.52E−01 | 16 | VPS35 | NM_018206 |
| 226153_s_at | 5.05E−04 | 1.0000 | −0.208 | 1.52E−01 | 15 | CNOT6L | NM_144571 |
| 1552978_a_at | 5.11E−04 | 1.0000 | −0.207 | 1.52E−01 | 5 | SCAMP1 | NM_004866 |
| 219126_at | 5.14E−04 | 1.0000 | −0.207 | 1.52E−01 | 6 | PHF10 | NM_018288 |
| 204417_at | 5.22E−04 | 1.0000 | −0.207 | 1.52E−01 | 14 | GALC | NM_000153 |
| 206056_x_at | 5.33E−04 | 1.0000 | 0.207 | 1.52E−01 | 16 | SPN | NM_001030288 |
| 219628_at | 5.34E−04 | 1.0000 | 0.207 | 1.52E−01 | 3 | ZMAT3 | NM_022470 |
| 218967_s_at | 5.35E−04 | 1.0000 | −0.207 | 1.52E−01 | 10 | PTER | NM_001001484 |
| 227376_at | 5.35E−04 | 1.0000 | 0.207 | 1.52E−01 | 7 | GLI3 | NM_000168 |
| 227447_at | 5.37E−04 | 1.0000 | −0.207 | 1.52E−01 | 5 | SKIV2L2 | NM_015360 |
| 201920_at | 5.38E−04 | 1.0000 | 0.207 | 1.52E−01 | 2 | SLC20A1 | NM_005415 |
| 218603_at | 5.39E−04 | 1.0000 | −0.207 | 1.52E−01 | 6 | HECA | NM_016217 |
| 203353_s_at | 5.39E−04 | 1.0000 | −0.207 | 1.52E−01 | 18 | MBD1 | NM_002384 |
| 206030_at | 5.43E−04 | 1.0000 | 0.207 | 1.52E−01 | 17 | ASPA | NM_000049 |
| 204444_at | 5.44E−04 | 1.0000 | −0.207 | 1.52E−01 | 10 | KIF11 | NM_004523 |
| 241040_at | 5.49E−04 | 1.0000 | 0.206 | 1.52E−01 | 8 | — | — |
| 1568949_at | 5.54E−04 | 1.0000 | −0.206 | 1.53E−01 | 17 | PITPNC1 | NM_012417 |
| 209583_s_at | 5.65E−04 | 1.0000 | 0.206 | 1.55E−01 | 3 | CD200 | NM_001004196 |
| 203856_at | 5.68E−04 | 1.0000 | −0.206 | 1.55E−01 | 14 | VRK1 | NM_003384 |
| 209016_s_at | 5.76E−04 | 1.0000 | 0.206 | 1.56E−01 | 12 | KRT7 | NM_005556 |
| 206220_s_at | 5.83E−04 | 1.0000 | −0.205 | 1.56E−01 | 13 | RASA3 | NM_007368 |
| 1564962_at | 5.86E−04 | 1.0000 | −0.205 | 1.56E−01 | 7 | ZNF92 | NM_007139 |
| 226345_at | 5.87E−04 | 1.0000 | −0.205 | 1.56E−01 | 10 | — | — |
| 218584_at | 5.88E−04 | 1.0000 | 0.205 | 1.56E−01 | 12 | TCTN1 | NM_001082537 |
| 213575_at | 5.91E−04 | 1.0000 | 0.205 | 1.56E−01 | 7 | TRA2A | NM_013293 |
| 225525_at | 6.04E−04 | 1.0000 | 0.205 | 1.58E−01 | 22 | CTA-221G9.4 /// | XM_001724436 |
| | | | | | | LOC100131004 | |
| 202071_at | 6.06E−04 | 1.0000 | 0.205 | 1.58E−01 | 20 | SDC4 | NM_002999 |
| 212552_at | 6.10E−04 | 1.0000 | −0.205 | 1.58E−01 | 2 | HPCAL1 | NM_002149 |
| 218875_s_at | 6.19E−04 | 1.0000 | −0.204 | 1.58E−01 | 6 | FBXO5 | NM_001142522 |
| 1554577_a_at | 6.21E−04 | 1.0000 | −0.204 | 1.58E−01 | 23 | PSMD10 | NM_002814 |
| 1562641_at | 6.21E−04 | 1.0000 | −0.204 | 1.58E−01 | 23 | FAM122C | NM_138819 |
| 218634_at | 6.21E−04 | 1.0000 | 0.204 | 1.58E−01 | 1 | PHLDA3 | NM_012396 |
| 219960_s_at | 6.25E−04 | 1.0000 | −0.204 | 1.58E−01 | 1 | UCHL5 | NM_015984 |
| 203767_s_at | 6.29E−04 | 1.0000 | −0.204 | 1.58E−01 | 23 | 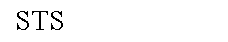 | NM_000351 |
| 221698_s_at | 6.37E−04 | 1.0000 | 0.204 | 1.59E−01 | 12 | CLEC7A | NM_022570 |
| 212959_s_at | 6.38E−04 | 1.0000 | 0.204 | 1.59E−01 | 12 | GNPTAB | NM_024312 |
| 221743_at | 6.45E−04 | 1.0000 | 0.204 | 1.59E−01 | 11 | CUGBP1 | NM_001025596 |
| 223785_at | 6.49E−04 | 1.0000 | −0.204 | 1.59E−01 | 15 | FANCI | NM_001113378 |
| 225951_s_at | 6.49E−04 | 1.0000 | −0.204 | 1.59E−01 | 15 | CHD2 | NM_001042572 |
| 1554989_at | 6.55E−04 | 1.0000 | 0.204 | 1.59E−01 | 14 | KIAA0317 | NM_001039479 |
| 231597_x_at | 6.56E−04 | 1.0000 | 0.204 | 1.59E−01 | NA | — | — |
| 225455_at | 6.58E−04 | 1.0000 | −0.203 | 1.59E−01 | 1 | TADA1L | NM_053053 |
| 211084_x_at | 6.63E−04 | 1.0000 | −0.203 | 1.59E−01 | 2 | PRKD3 | NM_005813 |
| 231878_at | 6.69E−04 | 1.0000 | −0.203 | 1.59E−01 | 16 | C16orf53 | NM_024516 |
| 222293_at | 6.73E−04 | 1.0000 | 0.203 | 1.59E−01 | 19 | CADM4 | NM_145296 |
| 243721_at | 6.73E−04 | 1.0000 | 0.203 | 1.59E−01 | 18 | — | — |
| 218957_s_at | 6.75E−04 | 1.0000 | −0.203 | 1.59E−01 | 11 | PAAF1 | NM_025155 |
| 231909_x_at | 6.76E−04 | 1.0000 | −0.203 | 1.59E−01 | 1 | ODF2L | NM_001007022 |
| 1555762_s_at | 6.79E−04 | 1.0000 | −0.203 | 1.59E−01 | 1 | RBM15 | NM_022768 |
| 212587_s_at | 6.84E−04 | 1.0000 | −0.203 | 1.59E−01 | 1 | PTPRC | NM_002838 |
| 33579_i_at | 6.88E−04 | 1.0000 | 0.203 | 1.59E−01 | 22 | GALR3 | NM_003614 |
| 236228_at | 6.93E−04 | 1.0000 | 0.203 | 1.59E−01 | 5 | LOC100130744 | XM_001720249 |
| 1559521_at | 6.93E−04 | 1.0000 | 0.203 | 1.59E−01 | 21 | — | — |
| 218558_s_at | 6.95E−04 | 1.0000 | −0.203 | 1.59E−01 | 21 | MRPL39 | NM_017446 |
| 221094_s_at | 7.10E−04 | 1.0000 | −0.202 | 1.61E−01 | 8 | ELP3 | NM_018091 |
| 235959_at | 7.16E−04 | 1.0000 | 0.202 | 1.62E−01 | 1 | — | — |
| 242738_s_at | 7.37E−04 | 1.0000 | 0.202 | 1.64E−01 | 16 | ZFHX3 | NM_006885 |
| 232937_at | 7.39E−04 | 1.0000 | 0.202 | 1.64E−01 | 13 | — | — |
| 222777_s_at | 7.41E−04 | 1.0000 | −0.202 | 1.64E−01 | 4 | WHSC1 | NM_001042424 |
| 228930_at | 7.47E−04 | 1.0000 | −0.201 | 1.64E−01 | 15 | — | — |
| 231873_at | 7.51E−04 | 1.0000 | 0.201 | 1.64E−01 | 2 | BMPR2 | NM_001204 |
| 214168_s_at | 7.54E−04 | 1.0000 | 0.201 | 1.64E−01 | 15 | TJP1 | NM_003257 |
| 203770_s_at | 7.56E−04 | 1.0000 | −0.201 | 1.64E−01 | 23 | 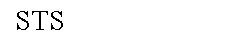 | NM_000351 |
| 219544_at | 7.56E−04 | 1.0000 | −0.201 | 1.64E−01 | 13 | 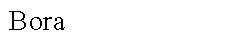 | NM_024808 |
| 210416_s_at | 7.57E−04 | 1.0000 | −0.201 | 1.64E−01 | 22 | CHEK2 | NM_001005735 |
| 200868_s_at | 7.61E−04 | 1.0000 | −0.201 | 1.64E−01 | 20 | RNF114 | NM_018683 |
| 202142_at | 7.65E−04 | 1.0000 | −0.201 | 1.64E−01 | 2 | COPS8 | NM_006710 |
| 212124_at | 7.67E−04 | 1.0000 | −0.201 | 1.64E−01 | 10 | ZMIZ1 | NM_020338 |
| 1555830_s_at | 7.70E−04 | 1.0000 | −0.201 | 1.64E−01 | 7 | 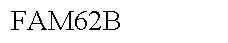 | NM_020728 |
| 207738_s_at | 7.73E−04 | 1.0000 | 0.201 | 1.64E−01 | 2 | NCKAP1 | NM_013436 |
| 227803_at | 7.90E−04 | 1.0000 | 0.201 | 1.67E−01 | 6 | ENPP5 | NM_021572 |
| 1555760_a_at | 8.02E−04 | 1.0000 | −0.200 | 1.69E−01 | 22 | RBM15 | NM_022768 |
| 201690_s_at | 8.12E−04 | 1.0000 | −0.200 | 1.69E−01 | 8 | 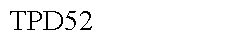 | NM_001025252 |
| 228378_at | 8.16E−04 | 1.0000 | −0.200 | 1.69E−01 | 12 | C12orf29 | NM_001009894 |
| 1564963_x_at | 8.22E−04 | 1.0000 | −0.200 | 1.69E−01 | 7 | ZNF92 | NM_007139 |
| 224883_at | 8.23E−04 | 1.0000 | −0.200 | 1.69E−01 | 15 | PLDN | NM_012388 |
| 239105_at | 8.28E−04 | 1.0000 | −0.200 | 1.69E−01 | 14 | — | — |
| 1561706_at | 8.32E−04 | 1.0000 | 0.200 | 1.69E−01 | NA | — | — |
| 219812_at | 8.37E−04 | 1.0000 | −0.200 | 1.69E−01 | 7 | PVRIG | NM_024070 |
| 1558794_at | 8.40E−04 | 1.0000 | 0.200 | 1.69E−01 | NA | LOC728190 | NR_024397 |
| 209708_at | 8.42E−04 | 1.0000 | 0.200 | 1.69E−01 | 6 | MOXD1 | NM_015529 |
| 233421_s_at | 8.44E−04 | 1.0000 | −0.199 | 1.69E−01 | 1 | NUP133 | NM_018230 |
| 206039_at | 8.46E−04 | 1.0000 | −0.199 | 1.69E−01 | 23 | RAB33A | NM_004794 |
| 232222_at | 8.46E−04 | 1.0000 | 0.199 | 1.69E−01 | 18 | C18orf49 | — |
| 226966_at | 8.48E−04 | 1.0000 | 0.199 | 1.69E−01 | 12 | PRPF40B | NM_001031698 |
| 209670_at | 8.63E−04 | 1.0000 | −0.199 | 1.70E−01 | 14 | TRAC | — |
| 231046_at | 8.72E−04 | 1.0000 | 0.199 | 1.70E−01 | 5 | — | — |
| 220885_s_at | 8.75E−04 | 1.0000 | −0.199 | 1.70E−01 | 13 | CENPJ | NM_018451 |
| 1554493_s_at | 8.77E−04 | 1.0000 | −0.199 | 1.70E−01 | 2 | THADA | NM_001083953 |
| 223740_at | 8.79E−04 | 1.0000 | 0.199 | 1.70E−01 | 6 | RIPPLY2 | NR_024277 |
| 1559059_s_at | 8.79E−04 | 1.0000 | −0.199 | 1.70E−01 | 19 | ZNF611 | NM_030972 |
| 224699_s_at | 8.79E−04 | 1.0000 | −0.199 | 1.70E−01 | 7 | 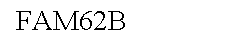 | NM_020728 |
| 230974_at | 8.81E−04 | 1.0000 | −0.199 | 1.70E−01 | 16 | DDX19B | NM_001014449 |
| 244470_at | 8.88E−04 | 1.0000 | 0.199 | 1.70E−01 | 23 | RNF12 | NM_016120 |
| 203214_x_at | 8.92E−04 | 1.0000 | −0.199 | 1.70E−01 | 10 | CDC2 | NM_001130829 |
| 231973_s_at | 8.93E−04 | 1.0000 | −0.199 | 1.70E−01 | 2 | ANAPC1 | NM_022662 |
| 238190_at | 8.94E−04 | 1.0000 | −0.199 | 1.70E−01 | 16 | TUFM | NM_003321 |
| 214521_at | 9.00E−04 | 1.0000 | 0.198 | 1.71E−01 | 1 | HES2 | NM_019089 |
| 237221_at | 9.11E−04 | 1.0000 | 0.198 | 1.72E−01 | 15 | — | — |
| 210559_s_at | 9.13E−04 | 1.0000 | −0.198 | 1.72E−01 | 10 | CDC2 | NM_001130829 |
| 223010_s_at | 9.21E−04 | 1.0000 | −0.198 | 1.73E−01 | 4 | OCIAD1 | NM_001079839 |
| 210742_at | 9.50E−04 | 1.0000 | 0.198 | 1.77E−01 | 1 | CDC14A | NM_003672 |
| 205393_s_at | 9.53E−04 | 1.0000 | −0.197 | 1.77E−01 | 11 | CHEK1 | NM_001114121 |
| 223289_s_at | 9.56E−04 | 1.0000 | −0.197 | 1.77E−01 | 4 | USP38 | NM_032557 |
| 217457_s_at | 9.71E−04 | 1.0000 | −0.197 | 1.79E−01 | 4 | RAP1GDS1 | NM_001100426 |
| 215750_at | 9.79E−04 | 1.0000 | 0.197 | 1.80E−01 | 22 | KIAA1659 | XM_001723799 |
| 205858_at | 9.83E−04 | 1.0000 | 0.197 | 1.80E−01 | 17 | NGFR | NM_002507 |
| 211318_s_at | 9.92E−04 | 1.0000 | −0.197 | 1.81E−01 | 20 | RAE1 | NM_001015885 |
| 218295_s_at | 9.95E−04 | 1.0000 | −0.197 | 1.81E−01 | 22 | NUP50 | NM_007172 |

*R values represent correlation coefficients for the association.
†Genes that were selected for siRNA screening are highlighted by shading.

Genome-wide SNP association with radiation AUC: An analysis of the association of genome-wide SNPs with radiation AUC also was performed (FIG. 5B). A total of 561,298 SNPs on the Illumina 550K SNP array and 493,750 SNPs on the Illumina 510S SNP array had been genotyped using DNA from each of these 277 cell lines. In addition, the Affymetrix 6.0 SNP array data was publically available. Quality control (QC) was performed for all of these SNPs prior to performing the statistical analysis. Specifically, for data obtained with the Illumina 550K array, 12,261 SNPs were removed that had call rates<95%, 32,550 SNPs were removed with minor allele frequencies (MAFs) <5%, and 4,676 SNPs were removed that deviated from Hardy-Weinberg Equilibrium (HWE), using a stringent threshold of p<0.001. Therefore, 511,811 Illumina 550K SNPs were used in the genome-wide SNP analysis. A similar approach was used for the QC analysis of SNPs on the Illumina 510S platform. After removing 10,353 SNPs with call rates<95%, 147,027 SNPs with a MAF<5% and 3,805 SNPs that deviated from HWE (p<0.001), a total of 332,565 Illumina 510S SNPs remained for analysis. For the publicly available Affymetrix 6.0 SNPs, SNPs that had already been genotyped using the Illumina platforms were first removed, resulting in 643,600 unique SNPs on the Affymetrix 6.0 SNP array for which the QC analysis was performed. After removing 26,140 SNPs with call rates<95%, 107,275 SNPs with a MAF<5% and 5,763 that deviated from HWE (p<0.001), 504,422 remained. Therefore, after combining data from the two platforms, 1,348,798 SNPs were available for use in the analysis (FIG. 5B).

The p value for the most significant SNP, Affymetrix marker SNP_A-8538282 (rs7000734), was 3.82×10⁻⁷ (r=0.309, MAF=0.081). The top 16 SNPs, all with p values<10⁻⁵, are listed in Table 6, and the 1335 SNPs that had p values<10⁻³ are listed in Table 7. Of these, 151 SNPs were significantly associated with AUC, with p values<10⁻⁴. These 151 SNPs were located within or close to 99 unique genes on the basis of the annotation of genome build 36.3. Among the 151 top SNPs, 3 were within coding regions, 45 within introns, and 36 and 67 were within 5′- or 3′-UTR or flanking regions, respectively.

TABLE 6
The top 16 SNPs that were associated with radiation AUC with p values <10⁻⁵.

| | | | | | | | | | Location |
|---|---|---|---|---|---|---|---|---|---|
| | | | | | | | | | Relative to |
| SNP ID | P value | R value* | Q value | MAF | Chr | Position | Gene Symbol | Location | Gene (bp) |
| rs7000734 | 3.82E−07 | 0.309 | 0.259 | 0.081 | 8 | 96,196,206 | PLEKHF2 | 5′-upstream | 19,002 |
| rs16885294 | 3.91E−07 | 0.310 | 0.259 | 0.071 | 6 | 54,373,355 | TINAG | 3′-downstream | 10,483 |
| rs1561715 | 9.58E−07 | 0.299 | 0.318 | 0.083 | 8 | 96,189,090 | PLEKHF2 | 5′-upstream | 26,118 |
| rs1610110 | 9.58E−07 | 0.299 | 0.318 | 0.083 | 8 | 96,185,038 | PLEKHF2 | 5′-upstream | 30,170 |
| rs2859631 | 1.20E−06 | 0.296 | 0.318 | 0.072 | 16 | 77,189,295 | WWOX | intron | 0 |
| rs12569163 | 1.78E−06 | 0.292 | 0.394 | 0.118 | 1 | 239,945,170 | WDR64 | intron | 0 |
| rs16889440 | 2.14E−06 | 0.290 | 0.405 | 0.175 | 6 | 24,652,946 | KIAA0319 | 3′-UTR | 0 |
| rs7554126 | 4.34E−06 | 0.287 | 0.667 | 0.297 | 1 | 239,953,804 | WDR64 | intron | 0 |
| rs4742269 | 4.73E−06 | 0.280 | 0.667 | 0.139 | 9 | 6,839,317 | JMJD2C | intron | 0 |
| rs7591064 | 5.03E−06 | 0.279 | 0.667 | 0.110 | 2 | 49,031,105 | FSHR | 3′-downstream | 12,051 |
| rs11250464 | 5.61E−06 | 0.279 | 0.676 | 0.175 | 10 | 1,396,364 | ADARB2 | intron | 0 |
| rs1853665 | 6.87E−06 | 0.276 | 0.696 | 0.344 | 6 | 150,340,535 | ULBP1 | 3′-downstream | 3,996 |
| rs4392868 | 8.24E−06 | 0.274 | 0.696 | 0.087 | 8 | 96,189,248 | PLEKHF2 | 5′-upstream | 25,960 |
| rs17598306 | 8.60E−06 | 0.273 | 0.696 | 0.079 | 7 | 96,419,747 | FLJ34048 | 3′-downstream | 51,148 |
| rs1471356 | 8.71E−06 | 0.273 | 0.696 | 0.230 | 14 | 97,373,084 | LOC100132612 | 5′-upstream | 86,450 |
| rs4554799 | 9.00E−06 | 0.272 | 0.696 | 0.170 | 10 | 1,397,728 | ADARB2 | intron | 0 |

*R values represent correlation coefficients for the association.

TABLE 7
Top 1335 SNPs associated with radiation AUC with p values <10⁻³.

| | | | | | | | | | Location |
|---|---|---|---|---|---|---|---|---|---|
| | | | | | | | | | Relative to |
| SNP ID | P value | R value* | Q value | MAF | Chr | Position | Gene Symbol | Location | Gene (bp) |
| rs7000734 | 3.82E−07 | 0.309 | 0.259 | 0.081 | 8 | 96,196,206 | PLEKHF2 | 5′-upstream | 19,002 |
| rs16885294 | 3.91E−07 | 0.310 | 0.259 | 0.071 | 6 | 54,373,355 | TINAG | 3′-downstream | 10,483 |
| rs1561715 | 9.58E−07 | 0.299 | 0.318 | 0.083 | 8 | 96,189,090 | PLEKHF2 | 5′-upstream | 26,118 |
| rs1610110 | 9.58E−07 | 0.299 | 0.318 | 0.083 | 8 | 96,185,038 | PLEKHF2 | 5′-upstream | 30,170 |
| rs2859631 | 1.20E−06 | 0.296 | 0.318 | 0.072 | 16 | 77,189,295 | WWOX | intron | 0 |
| rs12569163 | 1.78E−06 | 0.292 | 0.394 | 0.118 | 1 | 239,945,170 | WDR64 | intron | 0 |
| rs16889440 | 2.14E−06 | 0.290 | 0.405 | 0.175 | 6 | 24,652,946 | KIAA0319 | 3′-UTR | 0 |
| rs7554126 | 4.34E−06 | 0.287 | 0.667 | 0.297 | 1 | 239,953,804 | WDR64 | intron | 0 |
| rs4742269 | 4.73E−06 | 0.280 | 0.667 | 0.139 | 9 | 6,839,317 | JMJD2C | intron | 0 |
| rs7591064 | 5.03E−06 | 0.279 | 0.667 | 0.110 | 2 | 49,031,105 | FSHR | 3′-downstream | 12,051 |
| rs11250464 | 5.61E−06 | 0.279 | 0.676 | 0.175 | 10 | 1,396,364 | ADARB2 | intron | 0 |
| rs1853665 | 6.87E−06 | 0.276 | 0.696 | 0.344 | 6 | 150,340,535 | ULBP1 | 3′-downstream | 3,996 |
| rs4392868 | 8.24E−06 | 0.274 | 0.696 | 0.087 | 8 | 96,189,248 | PLEKHF2 | 5′-upstream | 25,960 |
| rs17598306 | 8.60E−06 | 0.273 | 0.696 | 0.079 | 7 | 96,419,747 | FLJ34048 | 3′-downstream | 51,148 |
| rs1471356 | 8.71E−06 | 0.273 | 0.696 | 0.230 | 14 | 97,373,084 | LOC100132612 | 5′-upstream | 86,450 |
| rs4554799 | 9.00E−06 | 0.272 | 0.696 | 0.170 | 10 | 1,397,728 | ADARB2 | intron | 0 |
| rs2409791 | 1.09E−05 | −0.270 | 0.696 | 0.280 | 5 | 59,920,074 | DEPDC1B | 3′-downstream | 8,422 |
| rs12657754 | 1.10E−05 | 0.270 | 0.696 | 0.221 | 5 | 3,875,893 | IRX1 | 3′-downstream | 221,376 |
| rs7633553 | 1.17E−05 | 0.273 | 0.696 | 0.291 | 3 | 58,415,276 | PDHB | 5′-upstream | 20,671 |
| rs898387 | 1.24E−05 | 0.268 | 0.696 | 0.235 | 1 | 202,845,996 | LRRN2 | 3′-downstream | 6,928 |
| rs6091189 | 1.27E−05 | 0.268 | 0.696 | 0.277 | 20 | 48,689,692 | C20orf175 | 5′-upstream | 2,859 |
| rs1715958 | 1.28E−05 | 0.267 | 0.696 | 0.244 | 15 | 53,051,004 | C15orf15 | 3′-downstream | 209,800 |
| rs10189510 | 1.30E−05 | 0.267 | 0.696 | 0.085 | 2 | 183,199,011 | DNAJC10 | 5′-upstream | 90,233 |
| rs3777645 | 1.42E−05 | 0.267 | 0.696 | 0.100 | 6 | 54,309,669 | TINAG | intron | 0 |
| rs10517564 | 1.44E−05 | 0.266 | 0.696 | 0.052 | 4 | 153,408,601 | FBXW7 | 3′-downstream | 53,259 |
| rs631411 | 1.47E−05 | 0.266 | 0.696 | 0.299 | 9 | 199,325 | C9orf66 | 3′-downstream | 4,137 |
| rs2498662 | 1.49E−05 | 0.266 | 0.696 | 0.065 | 6 | 139,467,599 | HECA | 5′-upstream | 30,343 |
| rs12553351 | 1.49E−05 | 0.265 | 0.696 | 0.117 | 9 | 6,828,348 | JMJD2C | intron | 0 |
| rs12380100 | 1.55E−05 | 0.265 | 0.696 | 0.097 | 9 | 11,177,013 | LOC646114 | 3′-downstream | 146,728 |
| rs869239 | 1.60E−05 | 0.265 | 0.696 | 0.495 | 4 | 183,690,033 | ODZ3 | intron | 0 |
| rs7676392 | 1.73E−05 | 0.264 | 0.696 | 0.281 | 4 | 183,613,233 | ODZ3 | intron | 0 |
| rs12705596 | 1.75E−05 | 0.263 | 0.696 | 0.161 | 7 | 108,893,619 | LOC100128056 | 5′-upstream | 372,799 |
| rs10183821 | 1.81E−05 | 0.264 | 0.696 | 0.120 | 2 | 128,753,139 | HS6ST1 | intron | 0 |
| rs7901632 | 1.87E−05 | 0.262 | 0.696 | 0.072 | 10 | 80,382,672 | ZMIZ1 | 5′-upstream | 248,675 |
| rs17598132 | 1.89E−05 | 0.263 | 0.696 | 0.076 | 7 | 96,417,275 | FLJ34048 | 3′-downstream | 53,620 |
| rs7193297 | 1.97E−05 | 0.267 | 0.696 | 0.447 | 16 | 71,551,332 | ZFHX3 | missense | 0 |
| rs10515853 | 2.05E−05 | 0.261 | 0.696 | 0.051 | 5 | 162,588,458 | MRP63P6 | 3′-downstream | 189,268 |
| rs1561714 | 2.14E−05 | 0.261 | 0.696 | 0.087 | 8 | 96,189,151 | PLEKHF2 | 5′-upstream | 26,057 |
| rs942679 | 2.17E−05 | 0.261 | 0.696 | 0.213 | 1 | 154,617,690 | RHBG | intron | 0 |
| rs12297511 | 2.20E−05 | 0.262 | 0.696 | 0.175 | 12 | 131,204,374 | NOC4L | 3′-downstream | 1,435 |
| rs10809359 | 2.22E−05 | 0.261 | 0.696 | 0.109 | 9 | 11,135,717 | LOC646114 | 3′-downstream | 105,432 |
| rs10084239 | 2.31E−05 | 0.260 | 0.696 | 0.117 | 2 | 42,107,250 | LOC91461 | 5′-upstream | 22,007 |
| rs17587765 | 2.33E−05 | 0.260 | 0.696 | 0.108 | 1 | 117,372,602 | IGSF2 | intron | 0 |
| rs672482 | 2.37E−05 | 0.259 | 0.696 | 0.092 | 1 | 143,543,612 | TRNAQ-CUG | 3′-downstream | 7,181 |
| rs13128522 | 2.38E−05 | 0.260 | 0.696 | 0.060 | 4 | 16,149,782 | LDB2 | intron | 0 |
| rs2249118 | 2.41E−05 | −0.260 | 0.696 | 0.364 | 21 | 36,277,829 | RPL23AP3 | 3′-downstream | 32,264 |
| rs10809395 | 2.50E−05 | 0.259 | 0.704 | 0.103 | 9 | 11,223,740 | LOC646114 | 3′-downstream | 193,455 |
| rs2212916 | 2.80E−05 | −0.257 | 0.711 | 0.366 | 21 | 36,281,638 | RPL23AP3 | 3′-downstream | 28,455 |
| rs927984 | 2.81E−05 | 0.257 | 0.711 | 0.130 | 6 | 25,520,966 | LRRC16A | intron | 0 |
| rs2826523 | 2.85E−05 | 0.258 | 0.711 | 0.068 | 21 | 21,100,816 | PPIAP | 5′-upstream | 21,182 |
| rs11731599 | 2.98E−05 | 0.257 | 0.711 | 0.489 | 4 | 183,691,126 | ODZ3 | intron | 0 |
| rs4314649 | 3.10E−05 | 0.257 | 0.711 | 0.242 | 8 | 35,052,669 | LOC100133273 | 3′-downstream | 200,898 |
| rs894013 | 3.19E−05 | 0.261 | 0.711 | 0.472 | 3 | 4,353,142 | SETMAR | 3′-downstream | 19,193 |
| rs35018391 | 3.23E−05 | 0.255 | 0.711 | 0.049 | 4 | 181,217,870 | hCG_2025798 | 3′-downstream | 1,004,366 |
| rs41333347 | 3.23E−05 | 0.255 | 0.711 | 0.049 | 4 | 181,227,186 | hCG_2025798 | 3′-downstream | 1,662,228 |
| rs7680285 | 3.23E−05 | 0.255 | 0.711 | 0.049 | 4 | 181,228,438 | hCG_2025798 | 3′-downstream | 993,798 |
| rs8089644 | 3.30E−05 | 0.255 | 0.711 | 0.099 | 18 | 2,942,642 | LPIN2 | intron | 0 |
| rs17657370 | 3.34E−05 | 0.255 | 0.711 | 0.078 | 7 | 96,444,428 | FLJ34048 | 3′-downstream | 26,467 |
| rs2812604 | 3.40E−05 | 0.255 | 0.711 | 0.118 | 10 | 34,089,448 | PARD3 | 3′-downstream | 350,655 |
| rs4326096 | 3.41E−05 | −0.255 | 0.711 | 0.270 | 5 | 59,926,035 | DEPDC1B | 3′-downstream | 2,461 |
| rs6570180 | 3.45E−05 | 0.255 | 0.711 | 0.196 | 6 | 137,987,617 | LOC391040 | 5′-upstream | 73,852 |
| rs2923661 | 3.46E−05 | 0.255 | 0.711 | 0.240 | 8 | 35,061,182 | LOC100133273 | 3′-downstream | 209,411 |
| rs16988828 | 3.47E−05 | −0.255 | 0.711 | 0.085 | 22 | 29,333,561 | TCN2 | intron | 0 |
| rs6079836 | 3.60E−05 | −0.254 | 0.711 | 0.339 | 20 | 15,383,564 | MACROD2 | intron | 0 |
| rs6079839 | 3.60E−05 | −0.254 | 0.711 | 0.339 | 20 | 15,384,282 | MACROD2 | intron | 0 |
| rs7990503 | 3.64E−05 | 0.254 | 0.711 | 0.195 | 13 | 79,518,643 | LOC729485 | 3′-downstream | 8,767 |
| rs6873333 | 3.64E−05 | 0.254 | 0.711 | 0.072 | 5 | 155,195,850 | LOC100131033 | 3′-downstream | 342,496 |
| rs2491142 | 3.66E−05 | 0.254 | 0.711 | 0.215 | 1 | 31,096,680 | SDC3 | 3′-downstream | 21,887 |
| rs12376660 | 3.71E−05 | 0.254 | 0.711 | 0.089 | 9 | 11,175,487 | LOC646114 | 3′-downstream | 145,202 |
| rs641372 | 3.75E−05 | 0.259 | 0.711 | 0.071 | 18 | 58,432,935 | ZCCHC2 | 3′-downstream | 36,137 |
| rs2075243 | 3.91E−05 | −0.254 | 0.725 | 0.327 | 12 | 15,552,430 | PTPRO | intron | 0 |
| rs1994179 | 4.08E−05 | 0.253 | 0.725 | 0.259 | 14 | 96,989,660 | LOC730217 | 5′-upstream | 3,769 |
| rs2862667 | 4.09E−05 | 0.252 | 0.725 | 0.266 | 11 | 43,099,468 | LOC100128134 | 5′-upstream | 44,525 |
| rs807301 | 4.11E−05 | 0.252 | 0.725 | 0.051 | 6 | 119,969,706 | LOC728727 | intron | 0 |
| rs1650385 | 4.15E−05 | 0.252 | 0.725 | 0.437 | 5 | 174,991,457 | HRH2 | 5′-upstream | 50,861 |
| rs6964059 | 4.15E−05 | 0.253 | 0.725 | 0.259 | 7 | 40,630,937 | C7orf10 | intron | 0 |
| rs898386 | 4.22E−05 | 0.253 | 0.727 | 0.229 | 1 | 202,846,320 | LRRN2 | 3′-downstream | 6,604 |
| rs3924198 | 4.36E−05 | 0.252 | 0.727 | 0.399 | 3 | 545,612 | LOC402123 | 3′-downstream | 88,860 |
| rs2837005 | 4.46E−05 | 0.251 | 0.727 | 0.318 | 21 | 39,691,677 | LCA5L | 3′-downstream | 7,963 |
| rs2045624 | 4.51E−05 | 0.251 | 0.727 | 0.258 | 1 | 202,851,008 | LRRN2 | 3′-downstream | 1,916 |
| rs10490857 | 4.53E−05 | 0.251 | 0.727 | 0.051 | 3 | 6,934,982 | GRM7 | intron | 0 |
| rs4880503 | 4.54E−05 | 0.251 | 0.727 | 0.171 | 10 | 1,426,858 | ADARB2 | intron | 0 |
| rs11976501 | 4.59E−05 | 0.250 | 0.727 | 0.134 | 7 | 76,746,424 | CCDC146 | intron | 0 |
| rs4817696 | 4.61E−05 | 0.250 | 0.727 | 0.262 | 21 | 35,155,046 | RUNX1 | intron | 0 |
| rs8101536 | 4.79E−05 | 0.250 | 0.740 | 0.208 | 19 | 2,580,845 | GNG7 | intron | 0 |
| rs12705601 | 4.80E−05 | 0.250 | 0.740 | 0.161 | 7 | 108,923,348 | LOC100128056 | 5′-upstream | 343,070 |
| rs7309722 | 4.97E−05 | −0.250 | 0.750 | 0.460 | 12 | 76,694,384 | NAV3 | 5′-upstream | 54,816 |
| rs10516301 | 4.97E−05 | 0.250 | 0.750 | 0.072 | 4 | 16,143,251 | LDB2 | intron | 0 |
| rs4487737 | 5.14E−05 | 0.249 | 0.757 | 0.218 | 8 | 141,296,740 | NIBP | intron | 0 |
| rs7387053 | 5.14E−05 | 0.249 | 0.757 | 0.218 | 8 | 141,295,342 | NIBP | intron | 0 |
| rs2309691 | 5.36E−05 | 0.248 | 0.777 | 0.491 | 4 | 183,691,063 | ODZ3 | intron | 0 |
| rs17549345 | 5.39E−05 | −0.248 | 0.777 | 0.336 | 3 | 88,163,269 | CGGBP1 | 3′-downstream | 20,521 |
| rs27564 | 5.55E−05 | −0.251 | 0.791 | 0.325 | 5 | 59,873,391 | PART1 | 3′-downstream | 20,403 |
| rs206789 | 5.74E−05 | −0.248 | 0.810 | 0.313 | 5 | 59,939,728 | DEPDC1B | intron | 0 |
| rs1693219 | 5.89E−05 | 0.247 | 0.823 | 0.289 | 1 | 231,807,892 | KCNK1 | 5′-upstream | 8,481 |
| rs11200175 | 6.20E−05 | 0.247 | 0.843 | 0.167 | 10 | 123,568,222 | ATE1 | intron | 0 |
| rs1693216 | 6.50E−05 | 0.247 | 0.843 | 0.286 | 1 | 231,808,517 | KCNK1 | 5′-upstream | 7,856 |
| rs11559201 | 6.57E−05 | 0.245 | 0.843 | 0.141 | 8 | 99,075,924 | MATN2 | synonymous | 0 |
| rs11559202 | 6.57E−05 | 0.245 | 0.843 | 0.141 | 8 | 99,075,912 | MATN2 | synonymous | 0 |
| rs2981309 | 6.62E−05 | 0.249 | 0.843 | 0.241 | 8 | 35,048,829 | LOC100133273 | 3′-downstream | 197,058 |
| rs13239088 | 6.64E−05 | 0.249 | 0.843 | 0.054 | 7 | 46,381,408 | LOC730338 | 3′-downstream | 313,968 |
| rs12121447 | 6.73E−05 | 0.246 | 0.843 | 0.055 | 1 | 120,018,494 | ZNF697 | 5′-upstream | 26,581 |
| rs4716858 | 6.74E−05 | −0.245 | 0.843 | 0.468 | 7 | 157,461,158 | PTPRN2 | intron | 0 |
| rs688858 | 6.74E−05 | −0.246 | 0.843 | 0.254 | 11 | 87,652,911 | CTSC | 3′-downstream | 13,497 |
| rs4570460 | 6.80E−05 | 0.245 | 0.843 | 0.058 | 1 | 200,955,651 | JARID1B | 3′-downstream | 7,504 |
| rs212551 | 6.85E−05 | 0.245 | 0.843 | 0.132 | 8 | 96,201,280 | PLEKHF2 | 5′-upstream | 13,928 |
| rs10118338 | 6.89E−05 | 0.245 | 0.843 | 0.139 | 9 | 11,226,474 | LOC646114 | 3′-downstream | 196,189 |
| rs2498090 | 6.97E−05 | −0.245 | 0.843 | 0.194 | 23 | 89,016,027 | LOC360018 | 5′-upstream | 19,984 |
| rs2243174 | 7.04E−05 | 0.244 | 0.843 | 0.444 | 1 | 205,078,108 | IL19 | intron | 0 |
| rs4948698 | 7.06E−05 | 0.245 | 0.843 | 0.062 | 10 | 42,859,465 | RET | 5′-upstream | 33,058 |
| rs2243188 | 7.11E−05 | 0.244 | 0.843 | 0.442 | 1 | 205,081,095 | IL19 | intron | 0 |
| rs13007983 | 7.12E−05 | 0.245 | 0.843 | 0.114 | 2 | 5,642,699 | SOX11 | 5′-upstream | 107,551 |
| rs10900601 | 7.37E−05 | 0.244 | 0.845 | 0.227 | 1 | 202,838,673 | LRRN2 | 3′-downstream | 14,251 |
| rs6578083 | 7.41E−05 | −0.244 | 0.845 | 0.374 | 8 | 141,299,161 | NIBP | intron | 0 |
| rs10240447 | 7.41E−05 | 0.244 | 0.845 | 0.495 | 7 | 157,460,585 | PTPRN2 | intron | 0 |
| rs11119670 | 7.45E−05 | 0.244 | 0.845 | 0.442 | 1 | 205,071,944 | IL19 | intron | 0 |
| rs12380191 | 7.47E−05 | 0.244 | 0.845 | 0.101 | 9 | 11,170,739 | LOC646114 | 3′-downstream | 140,454 |
| rs10838023 | 7.64E−05 | 0.243 | 0.845 | 0.267 | 11 | 43,113,488 | LOC100128134 | 5′-upstream | 58,545 |
| rs7378774 | 7.71E−05 | 0.245 | 0.845 | 0.064 | 5 | 155,221,392 | LOC100131033 | 3′-downstream | 368,038 |
| rs12908333 | 7.72E−05 | −0.243 | 0.845 | 0.298 | 15 | 32,219,109 | C15orf29 | 3′-downstream | 1,058 |
| rs9520592 | 7.80E−05 | 0.243 | 0.845 | 0.215 | 13 | 107,268,488 | FAM155A | intron | 0 |
| rs12705602 | 7.91E−05 | 0.243 | 0.845 | 0.166 | 7 | 108,929,460 | LOC100128056 | 5′-upstream | 336,958 |
| rs4738839 | 8.01E−05 | −0.243 | 0.845 | 0.227 | 8 | 61,960,444 | CHD7 | 3′-downstream | 18,425 |
| rs7835942 | 8.01E−05 | −0.243 | 0.845 | 0.227 | 8 | 61,960,345 | CHD7 | 3′-downstream | 18,326 |
| rs535161 | 8.01E−05 | 0.243 | 0.845 | 0.148 | 11 | 78,322,944 | ODZ4 | 5′-upstream | 122,111 |
| rs11788082 | 8.03E−05 | 0.243 | 0.845 | 0.150 | 9 | 11,208,170 | LOC646114 | 3′-downstream | 177,885 |
| rs308731 | 8.21E−05 | 0.242 | 0.853 | 0.365 | 3 | 4,354,374 | SETMAR | 3′-downstream | 20,425 |
| rs12301261 | 8.36E−05 | 0.242 | 0.853 | 0.081 | 12 | 83,102,293 | LOC100128335 | 3′-downstream | 264,229 |
| rs1518338 | 8.42E−05 | 0.242 | 0.853 | 0.288 | 3 | 117,912,015 | LOC285194 | 5′-upstream | 2,013 |
| rs11102357 | 8.63E−05 | 0.242 | 0.853 | 0.471 | 1 | 112,277,072 | KCND3 | intron | 0 |
| rs4131469 | 8.86E−05 | 0.241 | 0.853 | 0.350 | 1 | 200,237,371 | RNPEP | intron | 0 |
| rs4677119 | 8.96E−05 | 0.241 | 0.853 | 0.329 | 3 | 72,374,648 | RYBP | 3′-downstream | 131,790 |
| rs11888061 | 9.03E−05 | 0.241 | 0.853 | 0.199 | 2 | 66,217,781 | LOC729348 | 5′-upstream | 246,161 |
| rs2309341 | 9.04E−05 | −0.241 | 0.853 | 0.433 | 4 | 181,590,726 | hCG_2025798 | 3′-downstream | 631,510 |
| rs1268087 | 9.14E−05 | 0.241 | 0.853 | 0.147 | 6 | 126,155,515 | NCOA7 | intron | 0 |
| rs4234440 | 9.23E−05 | 0.246 | 0.853 | 0.185 | 3 | 43,925,045 | ABHD5 | 3′-downstream | 189,749 |
| rs12702213 | 9.29E−05 | 0.240 | 0.853 | 0.058 | 7 | 46,381,080 | LOC730338 | 3′-downstream | 314,296 |
| rs165808 | 9.41E−05 | 0.240 | 0.853 | 0.155 | 22 | 15,707,595 | ZNF402P | 3′-downstream | 10,431 |
| rs10007934 | 9.47E−05 | −0.240 | 0.853 | 0.087 | 4 | 138,044,051 | PCDH18 | 3′-downstream | 616,234 |
| rs2735784 | 9.48E−05 | 0.240 | 0.853 | 0.375 | 1 | 200,243,103 | ELF3 | 5′-upstream | 593 |
| rs10073856 | 9.49E−05 | 0.241 | 0.853 | 0.071 | 5 | 155,195,298 | LOC100131033 | 3′-downstream | 341,944 |
| rs17139113 | 9.50E−05 | 0.240 | 0.853 | 0.094 | 16 | 5,995,338 | A2BP1 | 5 ′-upstream | 13,795 |
| rs3088121 | 9.52E−05 | 0.241 | 0.853 | 0.105 | 8 | 99,117,225 | MATN2 | 3′-UTR | 0 |
| rs1319550 | 9.56E−05 | −0.240 | 0.853 | 0.475 | 2 | 80,253,062 | CTNNA2 | intron | 0 |
| rs7157518 | 9.58E−05 | −0.240 | 0.853 | 0.374 | 14 | 74,900,170 | LOC731223 | 3′-downstream | 67,076 |
| rs10770444 | 9.63E−05 | 0.240 | 0.853 | 0.243 | 12 | 8,341,081 | LOC100130149 | 3′-downstream | 22,953 |
| rs9388949 | 9.76E−05 | −0.240 | 0.853 | 0.484 | 6 | 132,319,155 | CTGF | 5′-upstream | 4,944 |
| rs2243193 | 9.77E−05 | 0.240 | 0.853 | 0.453 | 1 | 205,082,848 | IL19 | 3′-UTR | 0 |
| rs1991676 | 9.83E−05 | 0.241 | 0.853 | 0.055 | 18 | 64,903,434 | CCDC102B | 3′-downstream | 30,028 |
| rs7751415 | 9.85E−05 | 0.241 | 0.853 | 0.147 | 6 | 87,098,629 | LOC643926 | 5′-upstream | 100,055 |
| rs9852704 | 9.86E−05 | −0.240 | 0.853 | 0.406 | 3 | 88,171,034 | CGGBP1 | 3′-downstream | 12,756 |
| rs7379436 | 1.00E−04 | 0.239 | 0.853 | 0.074 | 5 | 155,158,726 | LOC100131033 | 3′-downstream | 305,372 |
| rs12356119 | 1.02E−04 | 0.239 | 0.853 | 0.114 | 10 | 128,017,485 | ADAM12 | intron | 0 |
| rs4662786 | 1.02E−04 | 0.240 | 0.853 | 0.111 | 2 | 128,752,362 | HS6ST1 | intron | 0 |
| rs10959622 | 1.02E−04 | 0.239 | 0.853 | 0.099 | 9 | 11,201,722 | LOC646114 | 3′-downstream | 171,437 |
| rs4397386 | 1.03E−04 | 0.242 | 0.853 | 0.214 | 8 | 141,294,034 | NIBP | intron | 0 |
| rs10486811 | 1.03E−04 | 0.239 | 0.853 | 0.254 | 7 | 40,617,380 | C7orf10 | intron | 0 |
| rs1829346 | 1.04E−04 | 0.239 | 0.853 | 0.287 | 3 | 117,911,597 | LOC285194 | 5′-upstream | 2,431 |
| rs1467166 | 1.04E−04 | −0.239 | 0.853 | 0.424 | 11 | 36,432,948 | FLJ14213 | intron | 0 |
| rs9450667 | 1.04E−04 | 0.239 | 0.853 | 0.312 | 6 | 88,151,105 | C6orf163 | 3′-downstream | 19,205 |
| rs10240312 | 1.04E−04 | 0.239 | 0.853 | 0.179 | 7 | 97,892,440 | BAIAP2L1 | 5′-upstream | 24,124 |
| rs2090111 | 1.05E−04 | 0.239 | 0.853 | 0.217 | 4 | 6,149,700 | JAKMIP1 | intron | 0 |
| rs12881650 | 1.06E−04 | 0.239 | 0.853 | 0.175 | 14 | 69,805,287 | ADAM21P | 5′-upstream | 21,016 |
| rs9311337 | 1.06E−04 | 0.239 | 0.853 | 0.478 | 3 | 4,356,787 | SUMF1 | 3′-downstream | 21,043 |
| rs10198712 | 1.07E−04 | −0.238 | 0.853 | 0.477 | 2 | 80,252,590 | CTNNA2 | intron | 0 |
| rs884108 | 1.07E−04 | 0.241 | 0.853 | 0.180 | 1 | 202,857,860 | LRRN2 | intron | 0 |
| rs10996977 | 1.08E−04 | 0.238 | 0.857 | 0.065 | 10 | 67,640,579 | CTNNA3 | intron | 0 |
| rs2837006 | 1.10E−04 | 0.238 | 0.857 | 0.352 | 21 | 39,694,306 | LCA5L | 3′-downstream | 5,334 |
| rs8076804 | 1.10E−04 | 0.238 | 0.857 | 0.188 | 17 | 75,890,077 | KIAA1618 | intron | 0 |
| rs9856655 | 1.10E−04 | 0.238 | 0.857 | 0.067 | 3 | 6,917,072 | GRM7 | intron | 0 |
| rs6744963 | 1.12E−04 | 0.238 | 0.857 | 0.087 | 2 | 20,311,091 | PUM2 | 3′-downstream | 843 |
| rs11952678 | 1.12E−04 | −0.238 | 0.857 | 0.209 | 5 | 62,013,385 | IPO11 | 3′-downstream | 53,220 |
| rs17149618 | 1.13E−04 | 0.238 | 0.857 | 0.105 | 8 | 9,140,544 | PPP1R3B | 5′-upstream | 94,914 |
| rs9467231 | 1.13E−04 | 0.238 | 0.857 | 0.188 | 6 | 24,667,566 | KIAA0319 | intron | 0 |
| rs1450705 | 1.14E−04 | 0.238 | 0.857 | 0.139 | 14 | 87,418,880 | GALC | 3′-downstream | 50,231 |
| rs11977013 | 1.15E−04 | 0.237 | 0.857 | 0.249 | 7 | 40,639,214 | C7orf10 | intron | 0 |
| rs297550 | 1.15E−04 | 0.237 | 0.857 | 0.063 | 8 | 96,170,970 | LOC100130098 | 5′-upstream | 15,945 |
| rs165790 | 1.17E−04 | 0.238 | 0.857 | 0.150 | 22 | 15,720,906 | ZNF402P | Unknown | — |
| rs7917910 | 1.18E−04 | 0.237 | 0.857 | 0.257 | 10 | 128,234,945 | LOC728152 | 3′-downstream | 28,706 |
| rs7302505 | 1.18E−04 | 0.237 | 0.857 | 0.162 | 12 | 63,304,388 | RASSF3 | intron | 0 |
| rs1358531 | 1.19E−04 | 0.240 | 0.857 | 0.166 | 15 | 53,069,902 | C15orf15 | 3′-downstream | 190,902 |
| rs11250461 | 1.19E−04 | 0.237 | 0.857 | 0.226 | 10 | 1,394,502 | ADARB2 | intron | 0 |
| rs4951088 | 1.20E−04 | 0.237 | 0.857 | 0.471 | 1 | 202,877,403 | LRRN2 | intron | 0 |
| rs41413447 | 1.20E−04 | 0.237 | 0.857 | 0.134 | 1 | 234,726,528 | ENO1P | 3′-downstream | 11,621 |
| rs10459842 | 1.21E−04 | −0.238 | 0.857 | 0.456 | 16 | 6,673,694 | A2BP1 | intron | 0 |
| rs2568654 | 1.21E−04 | 0.237 | 0.857 | 0.280 | 7 | 13,727,929 | LOC100131022 | 3′-downstream | 132,576 |
| rs11240239 | 1.22E−04 | 0.237 | 0.857 | 0.469 | 1 | 202,879,427 | LRRN2 | intron | 0 |
| rs1376519 | 1.22E−04 | 0.238 | 0.857 | 0.244 | 8 | 35,045,674 | LOC100133273 | 3′-downstream | 193,903 |
| rs2490588 | 1.25E−04 | 0.236 | 0.857 | 0.276 | 9 | 78,077,812 | LOC100133206 | 3′-downstream | 13,418 |
| rs17543709 | 1.26E−04 | 0.236 | 0.857 | 0.069 | 1 | 233,631,547 | TBCE | intron | 0 |
| rs165698 | 1.27E−04 | 0.236 | 0.857 | 0.152 | 22 | 15,719,068 | ZNF402P | Unknown | — |
| rs2988270 | 1.27E−04 | 0.236 | 0.857 | 0.112 | 1 | 50,428,660 | ELAVL4 | intron | 0 |
| rs6830998 | 1.28E−04 | 0.237 | 0.857 | 0.279 | 4 | 183,617,946 | ODZ3 | intron | 0 |
| rs384483 | 1.28E−04 | 0.238 | 0.857 | 0.127 | 5 | 122,289,472 | SNX24 | intron | 0 |
| rs2332277 | 1.33E−04 | 0.237 | 0.857 | 0.172 | 14 | 69,803,279 | ADAM21P | 5′-upstream | 19,008 |
| rs16994140 | 1.33E−04 | 0.236 | 0.857 | 0.092 | 4 | 37,687,960 | TBC1D1 | intron | 0 |
| rs10813853 | 1.33E−04 | 0.237 | 0.857 | 0.167 | 9 | 32,660,725 | TAF1L | 5′-upstream | 35,058 |
| rs286158 | 1.34E−04 | −0.235 | 0.857 | 0.309 | 5 | 59,970,010 | DEPDC1B | intron | 0 |
| rs2542443 | 1.34E−04 | 0.236 | 0.857 | 0.063 | 8 | 140,660,763 | KCNK9 | 3′-downstream | 33,223 |
| rs11781720 | 1.34E−04 | −0.235 | 0.857 | 0.327 | 8 | 4,038,118 | CSMD1 | intron | 0 |
| rs1773951 | 1.34E−04 | 0.235 | 0.857 | 0.070 | 1 | 231,802,262 | KCNK1 | 5′-upstream | 14,111 |
| rs16843867 | 1.35E−04 | 0.235 | 0.857 | 0.065 | 1 | 197,053,576 | MIRN181B1 | 3′-downstream | 41,049 |
| rs10419308 | 1.35E−04 | 0.235 | 0.857 | 0.143 | 19 | 60,431,625 | TMEM86B | intron | 0 |
| rs11250496 | 1.38E−04 | 0.235 | 0.857 | 0.166 | 10 | 1,437,105 | ADARB2 | intron | 0 |
| rs10486799 | 1.38E−04 | 0.235 | 0.857 | 0.074 | 7 | 39,619,751 | RALA | 5′-upstream | 9,936 |
| rs1513412 | 1.39E−04 | −0.235 | 0.857 | 0.379 | 3 | 147,081,068 | LOC389156 | 5′-upstream | 55,809 |
| rs1698888 | 1.40E−04 | 0.235 | 0.857 | 0.356 | 11 | 19,285,798 | LOC390102 | 5′-upstream | 32,407 |
| rs727695 | 1.42E−04 | 0.235 | 0.857 | 0.313 | 4 | 183,610,140 | ODZ3 | intron | 0 |
| rs6814420 | 1.42E−04 | −0.235 | 0.857 | 0.251 | 4 | 165,350,800 | ANP32C | 5′-upstream | 12,487 |
| rs4659246 | 1.43E−04 | 0.234 | 0.857 | 0.101 | 1 | 120,224,228 | ADAM30 | 3′-downstream | 13,451 |
| rs937131 | 1.44E−04 | 0.234 | 0.857 | 0.179 | 7 | 97,884,611 | BAIAP2L1 | 5′-upstream | 16,295 |
| rs11822678 | 1.45E−04 | 0.234 | 0.857 | 0.087 | 11 | 21,169,711 | NELL1 | intron | 0 |
| rs4786910 | 1.45E−04 | −0.234 | 0.857 | 0.365 | 16 | 6,674,599 | A2BP1 | intron | 0 |
| rs10095762 | 1.46E−04 | 0.236 | 0.857 | 0.291 | 8 | 129,567,766 | PVT1 | 3′-downstream | 385,085 |
| rs9605252 | 1.46E−04 | 0.236 | 0.857 | 0.148 | 22 | 16,170,349 | CECR1 | 5′-upstream | 99,570 |
| rs1038050 | 1.47E−04 | 0.234 | 0.857 | 0.291 | 9 | 1,704,233 | SMARCA2 | 5′-upstream | 301,109 |
| rs262453 | 1.47E−04 | −0.234 | 0.857 | 0.269 | 11 | 36,013,498 | LDLRAD3 | intron | 0 |
| rs10120246 | 1.47E−04 | 0.235 | 0.857 | 0.089 | 9 | 9,789,050 | PTPRD | intron | 0 |
| rs17778445 | 1.48E−04 | 0.234 | 0.857 | 0.056 | 4 | 90,590,957 | GPRIN3 | 5′-upstream | 142,773 |
| rs2290467 | 1.48E−04 | 0.234 | 0.857 | 0.108 | 8 | 99,109,330 | MATN2 | intron | 0 |
| rs1060681 | 1.49E−04 | 0.234 | 0.857 | 0.169 | 14 | 69,906,506 | SYNJ2BP | 3′-UTR | 0 |
| rs7852743 | 1.49E−04 | 0.237 | 0.857 | 0.350 | 9 | 132,855,205 | LAMC3 | 5′-upstream | 19,120 |
| rs11206538 | 1.49E−04 | 0.233 | 0.857 | 0.406 | 1 | 55,488,000 | LOC645506 | 3′-downstream | 26,367 |
| rs2836416 | 1.49E−04 | 0.233 | 0.857 | 0.170 | 21 | 38,755,361 | ERG | intron | 0 |
| rs11628576 | 1.50E−04 | −0.233 | 0.857 | 0.121 | 14 | 82,214,285 | ENSAP2 | 3′-downstream | 14,419 |
| rs6793337 | 1.50E−04 | 0.235 | 0.857 | 0.234 | 3 | 189,475,302 | LPP | intron | 0 |
| rs10803641 | 1.51E−04 | 0.234 | 0.857 | 0.190 | 2 | 236,038,499 | CENTG2 | 5′-upstream | 28,976 |
| rs6853164 | 1.52E−04 | 0.233 | 0.857 | 0.477 | 4 | 74,370,644 | ANKRD17 | 5′-upstream | 27,278 |
| rs7808549 | 1.54E−04 | −0.237 | 0.857 | 0.300 | 7 | 103,370,996 | RELN | intron | 0 |
| rs11822168 | 1.54E−04 | 0.233 | 0.857 | 0.256 | 11 | 105,136,013 | GRIA4 | intron | 0 |
| rs2887596 | 1.54E−04 | −0.235 | 0.857 | 0.423 | 12 | 76,678,888 | NAV3 | 5′-upstream | 70,312 |
| rs780266 | 1.55E−04 | 0.233 | 0.857 | 0.071 | 1 | 231,791,723 | KCNK1 | 5′-upstream | 24,650 |
| rs936361 | 1.56E−04 | 0.233 | 0.857 | 0.347 | 3 | 88,410,116 | LOC344653 | 5′-upstream | 38,880 |
| rs12830703 | 1.56E−04 | 0.235 | 0.857 | 0.055 | 12 | 18,061,759 | RERGL | 3′-downstream | 63,311 |
| rs1113599 | 1.56E−04 | −0.233 | 0.857 | 0.457 | 12 | 76,699,323 | NAV3 | 5′-upstream | 49,877 |
| rs10956429 | 1.57E−04 | 0.233 | 0.857 | 0.338 | 8 | 129,584,169 | PVT1 | 3′-downstream | 401,488 |
| rs1030016 | 1.59E−04 | 0.233 | 0.857 | 0.191 | 7 | 38,406,097 | AMPH | intron | 0 |
| rs8016862 | 1.59E−04 | −0.233 | 0.857 | 0.429 | 14 | 26,656,409 | RPS27AP4 | 3′-downstream | 141,224 |
| rs1015244 | 1.59E−04 | −0.233 | 0.857 | 0.105 | 1 | 213,437,193 | KCNK2 | intron | 0 |
| rs10159003 | 1.60E−04 | −0.233 | 0.857 | 0.097 | 1 | 213,440,307 | KCNK2 | intron | 0 |
| rs11240237 | 1.60E−04 | 0.232 | 0.857 | 0.475 | 1 | 202,879,141 | LRRN2 | intron | 0 |
| rs4951089 | 1.60E−04 | 0.232 | 0.857 | 0.475 | 1 | 202,877,521 | LRRN2 | intron | 0 |
| rs10157448 | 1.61E−04 | −0.235 | 0.857 | 0.111 | 1 | 213,438,479 | KCNK2 | intron | 0 |
| rs7058743 | 1.61E−04 | 0.233 | 0.857 | 0.145 | 23 | 110,742,481 | LOC643873 | 3′-downstream | 5,789 |
| rs16825358 | 1.62E−04 | 0.234 | 0.857 | 0.285 | 3 | 117,912,388 | LOC285194 | 5′-upstream | 1,640 |
| rs7043006 | 1.62E−04 | 0.233 | 0.857 | 0.129 | 9 | 100,140,610 | GABBR2 | intron | 0 |
| rs12548426 | 1.62E−04 | 0.232 | 0.857 | 0.222 | 8 | 96,174,362 | LOC100130098 | 5′-upstream | 19,337 |
| rs6905924 | 1.62E−04 | 0.234 | 0.857 | 0.226 | 6 | 22,763,578 | LOC389370 | intron | 0 |
| rs32608 | 1.63E−04 | 0.232 | 0.857 | 0.442 | 5 | 11,541,467 | CTNND2 | intron | 0 |
| rs6700381 | 1.63E−04 | 0.233 | 0.857 | 0.438 | 1 | 237,585,731 | CHRM3 | 5′-upstream | 273,265 |
| rs2306774 | 1.65E−04 | 0.232 | 0.857 | 0.220 | 3 | 19,898,826 | EFHB | intron | 0 |
| rs6426991 | 1.65E−04 | 0.232 | 0.857 | 0.214 | 1 | 164,553,110 | LOC284685 | 5′-upstream | 39,603 |
| rs7911417 | 1.65E−04 | −0.232 | 0.857 | 0.106 | 10 | 126,895,990 | LOC642622 | 3′-downstream | 9,917 |
| rs6051490 | 1.65E−04 | −0.232 | 0.857 | 0.475 | 20 | 293,864 | NRSN2 | 3′-downstream | 10,359 |
| rs4657537 | 1.66E−04 | 0.235 | 0.857 | 0.422 | 1 | 164,534,672 | LOC284685 | 5′-upstream | 21,165 |
| rs10221771 | 1.67E−04 | 0.232 | 0.857 | 0.231 | 2 | 236,034,695 | CENTG2 | 5′-upstream | 32,780 |
| rs10508213 | 1.68E−04 | −0.232 | 0.857 | 0.112 | 10 | 1,601,165 | ADARB2 | intron | 0 |
| rs2249971 | 1.69E−04 | −0.232 | 0.857 | 0.352 | 21 | 36,284,531 | RPL23AP3 | 3′-downstream | 25,562 |
| rs1648224 | 1.69E−04 | 0.232 | 0.857 | 0.372 | 11 | 19,269,478 | LOC390102 | 5′-upstream | 16,087 |
| rs17171730 | 1.70E−04 | 0.232 | 0.857 | 0.264 | 7 | 40,558,272 | C7orf10 | intron | 0 |
| rs13138387 | 1.70E−04 | 0.232 | 0.857 | 0.065 | 4 | 16,142,474 | LDB2 | intron | 0 |
| rs7079372 | 1.74E−04 | 0.232 | 0.857 | 0.295 | 10 | 33,613,812 | NRP1 | intron | 0 |
| rs4691114 | 1.75E−04 | −0.232 | 0.857 | 0.308 | 4 | 165,360,381 | ANP32C | 5′-upstream | 22,068 |
| rs1895373 | 1.76E−04 | 0.235 | 0.857 | 0.353 | 19 | 55,801,605 | SYT3 | 3′-downstream | 15,441 |
| rs9974828 | 1.77E−04 | 0.231 | 0.857 | 0.348 | 21 | 39,693,915 | LCA5L | 3′-downstream | 5,725 |
| rs12659182 | 1.77E−04 | 0.231 | 0.857 | 0.094 | 5 | 8,570,119 | LOC729506 | 5′-upstream | 55,351 |
| rs8083033 | 1.77E−04 | 0.231 | 0.857 | 0.125 | 18 | 17,973,918 | LOC100128893 | 3′-downstream | 26,939 |
| rs26953 | 1.78E−04 | −0.233 | 0.857 | 0.326 | 5 | 59,850,071 | PART1 | intron | 0 |
| rs524905 | 1.80E−04 | 0.231 | 0.857 | 0.155 | 5 | 152,862,559 | GRIA1 | intron | 0 |
| rs4707367 | 1.80E−04 | −0.232 | 0.857 | 0.307 | 6 | 88,157,907 | C6orf165 | 5′-upstream | 16,533 |
| rs2848848 | 1.80E−04 | 0.231 | 0.857 | 0.215 | 7 | 13,733,664 | LOC100131022 | 3′-downstream | 126,841 |
| rs1516980 | 1.80E−04 | 0.231 | 0.857 | 0.332 | 8 | 129,587,463 | PVT1 | 3′-downstream | 404,782 |
| rs12227066 | 1.80E−04 | −0.231 | 0.857 | 0.336 | 12 | 76,749,241 | NAV3 | 5′-UTR | 0 |
| rs26154 | 1.81E−04 | 0.232 | 0.857 | 0.442 | 5 | 11,546,913 | CTNND2 | intron | 0 |
| rs11955532 | 1.81E−04 | −0.231 | 0.857 | 0.200 | 5 | 62,016,738 | IPO11 | 3′-downstream | 56,573 |
| rs10850803 | 1.82E−04 | 0.231 | 0.857 | 0.117 | 12 | 116,166,212 | NOS1 | intron | 0 |
| rs11982022 | 1.82E−04 | 0.231 | 0.857 | 0.098 | 7 | 66,525,876 | STAG3L4 | 3′-downstream | 101,928 |
| rs6858372 | 1.83E−04 | −0.231 | 0.857 | 0.409 | 4 | 146,413,132 | LOC100131639 | 5′-upstream | 22,659 |
| rs8016418 | 1.83E−04 | 0.230 | 0.857 | 0.170 | 14 | 69,868,042 | C14orf112 | intron | 0 |
| rs9864031 | 1.83E−04 | −0.230 | 0.857 | 0.153 | 3 | 13,883,072 | WNT7A | intron | 0 |
| rs17044859 | 1.84E−04 | 0.230 | 0.857 | 0.061 | 2 | 23,221,744 | KLHL29 | 5′-upstream | 237,459 |
| rs11604121 | 1.84E−04 | 0.230 | 0.857 | 0.052 | 11 | 9,090,321 | FLJ46111 | 3′-downstream | 16,154 |
| rs9895521 | 1.85E−04 | −0.231 | 0.857 | 0.404 | 17 | 47,461,994 | CA10 | intron | 0 |
| rs10133631 | 1.88E−04 | −0.230 | 0.857 | 0.421 | 14 | 95,089,084 | GLRX5 | 3′-downstream | 8,276 |
| rs2835133 | 1.88E−04 | 0.231 | 0.857 | 0.289 | 21 | 36,055,327 | RPS20P1 | 5′-upstream | 35,893 |
| rs17710575 | 1.88E−04 | 0.230 | 0.857 | 0.208 | 4 | 6,146,988 | JAKMIP1 | intron | 0 |
| rs6851839 | 1.88E−04 | 0.230 | 0.857 | 0.094 | 4 | 153,432,222 | FBXW7 | 3′-downstream | 29,638 |
| rs1130790 | 1.88E−04 | 0.230 | 0.857 | 0.348 | 1 | 200,235,705 | RNPEP | synonymous | 0 |
| rs1698889 | 1.89E−04 | 0.230 | 0.857 | 0.353 | 11 | 19,285,890 | LOC390102 | 5′-upstream | 32,499 |
| rs17058081 | 1.89E−04 | 0.230 | 0.857 | 0.096 | 18 | 71,619,416 | C18orf62 | 5′-upstream | 350,839 |
| rs10028068 | 1.89E−04 | −0.230 | 0.857 | 0.426 | 4 | 66,479,717 | LOC728048 | 5′-upstream | 162,666 |
| rs4951401 | 1.90E−04 | 0.230 | 0.857 | 0.184 | 1 | 202,804,271 | MDM4 | 3′-downstream | 17,922 |
| rs11108379 | 1.90E−04 | 0.230 | 0.857 | 0.085 | 12 | 94,940,019 | LTA4H | intron | 0 |
| rs17025033 | 1.90E−04 | 0.230 | 0.857 | 0.085 | 12 | 94,924,591 | LTA4H | intron | 0 |
| rs12496773 | 1.93E−04 | 0.231 | 0.865 | 0.323 | 3 | 88,448,386 | LOC344653 | 5′-upstream | 610 |
| rs1543780 | 1.94E−04 | 0.230 | 0.865 | 0.199 | 22 | 20,878,522 | IGLVV-58 | Unknown | — |
| rs4631019 | 1.94E−04 | −0.230 | 0.865 | 0.489 | 4 | 148,586,417 | LOC100130537 | 5′-upstream | 19,464 |
| rs9614763 | 1.95E−04 | 0.229 | 0.865 | 0.051 | 22 | 44,475,636 | ATXN10 | intron | 0 |
| rs12923661 | 1.96E−04 | 0.231 | 0.865 | 0.055 | 16 | 10,018,192 | GRIN2A | intron | 0 |
| rs10898852 | 1.98E−04 | −0.231 | 0.865 | 0.266 | 11 | 71,960,572 | PDE2A | 3′-downstream | 4,261 |
| rs17734670 | 1.98E−04 | 0.229 | 0.865 | 0.294 | 15 | 23,390,489 | ATP10A | 3′-downstream | 84,463 |
| rs2786723 | 1.99E−04 | −0.230 | 0.865 | 0.230 | 1 | 197,787,994 | LOC647202 | 3′-downstream | 163,768 |
| rs10051752 | 1.99E−04 | −0.229 | 0.865 | 0.085 | 5 | 154,966,485 | LOC100131033 | 3′-downstream | 113,131 |
| rs1523250 | 1.99E−04 | 0.230 | 0.865 | 0.080 | 8 | 3,789,239 | CSMD1 | intron | 0 |
| rs2772227 | 2.00E−04 | 0.229 | 0.865 | 0.152 | 1 | 202,882,023 | LRRN2 | intron | 0 |
| rs1929409 | 2.00E−04 | 0.229 | 0.865 | 0.245 | 9 | 11,942,643 | LOC100049717 | 5′-upstream | 334,680 |
| rs6897849 | 2.00E−04 | 0.229 | 0.865 | 0.303 | 5 | 2,342,420 | LOC100133292 | 3′-downstream | 292,878 |
| rs1105297 | 2.02E−04 | −0.232 | 0.867 | 0.491 | 1 | 43,184,183 | SLC2A1 | intron | 0 |
| rs746168 | 2.03E−04 | 0.229 | 0.867 | 0.226 | 11 | 74,669,915 | ARRB1 | intron | 0 |
| rs10494959 | 2.04E−04 | 0.229 | 0.867 | 0.074 | 1 | 211,743,516 | LOC643330 | 5′-upstream | 28,312 |
| rs165890 | 2.04E−04 | 0.229 | 0.867 | 0.188 | 22 | 15,708,627 | ZNF402P | 3′-downstream | 9,399 |
| rs165778 | 2.04E−04 | 0.229 | 0.867 | 0.174 | 22 | 15,699,156 | CECR8 | 3′-downstream | 8,931 |
| rs4463400 | 2.06E−04 | 0.229 | 0.867 | 0.148 | 8 | 141,289,178 | NIBP | intron | 0 |
| rs6784029 | 2.06E−04 | 0.229 | 0.867 | 0.217 | 3 | 189,462,427 | LPP | intron | 0 |
| rs425545 | 2.07E−04 | 0.229 | 0.867 | 0.065 | 2 | 70,657,975 | TGFA | 5′-upstream | 23,362 |
| rs1877475 | 2.07E−04 | 0.229 | 0.867 | 0.184 | 13 | 95,335,275 | UGCGL2 | intron | 0 |
| rs9995176 | 2.07E−04 | 0.229 | 0.867 | 0.469 | 4 | 74,395,848 | ANKRD17 | 5′-upstream | 52,482 |
| rs751210 | 2.08E−04 | −0.229 | 0.867 | 0.482 | 1 | 43,183,446 | SLC2A1 | intron | 0 |
| rs718387 | 2.09E−04 | 0.232 | 0.870 | 0.337 | 21 | 36,049,947 | RPS20P1 | 5′-upstream | 30,513 |
| rs4283786 | 2.11E−04 | 0.229 | 0.871 | 0.335 | 5 | 122,128,949 | SNX2 | 5′-upstream | 9,700 |
| rs17031611 | 2.12E−04 | 0.229 | 0.871 | 0.196 | 2 | 66,216,728 | LOC729348 | 5′-upstream | 247,214 |
| rs2776278 | 2.13E−04 | −0.229 | 0.871 | 0.284 | 21 | 36,264,569 | RPL23AP3 | 3′-downstream | 45,524 |
| rs6501930 | 2.13E−04 | −0.229 | 0.871 | 0.138 | 17 | 72,520,666 | MGAT5B | 3′-downstream | 62,600 |
| rs2279120 | 2.13E−04 | 0.232 | 0.871 | 0.112 | 8 | 99,115,216 | MATN2 | intron | 0 |
| rs1450706 | 2.13E−04 | 0.229 | 0.871 | 0.140 | 14 | 87,418,971 | GALC | 3′-downstream | 50,140 |
| rs2348168 | 2.14E−04 | −0.230 | 0.872 | 0.452 | 4 | 160,444,786 | RAPGEF2 | intron | 0 |
| rs11643459 | 2.15E−04 | 0.228 | 0.872 | 0.278 | 16 | 77,355,588 | WWOX | intron | 0 |
| rs540375 | 2.16E−04 | 0.228 | 0.872 | 0.157 | 5 | 152,858,920 | GRIA1 | intron | 0 |
| rs2862465 | 2.22E−04 | 0.228 | 0.891 | 0.439 | 11 | 42,336,068 | LOC387761 | 5′-upstream | 103,940 |
| rs2885552 | 2.23E−04 | 0.227 | 0.891 | 0.096 | 7 | 77,909,577 | MAGI2 | intron | 0 |
| rs8064932 | 2.23E−04 | −0.228 | 0.891 | 0.167 | 17 | 65,095,146 | MAP2K6 | 3′-downstream | 45,081 |
| rs17698900 | 2.23E−04 | 0.227 | 0.891 | 0.054 | 10 | 49,616,272 | LOC100132924 | 3′-downstream | 6,981 |
| rs17616659 | 2.26E−04 | −0.228 | 0.891 | 0.130 | 14 | 82,230,662 | ENSAP2 | 5′-upstream | 1,285 |
| rs2631271 | 2.28E−04 | −0.227 | 0.891 | 0.310 | 4 | 103,133,419 | BANK1 | intron | 0 |
| rs5945353 | 2.28E−04 | 0.228 | 0.891 | 0.101 | 23 | 152,720,129 | PDZD4 | 3′-downstream | 688 |
| rs1949593 | 2.29E−04 | −0.227 | 0.891 | 0.413 | 12 | 76,686,005 | NAV3 | 5′-upstream | 63,195 |
| rs943560 | 2.31E−04 | 0.227 | 0.891 | 0.462 | 13 | 95,649,825 | HS6ST3 | intron | 0 |
| rs10030475 | 2.31E−04 | 0.227 | 0.891 | 0.350 | 4 | 70,842,360 | CSN1S1 | missense | 0 |
| rs12119699 | 2.31E−04 | 0.227 | 0.891 | 0.348 | 1 | 200,239,578 | RNPEP | intron | 0 |
| rs2819358 | 2.31E−04 | 0.227 | 0.891 | 0.348 | 1 | 200,243,025 | ELF3 | 5′-upstream | 671 |
| rs1329631 | 2.31E−04 | 0.227 | 0.891 | 0.307 | 1 | 202,883,013 | LRRN2 | intron | 0 |
| rs6756848 | 2.32E−04 | 0.227 | 0.891 | 0.191 | 2 | 66,215,108 | LOC729348 | 5′-upstream | 248,834 |
| rs2043166 | 2.32E−04 | 0.230 | 0.891 | 0.074 | 18 | 64,902,308 | CCDC102B | 3′-downstream | 28,902 |
| rs37846 | 2.32E−04 | 0.227 | 0.891 | 0.168 | 16 | 3,318,451 | ZNF75A | 3′-downstream | 9,876 |
| rs10959554 | 2.33E−04 | 0.227 | 0.891 | 0.076 | 9 | 11,121,371 | LOC646114 | 3′-downstream | 91,086 |
| rs4474187 | 2.33E−04 | −0.227 | 0.891 | 0.188 | 23 | 89,042,895 | LOC360018 | 3′-downstream | 7,681 |
| rs725224 | 2.33E−04 | 0.227 | 0.891 | 0.076 | 12 | 13,453,789 | C12orf36 | 5′-upstream | 32,877 |
| rs9309382 | 2.34E−04 | 0.227 | 0.891 | 0.202 | 2 | 66,218,009 | LOC729348 | 5′-upstream | 245,933 |
| rs9937123 | 2.35E−04 | 0.227 | 0.891 | 0.060 | 16 | 86,296,990 | KLHDC4 | 3′-downstream | 1,929 |
| rs17131659 | 2.36E−04 | 0.227 | 0.891 | 0.388 | 1 | 84,631,560 | UOX | intron | 0 |
| rs2981317 | 2.37E−04 | 0.227 | 0.891 | 0.200 | 8 | 35,069,374 | LOC100133273 | 3′-downstream | 217,603 |
| rs10943011 | 2.37E−04 | 0.227 | 0.891 | 0.170 | 6 | 73,166,765 | RIMS1 | intron | 0 |
| rs11973006 | 2.38E−04 | 0.226 | 0.891 | 0.253 | 7 | 40,604,890 | C7orf10 | intron | 0 |
| rs7040113 | 2.38E−04 | 0.226 | 0.891 | 0.202 | 9 | 3,413,469 | RFX3 | intron | 0 |
| rs12114930 | 2.41E−04 | 0.226 | 0.900 | 0.430 | 8 | 102,621,763 | GRHL2 | intron | 0 |
| rs2717200 | 2.43E−04 | 0.232 | 0.902 | 0.247 | 4 | 112,745,218 | LOC132719 | 5′-upstream | 472,872 |
| rs2329152 | 2.43E−04 | 0.226 | 0.902 | 0.238 | 13 | 79,509,454 | LOC729479 | 3′-downstream | 10,655 |
| rs17058093 | 2.43E−04 | 0.227 | 0.902 | 0.083 | 18 | 71,620,850 | C18orf62 | 5′-upstream | 352,273 |
| rs557874 | 2.45E−04 | 0.226 | 0.904 | 0.181 | 9 | 134,364,266 | RP11-738I14.8 | intron | 0 |
| rs5969778 | 2.46E−04 | −0.227 | 0.906 | 0.409 | 23 | 16,808,998 | RNU4P6 | 5′-upstream | 5,634 |
| rs9928219 | 2.47E−04 | 0.226 | 0.907 | 0.082 | 16 | 5,991,449 | A2BP1 | 5′-upstream | 17,684 |
| rs17344511 | 2.48E−04 | −0.226 | 0.907 | 0.094 | 1 | 165,421,751 | LOC391130 | 5′-upstream | 23,053 |
| rs2480679 | 2.51E−04 | 0.226 | 0.919 | 0.179 | 1 | 165,674,141 | CD247 | intron | 0 |
| rs1878269 | 2.53E−04 | 0.226 | 0.923 | 0.119 | 3 | 56,454,506 | ERC2 | intron | 0 |
| rs12127605 | 2.55E−04 | 0.228 | 0.925 | 0.066 | 1 | 120,029,707 | PHGDH | 5′-upstream | 26,326 |
| rs17024410 | 2.56E−04 | −0.226 | 0.926 | 0.100 | 1 | 213,439,776 | KCNK2 | intron | 0 |
| rs290255 | 2.58E−04 | 0.227 | 0.929 | 0.168 | 9 | 92,691,478 | SYK | intron | 0 |
| rs10513226 | 2.58E−04 | 0.226 | 0.929 | 0.144 | 3 | 145,065,153 | LOC257039 | intron | 0 |
| rs2836257 | 2.58E−04 | −0.227 | 0.929 | 0.419 | 21 | 38,557,419 | LOC441964 | intron | 0 |
| rs7720273 | 2.60E−04 | −0.226 | 0.931 | 0.230 | 5 | 113,130,084 | YTHDC2 | 3′-downstream | 171,204 |
| rs11006349 | 2.62E−04 | −0.225 | 0.932 | 0.392 | 10 | 60,457,808 | LOC644871 | 3′-downstream | 108,580 |
| rs10516939 | 2.63E−04 | 0.225 | 0.932 | 0.076 | 4 | 94,793,723 | GRID2 | intron | 0 |
| rs1435480 | 2.63E−04 | 0.225 | 0.932 | 0.076 | 4 | 94,769,500 | GRID2 | intron | 0 |
| rs1028182 | 2.65E−04 | 0.225 | 0.932 | 0.406 | 1 | 205,068,502 | IL19 | intron | 0 |
| rs10077572 | 2.66E−04 | 0.225 | 0.932 | 0.374 | 5 | 132,775,906 | FSTL4 | intron | 0 |
| rs780256 | 2.67E−04 | 0.225 | 0.932 | 0.288 | 1 | 231,809,726 | KCNK1 | 5′-upstream | 6,647 |
| rs16993361 | 2.68E−04 | 0.225 | 0.932 | 0.255 | 22 | 43,834,071 | PHF21B | 5′-upstream | 49,826 |
| rs4725434 | 2.69E−04 | −0.225 | 0.932 | 0.342 | 7 | 151,131,312 | PRKAG2 | intron | 0 |
| rs7038965 | 2.70E−04 | 0.225 | 0.932 | 0.099 | 9 | 111,004,281 | EPB41L4B | intron | 0 |
| rs710310 | 2.70E−04 | 0.226 | 0.932 | 0.443 | 14 | 98,719,466 | BCL11B | intron | 0 |
| rs1624327 | 2.71E−04 | 0.224 | 0.932 | 0.255 | 9 | 86,619,110 | NTRK2 | 3′-UTR | 0 |
| rs13282927 | 2.71E−04 | 0.226 | 0.932 | 0.212 | 8 | 35,031,277 | LOC100133273 | 3′-downstream | 179,506 |
| rs17078688 | 2.72E−04 | 0.225 | 0.932 | 0.054 | 6 | 117,394,116 | RFXDC1 | 3′-downstream | 34,109 |
| rs841571 | 2.72E−04 | −0.225 | 0.932 | 0.375 | 1 | 43,208,491 | SLC2A1 | 5′-upstream | 11,403 |
| rs4936507 | 2.74E−04 | 0.224 | 0.932 | 0.067 | 11 | 119,439,773 | TRIM29 | 3′-downstream | 47,431 |
| rs11823347 | 2.75E−04 | 0.225 | 0.932 | 0.084 | 11 | 21,170,916 | NELL1 | intron | 0 |
| rs11788925 | 2.76E−04 | 0.224 | 0.932 | 0.056 | 9 | 134,038,799 | NTNG2 | intron | 0 |
| rs953809 | 2.77E−04 | 0.225 | 0.932 | 0.177 | 1 | 165,670,249 | CD247 | intron | 0 |
| rs10506759 | 2.77E−04 | −0.224 | 0.932 | 0.426 | 12 | 76,670,293 | NAV3 | 5′-upstream | 78,907 |
| rs11665524 | 2.77E−04 | 0.224 | 0.932 | 0.088 | 18 | 2,968,535 | LPIN2 | intron | 0 |
| rs6778788 | 2.77E−04 | 0.229 | 0.932 | 0.321 | 3 | 88,422,500 | LOC344653 | 5′-upstream | 26,496 |
| rs17714061 | 2.78E−04 | 0.224 | 0.932 | 0.076 | 7 | 39,613,030 | RALA | 5′-upstream | 16,657 |
| rs3114315 | 2.80E−04 | −0.224 | 0.932 | 0.357 | 7 | 76,719,919 | CCDC146 | intron | 0 |
| rs12677635 | 2.80E−04 | 0.224 | 0.932 | 0.235 | 8 | 88,687,271 | LOC642461 | 5′-upstream | 4,113 |
| rs17475516 | 2.82E−04 | 0.224 | 0.932 | 0.173 | 14 | 69,902,232 | SYNJ2BP | 3′-downstream | 734 |
| rs17125238 | 2.82E−04 | −0.224 | 0.932 | 0.484 | 1 | 101,959,532 | RP11-556K13.1 | 3′-downstream | 47,413 |
| rs7556071 | 2.83E−04 | 0.225 | 0.932 | 0.060 | 1 | 188,241,377 | LOC647132 | Unknown | — |
| rs11620973 | 2.84E−04 | 0.225 | 0.932 | 0.400 | 14 | 95,087,805 | GLRX5 | 3′-downstream | 6,997 |
| rs17031608 | 2.85E−04 | 0.224 | 0.932 | 0.205 | 2 | 66,216,670 | LOC729348 | 5′-upstream | 247,272 |
| rs3848333 | 2.85E−04 | 0.225 | 0.932 | 0.055 | 16 | 10,025,056 | GRIN2A | intron | 0 |
| rs10982373 | 2.85E−04 | 0.224 | 0.932 | 0.411 | 9 | 116,503,083 | LOC100129633 | intron | 0 |
| rs13127174 | 2.86E−04 | 0.224 | 0.932 | 0.157 | 4 | 16,137,569 | LDB2 | intron | 0 |
| rs2816600 | 2.87E−04 | 0.224 | 0.932 | 0.139 | 1 | 43,039,717 | CCDC23 | 3′-downstream | 5,603 |
| rs655741 | 2.87E−04 | 0.224 | 0.932 | 0.139 | 1 | 43,046,340 | CCDC23 | intron | 0 |
| rs6441728 | 2.87E−04 | 0.224 | 0.932 | 0.354 | 3 | 104,551,408 | LOC644681 | 3′-downstream | 188,760 |
| rs10492841 | 2.87E−04 | −0.224 | 0.932 | 0.342 | 16 | 6,679,934 | A2BP1 | intron | 0 |
| rs6990941 | 2.89E−04 | −0.223 | 0.932 | 0.301 | 8 | 89,713,547 | LOC100129100 | 3′-downstream | 146,108 |
| rs6965558 | 2.89E−04 | 0.228 | 0.932 | 0.352 | 7 | 1,518,964 | INTS1 | 5′-upstream | 5,765 |
| rs6948724 | 2.90E−04 | 0.223 | 0.932 | 0.255 | 7 | 40,622,800 | C7orf10 | intron | 0 |
| rs4245739 | 2.90E−04 | 0.223 | 0.932 | 0.188 | 1 | 202,785,465 | MDM4 | 3′-UTR | 0 |
| rs1654531 | 2.91E−04 | 0.225 | 0.932 | 0.241 | 19 | 56,167,461 | KLK7 | 3′-downstream | 4,080 |
| rs12080720 | 2.92E−04 | −0.224 | 0.932 | 0.462 | 1 | 115,823,107 | LOC100132332 | 5′-upstream | 85,419 |
| rs10774976 | 2.93E−04 | 0.224 | 0.932 | 0.098 | 12 | 116,922,846 | RFC5 | 5′-upstream | 16,047 |
| rs7430477 | 2.93E−04 | 0.224 | 0.932 | 0.447 | 3 | 38,740,494 | SCN10A | intron | 0 |
| rs10841186 | 2.94E−04 | 0.224 | 0.932 | 0.249 | 12 | 8,345,682 | LOC100130149 | 3′-downstream | 27,554 |
| rs2498667 | 2.95E−04 | 0.223 | 0.932 | 0.251 | 6 | 139,472,289 | HECA | 5′-upstream | 25,653 |
| rs7717457 | 2.95E−04 | −0.223 | 0.932 | 0.310 | 5 | 40,923,538 | C7 | 5′-upstream | 21,818 |
| rs6884002 | 2.96E−04 | −0.223 | 0.932 | 0.099 | 5 | 154,872,015 | LOC100131033 | 3′-downstream | 18,661 |
| rs957583 | 2.97E−04 | 0.223 | 0.932 | 0.120 | 23 | 108,979,979 | LOC392520 | 5′-upstream | 3,220 |
| rs1400558 | 2.97E−04 | −0.223 | 0.932 | 0.419 | 4 | 148,600,255 | EDNRA | 5′-upstream | 21,320 |
| rs1400560 | 2.97E−04 | −0.223 | 0.932 | 0.419 | 4 | 148,599,582 | EDNRA | 5′-upstream | 21,993 |
| rs3826884 | 2.98E−04 | 0.223 | 0.932 | 0.139 | 19 | 60,429,993 | TMEM86B | 3′-UTR | 0 |
| rs717695 | 2.98E−04 | −0.223 | 0.932 | 0.408 | 4 | 156,634,854 | TRNAL-UAA | 5′-upstream | 30,352 |
| rs11587357 | 3.00E−04 | 0.223 | 0.932 | 0.074 | 1 | 95,890,849 | LOC729977 | 5′-upstream | 334,442 |
| rs11703864 | 3.00E−04 | 0.223 | 0.932 | 0.166 | 22 | 26,240,759 | MN1 | 3′-downstream | 233,506 |
| rs6962918 | 3.01E−04 | 0.223 | 0.932 | 0.126 | 7 | 36,081,241 | PP13004 | 5′-upstream | 3,978 |
| rs17253026 | 3.01E−04 | 0.223 | 0.932 | 0.136 | 14 | 51,899,472 | PTGER2 | 3′-downstream | 34,398 |
| rs11249617 | 3.01E−04 | 0.223 | 0.932 | 0.341 | 5 | 178,590,767 | ADAMTS2 | intron | 0 |
| rs6840083 | 3.01E−04 | 0.223 | 0.932 | 0.421 | 4 | 183,701,671 | ODZ3 | intron | 0 |
| rs1893636 | 3.04E−04 | 0.224 | 0.932 | 0.427 | 18 | 57,282,780 | CDH20 | 5′-upstream | 25,975 |
| rs6804165 | 3.06E−04 | 0.223 | 0.932 | 0.107 | 3 | 11,016,808 | SLC6A1 | intron | 0 |
| rs7688236 | 3.06E−04 | 0.223 | 0.932 | 0.099 | 4 | 87,023,102 | ARHGAP24 | intron | 0 |
| rs10133760 | 3.06E−04 | −0.223 | 0.932 | 0.388 | 14 | 95,088,964 | GLRX5 | 3′-downstream | 8,156 |
| rs1712563 | 3.08E−04 | 0.222 | 0.932 | 0.352 | 3 | 108,135,110 | LOC100128733 | 3′-downstream | 84,301 |
| rs10937476 | 3.08E−04 | 0.223 | 0.932 | 0.255 | 3 | 192,514,654 | CCDC50 | 5′-upstream | 14,914 |
| rs7539399 | 3.08E−04 | 0.222 | 0.932 | 0.269 | 1 | 202,856,667 | LRRN2 | intron | 0 |
| rs16531 | 3.09E−04 | 0.223 | 0.932 | 0.252 | 17 | 34,603,181 | CACNB1 | intron | 0 |
| rs11848605 | 3.11E−04 | 0.224 | 0.932 | 0.241 | 14 | 43,266,962 | ARHGAP16P | 3′-downstream | 131,674 |
| rs39637 | 3.13E−04 | 0.222 | 0.932 | 0.253 | 3 | 22,842,432 | LOC100129341 | 5′-upstream | 78,195 |
| rs7432616 | 3.13E−04 | 0.222 | 0.932 | 0.153 | 3 | 117,908,124 | LOC285194 | 5′-upstream | 5,904 |
| rs7758611 | 3.14E−04 | 0.222 | 0.932 | 0.078 | 6 | 54,407,429 | TINAG | 3′-downstream | 44,557 |
| rs11849993 | 3.14E−04 | 0.223 | 0.932 | 0.243 | 14 | 43,268,583 | ARHGAP16P | 3′-downstream | 133,295 |
| rs28410948 | 3.15E−04 | −0.222 | 0.932 | 0.469 | 1 | 19,061,043 | ALDH4A1 | 3′-downstream | 9,470 |
| rs2503661 | 3.16E−04 | 0.222 | 0.932 | 0.222 | 6 | 92,817,083 | LOC100129847 | 5′-upstream | 234,231 |
| rs2819360 | 3.17E−04 | 0.222 | 0.932 | 0.341 | 1 | 200,243,877 | ELF3 | 5′-UTR | 0 |
| rs644809 | 3.18E−04 | 0.222 | 0.932 | 0.264 | 9 | 198,549 | C9orf66 | 3′-downstream | 4,913 |
| rs2139108 | 3.18E−04 | −0.222 | 0.932 | 0.267 | 16 | 78,827,814 | LOC729847 | intron | 0 |
| rs4697063 | 3.19E−04 | 0.222 | 0.932 | 0.359 | 4 | 24,147,671 | DHX15 | intron | 0 |
| rs2521676 | 3.19E−04 | 0.223 | 0.932 | 0.287 | 16 | 26,802,777 | C16orf82 | 5′-upstream | 182,752 |
| rs6892211 | 3.19E−04 | 0.222 | 0.932 | 0.096 | 5 | 8,557,567 | LOC729506 | 5′-upstream | 42,799 |
| rs4733581 | 3.20E−04 | −0.222 | 0.932 | 0.371 | 8 | 129,016,957 | PVT1 | intron | 0 |
| rs7050429 | 3.20E−04 | 0.224 | 0.932 | 0.147 | 23 | 110,743,608 | LOC643873 | 3′-downstream | 4,662 |
| rs10222063 | 3.21E−04 | −0.222 | 0.932 | 0.310 | 21 | 41,216,672 | DSCAM | 5′-upstream | 75,763 |
| rs6869955 | 3.23E−04 | 0.222 | 0.932 | 0.099 | 5 | 55,400,449 | ANKRD55 | 3′-downstream | 30,815 |
| rs9478311 | 3.23E−04 | −0.222 | 0.932 | 0.413 | 6 | 150,328,828 | ULBP1 | intron | 0 |
| rs2501830 | 3.24E−04 | 0.222 | 0.932 | 0.119 | 23 | 108,965,630 | LOC392520 | 5′-upstream | 17,569 |
| rs966951 | 3.24E−04 | 0.222 | 0.932 | 0.119 | 23 | 108,979,489 | LOC392520 | 5′-upstream | 3,710 |
| rs2743218 | 3.24E−04 | 0.222 | 0.932 | 0.388 | 1 | 18,866,737 | PAX7 | intron | 0 |
| rs11025102 | 3.24E−04 | 0.222 | 0.932 | 0.332 | 11 | 19,307,344 | LOC390102 | 5′-upstream | 53,953 |
| rs6449478 | 3.26E−04 | −0.224 | 0.932 | 0.267 | 5 | 59,909,466 | DEPDC1B | 3′-downstream | 19,030 |
| rs2455377 | 3.26E−04 | −0.222 | 0.932 | 0.484 | 5 | 55,956,448 | LOC441073 | 5′-upstream | 119,755 |
| rs3915861 | 3.26E−04 | 0.222 | 0.932 | 0.368 | 9 | 78,082,346 | LOC100133206 | 3′-downstream | 8,884 |
| rs16942674 | 3.27E−04 | 0.222 | 0.932 | 0.063 | 17 | 53,542,806 | DYNLL2 | 3′-downstream | 20,996 |
| rs1872366 | 3.28E−04 | 0.225 | 0.932 | 0.169 | 12 | 30,580,467 | IPO8 | 3′-downstream | 92,722 |
| rs2812617 | 3.29E−04 | 0.221 | 0.932 | 0.123 | 10 | 34,113,351 | PARD3 | 3′-downstream | 326,752 |
| rs3910572 | 3.29E−04 | −0.221 | 0.932 | 0.397 | 13 | 79,518,672 | LOC729485 | 3′-downstream | 8,738 |
| rs812086 | 3.30E−04 | 0.222 | 0.932 | 0.353 | 11 | 19,279,293 | LOC390102 | 5′-upstream | 25,902 |
| rs6578080 | 3.31E−04 | 0.222 | 0.932 | 0.228 | 8 | 141,291,830 | NIBP | intron | 0 |
| rs6489900 | 3.31E−04 | −0.222 | 0.932 | 0.357 | 12 | 112,439,970 | LHX5 | 5′-upstream | 45,710 |
| rs7061836 | 3.32E−04 | 0.221 | 0.932 | 0.152 | 23 | 110,733,178 | LOC643873 | 3′-downstream | 15,092 |
| rs1376071 | 3.35E−04 | −0.221 | 0.932 | 0.422 | 15 | 35,027,798 | MEIS2 | intron | 0 |
| rs17151081 | 3.35E−04 | 0.222 | 0.932 | 0.101 | 7 | 76,755,796 | CCDC146 | intron | 0 |
| rs6979107 | 3.36E−04 | −0.223 | 0.932 | 0.147 | 7 | 64,508,216 | ZNF92 | 3′-downstream | 4,783 |
| rs1220854 | 3.36E−04 | 0.222 | 0.932 | 0.498 | 4 | 70,846,893 | CSN2 | 3′-downstream | 8,671 |
| rs12621560 | 3.36E−04 | 0.222 | 0.932 | 0.478 | 2 | 74,787,875 | SEMA4F | 3′-downstream | 25,182 |
| rs2243668 | 3.37E−04 | 0.221 | 0.932 | 0.157 | 10 | 33,603,899 | NRP1 | intron | 0 |
| rs7100693 | 3.37E−04 | 0.221 | 0.932 | 0.063 | 10 | 64,590,594 | JMJD1C | 3′-downstream | 6,397 |
| rs6567819 | 3.44E−04 | 0.221 | 0.932 | 0.119 | 23 | 108,991,343 | LOC392520 | 3′-downstream | 7,539 |
| rs283128 | 3.46E−04 | 0.221 | 0.932 | 0.464 | 5 | 32,102,829 | PDZD2 | intron | 0 |
| rs10962837 | 3.46E−04 | −0.221 | 0.932 | 0.312 | 9 | 1,704,649 | SMARCA2 | 5′-upstream | 300,693 |
| rs881790 | 3.46E−04 | 0.221 | 0.932 | 0.195 | 4 | 16,147,055 | LDB2 | intron | 0 |
| rs4455605 | 3.47E−04 | 0.221 | 0.932 | 0.265 | 5 | 40,184,520 | LOC285634 | 5′-upstream | 391,073 |
| rs1946944 | 3.47E−04 | 0.221 | 0.932 | 0.218 | 8 | 96,171,901 | LOC100130098 | 5′-upstream | 16,876 |
| rs4734298 | 3.47E−04 | 0.221 | 0.932 | 0.218 | 8 | 96,170,962 | LOC100130098 | 5′-upstream | 15,937 |
| rs10124468 | 3.48E−04 | 0.221 | 0.932 | 0.115 | 9 | 11,235,830 | LOC646114 | 3′-downstream | 205,545 |
| rs17060993 | 3.48E−04 | 0.221 | 0.932 | 0.058 | 8 | 26,978,029 | LOC100132229 | 5′-upstream | 52,134 |
| rs11696891 | 3.48E−04 | −0.221 | 0.932 | 0.422 | 20 | 54,876,477 | PTMAP6 | 5′-upstream | 166,484 |
| rs994772 | 3.49E−04 | 0.225 | 0.932 | 0.143 | 4 | 100,546,687 | ADH7 | 3′-downstream | 5,754 |
| rs1469095 | 3.49E−04 | −0.221 | 0.932 | 0.293 | 5 | 62,019,604 | IPO11 | 3′-downstream | 59,439 |
| rs2777508 | 3.49E−04 | 0.223 | 0.932 | 0.119 | 10 | 34,112,449 | PARD3 | 3′-downstream | 327,654 |
| rs8053650 | 3.50E−04 | −0.220 | 0.932 | 0.222 | 16 | 11,450,946 | LOC400499 | intron | 0 |
| rs10805350 | 3.50E−04 | 0.222 | 0.932 | 0.313 | 4 | 16,140,499 | LDB2 | intron | 0 |
| rs4845144 | 3.50E−04 | 0.222 | 0.932 | 0.458 | 1 | 205,083,271 | IL19 | 3′-downstream | 324 |
| rs1755938 | 3.51E−04 | 0.220 | 0.932 | 0.182 | 9 | 92,684,769 | SYK | intron | 0 |
| rs10833372 | 3.51E−04 | 0.221 | 0.932 | 0.478 | 11 | 20,628,328 | SLC6A5 | intron | 0 |
| rs1477357 | 3.51E−04 | −0.220 | 0.932 | 0.206 | 5 | 62,003,267 | IPO11 | 3′-downstream | 43,102 |
| rs12494229 | 3.52E−04 | 0.221 | 0.932 | 0.317 | 3 | 190,356,633 | LOC100132319 | 5′-upstream | 5,014 |
| rs649870 | 3.52E−04 | −0.220 | 0.932 | 0.469 | 11 | 118,476,461 | DPAGT1 | intron | 0 |
| rs17616768 | 3.52E−04 | −0.220 | 0.932 | 0.130 | 14 | 82,235,273 | ENSAP2 | 5′-upstream | 5,896 |
| rs12581982 | 3.53E−04 | 0.221 | 0.932 | 0.076 | 12 | 13,452,773 | C12orf36 | 5′-upstream | 31,861 |
| rs6492840 | 3.53E−04 | 0.220 | 0.932 | 0.464 | 13 | 95,637,435 | HS6ST3 | intron | 0 |
| rs2305089 | 3.54E−04 | −0.222 | 0.932 | 0.418 | 6 | 166,499,260 | T | missense | 0 |
| rs6678565 | 3.54E−04 | −0.220 | 0.932 | 0.087 | 1 | 213,429,084 | KCNK2 | intron | 0 |
| rs7657364 | 3.55E−04 | 0.220 | 0.932 | 0.419 | 4 | 24,181,029 | DHX15 | intron | 0 |
| rs11135305 | 3.56E−04 | 0.220 | 0.932 | 0.065 | 5 | 163,529,934 | LOC391844 | 5′-upstream | 402,292 |
| rs6500550 | 3.57E−04 | 0.221 | 0.932 | 0.486 | 16 | 3,686,242 | TRAP1 | intron | 0 |
| rs6907305 | 3.57E−04 | 0.220 | 0.932 | 0.121 | 6 | 21,505,616 | CDKAL1 | 3′-downstream | 165,873 |
| rs12606882 | 3.57E−04 | 0.221 | 0.932 | 0.404 | 18 | 27,743,193 | KIAA1012 | intron | 0 |
| rs4634366 | 3.59E−04 | −0.222 | 0.932 | 0.272 | 5 | 169,523,385 | KRT18P41 | 5′-upstream | 14,507 |
| rs7939727 | 3.59E−04 | 0.220 | 0.932 | 0.061 | 11 | 11,734,325 | USP47 | 5′-upstream | 85,221 |
| rs7397057 | 3.59E−04 | −0.220 | 0.932 | 0.422 | 12 | 131,201,474 | NOC4L | intron | 0 |
| rs17379645 | 3.60E−04 | 0.221 | 0.932 | 0.058 | 1 | 162,614,736 | LOC729952 | 3′-downstream | 20,655 |
| rs11252650 | 3.61E−04 | 0.222 | 0.932 | 0.097 | 10 | 4,704,053 | LOC338588 | 3′-downstream | 9,442 |
| rs1155097 | 3.63E−04 | 0.220 | 0.932 | 0.233 | 4 | 156,634,165 | TRNAL-UAA | 5′-upstream | 29,663 |
| rs10518301 | 3.64E−04 | −0.220 | 0.932 | 0.339 | 4 | 120,454,573 | FABP2 | 3′-downstream | 3,280 |
| rs290226 | 3.65E−04 | 0.220 | 0.932 | 0.178 | 9 | 92,677,103 | SYK | intron | 0 |
| rs6578084 | 3.66E−04 | −0.220 | 0.932 | 0.366 | 8 | 141,311,849 | NIBP | intron | 0 |
| rs10494457 | 3.66E−04 | 0.220 | 0.932 | 0.254 | 1 | 164,528,747 | LOC284685 | 5′-upstream | 15,240 |
| rs7552585 | 3.67E−04 | −0.220 | 0.932 | 0.097 | 1 | 203,675,794 | MIRN135B | 3′-downstream | 8,259 |
| rs1861595 | 3.67E−04 | −0.220 | 0.932 | 0.395 | 12 | 15,552,491 | PTPRO | intron | 0 |
| rs2631246 | 3.67E−04 | 0.220 | 0.932 | 0.356 | 4 | 103,098,344 | BANK1 | intron | 0 |
| rs12650712 | 3.68E−04 | 0.220 | 0.932 | 0.159 | 4 | 165,672,051 | LOC100133261 | 5′-upstream | 65,239 |
| rs3091377 | 3.68E−04 | −0.220 | 0.932 | 0.101 | 22 | 38,527,581 | ENTHD1 | intron | 0 |
| rs2835680 | 3.69E−04 | 0.220 | 0.932 | 0.415 | 21 | 37,526,440 | DSCR3 | intron | 0 |
| rs16878245 | 3.69E−04 | −0.220 | 0.932 | 0.137 | 4 | 10,730,190 | LOC643446 | 5′-upstream | 241,891 |
| rs9806917 | 3.71E−04 | 0.220 | 0.936 | 0.054 | 16 | 23,298,357 | SCNN1B | intron | 0 |
| rs16907672 | 3.72E−04 | 0.220 | 0.936 | 0.089 | 11 | 21,184,140 | NELL1 | intron | 0 |
| rs10965572 | 3.73E−04 | 0.219 | 0.936 | 0.191 | 9 | 23,032,805 | LOC646609 | 3′-downstream | 294,529 |
| rs9651539 | 3.73E−04 | −0.219 | 0.936 | 0.341 | 10 | 13,519,502 | C10orf30 | 3′-downstream | 988 |
| rs12581742 | 3.75E−04 | 0.220 | 0.938 | 0.074 | 12 | 13,453,060 | C12orf36 | 5′-upstream | 32,148 |
| rs2842721 | 3.76E−04 | 0.220 | 0.939 | 0.396 | 1 | 205,254,830 | C1orf116 | 3′-downstream | 3,659 |
| rs13114034 | 3.80E−04 | 0.219 | 0.944 | 0.076 | 4 | 6,575,335 | PPP2R2C | 5′-upstream | 50,108 |
| rs17397811 | 3.81E−04 | 0.220 | 0.944 | 0.373 | 8 | 102,618,040 | GRHL2 | intron | 0 |
| rs1552290 | 3.81E−04 | −0.219 | 0.944 | 0.179 | 1 | 101,873,811 | RP11-556K13.1 | 3′-downstream | 133,134 |
| rs3848332 | 3.81E−04 | 0.220 | 0.944 | 0.058 | 16 | 10,025,038 | GRIN2A | intron | 0 |
| rs2467383 | 3.82E−04 | 0.219 | 0.944 | 0.278 | 11 | 34,153,060 | ABTB2 | intron | 0 |
| rs6918518 | 3.83E−04 | −0.222 | 0.944 | 0.487 | 6 | 158,850,684 | TULP4 | 3′-UTR | 0 |
| rs2922388 | 3.84E−04 | −0.222 | 0.944 | 0.101 | 8 | 137,005,767 | KHDRBS3 | 3′-downstream | 276,736 |
| rs11627491 | 3.85E−04 | −0.220 | 0.944 | 0.113 | 14 | 82,278,510 | LOC730105 | 3′-downstream | 46,045 |
| rs2969778 | 3.86E−04 | 0.219 | 0.944 | 0.164 | 2 | 47,759,696 | LOC285053 | 3′-downstream | 11,587 |
| rs12546486 | 3.86E−04 | 0.219 | 0.944 | 0.327 | 8 | 95,195,882 | CDH17 | 3′-downstream | 12,684 |
| rs856119 | 3.86E−04 | 0.220 | 0.944 | 0.195 | 1 | 157,166,371 | PYHIN1 | 5′-upstream | 1,595 |
| rs7690945 | 3.86E−04 | −0.221 | 0.944 | 0.425 | 4 | 155,213,440 | DCHS2 | 3′-downstream | 161,698 |
| rs10509583 | 3.88E−04 | 0.219 | 0.944 | 0.097 | 10 | 91,634,406 | hCG_2024094 | 3′-downstream | 46,965 |
| rs12932835 | 3.90E−04 | 0.219 | 0.944 | 0.058 | 16 | 10,019,526 | GRIN2A | intron | 0 |
| rs1046623 | 3.92E−04 | 0.219 | 0.944 | 0.372 | 16 | 83,157,772 | COTL1 | 3′-UTR | 0 |
| rs1328379 | 3.95E−04 | 0.219 | 0.944 | 0.170 | 6 | 165,923,631 | PDE10A | intron | 0 |
| rs12291695 | 3.95E−04 | −0.219 | 0.944 | 0.317 | 11 | 12,752,238 | TEAD1 | intron | 0 |
| rs9324519 | 3.96E−04 | −0.219 | 0.944 | 0.375 | 8 | 141,312,047 | NIBP | intron | 0 |
| rs5888 | 3.97E−04 | 0.221 | 0.944 | 0.304 | 12 | 123,850,701 | SCARB1 | synonymous | 0 |
| rs2883036 | 3.99E−04 | −0.221 | 0.944 | 0.361 | 1 | 205,091,385 | IL19 | 3′-downstream | 8,438 |
| rs1426227 | 3.99E−04 | 0.219 | 0.944 | 0.107 | 18 | 2,157,478 | LOC100128360 | 5′-upstream | 235,792 |
| rs11643910 | 3.99E−04 | 0.218 | 0.944 | 0.065 | 16 | 80,997,215 | CDH13 | 5′-upstream | 220,864 |
| rs1032915 | 4.02E−04 | 0.218 | 0.944 | 0.397 | 18 | 73,608,817 | BDP1P | 5′-upstream | 330,525 |
| rs2381623 | 4.02E−04 | −0.218 | 0.944 | 0.339 | 9 | 7,502,409 | LOC158345 | 5′-upstream | 34,028 |
| rs12331227 | 4.02E−04 | −0.221 | 0.944 | 0.463 | 4 | 30,827,466 | LOC645716 | 3′-downstream | 5,071 |
| rs870522 | 4.02E−04 | 0.218 | 0.944 | 0.363 | 14 | 51,028,639 | FRMD6 | intron | 0 |
| rs12154459 | 4.02E−04 | 0.219 | 0.944 | 0.316 | 7 | 145,885,441 | CNTNAP2 | intron | 0 |
| rs1528474 | 4.03E−04 | 0.218 | 0.944 | 0.150 | 15 | 53,130,237 | C15orf15 | 3′-downstream | 130,567 |
| rs1699195 | 4.04E−04 | 0.218 | 0.944 | 0.072 | 1 | 211,596,070 | LOC643330 | 3′-downstream | 72,604 |
| rs1455096 | 4.04E−04 | 0.218 | 0.944 | 0.287 | 6 | 67,460,312 | NUFIP1P | 5′-upstream | 598,780 |
| rs895851 | 4.04E−04 | −0.218 | 0.944 | 0.110 | 8 | 137,012,462 | KHDRBS3 | 3′-downstream | 283,431 |
| rs16944718 | 4.04E−04 | −0.219 | 0.944 | 0.133 | 17 | 11,274,781 | FLJ45455 | intron | 0 |
| rs16952353 | 4.05E−04 | 0.219 | 0.944 | 0.112 | 17 | 2,588,038 | LOC100128142 | 5′-upstream | 2,849 |
| rs999787 | 4.06E−04 | 0.219 | 0.944 | 0.158 | 15 | 53,079,743 | C15orf15 | 3′-downstream | 181,061 |
| rs1379694 | 4.06E−04 | 0.219 | 0.944 | 0.346 | 3 | 88,448,629 | LOC344653 | 5′-upstream | 367 |
| rs431791 | 4.07E−04 | 0.218 | 0.944 | 0.179 | 9 | 135,841,095 | VAV2 | intron | 0 |
| rs1413730 | 4.07E−04 | 0.218 | 0.944 | 0.065 | 6 | 117,389,088 | RFXDC1 | 3′-downstream | 29,081 |
| rs9979015 | 4.09E−04 | 0.218 | 0.944 | 0.278 | 21 | 35,157,438 | RUNX1 | intron | 0 |
| rs9866355 | 4.09E−04 | 0.218 | 0.944 | 0.356 | 3 | 117,909,276 | LOC285194 | 5′-upstream | 4,752 |
| rs3886706 | 4.09E−04 | 0.218 | 0.944 | 0.491 | 1 | 111,590,302 | CHI3L2 | 3′-downstream | 2,717 |
| rs17047903 | 4.12E−04 | 0.218 | 0.944 | 0.428 | 3 | 68,785,403 | PSMC1P | 3′-downstream | 16,220 |
| rs302828 | 4.12E−04 | −0.219 | 0.944 | 0.117 | 19 | 61,102,152 | NLRP13 | intron | 0 |
| rs10499617 | 4.16E−04 | 0.219 | 0.944 | 0.049 | 7 | 40,749,097 | C7orf10 | intron | 0 |
| rs4733583 | 4.16E−04 | −0.218 | 0.944 | 0.379 | 8 | 129,017,407 | PVT1 | intron | 0 |
| rs11731134 | 4.17E−04 | −0.218 | 0.944 | 0.491 | 4 | 148,599,064 | EDNRA | 5′-upstream | 22,511 |
| rs4835405 | 4.17E−04 | −0.218 | 0.944 | 0.491 | 4 | 148,598,134 | EDNRA | 5′-upstream | 23,441 |
| rs10117265 | 4.18E−04 | 0.218 | 0.944 | 0.110 | 9 | 100,135,923 | GABBR2 | intron | 0 |
| rs4384204 | 4.18E−04 | −0.219 | 0.944 | 0.345 | 1 | 213,478,228 | KCNK2 | 3′-downstream | 1,169 |
| rs12678629 | 4.19E−04 | 0.218 | 0.944 | 0.215 | 8 | 35,025,990 | LOC100133273 | 3′-downstream | 174,219 |
| rs7547418 | 4.19E−04 | 0.218 | 0.944 | 0.233 | 1 | 164,542,624 | LOC284685 | 5′-upstream | 29,117 |
| rs4953672 | 4.20E−04 | −0.220 | 0.944 | 0.482 | 2 | 42,953,942 | LOC100128048 | 5′-upstream | 45,681 |
| rs10900418 | 4.20E−04 | 0.218 | 0.944 | 0.309 | 1 | 202,880,789 | LRRN2 | intron | 0 |
| rs7920683 | 4.21E−04 | 0.218 | 0.944 | 0.435 | 10 | 20,429,242 | PLXDC2 | intron | 0 |
| rs7157515 | 4.21E−04 | 0.218 | 0.944 | 0.128 | 14 | 53,558,366 | LOC645560 | 5′-upstream | 29,903 |
| rs6901033 | 4.21E−04 | 0.218 | 0.944 | 0.061 | 6 | 117,373,420 | RFXDC1 | 3′-downstream | 13,413 |
| rs7765150 | 4.25E−04 | 0.217 | 0.944 | 0.056 | 6 | 50,271,304 | DEFB112 | 5′-upstream | 146,981 |
| rs12810670 | 4.26E−04 | −0.219 | 0.944 | 0.278 | 12 | 76,732,425 | NAV3 | 5′-upstream | 16,775 |
| rs9550637 | 4.27E−04 | −0.218 | 0.944 | 0.442 | 13 | 19,810,527 | CRYL1 | 3′-downstream | 65,279 |
| rs11722847 | 4.28E−04 | −0.218 | 0.944 | 0.339 | 4 | 120,452,569 | FABP2 | 3′-downstream | 5,284 |
| rs2197164 | 4.29E−04 | 0.217 | 0.944 | 0.065 | 18 | 65,279,551 | DOK6 | intron | 0 |
| rs6578233 | 4.32E−04 | −0.218 | 0.944 | 0.221 | 8 | 136,447,775 | LOC286094 | 3′-downstream | 69,795 |
| rs247325 | 4.33E−04 | 0.218 | 0.944 | 0.276 | 12 | 14,812,901 | HIST4H4 | 3′-downstream | 2,020 |
| rs784235 | 4.34E−04 | −0.217 | 0.944 | 0.112 | 18 | 51,574,142 | LOC100132501 | 3′-downstream | 25,133 |
| rs871503 | 4.36E−04 | 0.217 | 0.944 | 0.330 | 5 | 173,534,310 | HMP19 | 3′-downstream | 65,522 |
| rs4739519 | 4.36E−04 | 0.217 | 0.944 | 0.347 | 8 | 37,532,017 | LOC100128034 | intron | 0 |
| rs6005031 | 4.40E−04 | 0.217 | 0.944 | 0.309 | 22 | 25,095,167 | SEZ6L | intron | 0 |
| rs2387137 | 4.40E−04 | 0.217 | 0.944 | 0.375 | 19 | 55,829,472 | SYT3 | intron | 0 |
| rs2564942 | 4.41E−04 | −0.217 | 0.944 | 0.430 | 3 | 53,114,546 | RFT1 | intron | 0 |
| rs2559658 | 4.42E−04 | 0.217 | 0.944 | 0.088 | 10 | 79,415,275 | POLR3A | intron | 0 |
| rs17678419 | 4.44E−04 | 0.217 | 0.944 | 0.054 | 12 | 113,486,329 | LOC652191 | 3′-downstream | 14,280 |
| rs7866090 | 4.45E−04 | 0.217 | 0.944 | 0.103 | 9 | 11,130,883 | LOC646114 | 3′-downstream | 100,598 |
| rs10075142 | 4.46E−04 | 0.217 | 0.944 | 0.074 | 5 | 73,962,740 | ENC1 | intron | 0 |
| rs1393155 | 4.47E−04 | −0.222 | 0.944 | 0.319 | 5 | 40,925,894 | C7 | 5′-upstream | 19,462 |
| rs9992415 | 4.48E−04 | −0.217 | 0.944 | 0.460 | 4 | 30,827,933 | LOC645716 | 3′-downstream | 5,538 |
| rs7775551 | 4.48E−04 | 0.217 | 0.944 | 0.162 | 6 | 17,155,652 | FLJ23152 | 5′-upstream | 80,496 |
| rs11190790 | 4.49E−04 | −0.217 | 0.944 | 0.294 | 10 | 102,762,273 | PDZD7 | 3′-downstream | 4,806 |
| rs9405834 | 4.50E−04 | 0.220 | 0.944 | 0.325 | 6 | 5,402,826 | FARS2 | intron | 0 |
| rs10125981 | 4.52E−04 | 0.217 | 0.944 | 0.465 | 9 | 101,735,129 | STX17 | intron | 0 |
| rs2839025 | 4.53E−04 | 0.216 | 0.944 | 0.458 | 21 | 46,112,881 | PCBP3 | intron | 0 |
| rs633959 | 4.54E−04 | −0.217 | 0.944 | 0.388 | 23 | 120,477,514 | LOC158714 | 5′-upstream | 310,479 |
| rs17138853 | 4.54E−04 | 0.216 | 0.944 | 0.076 | 5 | 115,450,634 | COMMD10 | intron | 0 |
| rs1333165 | 4.55E−04 | 0.217 | 0.944 | 0.359 | 13 | 64,944,921 | STARP1 | 5′-upstream | 161,834 |
| rs6958942 | 4.55E−04 | 0.217 | 0.944 | 0.114 | 7 | 76,755,130 | CCDC146 | intron | 0 |
| rs3831958 | 4.55E−04 | 0.216 | 0.944 | 0.171 | 1 | 165,666,815 | CD247 | 3′-UTR | 0 |
| rs13066320 | 4.55E−04 | 0.216 | 0.944 | 0.209 | 3 | 117,888,889 | LOC285194 | 5′-upstream | 25,139 |
| rs10500136 | 4.58E−04 | 0.216 | 0.944 | 0.166 | 7 | 142,724,366 | CLCN1 | intron | 0 |
| rs17397776 | 4.58E−04 | 0.216 | 0.944 | 0.319 | 8 | 102,617,910 | GRHL2 | intron | 0 |
| rs16948648 | 4.58E−04 | 0.216 | 0.944 | 0.433 | 17 | 45,510,770 | ITGA3 | intron | 0 |
| rs3755913 | 4.59E−04 | −0.216 | 0.944 | 0.137 | 4 | 81,324,786 | PRDM8 | 5′-upstream | 662 |
| rs824725 | 4.60E−04 | 0.218 | 0.944 | 0.107 | 12 | 40,772,739 | GLT8D3 | intron | 0 |
| rs1286147 | 4.60E−04 | 0.217 | 0.944 | 0.261 | 14 | 90,537,320 | RPS6KA5 | intron | 0 |
| rs2718016 | 4.60E−04 | 0.217 | 0.944 | 0.162 | 7 | 36,051,448 | LOC100129326 | Unknown | — |
| rs9518363 | 4.62E−04 | 0.216 | 0.944 | 0.408 | 13 | 100,733,288 | NALCN | intron | 0 |
| rs12632003 | 4.65E−04 | 0.216 | 0.944 | 0.303 | 3 | 72,371,435 | RYBP | 3′-downstream | 135,003 |
| rs6688859 | 4.65E−04 | 0.216 | 0.944 | 0.229 | 1 | 113,577,284 | LOC643441 | 5′-upstream | 26,886 |
| rs12284447 | 4.66E−04 | −0.216 | 0.944 | 0.097 | 11 | 4,507,612 | OR52M2P | 5′-upstream | 13,782 |
| rs12347985 | 4.66E−04 | 0.216 | 0.944 | 0.203 | 9 | 3,392,590 | RFX3 | intron | 0 |
| rs1414402 | 4.66E−04 | 0.216 | 0.944 | 0.458 | 9 | 132,911,893 | LAMC3 | intron | 0 |
| rs10899613 | 4.67E−04 | 0.218 | 0.944 | 0.328 | 11 | 69,570,122 | LOC100127946 | 3′-downstream | 29,518 |
| rs6968662 | 4.67E−04 | 0.216 | 0.944 | 0.060 | 7 | 22,487,980 | MGC87042 | 3′-downstream | 4,967 |
| rs1603165 | 4.68E−04 | 0.217 | 0.944 | 0.158 | 4 | 135,308,676 | PABPC4L | 3′-downstream | 28,262 |
| rs11786195 | 4.68E−04 | 0.216 | 0.944 | 0.204 | 8 | 35,020,843 | LOC100133273 | 3′-downstream | 169,072 |
| rs9323262 | 4.69E−04 | 0.216 | 0.944 | 0.274 | 14 | 53,863,964 | CDKN3 | 5′-upstream | 69,512 |
| rs10849083 | 4.69E−04 | −0.217 | 0.944 | 0.185 | 12 | 4,512,833 | C12orf4 | intron | 0 |
| rs919676 | 4.70E−04 | 0.217 | 0.944 | 0.297 | 1 | 164,534,461 | LOC284685 | 5′-upstream | 20,954 |
| rs11022188 | 4.70E−04 | 0.216 | 0.944 | 0.129 | 11 | 12,105,328 | MICAL2 | intron | 0 |
| rs35749343 | 4.71E−04 | 0.216 | 0.944 | 0.056 | 17 | 39,942,006 | GPATCH8 | 5′-upstream | 5,678 |
| rs6474174 | 4.71E−04 | 0.217 | 0.944 | 0.078 | 8 | 39,718,465 | ADAM2 | 3′-downstream | 1,946 |
| rs17046758 | 4.74E−04 | −0.216 | 0.944 | 0.085 | 4 | 166,716,836 | LOC402191 | 5′-upstream | 25,192 |
| rs6489016 | 4.74E−04 | 0.216 | 0.944 | 0.428 | 12 | 124,862,044 | TMEM132B | 3′-downstream | 152,502 |
| rs7846979 | 4.75E−04 | 0.216 | 0.944 | 0.424 | 9 | 116,472,765 | LOC100129633 | intron | 0 |
| rs10886992 | 4.76E−04 | 0.216 | 0.944 | 0.316 | 10 | 123,572,169 | ATE1 | intron | 0 |
| rs246274 | 4.77E−04 | 0.216 | 0.944 | 0.125 | 5 | 122,308,702 | SNX24 | intron | 0 |
| rs647535 | 4.78E−04 | 0.217 | 0.944 | 0.418 | 18 | 57,295,795 | CDH20 | 5′-upstream | 12,960 |
| rs6669771 | 4.78E−04 | −0.218 | 0.944 | 0.064 | 1 | 114,421,345 | SYT6 | 3′-downstream | 12,092 |
| rs12145049 | 4.79E−04 | 0.215 | 0.944 | 0.114 | 1 | 202,044,364 | ZC3H11A | intron | 0 |
| rs6817351 | 4.79E−04 | 0.215 | 0.944 | 0.085 | 4 | 94,761,025 | GRID2 | intron | 0 |
| rs6842880 | 4.79E−04 | 0.215 | 0.944 | 0.085 | 4 | 94,761,175 | GRID2 | intron | 0 |
| rs7945719 | 4.79E−04 | −0.217 | 0.944 | 0.323 | 11 | 12,705,396 | TEAD1 | intron | 0 |
| rs12331869 | 4.80E−04 | 0.216 | 0.944 | 0.215 | 4 | 55,706,906 | KDR | 5′-upstream | 20,387 |
| rs12962880 | 4.80E−04 | −0.217 | 0.944 | 0.097 | 18 | 45,714,978 | MYO5B | intron | 0 |
| rs7814976 | 4.80E−04 | −0.221 | 0.944 | 0.334 | 8 | 141,306,070 | NIBP | intron | 0 |
| rs1873418 | 4.80E−04 | 0.215 | 0.944 | 0.087 | 3 | 195,146,719 | LOC100128023 | 3′-downstream | 46,724 |
| rs12889132 | 4.80E−04 | 0.215 | 0.944 | 0.076 | 14 | 51,826,994 | LOC100131689 | 3′-downstream | 1,177 |
| rs366178 | 4.80E−04 | −0.215 | 0.944 | 0.462 | 8 | 8,808,564 | MFHAS1 | 5′-upstream | 20,023 |
| rs400404 | 4.80E−04 | −0.215 | 0.944 | 0.462 | 8 | 8,807,864 | MFHAS1 | 5′-upstream | 19,323 |
| rs6788478 | 4.81E−04 | 0.217 | 0.944 | 0.354 | 3 | 104,561,945 | LOC644681 | 3′-downstream | 178,223 |
| rs17095581 | 4.81E−04 | 0.215 | 0.944 | 0.056 | 14 | 97,098,999 | LOC730217 | intron | 0 |
| rs10468282 | 4.81E−04 | 0.215 | 0.944 | 0.135 | 16 | 53,547,149 | LOC100132339 | 5′-upstream | 15,852 |
| rs8054030 | 4.81E−04 | 0.215 | 0.944 | 0.135 | 16 | 53,545,075 | LOC100132339 | 5′-upstream | 13,778 |
| rs2140501 | 4.82E−04 | 0.215 | 0.944 | 0.164 | 2 | 167,911,362 | hCG_1660379 | intron | 0 |
| rs1627784 | 4.82E−04 | 0.215 | 0.944 | 0.338 | 9 | 86,618,686 | NTRK2 | 3′-UTR | 0 |
| rs17138063 | 4.82E−04 | 0.216 | 0.944 | 0.181 | 17 | 32,264,382 | LOC727862 | 3′-downstream | 28,667 |
| rs2044002 | 4.85E−04 | −0.215 | 0.944 | 0.399 | 10 | 55,708,837 | PCDH15 | intron | 0 |
| rs9691998 | 4.86E−04 | 0.215 | 0.944 | 0.166 | 7 | 77,986,916 | MAGI2 | intron | 0 |
| rs2016644 | 4.86E−04 | 0.216 | 0.944 | 0.087 | 7 | 96,433,985 | FLJ34048 | 3′-downstream | 36,910 |
| rs10515468 | 4.87E−04 | 0.217 | 0.944 | 0.044 | 5 | 134,572,389 | H2AFY | 3′-downstream | 125,581 |
| rs4129316 | 4.88E−04 | 0.218 | 0.944 | 0.172 | 18 | 20,937,539 | ZNF521 | intron | 0 |
| rs6490281 | 4.88E−04 | 0.215 | 0.944 | 0.392 | 12 | 118,937,209 | CCDC64 | intron | 0 |
| rs2110586 | 4.88E−04 | 0.215 | 0.944 | 0.099 | 19 | 17,818,309 | JAK3 | intron | 0 |
| rs4645284 | 4.91E−04 | −0.215 | 0.944 | 0.469 | 4 | 118,614,104 | NT5C3P1 | 3′-downstream | 101,283 |
| rs838623 | 4.92E−04 | 0.215 | 0.944 | 0.148 | 3 | 144,671,616 | SLC9A9 | intron | 0 |
| rs3740873 | 4.92E−04 | −0.216 | 0.944 | 0.495 | 11 | 20,626,016 | SLC6A5 | intron | 0 |
| rs2047422 | 4.93E−04 | 0.215 | 0.944 | 0.350 | 1 | 55,484,555 | LOC645506 | 3′-downstream | 22,922 |
| rs7142654 | 4.94E−04 | 0.215 | 0.944 | 0.087 | 14 | 88,473,343 | TRNAA-AGC | 5′-upstream | 41,852 |
| rs7257500 | 4.95E−04 | 0.215 | 0.944 | 0.193 | 19 | 2,570,143 | GNG7 | intron | 0 |
| rs7182363 | 4.95E−04 | −0.215 | 0.944 | 0.184 | 15 | 68,295,072 | TLE3 | 5′-upstream | 117,762 |
| rs2836994 | 4.95E−04 | 0.216 | 0.944 | 0.311 | 21 | 39,669,876 | WRB | 5′-upstream | 4,264 |
| rs2815832 | 4.95E−04 | 0.215 | 0.944 | 0.168 | 1 | 202,883,863 | LRRN2 | intron | 0 |
| rs17734826 | 4.96E−04 | 0.216 | 0.944 | 0.071 | 12 | 113,473,404 | LOC652191 | 3′-downstream | 1,355 |
| rs33219 | 4.96E−04 | 0.218 | 0.944 | 0.165 | 12 | 30,960,265 | TSPAN11 | 5′-upstream | 10,840 |
| rs10202724 | 4.97E−04 | 0.216 | 0.944 | 0.364 | 2 | 222,880,471 | LOC440934 | 5′-upstream | 9,168 |
| rs13222164 | 4.97E−04 | 0.215 | 0.944 | 0.150 | 7 | 108,885,600 | LOC100128056 | 5′-upstream | 380,818 |
| rs6432096 | 4.97E−04 | 0.215 | 0.944 | 0.383 | 2 | 10,478,834 | HPCAL1 | intron | 0 |
| rs2581829 | 4.98E−04 | −0.215 | 0.944 | 0.430 | 3 | 53,108,566 | RFT1 | intron | 0 |
| rs10790715 | 4.99E−04 | 0.215 | 0.944 | 0.161 | 11 | 124,298,892 | HEPACAM | missense | 0 |
| rs488787 | 5.00E−04 | 0.215 | 0.944 | 0.293 | 9 | 202,908 | C9orf66 | 3′-downstream | 554 |
| rs7234341 | 5.00E−04 | 0.215 | 0.944 | 0.404 | 18 | 27,768,263 | KIAA1012 | intron | 0 |
| rs7234798 | 5.00E−04 | 0.215 | 0.944 | 0.404 | 18 | 27,731,401 | KIAA1012 | intron | 0 |
| rs838636 | 5.00E−04 | 0.215 | 0.944 | 0.146 | 3 | 144,681,492 | SLC9A9 | intron | 0 |
| rs838637 | 5.00E−04 | 0.215 | 0.944 | 0.146 | 3 | 144,681,741 | SLC9A9 | intron | 0 |
| rs9525149 | 5.00E−04 | 0.215 | 0.944 | 0.491 | 13 | 95,653,783 | HS6ST3 | intron | 0 |
| rs640520 | 5.01E−04 | −0.215 | 0.944 | 0.234 | 11 | 87,646,776 | CTSC | 3′-downstream | 19,632 |
| rs17139455 | 5.03E−04 | 0.215 | 0.944 | 0.078 | 5 | 115,668,006 | COMMD10 | 3′-downstream | 11,126 |
| rs17139495 | 5.03E−04 | 0.215 | 0.944 | 0.078 | 5 | 115,684,720 | COMMD10 | 3′-downstream | 27,840 |
| rs838622 | 5.03E−04 | 0.215 | 0.944 | 0.149 | 3 | 144,671,246 | SLC9A9 | intron | 0 |
| rs9549100 | 5.03E−04 | 0.215 | 0.944 | 0.177 | 13 | 39,666,838 | FOXO1 | 3′-downstream | 360,963 |
| rs7248157 | 5.04E−04 | 0.215 | 0.944 | 0.107 | 19 | 41,026,442 | NPHS1 | intron | 0 |
| rs6917844 | 5.04E−04 | 0.215 | 0.944 | 0.182 | 6 | 165,928,377 | PDE10A | intron | 0 |
| rs2836431 | 5.05E−04 | 0.215 | 0.944 | 0.113 | 21 | 38,771,787 | ERG | intron | 0 |
| rs134784 | 5.05E−04 | −0.215 | 0.944 | 0.224 | 22 | 25,982,075 | LOC100130624 | 5′-upstream | 368,531 |
| rs11033637 | 5.06E−04 | −0.215 | 0.944 | 0.464 | 11 | 36,436,365 | FLJ14213 | intron | 0 |
| rs1256066 | 5.07E−04 | 0.215 | 0.944 | 0.193 | 14 | 63,768,644 | ESR2 | intron | 0 |
| rs11700554 | 5.08E−04 | −0.215 | 0.944 | 0.435 | 21 | 46,203,918 | PCBP3 | 3′-downstream | 17,123 |
| rs7102231 | 5.09E−04 | 0.215 | 0.944 | 0.127 | 11 | 3,359,053 | LOC650368 | intron | 0 |
| rs7966890 | 5.09E−04 | 0.217 | 0.944 | 0.338 | 12 | 82,112,052 | LOC401725 | 3′-downstream | 41,952 |
| rs4786181 | 5.09E−04 | −0.215 | 0.944 | 0.399 | 16 | 7,535,679 | A2BP1 | intron | 0 |
| rs9635917 | 5.09E−04 | 0.214 | 0.944 | 0.363 | 18 | 27,760,837 | KIAA1012 | intron | 0 |
| rs1246386 | 5.10E−04 | 0.214 | 0.944 | 0.469 | 1 | 84,639,736 | DNASE2B | intron | 0 |
| rs1645519 | 5.10E−04 | −0.216 | 0.944 | 0.267 | 5 | 40,913,936 | CARD6 | 3′-downstream | 22,723 |
| rs5957355 | 5.10E−04 | 0.214 | 0.944 | 0.213 | 23 | 119,338,041 | FAM70A | 5′-upstream | 8,622 |
| rs6433815 | 5.11E−04 | −0.214 | 0.944 | 0.368 | 2 | 180,517,361 | KIAA1604 | 3′-downstream | 488 |
| rs17655198 | 5.15E−04 | 0.214 | 0.944 | 0.181 | 8 | 139,244,364 | FAM135B | intron | 0 |
| rs11726299 | 5.15E−04 | −0.214 | 0.944 | 0.190 | 4 | 183,556,319 | ODZ3 | intron | 0 |
| rs2930579 | 5.16E−04 | 0.214 | 0.944 | 0.238 | 15 | 23,392,329 | ATP10A | 3′-downstream | 82,623 |
| rs11004392 | 5.17E−04 | 0.220 | 0.944 | 0.091 | 10 | 56,002,909 | PCDH15 | intron | 0 |
| rs12277777 | 5.17E−04 | 0.214 | 0.944 | 0.097 | 11 | 21,891,492 | TMEM16E | 5′-upstream | 279,806 |
| rs10584 | 5.17E−04 | −0.215 | 0.944 | 0.469 | 15 | 88,129,555 | ANPEP | 3′-UTR | 0 |
| rs16847359 | 5.18E−04 | 0.214 | 0.944 | 0.056 | 2 | 163,379,281 | KCNH7 | intron | 0 |
| rs10735299 | 5.18E−04 | 0.214 | 0.944 | 0.058 | 12 | 76,695,128 | NAV3 | 5′-upstream | 54,072 |
| rs2249599 | 5.19E−04 | −0.215 | 0.944 | 0.251 | 21 | 36,281,873 | RPL23AP3 | 3′-downstream | 28,220 |
| rs2535358 | 5.19E−04 | 0.215 | 0.944 | 0.413 | 18 | 57,293,930 | CDH20 | 5′-upstream | 14,825 |
| rs363624 | 5.20E−04 | 0.214 | 0.944 | 0.487 | 2 | 74,784,350 | SEMA4F | 3′-downstream | 21,657 |
| rs323719 | 5.22E−04 | 0.214 | 0.944 | 0.132 | 1 | 43,043,515 | CCDC23 | 3′-downstream | 1,805 |
| rs4140770 | 5.22E−04 | 0.214 | 0.944 | 0.267 | 7 | 55,108,970 | EGFR | intron | 0 |
| rs3927807 | 5.23E−04 | −0.216 | 0.944 | 0.311 | 11 | 66,755,431 | FBXL11 | intron | 0 |
| rs12712919 | 5.24E−04 | 0.214 | 0.944 | 0.381 | 2 | 44,629,187 | C2orf34 | intron | 0 |
| rs139915 | 5.26E−04 | −0.214 | 0.944 | 0.395 | 22 | 39,050,909 | TNRC6B | 3′-UTR | 0 |
| rs4799933 | 5.26E−04 | −0.214 | 0.944 | 0.141 | 18 | 33,322,457 | BRUNOL4 | intron | 0 |
| rs3114316 | 5.27E−04 | −0.214 | 0.944 | 0.341 | 7 | 76,720,858 | CCDC146 | intron | 0 |
| rs17783784 | 5.27E−04 | −0.214 | 0.944 | 0.154 | 7 | 64,539,287 | ZNF92 | 3′-downstream | 35,854 |
| rs1994474 | 5.30E−04 | 0.214 | 0.944 | 0.397 | 12 | 26,838,121 | ITPR2 | intron | 0 |
| rs4461463 | 5.30E−04 | 0.214 | 0.944 | 0.054 | 3 | 152,201,282 | CLRN1 | 5′-upstream | 27,806 |
| rs966957 | 5.31E−04 | 0.214 | 0.944 | 0.305 | 17 | 47,543,904 | CA10 | intron | 0 |
| rs12921960 | 5.32E−04 | 0.214 | 0.944 | 0.209 | 16 | 6,639,750 | A2BP1 | intron | 0 |
| rs7695937 | 5.32E−04 | −0.214 | 0.944 | 0.284 | 4 | 7,584,470 | SORCS2 | intron | 0 |
| rs7026133 | 5.32E−04 | 0.214 | 0.944 | 0.130 | 9 | 32,409,461 | ACO1 | intron | 0 |
| rs658312 | 5.33E−04 | 0.215 | 0.944 | 0.163 | 11 | 78,308,368 | ODZ4 | 5′-upstream | 107,535 |
| rs271970 | 5.33E−04 | 0.214 | 0.944 | 0.373 | 20 | 57,469,557 | LOC645605 | intron | 0 |
| rs11771918 | 5.34E−04 | −0.214 | 0.944 | 0.153 | 7 | 64,538,847 | ZNF92 | 3′-downstream | 35,414 |
| rs2729076 | 5.35E−04 | 0.214 | 0.944 | 0.413 | 3 | 491,255 | CHL1 | 3′-downstream | 65,157 |
| rs3114314 | 5.36E−04 | −0.215 | 0.944 | 0.338 | 7 | 76,719,565 | CCDC146 | intron | 0 |
| rs2837002 | 5.36E−04 | 0.214 | 0.944 | 0.308 | 21 | 39,685,856 | WRB | intron | 0 |
| rs10124457 | 5.36E−04 | 0.214 | 0.944 | 0.400 | 9 | 2,338,820 | SMARCA2 | 3′-downstream | 155,196 |
| rs2226567 | 5.37E−04 | 0.214 | 0.944 | 0.179 | 11 | 105,186,923 | GRIA4 | intron | 0 |
| rs12612483 | 5.37E−04 | 0.214 | 0.944 | 0.193 | 2 | 122,770,055 | LOC728241 | 3′-downstream | 91,149 |
| rs1808458 | 5.39E−04 | 0.214 | 0.944 | 0.054 | 2 | 118,595,723 | INSIG2 | 3′-downstream | 11,656 |
| rs6948900 | 5.40E−04 | 0.214 | 0.944 | 0.289 | 7 | 40,585,520 | C7orf10 | intron | 0 |
| rs10088287 | 5.40E−04 | 0.216 | 0.944 | 0.068 | 8 | 39,718,049 | ADAM2 | 3′-downstream | 2,362 |
| rs11146187 | 5.41E−04 | 0.214 | 0.944 | 0.144 | 10 | 133,771,375 | JAKMIP3 | intron | 0 |
| rs2143361 | 5.41E−04 | 0.214 | 0.944 | 0.402 | 6 | 9,278,882 | LOC389365 | 3′-downstream | 79,742 |
| rs7749385 | 5.42E−04 | 0.214 | 0.944 | 0.346 | 6 | 141,350,701 | LOC729076 | 5′-upstream | 498,334 |
| rs7090540 | 5.43E−04 | −0.215 | 0.944 | 0.062 | 10 | 20,958,985 | NEBL | 3′-downstream | 151,108 |
| rs3776362 | 5.43E−04 | 0.214 | 0.944 | 0.080 | 5 | 142,379,050 | ARHGAP26 | intron | 0 |
| rs3934816 | 5.44E−04 | 0.213 | 0.944 | 0.399 | 11 | 11,488,807 | GALNTL4 | intron | 0 |
| rs857395 | 5.46E−04 | 0.214 | 0.944 | 0.433 | 10 | 55,759,269 | PCDH15 | intron | 0 |
| rs4606763 | 5.46E−04 | −0.213 | 0.944 | 0.220 | 17 | 6,017,765 | LOC100132641 | 3′-downstream | 41,585 |
| rs4357753 | 5.47E−04 | −0.213 | 0.944 | 0.141 | 12 | 111,225,092 | C12orf51 | intron | 0 |
| rs2331548 | 5.47E−04 | 0.213 | 0.944 | 0.101 | 4 | 170,199,179 | CBR4 | 5′-upstream | 31,182 |
| rs294782 | 5.48E−04 | −0.214 | 0.944 | 0.467 | 3 | 22,858,039 | LOC100129341 | 5′-upstream | 62,588 |
| rs2651460 | 5.48E−04 | 0.215 | 0.944 | 0.319 | 8 | 97,706,848 | SDC2 | 3′-downstream | 13,635 |
| rs7931660 | 5.49E−04 | 0.215 | 0.944 | 0.116 | 11 | 3,356,910 | ZNF195 | 5′-UTR | 0 |
| rs9577858 | 5.51E−04 | 0.213 | 0.944 | 0.359 | 13 | 112,549,621 | ATP11A | intron | 0 |
| rs6930152 | 5.52E−04 | 0.214 | 0.944 | 0.082 | 6 | 15,830,078 | DTNBP1 | 5′-upstream | 58,828 |
| rs4252718 | 5.53E−04 | 0.213 | 0.944 | 0.482 | 1 | 202,778,818 | MDM4 | intron | 0 |
| rs11081718 | 5.55E−04 | 0.213 | 0.944 | 0.404 | 18 | 27,670,175 | KIAA1012 | intron | 0 |
| rs10954975 | 5.56E−04 | 0.213 | 0.944 | 0.217 | 8 | 35,019,032 | LOC100133273 | 3′-downstream | 167,261 |
| rs6887101 | 5.59E−04 | 0.213 | 0.944 | 0.335 | 5 | 122,137,106 | SNX2 | 5′-upstream | 1,543 |
| rs1542188 | 5.60E−04 | 0.213 | 0.944 | 0.128 | 11 | 3,081,011 | OSBPL5 | intron | 0 |
| rs9418078 | 5.60E−04 | 0.213 | 0.944 | 0.168 | 10 | 34,077,361 | PARD3 | 3′-downstream | 362,742 |
| rs2131858 | 5.62E−04 | 0.213 | 0.944 | 0.350 | 8 | 104,397,766 | FZD6 | intron | 0 |
| rs2276433 | 5.63E−04 | 0.213 | 0.944 | 0.480 | 11 | 20,630,443 | SLC6A5 | synonymous | 0 |
| rs1935704 | 5.63E−04 | 0.213 | 0.944 | 0.184 | 6 | 112,828,477 | LOC643859 | 5′-upstream | 33,138 |
| rs12039454 | 5.64E−04 | 0.213 | 0.944 | 0.484 | 1 | 202,809,203 | MDM4 | 3′-downstream | 22,854 |
| rs2369244 | 5.64E−04 | 0.213 | 0.944 | 0.484 | 1 | 202,781,922 | MDM4 | intron | 0 |
| rs17758104 | 5.65E−04 | 0.213 | 0.944 | 0.052 | 12 | 106,551,651 | BTBD11 | intron | 0 |
| rs10902900 | 5.67E−04 | 0.213 | 0.944 | 0.199 | 10 | 124,954,909 | LOC100131719 | 3′-downstream | 14,586 |
| rs4702401 | 5.68E−04 | −0.216 | 0.944 | 0.319 | 5 | 6,961,507 | POLS | 3′-downstream | 151,346 |
| rs9423652 | 5.69E−04 | 0.213 | 0.944 | 0.078 | 10 | 5,590,140 | CALML3 | 3′-downstream | 31,915 |
| rs12143625 | 5.70E−04 | −0.213 | 0.944 | 0.347 | 1 | 213,458,463 | KCNK2 | intron | 0 |
| rs1471173 | 5.70E−04 | 0.213 | 0.944 | 0.134 | 4 | 170,199,727 | CBR4 | 5′-upstream | 31,730 |
| rs4845804 | 5.73E−04 | −0.213 | 0.944 | 0.353 | 1 | 5,568,030 | NPHP4 | 3′-downstream | 277,427 |
| rs9905727 | 5.73E−04 | 0.216 | 0.944 | 0.194 | 17 | 75,887,409 | KIAA1618 | intron | 0 |
| rs1108734 | 5.73E−04 | 0.213 | 0.944 | 0.112 | 1 | 43,050,829 | CCDC23 | intron | 0 |
| rs11210721 | 5.73E−04 | 0.213 | 0.944 | 0.112 | 1 | 43,044,833 | CCDC23 | 3′-downstream | 487 |
| rs10039039 | 5.76E−04 | −0.216 | 0.944 | 0.091 | 5 | 169,847,542 | KCNIP1 | intron | 0 |
| rs13185531 | 5.77E−04 | 0.212 | 0.944 | 0.137 | 5 | 40,179,748 | LOC285634 | 5′-upstream | 386,301 |
| rs11785367 | 5.77E−04 | 0.212 | 0.944 | 0.083 | 8 | 51,974,064 | LOC100128686 | 3′-downstream | 38,889 |
| rs2290854 | 5.77E−04 | 0.212 | 0.944 | 0.478 | 1 | 202,782,648 | MDM4 | intron | 0 |
| rs4951389 | 5.77E−04 | 0.212 | 0.944 | 0.478 | 1 | 202,742,457 | TRNAK-UUU | 3′-downstream | 324 |
| rs2812599 | 5.78E−04 | 0.212 | 0.944 | 0.191 | 10 | 34,080,299 | PARD3 | 3′-downstream | 359,804 |
| rs1361697 | 5.78E−04 | 0.212 | 0.944 | 0.134 | 6 | 22,676,513 | HDGFL1 | 5′-upstream | 1,144 |
| rs6942987 | 5.78E−04 | 0.212 | 0.944 | 0.101 | 7 | 50,996,595 | COBL | 3′-downstream | 54,809 |
| rs7337058 | 5.78E−04 | 0.213 | 0.944 | 0.360 | 13 | 112,550,584 | ATP11A | intron | 0 |
| rs1019287 | 5.79E−04 | 0.212 | 0.944 | 0.202 | 7 | 38,427,071 | AMPH | intron | 0 |
| rs6427140 | 5.81E−04 | 0.214 | 0.944 | 0.432 | 1 | 166,885,640 | DPT | 3′-downstream | 45,690 |
| rs7841539 | 5.82E−04 | −0.212 | 0.944 | 0.496 | 8 | 141,308,196 | NIBP | intron | 0 |
| rs10516562 | 5.82E−04 | 0.212 | 0.944 | 0.076 | 4 | 111,525,712 | LOC100133103 | 3′-downstream | 14,666 |
| rs1240375 | 5.83E−04 | 0.214 | 0.944 | 0.254 | 10 | 88,723,172 | KIAA1975 | intron | 0 |
| rs13061155 | 5.83E−04 | 0.212 | 0.944 | 0.417 | 3 | 60,244,988 | FHIT | intron | 0 |
| rs5003114 | 5.83E−04 | −0.213 | 0.944 | 0.198 | 1 | 166,727,502 | LOC730016 | 5′-upstream | 40,429 |
| rs6530113 | 5.84E−04 | −0.213 | 0.944 | 0.185 | 23 | 7,705,276 | VCX | 5′-upstream | 65,027 |
| rs7713342 | 5.86E−04 | 0.213 | 0.944 | 0.133 | 5 | 40,144,931 | LOC285634 | 5′-upstream | 351,484 |
| rs12895027 | 5.89E−04 | 0.213 | 0.944 | 0.055 | 14 | 51,812,394 | PTGDR | 3′-UTR | 0 |
| rs13239901 | 5.90E−04 | 0.213 | 0.944 | 0.232 | 7 | 108,935,937 | LOC100128056 | 5′-upstream | 330,481 |
| rs2078395 | 5.90E−04 | 0.213 | 0.944 | 0.443 | 4 | 181,140,358 | hCG_2025798 | 3′-downstream | 1,081,878 |
| rs1237375 | 5.91E−04 | −0.217 | 0.944 | 0.500 | 4 | 70,846,395 | CSN1S1 | intron | 0 |
| rs30045 | 5.92E−04 | 0.212 | 0.944 | 0.127 | 5 | 122,299,518 | SNX24 | intron | 0 |
| rs10245778 | 5.97E−04 | 0.212 | 0.944 | 0.097 | 7 | 142,460,269 | OR6V1 | missense | 0 |
| rs12668105 | 5.97E−04 | 0.212 | 0.944 | 0.097 | 7 | 142,470,988 | OR6W1P | Unknown | — |
| rs11126440 | 5.98E−04 | 0.212 | 0.944 | 0.486 | 2 | 74,782,998 | SEMA4F | 3′-downstream | 20,305 |
| rs363620 | 5.98E−04 | 0.212 | 0.944 | 0.486 | 2 | 74,781,858 | SEMA4F | 3′-downstream | 19,165 |
| rs2392518 | 5.98E−04 | 0.214 | 0.944 | 0.366 | 7 | 37,800,734 | GPR141 | 3′-downstream | 53,296 |
| rs12541355 | 5.99E−04 | −0.213 | 0.944 | 0.176 | 8 | 8,486,098 | CLDN23 | 5′-upstream | 110,978 |
| rs7707152 | 6.00E−04 | −0.212 | 0.944 | 0.191 | 5 | 112,004,964 | APC | 5′-upstream | 96,519 |
| rs11831152 | 6.01E−04 | 0.212 | 0.944 | 0.081 | 12 | 130,300,928 | LOC116437 | 3′-downstream | 42,704 |
| rs4878782 | 6.03E−04 | 0.213 | 0.944 | 0.203 | 9 | 38,293,243 | ALDH1B1 | 5′-upstream | 89,459 |
| rs2836999 | 6.03E−04 | 0.212 | 0.944 | 0.316 | 21 | 39,683,415 | WRB | intron | 0 |
| rs2817738 | 6.04E−04 | 0.212 | 0.944 | 0.426 | 6 | 24,899,079 | GMNN | 3′-downstream | 4,822 |
| rs378437 | 6.05E−04 | 0.212 | 0.944 | 0.103 | 1 | 55,782,058 | LOC645538 | 3′-downstream | 175,023 |
| rs6869344 | 6.07E−04 | 0.213 | 0.944 | 0.053 | 5 | 59,642,077 | LOC653198 | intron | 0 |
| rs1496412 | 6.07E−04 | −0.212 | 0.944 | 0.317 | 5 | 6,986,887 | POLS | 3′-downstream | 176,726 |
| rs4673021 | 6.08E−04 | 0.212 | 0.944 | 0.209 | 2 | 223,013,767 | SGPP2 | intron | 0 |
| rs4908425 | 6.09E−04 | 0.212 | 0.944 | 0.129 | 1 | 6,823,605 | CAMTA1 | intron | 0 |
| rs12154354 | 6.09E−04 | −0.212 | 0.944 | 0.149 | 7 | 64,540,471 | ZNF92 | 3′-downstream | 37,038 |
| rs4501594 | 6.10E−04 | 0.212 | 0.944 | 0.238 | 8 | 88,681,411 | LOC642461 | Unknown | — |
| rs2972588 | 6.11E−04 | 0.212 | 0.944 | 0.105 | 19 | 8,814,922 | MBD3L1 | missense | 0 |
| rs7149245 | 6.12E−04 | 0.212 | 0.944 | 0.260 | 14 | 53,863,105 | CDKN3 | 5′-upstream | 70,371 |
| rs17091512 | 6.13E−04 | 0.211 | 0.944 | 0.060 | 14 | 56,013,748 | C14orf101 | 5′-upstream | 102,516 |
| rs2101018 | 6.14E−04 | 0.211 | 0.944 | 0.199 | 4 | 136,714,417 | LOC100132574 | 5′-upstream | 6,655 |
| rs1327634 | 6.14E−04 | 0.211 | 0.944 | 0.489 | 13 | 95,659,303 | HS6ST3 | intron | 0 |
| rs2213325 | 6.15E−04 | 0.212 | 0.944 | 0.147 | 22 | 32,804,560 | LARGE | 5′-upstream | 158,150 |
| rs6743895 | 6.15E−04 | 0.211 | 0.944 | 0.090 | 2 | 56,106,433 | MIRN216B | 5′-upstream | 24,999 |
| rs555864 | 6.18E−04 | 0.211 | 0.944 | 0.052 | 5 | 178,745,813 | ADAMTS2 | 5′-upstream | 40,878 |
| rs2461935 | 6.19E−04 | 0.211 | 0.944 | 0.426 | 10 | 20,447,496 | PLXDC2 | intron | 0 |
| rs2021306 | 6.19E−04 | 0.212 | 0.944 | 0.165 | 9 | 14,355,307 | NFIB | 5′-upstream | 51,362 |
| rs4786182 | 6.19E−04 | −0.212 | 0.944 | 0.313 | 16 | 7,535,702 | A2BP1 | intron | 0 |
| rs1341936 | 6.20E−04 | −0.212 | 0.944 | 0.253 | 23 | 93,118,684 | LOC100127890 | 5′-upstream | 156,422 |
| rs11949163 | 6.20E−04 | 0.212 | 0.944 | 0.143 | 5 | 40,210,503 | LOC285634 | 5′-upstream | 417,056 |
| rs2837007 | 6.21E−04 | 0.211 | 0.944 | 0.347 | 21 | 39,694,354 | LCA5L | 3′-downstream | 5,286 |
| rs2814609 | 6.22E−04 | 0.211 | 0.944 | 0.108 | 1 | 189,872,382 | RGS18 | 5′-upstream | 521,833 |
| rs4918047 | 6.24E−04 | 0.211 | 0.944 | 0.081 | 10 | 105,512,330 | SH3PXD2A | intron | 0 |
| rs2839034 | 6.25E−04 | −0.212 | 0.944 | 0.411 | 21 | 46,121,013 | PCBP3 | intron | 0 |
| rs7895784 | 6.25E−04 | 0.211 | 0.944 | 0.471 | 10 | 18,912,878 | NSUN6 | intron | 0 |
| rs12624214 | 6.25E−04 | 0.211 | 0.944 | 0.128 | 2 | 124,348,173 | LOC100131284 | 3′-downstream | 111,052 |
| rs4951409 | 6.26E−04 | 0.211 | 0.944 | 0.475 | 1 | 202,827,613 | LRRN2 | 3′-downstream | 25,311 |
| rs7532236 | 6.26E−04 | 0.211 | 0.944 | 0.475 | 1 | 202,828,240 | LRRN2 | 3′-downstream | 24,684 |
| rs10489218 | 6.27E−04 | 0.212 | 0.944 | 0.458 | 1 | 166,883,302 | DPT | 3′-downstream | 48,028 |
| rs1501254 | 6.27E−04 | −0.212 | 0.944 | 0.445 | 17 | 47,435,164 | CA10 | intron | 0 |
| rs11240764 | 6.28E−04 | 0.211 | 0.944 | 0.475 | 1 | 202,835,999 | LRRN2 | 3′-downstream | 16,925 |
| rs10506537 | 6.30E−04 | 0.211 | 0.944 | 0.150 | 12 | 63,317,157 | RASSF3 | intron | 0 |
| rs6983491 | 6.30E−04 | 0.211 | 0.944 | 0.052 | 8 | 74,338,532 | LOC100130301 | intron | 0 |
| rs2296948 | 6.31E−04 | 0.211 | 0.944 | 0.415 | 9 | 116,473,506 | LOC100129633 | intron | 0 |
| rs5752014 | 6.31E−04 | −0.211 | 0.944 | 0.375 | 22 | 23,617,889 | SGSM1 | intron | 0 |
| rs9695517 | 6.31E−04 | 0.211 | 0.944 | 0.430 | 9 | 116,479,676 | LOC100129633 | intron | 0 |
| rs6853611 | 6.31E−04 | 0.211 | 0.944 | 0.480 | 4 | 70,951,620 | HTN1 | intron | 0 |
| rs2812669 | 6.33E−04 | 0.211 | 0.944 | 0.168 | 10 | 34,076,104 | PARD3 | 3′-downstream | 363,999 |
| rs6882373 | 6.37E−04 | 0.211 | 0.944 | 0.081 | 5 | 14,531,601 | TRIO | intron | 0 |
| rs352517 | 6.37E−04 | 0.211 | 0.944 | 0.267 | 6 | 69,250,301 | LOC728052 | 3′-downstream | 150,609 |
| rs7117175 | 6.37E−04 | 0.211 | 0.944 | 0.130 | 11 | 3,078,716 | OSBPL5 | intron | 0 |
| rs7680113 | 6.38E−04 | 0.211 | 0.944 | 0.284 | 4 | 188,911,848 | LOC644325 | 5′-upstream | 5,495 |
| rs7994097 | 6.40E−04 | 0.211 | 0.944 | 0.177 | 13 | 19,831,064 | CRYL1 | 3′-downstream | 44,742 |
| rs459552 | 6.40E−04 | 0.211 | 0.944 | 0.114 | 5 | 112,204,655 | APC | missense | 0 |
| rs478859 | 6.41E−04 | −0.211 | 0.944 | 0.363 | 1 | 100,568,708 | LOC646970 | 3′-downstream | 564 |
| rs587058 | 6.41E−04 | −0.211 | 0.944 | 0.363 | 1 | 100,568,472 | LOC646970 | 3′-downstream | 800 |
| rs11242152 | 6.41E−04 | 0.211 | 0.944 | 0.344 | 5 | 132,770,349 | FSTL4 | intron | 0 |
| rs6576425 | 6.41E−04 | −0.212 | 0.944 | 0.438 | 15 | 23,254,330 | UBE3A | 5′-upstream | 19,109 |
| rs2291787 | 6.42E−04 | 0.213 | 0.944 | 0.496 | 8 | 86,428,972 | CA1 | intron | 0 |
| rs2073925 | 6.43E−04 | −0.211 | 0.944 | 0.263 | 9 | 135,027,080 | GBGT1 | intron | 0 |
| rs246285 | 6.44E−04 | 0.216 | 0.944 | 0.179 | 5 | 122,272,867 | SNX24 | intron | 0 |
| rs1775416 | 6.44E−04 | −0.211 | 0.944 | 0.424 | 1 | 5,568,851 | NPHP4 | 3′-downstream | 276,606 |
| rs7280598 | 6.44E−04 | −0.211 | 0.944 | 0.285 | 21 | 36,295,016 | RPL23AP3 | 3′-downstream | 15,077 |
| rs183108 | 6.44E−04 | 0.211 | 0.944 | 0.082 | 6 | 54,418,757 | CLNS1B | 3′-downstream | 38,420 |
| rs1369802 | 6.44E−04 | 0.211 | 0.944 | 0.158 | 4 | 28,856,822 | LOC100131674 | 3′-downstream | 57,831 |
| rs869546 | 6.47E−04 | −0.211 | 0.944 | 0.110 | 3 | 190,999,791 | TP63 | intron | 0 |
| rs10225135 | 6.48E−04 | 0.211 | 0.944 | 0.264 | 7 | 107,965,198 | PNPLA8 | 5′-upstream | 11,324 |
| rs7144713 | 6.48E−04 | −0.212 | 0.944 | 0.130 | 14 | 56,969,647 | NAT12 | 3′-downstream | 22,011 |
| rs11040173 | 6.48E−04 | 0.214 | 0.944 | 0.227 | 11 | 5,965,954 | OR52L1 | 5′-upstream | 1,218 |
| rs16851729 | 6.48E−04 | −0.211 | 0.944 | 0.094 | 3 | 130,061,684 | LOC653712 | 3′-downstream | 1,357 |
| rs7044279 | 6.49E−04 | 0.212 | 0.944 | 0.084 | 9 | 111,009,022 | EPB41L4B | intron | 0 |
| rs9297854 | 6.49E−04 | −0.211 | 0.944 | 0.426 | 8 | 133,724,160 | LRRC6 | intron | 0 |
| rs6716756 | 6.49E−04 | 0.211 | 0.944 | 0.125 | 2 | 102,667,630 | SLC9A2 | intron | 0 |
| rs34823274 | 6.51E−04 | −0.211 | 0.944 | 0.118 | 4 | 14,805,437 | LOC100129903 | intron | 0 |
| rs7009336 | 6.51E−04 | 0.210 | 0.944 | 0.478 | 8 | 91,453,737 | TMEM64 | 3′-downstream | 251,041 |
| rs3828558 | 6.51E−04 | 0.210 | 0.944 | 0.251 | 4 | 24,143,785 | DHX15 | intron | 0 |
| rs11668269 | 6.52E−04 | 0.212 | 0.944 | 0.434 | 19 | 22,305,800 | LOC100130518 | 3′-downstream | 13,982 |
| rs1269683 | 6.53E−04 | 0.210 | 0.944 | 0.072 | 7 | 107,710,367 | NRCAM | intron | 0 |
| rs6672295 | 6.53E−04 | 0.211 | 0.944 | 0.162 | 1 | 50,487,276 | ELAVL4 | 3′-downstream | 47,628 |
| rs17171172 | 6.53E−04 | 0.210 | 0.944 | 0.222 | 7 | 37,839,175 | TXNDC3 | 5′-upstream | 15,549 |
| rs4776290 | 6.55E−04 | 0.211 | 0.944 | 0.433 | 15 | 64,279,015 | MEGF11 | intron | 0 |
| rs6594018 | 6.56E−04 | 0.210 | 0.944 | 0.478 | 1 | 202,834,775 | LRRN2 | 3′-downstream | 18,149 |
| rs1460040 | 6.56E−04 | 0.210 | 0.944 | 0.116 | 5 | 122,133,131 | SNX2 | 5′-upstream | 5,518 |
| rs1383415 | 6.56E−04 | −0.210 | 0.944 | 0.368 | 2 | 180,565,958 | KIAA1604 | intron | 0 |
| rs12208513 | 6.58E−04 | 0.210 | 0.944 | 0.063 | 6 | 154,942,722 | LOC646269 | 3′-downstream | 2,801 |
| rs7049523 | 6.58E−04 | 0.211 | 0.944 | 0.092 | 23 | 110,697,230 | LOC643873 | 3′-downstream | 51,040 |
| rs7162388 | 6.59E−04 | 0.210 | 0.944 | 0.090 | 15 | 59,117,034 | RORA | intron | 0 |
| rs2984610 | 6.60E−04 | 0.212 | 0.944 | 0.060 | 1 | 167,195,740 | LOC391132 | 3′-downstream | 20,884 |
| rs6785072 | 6.61E−04 | −0.210 | 0.944 | 0.464 | 3 | 198,552,075 | DLG1 | 5′-upstream | 42,231 |
| rs994377 | 6.62E−04 | 0.210 | 0.944 | 0.278 | 6 | 67,448,299 | NUFIP1P | 5′-upstream | 586,767 |
| rs2406623 | 6.63E−04 | 0.211 | 0.944 | 0.116 | 4 | 153,403,766 | FBXW7 | 3′-downstream | 58,094 |
| rs9833267 | 6.63E−04 | 0.211 | 0.944 | 0.353 | 3 | 104,553,964 | LOC644681 | 3′-downstream | 186,204 |
| rs4739296 | 6.64E−04 | 0.210 | 0.944 | 0.215 | 8 | 35,073,349 | LOC100133273 | 3′-downstream | 221,578 |
| rs12509760 | 6.64E−04 | 0.210 | 0.944 | 0.357 | 4 | 24,163,182 | DHX15 | intron | 0 |
| rs6103592 | 6.67E−04 | −0.210 | 0.944 | 0.101 | 20 | 42,124,801 | TOX2 | intron | 0 |
| rs3902916 | 6.68E−04 | 0.211 | 0.944 | 0.284 | 4 | 188,907,021 | LOC644325 | 5′-upstream | 668 |
| rs175154 | 6.68E−04 | 0.210 | 0.944 | 0.183 | 22 | 15,706,668 | ZNF402P | 3′-downstream | 11,358 |
| rs2387653 | 6.69E−04 | 0.212 | 0.944 | 0.130 | 10 | 1,397,826 | ADARB2 | intron | 0 |
| rs6977083 | 6.69E−04 | 0.210 | 0.944 | 0.412 | 7 | 70,893,394 | CALN1 | intron | 0 |
| rs17724918 | 6.70E−04 | −0.210 | 0.944 | 0.129 | 14 | 57,193,866 | LOC401777 | 3′-downstream | 26,706 |
| rs7626560 | 6.72E−04 | 0.210 | 0.944 | 0.101 | 3 | 12,450,088 | PPARG | intron | 0 |
| rs2251741 | 6.74E−04 | 0.210 | 0.944 | 0.449 | 8 | 95,227,558 | CDH17 | synonymous | 0 |
| rs12109819 | 6.76E−04 | −0.210 | 0.944 | 0.460 | 5 | 32,103,783 | PDZD2 | intron | 0 |
| rs11640308 | 6.77E−04 | 0.210 | 0.944 | 0.129 | 16 | 55,497,470 | SLC12A3 | intron | 0 |
| rs2111211 | 6.78E−04 | 0.210 | 0.944 | 0.314 | 12 | 107,563,998 | CORO1C | 3′-UTR | 0 |
| rs17632029 | 6.81E−04 | 0.212 | 0.944 | 0.072 | 10 | 14,008,131 | FRMD4A | intron | 0 |
| rs13263539 | 6.81E−04 | 0.210 | 0.944 | 0.292 | 8 | 102,613,455 | GRHL2 | intron | 0 |
| rs10906218 | 6.82E−04 | 0.211 | 0.944 | 0.103 | 10 | 12,837,291 | CAMK1D | intron | 0 |
| rs6421114 | 6.82E−04 | 0.212 | 0.944 | 0.172 | 23 | 113,479,287 | LOC100131029 | 5′-upstream | 50,581 |
| rs9495256 | 6.82E−04 | 0.210 | 0.944 | 0.165 | 6 | 139,161,606 | CCDC28A | 3′-downstream | 5,457 |
| rs9954037 | 6.82E−04 | −0.213 | 0.944 | 0.131 | 18 | 57,564,436 | RNF152 | 3′-downstream | 68,848 |
| rs3744950 | 6.83E−04 | −0.210 | 0.944 | 0.408 | 18 | 59,073,256 | BCL2 | intron | 0 |
| rs12656478 | 6.83E−04 | 0.210 | 0.944 | 0.078 | 5 | 123,769,231 | ZNF608 | 3′-downstream | 231,278 |
| rs10760699 | 6.84E−04 | 0.210 | 0.944 | 0.464 | 9 | 101,707,078 | LOC441461 | intron | 0 |
| rs1962051 | 6.84E−04 | 0.210 | 0.944 | 0.464 | 9 | 101,709,698 | STX17 | intron | 0 |
| rs4965473 | 6.84E−04 | 0.210 | 0.944 | 0.067 | 15 | 97,679,431 | LRRC28 | intron | 0 |
| rs4867950 | 6.85E−04 | −0.210 | 0.944 | 0.345 | 5 | 169,593,380 | LOC133874 | intron | 0 |
| rs2186184 | 6.85E−04 | −0.210 | 0.944 | 0.409 | 1 | 90,070,893 | LRRC8D | intron | 0 |
| rs9516644 | 6.86E−04 | 0.210 | 0.944 | 0.489 | 13 | 95,680,681 | HS6ST3 | intron | 0 |
| rs2291239 | 6.88E−04 | 0.210 | 0.944 | 0.262 | 18 | 27,698,809 | KIAA1012 | intron | 0 |
| rs11611384 | 6.88E−04 | 0.210 | 0.944 | 0.171 | 12 | 113,454,146 | LOC652191 | 5′-upstream | 17,697 |
| rs6679717 | 6.89E−04 | 0.212 | 0.944 | 0.480 | 1 | 202,827,300 | LRRN2 | 3′-downstream | 25,624 |
| rs1092656 | 6.92E−04 | −0.213 | 0.944 | 0.319 | 6 | 113,679,429 | LOC643884 | 3′-downstream | 27,462 |
| rs10260248 | 6.95E−04 | 0.209 | 0.944 | 0.083 | 7 | 142,629,853 | TAS2R40 | missense | 0 |
| rs2581790 | 6.96E−04 | 0.209 | 0.944 | 0.359 | 3 | 53,076,820 | LOC553148 | 5′-upstream | 2,579 |
| rs2258497 | 6.96E−04 | 0.209 | 0.944 | 0.181 | 1 | 165,674,697 | CD247 | intron | 0 |
| rs2744603 | 6.96E−04 | 0.209 | 0.944 | 0.267 | 6 | 24,645,844 | KIAA0319 | 3′-downstream | 6,467 |
| rs17741796 | 6.96E−04 | 0.210 | 0.944 | 0.256 | 18 | 27,663,317 | KIAA1012 | 3′-UTR | 0 |
| rs7199337 | 6.97E−04 | 0.211 | 0.944 | 0.318 | 16 | 77,356,261 | WWOX | intron | 0 |
| rs10447342 | 6.97E−04 | 0.209 | 0.944 | 0.049 | 6 | 92,809,429 | LOC100129847 | 5′-upstream | 226,577 |
| rs2900886 | 6.98E−04 | 0.209 | 0.944 | 0.067 | 1 | 167,182,814 | LOC391132 | 3′-downstream | 7,958 |
| rs2984605 | 6.98E−04 | 0.209 | 0.944 | 0.067 | 1 | 167,194,079 | LOC391132 | 3′-downstream | 19,223 |
| rs2984608 | 6.98E−04 | 0.209 | 0.944 | 0.067 | 1 | 167,195,678 | LOC391132 | 3′-downstream | 20,822 |
| rs2984609 | 6.98E−04 | 0.209 | 0.944 | 0.067 | 1 | 167,195,711 | LOC391132 | 3′-downstream | 20,855 |
| rs2984612 | 6.98E−04 | 0.209 | 0.944 | 0.067 | 1 | 167,199,277 | LOC391132 | 3′-downstream | 24,421 |
| rs3735478 | 6.99E−04 | −0.209 | 0.944 | 0.166 | 7 | 44,766,701 | ZMIZ2 | missense | 0 |
| rs552760 | 6.99E−04 | 0.210 | 0.944 | 0.174 | 1 | 205,099,462 | IL20 | 5′-upstream | 6,315 |
| rs11823077 | 6.99E−04 | −0.210 | 0.944 | 0.096 | 11 | 4,505,525 | OR52M2P | 5′-upstream | 11,695 |
| rs6986126 | 7.01E−04 | 0.210 | 0.944 | 0.105 | 8 | 14,335,688 | SGCZ | intron | 0 |
| rs7961254 | 7.01E−04 | 0.209 | 0.944 | 0.058 | 12 | 95,913,090 | NEDD1 | 3′-downstream | 42,918 |
| rs4505038 | 7.02E−04 | 0.211 | 0.944 | 0.327 | 11 | 45,891,273 | PEX16 | intron | 0 |
| rs7704817 | 7.04E−04 | −0.210 | 0.944 | 0.223 | 5 | 154,968,300 | LOC100131033 | 3′-downstream | 114,946 |
| rs9896168 | 7.04E−04 | −0.209 | 0.944 | 0.431 | 17 | 47,453,436 | CA10 | intron | 0 |
| rs12767108 | 7.04E−04 | 0.209 | 0.944 | 0.123 | 10 | 72,784,238 | SLC29A3 | intron | 0 |
| rs12428610 | 7.04E−04 | 0.210 | 0.944 | 0.491 | 13 | 95,663,351 | HS6ST3 | intron | 0 |
| rs1250220 | 7.04E−04 | −0.210 | 0.944 | 0.368 | 2 | 216,028,295 | FN1 | 5′-upstream | 19,259 |
| rs4954723 | 7.05E−04 | −0.209 | 0.944 | 0.273 | 2 | 139,285,272 | NXPH2 | 5′-upstream | 30,991 |
| rs1938969 | 7.05E−04 | 0.210 | 0.944 | 0.254 | 11 | 105,143,204 | GRIA4 | intron | 0 |
| rs5761495 | 7.06E−04 | 0.213 | 0.944 | 0.360 | 22 | 25,089,278 | SEZ6L | intron | 0 |
| rs5769445 | 7.06E−04 | 0.210 | 0.944 | 0.191 | 22 | 47,964,086 | LOC643653 | 3′-downstream | 31,701 |
| rs2836248 | 7.06E−04 | −0.210 | 0.944 | 0.465 | 21 | 38,551,067 | LOC441964 | intron | 0 |
| rs7406705 | 7.07E−04 | 0.210 | 0.944 | 0.306 | 17 | 77,222,329 | TSPAN10 | intron | 0 |
| rs10494791 | 7.09E−04 | 0.214 | 0.944 | 0.066 | 1 | 197,626,453 | LOC647202 | 3′-downstream | 2,227 |
| rs7813136 | 7.09E−04 | −0.210 | 0.944 | 0.218 | 8 | 136,444,840 | LOC286094 | 3′-downstream | 66,860 |
| rs17599976 | 7.09E−04 | 0.209 | 0.944 | 0.112 | 17 | 72,352,169 | MGAT5B | 5′-upstream | 24,224 |
| rs12247156 | 7.11E−04 | −0.209 | 0.944 | 0.155 | 10 | 34,675,535 | PARD3 | intron | 0 |
| rs2673942 | 7.12E−04 | −0.209 | 0.944 | 0.149 | 1 | 233,060,091 | PP2672 | 5′-upstream | 97,869 |
| rs16973748 | 7.12E−04 | 0.211 | 0.944 | 0.172 | 16 | 85,387,316 | LOC729979 | 3′-downstream | 58,383 |
| rs17416875 | 7.14E−04 | 0.209 | 0.944 | 0.060 | 7 | 39,662,643 | RALA | intron | 0 |
| rs3779203 | 7.14E−04 | 0.209 | 0.944 | 0.060 | 7 | 39,703,321 | RALA | intron | 0 |
| rs8090593 | 7.14E−04 | 0.209 | 0.944 | 0.356 | 18 | 69,667,938 | FBXO15 | 3′-downstream | 223,647 |
| rs11754612 | 7.14E−04 | 0.209 | 0.944 | 0.052 | 6 | 35,584,540 | TULP1 | intron | 0 |
| rs2032319 | 7.16E−04 | −0.209 | 0.944 | 0.283 | 21 | 36,295,592 | RPL23AP3 | 3′-downstream | 14,501 |
| rs560642 | 7.17E−04 | 0.209 | 0.944 | 0.319 | 12 | 24,338,715 | SOX5 | intron | 0 |
| rs2304733 | 7.18E−04 | 0.209 | 0.944 | 0.489 | 11 | 12,860,019 | TEAD1 | synonymous | 0 |
| rs1975174 | 7.18E−04 | 0.210 | 0.944 | 0.398 | 19 | 22,307,091 | LOC100130518 | 3′-downstream | 15,273 |
| rs2834652 | 7.20E−04 | 0.209 | 0.944 | 0.199 | 21 | 35,151,507 | RUNX1 | intron | 0 |
| rs6986563 | 7.21E−04 | −0.209 | 0.944 | 0.199 | 8 | 101,677,135 | SNX31 | intron | 0 |
| rs12495344 | 7.22E−04 | 0.209 | 0.944 | 0.177 | 3 | 192,516,457 | CCDC50 | 5′-upstream | 13,111 |
| rs17386017 | 7.22E−04 | 0.209 | 0.944 | 0.132 | 1 | 43,069,669 | ERMAP | intron | 0 |
| rs7198511 | 7.22E−04 | 0.209 | 0.944 | 0.287 | 16 | 77,355,868 | WWOX | intron | 0 |
| rs4075410 | 7.22E−04 | 0.209 | 0.944 | 0.446 | 8 | 144,300,478 | LY6H | 3′-downstream | 10,228 |
| rs6015519 | 7.23E−04 | −0.209 | 0.944 | 0.264 | 20 | 57,532,222 | LOC645605 | 5′-upstream | 28,985 |
| rs2514788 | 7.23E−04 | 0.209 | 0.944 | 0.453 | 8 | 95,212,920 | CDH17 | intron | 0 |
| rs655205 | 7.25E−04 | 0.209 | 0.944 | 0.478 | 1 | 189,766,343 | RGS18 | 5′-upstream | 627,872 |
| rs10483200 | 7.25E−04 | −0.209 | 0.944 | 0.097 | 22 | 38,565,782 | ENTHD1 | intron | 0 |
| rs1469569 | 7.28E−04 | −0.209 | 0.944 | 0.421 | 3 | 88,158,887 | CGGBP1 | 3′-downstream | 24,903 |
| rs7516412 | 7.28E−04 | −0.209 | 0.944 | 0.305 | 1 | 200,242,824 | ELF3 | 5′-upstream | 872 |
| rs788460 | 7.29E−04 | 0.210 | 0.944 | 0.209 | 12 | 28,041,539 | PTHLH | 5′-upstream | 25,356 |
| rs6100472 | 7.29E−04 | 0.209 | 0.944 | 0.359 | 20 | 57,523,252 | LOC645605 | 5′-upstream | 20,015 |
| rs17025231 | 7.31E−04 | −0.209 | 0.944 | 0.166 | 2 | 101,043,095 | TBC1D8 | intron | 0 |
| rs9893317 | 7.32E−04 | 0.209 | 0.944 | 0.299 | 17 | 47,547,164 | CA10 | intron | 0 |
| rs6427139 | 7.32E−04 | 0.210 | 0.944 | 0.442 | 1 | 166,879,549 | DPT | 3′-downstream | 51,781 |
| rs1918981 | 7.34E−04 | −0.209 | 0.944 | 0.184 | 23 | 7,702,055 | VCX | 5′-upstream | 68,248 |
| rs5980147 | 7.34E−04 | −0.209 | 0.944 | 0.184 | 23 | 7,701,717 | VCX | 5′-upstream | 68,586 |
| rs6863920 | 7.37E−04 | −0.209 | 0.944 | 0.304 | 5 | 59,898,210 | DEPDC1B | 3′-downstream | 30,286 |
| rs11100879 | 7.37E−04 | 0.208 | 0.944 | 0.173 | 4 | 146,579,862 | LOC100132841 | 5′-upstream | 16,745 |
| rs7588830 | 7.38E−04 | 0.208 | 0.944 | 0.464 | 2 | 215,985,917 | FN1 | intron | 0 |
| rs17049639 | 7.38E−04 | 0.209 | 0.944 | 0.200 | 3 | 8,858,603 | RAD18 | 3′-downstream | 37,957 |
| rs12223997 | 7.38E−04 | 0.208 | 0.944 | 0.374 | 11 | 7,570,138 | PPFIBP2 | intron | 0 |
| rs6862076 | 7.39E−04 | −0.209 | 0.944 | 0.435 | 5 | 32,105,667 | PDZD2 | intron | 0 |
| rs5949475 | 7.43E−04 | −0.210 | 0.944 | 0.070 | 23 | 93,699,769 | LOC780816 | 3′-downstream | 78,290 |
| rs12517436 | 7.44E−04 | 0.208 | 0.944 | 0.052 | 5 | 3,867,700 | IRX1 | 3′-downstream | 213,183 |
| rs2635462 | 7.45E−04 | −0.208 | 0.944 | 0.276 | 6 | 152,558,808 | SYNE1 | intron | 0 |
| rs1297214 | 7.46E−04 | 0.209 | 0.944 | 0.132 | 21 | 15,253,724 | NRIP1 | 3′-downstream | 1,703 |
| rs12819780 | 7.47E−04 | 0.208 | 0.944 | 0.265 | 12 | 29,659,102 | TMTC1 | intron | 0 |
| rs11796119 | 7.47E−04 | −0.208 | 0.944 | 0.217 | 23 | 9,996,515 | WWC3 | intron | 0 |
| rs266585 | 7.47E−04 | −0.208 | 0.944 | 0.134 | 5 | 64,364,371 | SDCCAG10 | 3′-downstream | 14,025 |
| rs11965065 | 7.48E−04 | 0.209 | 0.944 | 0.051 | 6 | 15,838,249 | DTNBP1 | 5′-upstream | 66,999 |
| rs12789020 | 7.48E−04 | 0.208 | 0.944 | 0.103 | 11 | 21,902,827 | TMEM16E | 5′-upstream | 268,471 |
| rs1861594 | 7.48E−04 | −0.208 | 0.944 | 0.397 | 12 | 15,552,237 | PTPRO | intron | 0 |
| rs16893606 | 7.49E−04 | 0.208 | 0.944 | 0.173 | 4 | 16,146,608 | LDB2 | intron | 0 |
| rs4330653 | 7.49E−04 | 0.208 | 0.944 | 0.079 | 8 | 10,483,001 | RP1L1 | 3′-downstream | 18,268 |
| rs1342782 | 7.50E−04 | 0.208 | 0.944 | 0.426 | 1 | 90,908,805 | BARHL2 | 3′-downstream | 41,362 |
| rs12814885 | 7.52E−04 | 0.209 | 0.944 | 0.089 | 12 | 7,396,641 | CD163L1 | 3′-downstream | 2,182 |
| rs1520459 | 7.52E−04 | 0.213 | 0.944 | 0.425 | 15 | 44,730,699 | SQRDL | 3′-downstream | 959,928 |
| rs6848945 | 7.53E−04 | 0.208 | 0.944 | 0.103 | 4 | 30,258,917 | PCDH7 | 5′-upstream | 72,218 |
| rs2047324 | 7.54E−04 | −0.208 | 0.944 | 0.327 | 5 | 6,958,147 | POLS | 3′-downstream | 147,986 |
| rs517480 | 7.54E−04 | −0.208 | 0.944 | 0.285 | 11 | 113,588,679 | ZBTB16 | intron | 0 |
| rs17056169 | 7.57E−04 | 0.208 | 0.944 | 0.072 | 9 | 72,806,374 | TRPM3 | intron | 0 |
| rs926571 | 7.57E−04 | −0.209 | 0.944 | 0.301 | 6 | 22,781,432 | LOC389370 | intron | 0 |
| rs8047997 | 7.58E−04 | −0.208 | 0.944 | 0.484 | 16 | 1,253,302 | TPSD1 | 3′-downstream | 4,807 |
| rs4758198 | 7.58E−04 | 0.209 | 0.944 | 0.365 | 11 | 7,556,800 | PPFIBP2 | intron | 0 |
| rs41535144 | 7.60E−04 | 0.208 | 0.944 | 0.129 | 14 | 53,626,570 | LOC645560 | 5′-upstream | 98,107 |
| rs4909741 | 7.60E−04 | 0.208 | 0.944 | 0.197 | 8 | 139,253,233 | FAM135B | intron | 0 |
| rs7750712 | 7.61E−04 | 0.208 | 0.944 | 0.134 | 6 | 22,673,295 | HDGFL1 | 5′-upstream | 4,362 |
| rs17066591 | 7.61E−04 | −0.208 | 0.944 | 0.354 | 5 | 165,645,102 | LOC441114 | 5′-upstream | 96,686 |
| rs9912487 | 7.62E−04 | 0.213 | 0.944 | 0.182 | 17 | 52,863,276 | MSI2 | intron | 0 |
| rs30038 | 7.63E−04 | 0.209 | 0.944 | 0.126 | 5 | 122,305,261 | SNX24 | intron | 0 |
| rs7221855 | 7.64E−04 | 0.208 | 0.944 | 0.310 | 17 | 72,176,449 | MXRA7 | 3′-downstream | 6,955 |
| rs1563828 | 7.65E−04 | 0.208 | 0.944 | 0.484 | 1 | 202,783,200 | MDM4 | intron | 0 |
| rs6676563 | 7.67E−04 | 0.208 | 0.944 | 0.440 | 1 | 55,425,571 | USP24 | intron | 0 |
| rs7536204 | 7.67E−04 | 0.208 | 0.944 | 0.440 | 1 | 55,439,065 | USP24 | intron | 0 |
| rs6992422 | 7.67E−04 | 0.208 | 0.944 | 0.365 | 8 | 5,054,945 | LOC648237 | 5′-upstream | 81,705 |
| rs3747961 | 7.68E−04 | 0.209 | 0.944 | 0.092 | 1 | 94,231,009 | ABCA4 | 3′-UTR | 0 |
| rs1462839 | 7.70E−04 | 0.208 | 0.944 | 0.128 | 10 | 67,626,355 | CTNNA3 | intron | 0 |
| rs16907660 | 7.70E−04 | 0.208 | 0.944 | 0.092 | 11 | 21,172,953 | NELL1 | intron | 0 |
| rs9828276 | 7.70E−04 | 0.208 | 0.944 | 0.361 | 3 | 144,723,903 | SLC9A9 | intron | 0 |
| rs7607788 | 7.71E−04 | 0.208 | 0.944 | 0.209 | 2 | 236,020,154 | CENTG2 | 5′-upstream | 47,321 |
| rs10503974 | 7.71E−04 | 0.208 | 0.944 | 0.218 | 8 | 35,018,934 | LOC100133273 | 3′-downstream | 167,163 |
| rs17030975 | 7.71E−04 | 0.208 | 0.944 | 0.329 | 3 | 53,743,245 | CACNA1D | intron | 0 |
| rs1795846 | 7.74E−04 | 0.208 | 0.944 | 0.444 | 12 | 53,175,347 | LOC100130272 | intron | 0 |
| rs1992044 | 7.75E−04 | 0.208 | 0.944 | 0.141 | 8 | 59,003,462 | FAM110B | 5′-upstream | 66,205 |
| rs1992045 | 7.75E−04 | 0.208 | 0.944 | 0.141 | 8 | 59,003,478 | FAM110B | 5′-upstream | 66,189 |
| rs9650192 | 7.75E−04 | 0.208 | 0.944 | 0.141 | 8 | 59,004,444 | FAM110B | 5′-upstream | 65,223 |
| rs8007000 | 7.75E−04 | 0.208 | 0.944 | 0.130 | 14 | 53,616,199 | LOC645560 | 5′-upstream | 87,736 |
| rs6095882 | 7.75E−04 | 0.208 | 0.944 | 0.060 | 20 | 48,409,292 | TRNAL-AAG | 5′-upstream | 23,462 |
| rs10094861 | 7.76E−04 | 0.208 | 0.944 | 0.192 | 8 | 1,254,336 | C8orf68 | 3′-downstream | 179,152 |
| rs12956309 | 7.77E−04 | 0.208 | 0.944 | 0.227 | 18 | 5,752,597 | LOC645355 | intron | 0 |
| rs10098626 | 7.78E−04 | 0.208 | 0.944 | 0.442 | 8 | 35,008,456 | LOC100133273 | 3′-downstream | 156,685 |
| rs10900596 | 7.79E−04 | 0.208 | 0.944 | 0.477 | 1 | 202,789,080 | MDM4 | 3′-downstream | 2,731 |
| rs595578 | 7.79E−04 | −0.208 | 0.944 | 0.098 | 18 | 75,554,323 | CTDP1 | intron | 0 |
| rs12776025 | 7.80E−04 | 0.209 | 0.944 | 0.168 | 10 | 27,279,372 | LOC100131912 | 5′-upstream | 6,019 |
| rs10519649 | 7.80E−04 | 0.208 | 0.944 | 0.419 | 4 | 143,674,003 | INPP4B | intron | 0 |
| rs11765212 | 7.80E−04 | 0.207 | 0.944 | 0.076 | 7 | 70,053,725 | AUTS2 | 3′-downstream | 157,935 |
| rs4758930 | 7.80E−04 | −0.207 | 0.944 | 0.085 | 12 | 132,088,232 | ZNF26 | intron | 0 |
| rs12450865 | 7.80E−04 | −0.207 | 0.944 | 0.403 | 17 | 47,525,332 | CA10 | intron | 0 |
| rs661322 | 7.81E−04 | 0.209 | 0.944 | 0.482 | 1 | 189,778,133 | RGS18 | 5′-upstream | 616,082 |
| rs7488840 | 7.82E−04 | 0.208 | 0.944 | 0.102 | 12 | 7,394,106 | CD163L1 | 3′-downstream | 4,717 |
| rs10900799 | 7.84E−04 | −0.207 | 0.944 | 0.327 | 5 | 130,567,627 | LYRM7 | 3′-UTR | 0 |
| rs10870249 | 7.84E−04 | 0.207 | 0.944 | 0.144 | 10 | 133,767,689 | JAKMIP3 | 5′-upstream | 614 |
| rs2040622 | 7.86E−04 | −0.207 | 0.944 | 0.161 | 17 | 65,096,191 | MAP2K6 | 3′-downstream | 46,126 |
| rs7161490 | 7.86E−04 | 0.207 | 0.944 | 0.105 | 14 | 95,083,965 | GLRX5 | 3′-downstream | 3,157 |
| rs2279700 | 7.87E−04 | 0.207 | 0.944 | 0.444 | 8 | 86,398,102 | CA13 | 3′-downstream | 14,548 |
| rs6530110 | 7.90E−04 | −0.208 | 0.944 | 0.184 | 23 | 7,704,976 | VCX | 5′-upstream | 65,327 |
| rs11608789 | 7.92E−04 | 0.207 | 0.944 | 0.085 | 12 | 106,998,822 | LOC100129448 | intron | 0 |
| rs4252685 | 7.92E−04 | 0.208 | 0.944 | 0.482 | 1 | 202,763,479 | MDM4 | intron | 0 |
| rs17147844 | 7.92E−04 | 0.207 | 0.944 | 0.074 | 4 | 70,813,132 | CSN1S1 | 5′-upstream | 18,256 |
| rs4951393 | 7.92E−04 | 0.210 | 0.944 | 0.487 | 1 | 202,756,180 | MDM4 | intron | 0 |
| rs4984643 | 7.93E−04 | 0.209 | 0.944 | 0.403 | 16 | 1,279,756 | LOC650474 | Unknown | — |
| rs2185416 | 7.93E−04 | 0.210 | 0.944 | 0.178 | 10 | 72,796,733 | SLC29A3 | 3′-downstream | 3,580 |
| rs9447086 | 7.93E−04 | −0.208 | 0.944 | 0.135 | 6 | 74,741,057 | CD109 | 3′-downstream | 146,296 |
| rs4353793 | 7.93E−04 | 0.208 | 0.944 | 0.056 | 3 | 1,756,437 | LOC391504 | 5′-upstream | 143,508 |
| rs7334785 | 7.94E−04 | 0.207 | 0.944 | 0.215 | 13 | 39,669,985 | FOXO1 | 3′-downstream | 357,816 |
| rs250851 | 7.94E−04 | 0.208 | 0.944 | 0.355 | 5 | 132,783,374 | FSTL4 | intron | 0 |
| rs619491 | 7.97E−04 | −0.209 | 0.944 | 0.305 | 5 | 40,920,837 | C7 | 5′-upstream | 24,519 |
| rs11762157 | 7.98E−04 | 0.207 | 0.944 | 0.394 | 7 | 17,066,715 | LOC100131425 | 3′-downstream | 171,675 |
| rs7526462 | 7.98E−04 | 0.208 | 0.944 | 0.065 | 1 | 167,176,576 | LOC391132 | 3′-downstream | 1,720 |
| rs1420741 | 7.98E−04 | 0.207 | 0.944 | 0.063 | 12 | 106,545,252 | BTBD11 | intron | 0 |
| rs10927390 | 7.98E−04 | 0.208 | 0.944 | 0.315 | 1 | 243,320,667 | EFCAB2 | 3′-downstream | 2,906 |
| rs6785333 | 8.00E−04 | −0.209 | 0.944 | 0.414 | 3 | 88,168,323 | CGGBP1 | 3′-downstream | 15,467 |
| rs1439566 | 8.01E−04 | 0.208 | 0.944 | 0.192 | 5 | 124,577,237 | LOC644659 | Unknown | — |
| rs2718019 | 8.05E−04 | 0.207 | 0.944 | 0.177 | 7 | 36,047,961 | LOC100129326 | Unknown | — |
| rs11027159 | 8.05E−04 | −0.207 | 0.944 | 0.384 | 11 | 23,318,586 | LOC100131557 | 3′-downstream | 29,676 |
| rs11151507 | 8.05E−04 | 0.207 | 0.944 | 0.162 | 18 | 65,277,685 | DOK6 | intron | 0 |
| rs10251707 | 8.06E−04 | 0.207 | 0.944 | 0.222 | 7 | 40,664,407 | C7orf10 | intron | 0 |
| rs1339408 | 8.06E−04 | −0.207 | 0.944 | 0.087 | 1 | 213,432,681 | KCNK2 | intron | 0 |
| rs6780419 | 8.07E−04 | 0.208 | 0.944 | 0.474 | 3 | 191,539,016 | CLDN1 | 5′-upstream | 16,107 |
| rs4304370 | 8.07E−04 | −0.207 | 0.944 | 0.301 | 9 | 1,725,187 | SMARCA2 | 5′-upstream | 280,155 |
| rs6488556 | 8.07E−04 | 0.207 | 0.944 | 0.256 | 12 | 12,818,994 | MIRN613 | 3′-downstream | 10,050 |
| rs7390284 | 8.07E−04 | 0.207 | 0.944 | 0.359 | 9 | 18,379,998 | ADAMTSL1 | 5′-upstream | 84,106 |
| rs11052699 | 8.07E−04 | 0.207 | 0.944 | 0.092 | 12 | 7,395,698 | CD163L1 | 3′-downstream | 3,125 |
| rs6517434 | 8.07E−04 | 0.207 | 0.944 | 0.090 | 21 | 38,109,926 | KCNJ6 | intron | 0 |
| rs41434745 | 8.08E−04 | −0.207 | 0.944 | 0.397 | 1 | 205,098,415 | IL20 | 5′-upstream | 7,362 |
| rs26147 | 8.08E−04 | 0.211 | 0.944 | 0.396 | 5 | 11,535,412 | CTNND2 | intron | 0 |
| rs2132482 | 8.08E−04 | 0.207 | 0.944 | 0.065 | 11 | 43,231,114 | LOC399881 | 3′-downstream | 8,516 |
| rs636811 | 8.09E−04 | 0.207 | 0.944 | 0.090 | 1 | 231,815,644 | KCNK1 | 5′-upstream | 729 |
| rs7677823 | 8.10E−04 | −0.207 | 0.944 | 0.458 | 4 | 160,416,083 | RAPGEF2 | intron | 0 |
| rs9910052 | 8.10E−04 | 0.207 | 0.944 | 0.323 | 17 | 72,180,396 | MXRA7 | 3′-downstream | 3,008 |
| rs17423995 | 8.11E−04 | 0.207 | 0.944 | 0.051 | 4 | 37,416,631 | RELL1 | 5′-upstream | 52,237 |
| rs2969208 | 8.12E−04 | −0.207 | 0.944 | 0.116 | 17 | 11,279,499 | FLJ45455 | intron | 0 |
| rs9525158 | 8.12E−04 | 0.207 | 0.944 | 0.489 | 13 | 95,675,242 | HS6ST3 | intron | 0 |
| rs332433 | 8.13E−04 | −0.207 | 0.944 | 0.235 | 11 | 36,677,824 | C11orf74 | 3′-downstream | 40,431 |
| rs332434 | 8.13E−04 | −0.207 | 0.944 | 0.235 | 11 | 36,677,733 | C11orf74 | 3′-downstream | 40,340 |
| rs11055065 | 8.13E−04 | 0.210 | 0.944 | 0.224 | 12 | 12,825,721 | APOLD1 | 5′-upstream | 4,161 |
| rs10900594 | 8.14E−04 | 0.207 | 0.944 | 0.478 | 1 | 202,736,752 | TRNAK-UUU | 5′-upstream | 5,526 |
| rs1265181 | 8.16E−04 | 0.207 | 0.944 | 0.123 | 6 | 31,263,764 | HCG27 | 5′-upstream | 9,814 |
| rs776858 | 8.16E−04 | 0.207 | 0.944 | 0.495 | 4 | 70,860,054 | CSN2 | intron | 0 |
| rs6713893 | 8.18E−04 | 0.207 | 0.944 | 0.119 | 2 | 2,649,185 | MYT1L | 5′-upstream | 335,133 |
| rs13028443 | 8.19E−04 | 0.207 | 0.944 | 0.460 | 2 | 63,457,221 | LOC51057 | intron | 0 |
| rs993794 | 8.19E−04 | −0.207 | 0.944 | 0.159 | 10 | 108,081,693 | LOC100132397 | 5′-upstream | 200,523 |
| rs1884452 | 8.21E−04 | 0.209 | 0.944 | 0.048 | 23 | 145,413,585 | LOC100128690 | 5′-upstream | 22,445 |
| rs11651547 | 8.21E−04 | 0.207 | 0.944 | 0.119 | 17 | 39,376,774 | PYY | 3′-downstream | 8,858 |
| rs1418561 | 8.22E−04 | 0.209 | 0.944 | 0.182 | 6 | 112,809,914 | LOC643859 | 5′-upstream | 14,575 |
| rs185009 | 8.24E−04 | 0.207 | 0.944 | 0.126 | 5 | 122,301,666 | SNX24 | intron | 0 |
| rs246287 | 8.24E−04 | 0.207 | 0.944 | 0.126 | 5 | 122,273,325 | SNX24 | intron | 0 |
| rs246317 | 8.24E−04 | 0.207 | 0.944 | 0.126 | 5 | 122,337,759 | SNX24 | intron | 0 |
| rs246322 | 8.24E−04 | 0.207 | 0.944 | 0.126 | 5 | 122,335,168 | SNX24 | intron | 0 |
| rs30029 | 8.24E−04 | 0.207 | 0.944 | 0.126 | 5 | 122,311,069 | SNX24 | intron | 0 |
| rs30039 | 8.24E−04 | 0.207 | 0.944 | 0.126 | 5 | 122,305,078 | SNX24 | intron | 0 |
| rs30049 | 8.24E−04 | 0.207 | 0.944 | 0.126 | 5 | 122,333,996 | SNX24 | intron | 0 |
| rs431878 | 8.24E−04 | 0.207 | 0.944 | 0.126 | 5 | 122,289,690 | SNX24 | intron | 0 |
| rs1927807 | 8.25E−04 | 0.207 | 0.944 | 0.487 | 13 | 95,672,758 | HS6ST3 | intron | 0 |
| rs11248542 | 8.25E−04 | 0.207 | 0.944 | 0.129 | 10 | 125,218,584 | GPR26 | 5′-upstream | 197,277 |
| rs1496533 | 8.25E−04 | 0.207 | 0.944 | 0.496 | 8 | 86,420,726 | CA1 | 3′-downstream | 6,983 |
| rs701556 | 8.28E−04 | −0.206 | 0.944 | 0.238 | 13 | 102,406,882 | LOC121952 | 3′-downstream | 59,838 |
| rs1391240 | 8.28E−04 | −0.208 | 0.944 | 0.498 | 6 | 164,885,480 | LOC728275 | 3′-downstream | 206,903 |
| rs10985459 | 8.28E−04 | −0.208 | 0.944 | 0.166 | 9 | 123,743,153 | TTLL11 | 3′-downstream | 47,857 |
| rs2850377 | 8.29E−04 | 0.206 | 0.944 | 0.338 | 4 | 103,131,182 | BANK1 | intron | 0 |
| rs2850378 | 8.29E−04 | 0.206 | 0.944 | 0.338 | 4 | 103,134,990 | BANK1 | intron | 0 |
| rs10485526 | 8.30E−04 | −0.206 | 0.944 | 0.361 | 20 | 15,383,203 | MACROD2 | intron | 0 |
| rs11015427 | 8.30E−04 | 0.206 | 0.944 | 0.173 | 10 | 27,291,815 | LOC100131912 | 5′-upstream | 18,462 |
| rs11015433 | 8.30E−04 | 0.206 | 0.944 | 0.173 | 10 | 27,296,142 | LOC100131912 | 5′-upstream | 22,789 |
| rs6488592 | 8.32E−04 | 0.206 | 0.944 | 0.121 | 12 | 7,739,894 | GDF3 | 5′-upstream | 267 |
| rs9292290 | 8.33E−04 | −0.207 | 0.944 | 0.149 | 5 | 28,755,133 | LOC729862 | 5′-upstream | 206,329 |
| rs7332561 | 8.34E−04 | 0.208 | 0.944 | 0.201 | 13 | 28,860,516 | KIAA0774 | intron | 0 |
| rs4659221 | 8.35E−04 | −0.206 | 0.944 | 0.274 | 1 | 119,969,876 | LOC100130667 | intron | 0 |
| rs10503973 | 8.36E−04 | 0.207 | 0.944 | 0.219 | 8 | 35,018,804 | LOC100133273 | 3′-downstream | 167,033 |
| rs10993511 | 8.37E−04 | 0.206 | 0.944 | 0.078 | 9 | 92,259,202 | LOC340515 | 3′-downstream | 5,332 |
| rs12022129 | 8.37E−04 | 0.206 | 0.944 | 0.394 | 1 | 205,069,997 | IL19 | intron | 0 |
| rs2124761 | 8.39E−04 | 0.207 | 0.944 | 0.053 | 11 | 100,441,556 | PGR | intron | 0 |
| rs4964848 | 8.39E−04 | 0.207 | 0.944 | 0.199 | 12 | 128,198,132 | TMEM132D | intron | 0 |
| rs6745008 | 8.41E−04 | −0.206 | 0.944 | 0.233 | 2 | 230,069,842 | DNER | intron | 0 |
| rs363625 | 8.41E−04 | 0.206 | 0.944 | 0.489 | 2 | 74,784,407 | SEMA4F | 3′-downstream | 21,714 |
| rs17154379 | 8.42E−04 | −0.207 | 0.944 | 0.185 | 8 | 8,506,621 | CLDN23 | 5′-upstream | 90,455 |
| rs3015224 | 8.43E−04 | −0.206 | 0.944 | 0.309 | 9 | 71,918,208 | MAMDC2 | intron | 0 |
| rs6436753 | 8.44E−04 | −0.206 | 0.944 | 0.222 | 2 | 228,703,258 | SPHKAP | intron | 0 |
| rs2717329 | 8.44E−04 | 0.207 | 0.944 | 0.471 | 7 | 19,029,300 | HDAC9 | 3′-downstream | 25,782 |
| rs13275618 | 8.46E−04 | −0.207 | 0.944 | 0.382 | 8 | 141,298,387 | NIBP | intron | 0 |
| rs2837008 | 8.49E−04 | 0.206 | 0.944 | 0.305 | 21 | 39,695,357 | LCA5L | 3′-downstream | 4,283 |
| rs9392684 | 8.49E−04 | 0.208 | 0.944 | 0.434 | 6 | 5,371,264 | FARS2 | intron | 0 |
| rs7545496 | 8.50E−04 | −0.206 | 0.944 | 0.087 | 1 | 190,777,580 | RGS1 | 5′-upstream | 33,900 |
| rs897405 | 8.52E−04 | 0.206 | 0.944 | 0.338 | 6 | 68,867,259 | LOC100128293 | 3′-downstream | 165,964 |
| rs10831908 | 8.53E−04 | −0.208 | 0.944 | 0.291 | 11 | 12,768,995 | TEAD1 | intron | 0 |
| rs2581830 | 8.53E−04 | −0.207 | 0.944 | 0.429 | 3 | 53,109,138 | RFT1 | intron | 0 |
| rs12701867 | 8.54E−04 | −0.208 | 0.944 | 0.114 | 7 | 41,197,194 | C7orf10 | 3′-downstream | 330,312 |
| rs13428208 | 8.54E−04 | 0.206 | 0.944 | 0.051 | 2 | 128,744,772 | HS6ST1 | intron | 0 |
| rs17814562 | 8.55E−04 | 0.206 | 0.944 | 0.054 | 12 | 106,553,998 | BTBD11 | intron | 0 |
| rs12417886 | 8.59E−04 | −0.206 | 0.944 | 0.291 | 11 | 12,768,307 | TEAD1 | intron | 0 |
| rs12607786 | 8.59E−04 | 0.206 | 0.944 | 0.264 | 18 | 27,672,175 | KIAA1012 | intron | 0 |
| rs437168 | 8.60E−04 | 0.206 | 0.944 | 0.225 | 19 | 41,026,259 | NPHS1 | synonymous | 0 |
| rs1392934 | 8.64E−04 | −0.206 | 0.944 | 0.417 | 17 | 47,425,763 | CA10 | intron | 0 |
| rs11674390 | 8.64E−04 | 0.206 | 0.944 | 0.074 | 2 | 45,258,664 | SIX2 | 5′-upstream | 168,638 |
| rs4758578 | 8.64E−04 | 0.206 | 0.944 | 0.078 | 11 | 3,354,643 | ZNF195 | intron | 0 |
| rs2663989 | 8.67E−04 | 0.206 | 0.944 | 0.143 | 15 | 42,325,952 | CASC4 | 5′-upstream | 42,269 |
| rs828560 | 8.69E−04 | 0.206 | 0.944 | 0.374 | 6 | 165,929,442 | PDE10A | intron | 0 |
| rs4909747 | 8.69E−04 | 0.206 | 0.944 | 0.191 | 8 | 139,260,139 | FAM135B | intron | 0 |
| rs6682208 | 8.70E−04 | 0.206 | 0.944 | 0.478 | 1 | 202,832,806 | LRRN2 | 3′-downstream | 20,118 |
| rs2252505 | 8.73E−04 | −0.206 | 0.944 | 0.400 | 5 | 10,292,278 | LOC134145 | synonymous | 0 |
| rs875521 | 8.73E−04 | 0.206 | 0.944 | 0.219 | 3 | 124,992,028 | MYLK | intron | 0 |
| rs11629141 | 8.75E−04 | 0.206 | 0.944 | 0.125 | 14 | 96,597,819 | LOC730133 | 3′-downstream | 116,339 |
| rs3820439 | 8.77E−04 | −0.206 | 0.944 | 0.319 | 1 | 200,240,188 | RNPEP | missense | 0 |
| rs17023187 | 8.77E−04 | 0.206 | 0.944 | 0.045 | 4 | 148,542,379 | LOC100130537 | 3′-downstream | 24,197 |
| rs6460688 | 8.78E−04 | 0.207 | 0.944 | 0.431 | 7 | 70,893,725 | CALN1 | intron | 0 |
| rs1981828 | 8.80E−04 | −0.205 | 0.944 | 0.388 | 19 | 38,468,052 | CEBPA | 3′-downstream | 14,724 |
| rs17742014 | 8.80E−04 | 0.205 | 0.944 | 0.262 | 18 | 27,728,798 | KIAA1012 | intron | 0 |
| rs1494826 | 8.80E−04 | 0.207 | 0.944 | 0.134 | 5 | 116,553,682 | LOC728342 | 5′-upstream | 225,422 |
| rs7120935 | 8.81E−04 | 0.208 | 0.944 | 0.270 | 11 | 21,036,518 | NELL1 | intron | 0 |
| rs4951407 | 8.84E−04 | 0.206 | 0.944 | 0.489 | 1 | 202,824,693 | LRRN2 | 3′-downstream | 28,231 |
| rs560279 | 8.84E−04 | 0.205 | 0.944 | 0.352 | 11 | 121,528,721 | MIRNLET7A2 | 5′-upstream | 6,210 |
| rs10954881 | 8.85E−04 | 0.205 | 0.944 | 0.406 | 8 | 30,134,238 | DCTN6 | intron | 0 |
| rs3935679 | 8.86E−04 | 0.205 | 0.944 | 0.334 | 5 | 97,504,067 | PSME2P1 | 5′-upstream | 70,679 |
| rs12882488 | 8.87E−04 | 0.205 | 0.944 | 0.079 | 14 | 94,950,974 | C14orf49 | 3′-downstream | 2,610 |
| rs16865402 | 8.89E−04 | 0.205 | 0.944 | 0.081 | 3 | 191,536,737 | CLDN1 | 5′-upstream | 13,828 |
| rs6789805 | 8.89E−04 | 0.205 | 0.944 | 0.081 | 3 | 191,537,787 | CLDN1 | 5′-upstream | 14,878 |
| rs6801146 | 8.89E−04 | 0.205 | 0.944 | 0.081 | 3 | 191,537,856 | CLDN1 | 5′-upstream | 14,947 |
| rs17152434 | 8.89E−04 | 0.208 | 0.944 | 0.199 | 5 | 124,579,475 | LOC644659 | Unknown | — |
| rs9590371 | 8.90E−04 | −0.209 | 0.944 | 0.356 | 13 | 95,658,077 | HS6ST3 | intron | 0 |
| rs6969427 | 8.91E−04 | 0.208 | 0.944 | 0.333 | 7 | 152,673,499 | FLJ42291 | 3′-downstream | 40,448 |
| rs12815715 | 8.92E−04 | 0.206 | 0.944 | 0.093 | 12 | 7,380,620 | ACSM4 | 3′-downstream | 8,384 |
| rs10808355 | 8.92E−04 | 0.205 | 0.944 | 0.412 | 8 | 98,455,047 | TSPYL5 | 5′-upstream | 95,695 |
| rs10868721 | 8.93E−04 | 0.207 | 0.944 | 0.062 | 9 | 89,974,849 | LOC389768 | 5′-upstream | 6,279 |
| rs1882055 | 8.94E−04 | 0.205 | 0.944 | 0.153 | 7 | 36,062,475 | LOC100129326 | Unknown | — |
| rs10205005 | 8.94E−04 | 0.205 | 0.944 | 0.132 | 2 | 42,102,217 | LOC91461 | 5′-upstream | 27,040 |
| rs9599779 | 8.94E−04 | 0.208 | 0.944 | 0.246 | 13 | 70,578,186 | DACH1 | 3′-downstream | 331,913 |
| rs4451621 | 8.94E−04 | 0.205 | 0.944 | 0.412 | 10 | 12,471,373 | CAMK1D | intron | 0 |
| rs3217916 | 8.95E−04 | 0.206 | 0.944 | 0.397 | 12 | 4,278,934 | CCND2 | intron | 0 |
| rs2535357 | 8.95E−04 | 0.205 | 0.944 | 0.431 | 18 | 57,293,491 | CDH20 | 5′-upstream | 15,264 |
| rs6560606 | 8.96E−04 | 0.205 | 0.944 | 0.236 | 9 | 79,352,869 | GNA14 | intron | 0 |
| rs11997340 | 8.97E−04 | −0.206 | 0.944 | 0.388 | 8 | 3,822,506 | CSMD1 | intron | 0 |
| rs6770415 | 8.98E−04 | −0.205 | 0.944 | 0.255 | 3 | 13,242,771 | NUP210 | 3′-downstream | 89,966 |
| rs4304369 | 8.98E−04 | −0.207 | 0.944 | 0.335 | 9 | 1,725,113 | SMARCA2 | 5′-upstream | 280,229 |
| rs10900597 | 8.99E−04 | 0.205 | 0.944 | 0.500 | 1 | 202,789,112 | MDM4 | 3′-downstream | 2,763 |
| rs13152233 | 9.01E−04 | 0.205 | 0.944 | 0.072 | 4 | 143,296,300 | INPP4B | intron | 0 |
| rs8070612 | 9.01E−04 | 0.205 | 0.944 | 0.056 | 17 | 18,840,335 | SLC5A10 | intron | 0 |
| rs12474898 | 9.01E−04 | 0.205 | 0.944 | 0.199 | 2 | 236,032,106 | CENTG2 | 5′-upstream | 35,369 |
| rs7279643 | 9.02E−04 | −0.205 | 0.944 | 0.424 | 21 | 46,200,229 | PCBP3 | 3′-downstream | 13,434 |
| rs6959836 | 9.04E−04 | −0.205 | 0.944 | 0.112 | 7 | 5,065,893 | RBAK | intron | 0 |
| rs12702227 | 9.04E−04 | 0.205 | 0.944 | 0.052 | 7 | 46,519,896 | LOC730338 | 3′-downstream | 175,480 |
| rs12702228 | 9.04E−04 | 0.205 | 0.944 | 0.052 | 7 | 46,522,406 | LOC730338 | 3′-downstream | 172,970 |
| rs1265178 | 9.06E−04 | 0.205 | 0.944 | 0.120 | 6 | 31,269,208 | HCG27 | 5′-upstream | 4,370 |
| rs920499 | 9.09E−04 | −0.205 | 0.944 | 0.366 | 14 | 104,437,544 | KIAA0284 | 3′-downstream | 3,412 |
| rs5985614 | 9.09E−04 | 0.205 | 0.944 | 0.235 | 23 | 110,696,134 | LOC643873 | 3′-downstream | 52,136 |
| rs1684149 | 9.11E−04 | 0.205 | 0.944 | 0.187 | 12 | 32,162,607 | BICD1 | intron | 0 |
| rs17708126 | 9.11E−04 | 0.205 | 0.944 | 0.056 | 7 | 14,221,389 | DGKB | intron | 0 |
| rs2018445 | 9.12E−04 | 0.211 | 0.944 | 0.268 | 11 | 43,117,351 | LOC100128134 | 5′-upstream | 62,408 |
| rs1798 | 9.12E−04 | 0.205 | 0.944 | 0.195 | 1 | 205,082,748 | IL19 | 3′-UTR | 0 |
| rs7524812 | 9.12E−04 | 0.205 | 0.944 | 0.081 | 1 | 97,342,695 | DPYD | intron | 0 |
| rs12672751 | 9.12E−04 | −0.208 | 0.944 | 0.341 | 7 | 133,745,915 | AKR1B1 | 3′-downstream | 31,732 |
| rs9302094 | 9.13E−04 | −0.207 | 0.944 | 0.371 | 13 | 95,650,741 | HS6ST3 | intron | 0 |
| rs8076286 | 9.14E−04 | −0.205 | 0.944 | 0.431 | 17 | 47,451,755 | CA10 | intron | 0 |
| rs3775763 | 9.14E−04 | 0.206 | 0.944 | 0.482 | 4 | 70,929,238 | HTN3 | intron | 0 |
| rs2804875 | 9.14E−04 | −0.205 | 0.944 | 0.242 | 10 | 33,883,553 | NRP1 | 5′-upstream | 219,714 |
| rs4556834 | 9.15E−04 | 0.205 | 0.944 | 0.300 | 17 | 47,549,854 | CA10 | intron | 0 |
| rs11023907 | 9.16E−04 | 0.209 | 0.944 | 0.152 | 11 | 16,281,025 | SOX6 | intron | 0 |
| rs2974000 | 9.18E−04 | 0.206 | 0.944 | 0.191 | 5 | 119,230,346 | LOC348958 | 3′-downstream | 184,639 |
| rs7307293 | 9.18E−04 | 0.205 | 0.944 | 0.094 | 12 | 67,114,413 | LOC729376 | intron | 0 |
| rs9323880 | 9.18E−04 | −0.205 | 0.944 | 0.271 | 14 | 92,199,563 | RIN3 | intron | 0 |
| rs10022167 | 9.19E−04 | 0.206 | 0.944 | 0.460 | 4 | 74,497,673 | ALB | intron | 0 |
| rs6896653 | 9.20E−04 | 0.206 | 0.944 | 0.164 | 5 | 107,732,780 | FBXL17 | intron | 0 |
| rs1727387 | 9.20E−04 | 0.205 | 0.944 | 0.079 | 12 | 118,356,085 | CCDC60 | intron | 0 |
| rs831571 | 9.20E−04 | 0.205 | 0.944 | 0.253 | 3 | 64,023,337 | PRICKLE2 | 3′-downstream | 31,250 |
| rs1235399 | 9.20E−04 | 0.208 | 0.944 | 0.074 | 5 | 136,761,637 | SPOCK1 | intron | 0 |
| rs1428280 | 9.20E−04 | 0.205 | 0.944 | 0.067 | 5 | 13,640,660 | LOC391738 | 3′-downstream | 50,364 |
| rs9542594 | 9.22E−04 | 0.205 | 0.944 | 0.206 | 13 | 70,617,166 | DACH1 | 3′-downstream | 292,933 |
| rs17540362 | 9.23E−04 | −0.205 | 0.944 | 0.112 | 7 | 11,263,913 | PHF14 | 3′-downstream | 88,143 |
| rs5751080 | 9.23E−04 | −0.205 | 0.944 | 0.493 | 22 | 40,066,213 | ZC3H7B | intron | 0 |
| rs12955535 | 9.23E−04 | 0.205 | 0.944 | 0.090 | 18 | 74,145,153 | LOC100132713 | 5′-upstream | 214,048 |
| rs896088 | 9.25E−04 | 0.205 | 0.944 | 0.419 | 5 | 165,875,957 | LOC441114 | 3′-downstream | 133,588 |
| rs447278 | 9.27E−04 | 0.205 | 0.944 | 0.406 | 6 | 5,398,289 | FARS2 | intron | 0 |
| rs7773352 | 9.29E−04 | 0.205 | 0.944 | 0.273 | 6 | 108,423,962 | LOC642741 | 3′-downstream | 8,220 |
| rs1887309 | 9.30E−04 | −0.205 | 0.944 | 0.431 | 9 | 136,448,063 | RXRA | intron | 0 |
| rs7283239 | 9.32E−04 | 0.205 | 0.944 | 0.070 | 21 | 21,062,388 | PPIAP | 5′-upstream | 59,610 |
| rs361488 | 9.32E−04 | 0.205 | 0.944 | 0.487 | 7 | 141,727,289 | TRBV8-1 | Unknown | — |
| rs10508612 | 9.32E−04 | 0.206 | 0.944 | 0.440 | 10 | 20,425,576 | PLXDC2 | intron | 0 |
| rs7256207 | 9.33E−04 | 0.205 | 0.944 | 0.149 | 19 | 33,539,522 | LOC727780 | 5′-upstream | 43,275 |
| rs838600 | 9.33E−04 | −0.205 | 0.944 | 0.496 | 3 | 144,698,162 | SLC9A9 | intron | 0 |
| rs11002468 | 9.35E−04 | 0.208 | 0.944 | 0.307 | 10 | 79,682,057 | LOC100132987 | 5′-upstream | 154,250 |
| rs10921053 | 9.35E−04 | 0.204 | 0.944 | 0.215 | 1 | 190,234,692 | RGS18 | 5′-upstream | 159,523 |
| rs674424 | 9.35E−04 | 0.204 | 0.944 | 0.146 | 11 | 118,535,962 | ABCG4 | intron | 0 |
| rs9447089 | 9.36E−04 | −0.204 | 0.944 | 0.139 | 6 | 74,751,120 | CD109 | 3′-downstream | 156,359 |
| rs2290 | 9.39E−04 | 0.204 | 0.944 | 0.056 | 4 | 121,875,517 | PRDM5 | intron | 0 |
| rs2409758 | 9.41E−04 | 0.205 | 0.944 | 0.380 | 21 | 36,412,525 | LOC100133286 | intron | 0 |
| rs10019178 | 9.42E−04 | −0.204 | 0.944 | 0.424 | 4 | 16,139,177 | LDB2 | intron | 0 |
| rs7050164 | 9.42E−04 | −0.204 | 0.944 | 0.159 | 23 | 7,668,041 | VCX | 5′-upstream | 102,262 |
| rs12740426 | 9.42E−04 | −0.204 | 0.944 | 0.482 | 1 | 6,237,066 | GPR153 | intron | 0 |
| rs4375446 | 9.43E−04 | −0.204 | 0.944 | 0.314 | 11 | 66,667,402 | FBXL11 | intron | 0 |
| rs2581806 | 9.44E−04 | 0.204 | 0.944 | 0.338 | 3 | 53,038,400 | SFMBT1 | intron | 0 |
| rs1778913 | 9.44E−04 | 0.205 | 0.944 | 0.266 | 9 | 86,625,557 | NTRK2 | intron | 0 |
| rs2854541 | 9.44E−04 | 0.204 | 0.944 | 0.486 | 7 | 141,726,054 | TRBV8-1 | Unknown | — |
| rs12007499 | 9.44E−04 | 0.205 | 0.944 | 0.173 | 23 | 23,157,121 | LOC653707 | 3′-downstream | 30,532 |
| rs4948700 | 9.45E−04 | 0.204 | 0.944 | 0.206 | 10 | 42,869,213 | RET | 5′-upstream | 23,310 |
| rs17711345 | 9.45E−04 | −0.205 | 0.944 | 0.170 | 7 | 64,543,602 | ZNF92 | 3′-downstream | 40,169 |
| rs9849264 | 9.45E−04 | −0.205 | 0.944 | 0.409 | 3 | 126,651,497 | SNX4 | intron | 0 |
| rs2784846 | 9.47E−04 | 0.204 | 0.944 | 0.164 | 10 | 86,264,851 | KIAA1128 | 3′-UTR | 0 |
| rs13065032 | 9.47E−04 | −0.204 | 0.944 | 0.083 | 3 | 129,993,284 | RAB7A | intron | 0 |
| rs354113 | 9.47E−04 | −0.205 | 0.944 | 0.174 | 12 | 30,425,805 | IPO8 | 3′-downstream | 247,384 |
| rs7943820 | 9.48E−04 | 0.204 | 0.944 | 0.280 | 11 | 107,894,495 | EXPH5 | intron | 0 |
| rs10749486 | 9.48E−04 | 0.204 | 0.944 | 0.141 | 10 | 86,101,607 | KIAA1128 | intron | 0 |
| rs1209475 | 9.48E−04 | 0.204 | 0.944 | 0.141 | 10 | 86,248,339 | KIAA1128 | intron | 0 |
| rs4261230 | 9.48E−04 | 0.204 | 0.944 | 0.141 | 10 | 86,109,189 | KIAA1128 | intron | 0 |
| rs11584409 | 9.49E−04 | 0.204 | 0.944 | 0.058 | 1 | 95,957,886 | LOC729977 | 5′-upstream | 401,479 |
| rs10243011 | 9.49E−04 | −0.205 | 0.944 | 0.170 | 7 | 121,070,149 | tcag7.23 | 5′-upstream | 118,405 |
| rs4845143 | 9.49E−04 | 0.209 | 0.944 | 0.379 | 1 | 205,069,942 | IL19 | intron | 0 |
| rs10493976 | 9.49E−04 | 0.204 | 0.944 | 0.181 | 1 | 102,036,497 | OLFM3 | 3′-downstream | 4,221 |
| rs10249085 | 9.50E−04 | −0.204 | 0.944 | 0.370 | 7 | 34,842,539 | NPSR1 | intron | 0 |
| rs3924091 | 9.52E−04 | 0.205 | 0.944 | 0.283 | 4 | 188,905,046 | LOC644325 | Unknown | — |
| rs12028349 | 9.52E−04 | 0.204 | 0.944 | 0.309 | 1 | 75,293,188 | LHX8 | 5′-upstream | 73,519 |
| rs10861956 | 9.53E−04 | 0.204 | 0.944 | 0.296 | 12 | 107,578,604 | CORO1C | intron | 0 |
| rs7300726 | 9.53E−04 | 0.204 | 0.944 | 0.296 | 12 | 107,577,991 | CORO1C | intron | 0 |
| rs2405747 | 9.53E−04 | 0.204 | 0.944 | 0.054 | 2 | 128,748,295 | HS6ST1 | intron | 0 |
| rs6717511 | 9.53E−04 | 0.204 | 0.944 | 0.054 | 2 | 128,747,543 | HS6ST1 | intron | 0 |
| rs7229568 | 9.54E−04 | 0.204 | 0.944 | 0.184 | 18 | 4,814,081 | PPIAP14 | 3′-downstream | 178,430 |
| rs17152492 | 9.54E−04 | 0.204 | 0.944 | 0.090 | 8 | 10,705,344 | PINX1 | intron | 0 |
| rs3752558 | 9.55E−04 | 0.204 | 0.944 | 0.108 | 20 | 41,621,277 | SGK2 | intron | 0 |
| rs6929812 | 9.56E−04 | −0.205 | 0.944 | 0.473 | 6 | 27,492,499 | MCFD2L | 3′-downstream | 8,745 |
| rs2835623 | 9.58E−04 | 0.204 | 0.944 | 0.146 | 21 | 37,433,401 | TTC3 | intron | 0 |
| rs165190 | 9.60E−04 | −0.204 | 0.944 | 0.391 | 5 | 139,126,284 | PSD2 | 5′-upstream | 29,306 |
| rs11012265 | 9.60E−04 | −0.204 | 0.944 | 0.289 | 10 | 21,010,483 | NEBL | 3′-downstream | 99,610 |
| rs11929275 | 9.60E−04 | 0.206 | 0.944 | 0.082 | 3 | 72,807,027 | SHQ1 | 3′-downstream | 74,091 |
| rs981213 | 9.61E−04 | 0.204 | 0.944 | 0.341 | 12 | 77,115,380 | NAV3 | intron | 0 |
| rs11038044 | 9.62E−04 | 0.204 | 0.944 | 0.083 | 11 | 44,497,871 | LOC646535 | 3′-downstream | 20,034 |
| rs1485803 | 9.62E−04 | −0.206 | 0.944 | 0.385 | 18 | 22,880,887 | CHST9 | intron | 0 |
| rs12614728 | 9.62E−04 | −0.204 | 0.944 | 0.237 | 2 | 76,502,501 | LOC647275 | 5′-upstream | 162,970 |
| rs202124 | 9.63E−04 | 0.205 | 0.944 | 0.265 | 17 | 47,342,798 | CA10 | intron | 0 |
| rs748353 | 9.64E−04 | 0.204 | 0.944 | 0.183 | 11 | 105,164,699 | GRIA4 | intron | 0 |
| rs1250249 | 9.64E−04 | 0.204 | 0.944 | 0.464 | 2 | 215,995,521 | FN1 | intron | 0 |
| rs922140 | 9.65E−04 | 0.204 | 0.944 | 0.316 | 5 | 122,116,789 | SNX2 | 5′-upstream | 21,860 |
| rs10268122 | 9.65E−04 | −0.204 | 0.944 | 0.108 | 7 | 5,066,937 | RBAK | intron | 0 |
| rs1989614 | 9.68E−04 | −0.204 | 0.944 | 0.421 | 2 | 80,259,773 | CTNNA2 | intron | 0 |
| rs4878192 | 9.68E−04 | 0.205 | 0.944 | 0.222 | 9 | 38,288,804 | ALDH1B1 | 5′-upstream | 93,898 |
| rs4395807 | 9.69E−04 | 0.204 | 0.944 | 0.106 | 7 | 142,457,413 | OR6V1 | 5′-upstream | 2,147 |
| rs16912758 | 9.69E−04 | 0.204 | 0.944 | 0.114 | 10 | 60,300,047 | BICC1 | 3′-downstream | 41,196 |
| rs12204014 | 9.70E−04 | 0.204 | 0.944 | 0.341 | 6 | 67,456,669 | NUFIP1P | 5′-upstream | 595,137 |
| rs4105713 | 9.71E−04 | 0.205 | 0.944 | 0.425 | 12 | 118,937,703 | CCDC64 | intron | 0 |
| rs16834635 | 9.71E−04 | −0.204 | 0.944 | 0.112 | 1 | 163,006,475 | PBX1 | intron | 0 |
| rs2816567 | 9.71E−04 | 0.204 | 0.944 | 0.237 | 6 | 87,065,440 | LOC643916 | 5′-upstream | 84,418 |
| rs10494980 | 9.72E−04 | 0.204 | 0.944 | 0.144 | 1 | 212,725,200 | PTPN14 | intron | 0 |
| rs10839797 | 9.73E−04 | 0.204 | 0.944 | 0.442 | 11 | 7,502,978 | PPFIBP2 | intron | 0 |
| rs444386 | 9.76E−04 | 0.205 | 0.944 | 0.215 | 10 | 90,518,158 | LIPN | intron | 0 |
| rs4252725 | 9.77E−04 | 0.205 | 0.944 | 0.476 | 1 | 202,779,879 | MDM4 | intron | 0 |
| rs9868005 | 9.78E−04 | 0.204 | 0.944 | 0.433 | 3 | 64,607,454 | ADAMTS9 | intron | 0 |
| rs9383810 | 9.78E−04 | 0.204 | 0.944 | 0.081 | 6 | 156,895,326 | ARID1B | 5′-upstream | 245,452 |
| rs6913087 | 9.79E−04 | 0.204 | 0.944 | 0.208 | 6 | 54,245,008 | C6orf142 | 3′-downstream | 5,971 |
| rs9687159 | 9.80E−04 | −0.204 | 0.944 | 0.243 | 5 | 15,077,237 | LOC402198 | 3′-downstream | 19,353 |
| rs2426278 | 9.81E−04 | 0.204 | 0.944 | 0.094 | 20 | 49,236,052 | RPSAP1 | 5′-upstream | 54,341 |
| rs12197200 | 9.81E−04 | 0.204 | 0.944 | 0.054 | 6 | 163,503,111 | PACRG | intron | 0 |
| rs6965133 | 9.81E−04 | −0.204 | 0.944 | 0.232 | 7 | 103,368,862 | RELN | intron | 0 |
| rs4806332 | 9.82E−04 | 0.204 | 0.944 | 0.173 | 19 | 33,548,847 | LOC727780 | 5′-upstream | 52,600 |
| rs1019286 | 9.84E−04 | 0.204 | 0.944 | 0.188 | 7 | 38,426,876 | AMPH | intron | 0 |
| rs6520403 | 9.85E−04 | −0.205 | 0.944 | 0.208 | 23 | 48,927,746 | PRICKLE3 | intron | 0 |
| rs17025652 | 9.85E−04 | 0.204 | 0.944 | 0.365 | 3 | 88,441,414 | LOC344653 | 5′-upstream | 7,582 |
| rs8139876 | 9.85E−04 | 0.204 | 0.944 | 0.109 | 22 | 20,865,973 | IGLVV-58 | Unknown | — |
| rs4146616 | 9.85E−04 | 0.205 | 0.944 | 0.051 | 10 | 67,550,131 | CTNNA3 | intron | 0 |
| rs4767867 | 9.85E−04 | 0.204 | 0.944 | 0.412 | 12 | 118,906,208 | CCDC64 | 5′-upstream | 5,823 |
| rs1280374 | 9.86E−04 | −0.204 | 0.944 | 0.173 | 15 | 55,542,924 | CGNL1 | intron | 0 |
| rs2073505 | 9.87E−04 | 0.205 | 0.944 | 0.100 | 4 | 3,414,301 | HGFAC | synonymous | 0 |
| rs12410848 | 9.89E−04 | 0.204 | 0.944 | 0.306 | 1 | 111,636,507 | CHIA | intron | 0 |
| rs9562045 | 9.89E−04 | 0.204 | 0.944 | 0.466 | 13 | 95,638,906 | HS6ST3 | intron | 0 |
| rs34922583 | 9.90E−04 | 0.203 | 0.944 | 0.139 | 5 | 40,169,516 | LOC285634 | 5′-upstream | 376,069 |
| rs12890069 | 9.90E−04 | 0.204 | 0.944 | 0.073 | 14 | 51,827,323 | LOC100131689 | 3′-downstream | 1,506 |
| rs5023021 | 9.91E−04 | 0.204 | 0.944 | 0.120 | 9 | 11,094,151 | LOC646114 | 3′-downstream | 63,866 |
| rs17134115 | 9.93E−04 | 0.204 | 0.944 | 0.091 | 7 | 51,008,773 | COBL | 3′-downstream | 42,631 |
| rs13021679 | 9.93E−04 | 0.203 | 0.944 | 0.446 | 2 | 215,984,620 | FN1 | intron | 0 |
| rs10962894 | 9.93E−04 | −0.205 | 0.944 | 0.297 | 9 | 1,716,601 | SMARCA2 | 5′-upstream | 288,741 |

*R values represent correlation coefficients for the association.

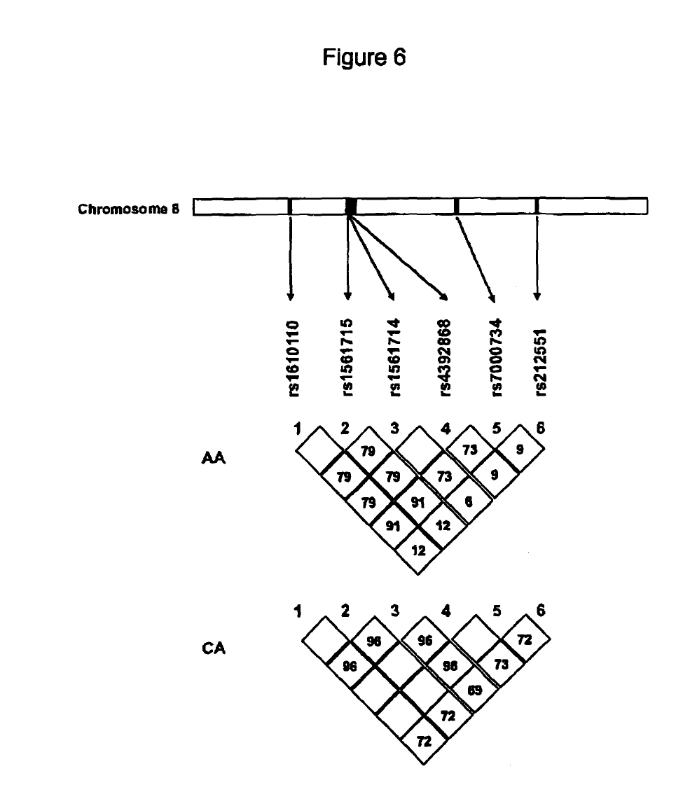

“Integrated analyses” of SNPs with both expression array and cytotoxicity data were started with the 1335 SNPs that had p values<10⁻³. When the locations of these 1335 SNPs were determined, 27 regions were identified that each contained at least 2 SNPs with p values<10⁻⁴ within 50 kb (a total of 175 SNPs). For purposes of this analysis, each of these regions was defined as a “locus” or a “SNP peak region.” All 27 of these loci, and the SNPs within each locus, are listed in Table 8. These “loci” were the focus of the integrated analyses. The most significant locus, locus 8C, mapped to chromosome 8 (FIG. 5C and Table 8). This region had 6 SNPs within 50 kb with p values<10⁻⁴, including the SNP with the lowest p value (3.82×10⁻⁷). The gene closest to this region encoded PLEKHF2. All 6 of the SNPs were in tight linkage disequilibrium (LD), with R² values that ranged from 0.7 to 1.0 in CAs, with slightly lower values in the AA subjects (FIG. 6). The allele frequencies for these SNPs also were higher in CA and AA subjects than in HCA subjects. SNPs not on the genotyping platforms that were located 200 kb up or downstream of SNP A-8538282, the most significant SNP on chromosome 8 (see FIG. 5C) also were imputed. That area contained 222 “observed SNPs” that were on the combined Illumina and Affymetrix platforms, plus 104 SNPs that were imputed (FIG. 5C).

TABLE 8
The 27 “loci” associated with radiation AUC

| | | | | | | | | | | Location |
|---|---|---|---|---|---|---|---|---|---|---|
| | | P | R | Q | | | | | | Relative To |
| Locus | SNP ID | value | value* | value | MAF | Chr | Position | GeneSymbol^{†} | Location | Gene (bp) |
| 1A | rs1130790 | 1.88E-04 | 0.230 | 0.857 | 0.348 | 1 | 200,235,705 | RNPEP | exon | 0 |
| | rs4131469 | 8.86E-05 | 0.241 | 0.853 | 0.350 | 1 | 200,237,705 | RNPEP | intron | 0 |
| | rs12119699 | 2.31E-04 | 0.227 | 0.891 | 0.348 | 1 | 200,239,578 | RNPEP | intron | 0 |
| | rs3820439 | 8.77E-04 | −0.206 | 0.944 | 0.319 | 1 | 200,240,188 | RNPEP | exon | 0 |
| | rs7516412 | 7.28E-04 | −0.209 | 0.944 | 0.305 | 1 | 200,242,824 | ELF3 | 5'-upstream | 872 |
| | rs2819358 | 2.31E-04 | 0.227 | 0.891 | 0.348 | 1 | 200,243,025 | ELF3 | 5'-upstream | 671 |
| | rs2735784 | 9.48E-05 | 0.240 | 0.853 | 0.375 | 1 | 200,243,103 | ELF3 | 5'-upstream | 593 |
| | rs2819360 | 3.17E-04 | 0.222 | 0.932 | 0.341 | 1 | 200,243,877 | ELF3 | 5'-UTR | 0 |
| 1B | rs4951407 | 8.84E-04 | 0.206 | 0.944 | 0.489 | 1 | 202,824,693 | LRRN2 | 3'-downstream | 28,231 |
| | rs6679717 | 6.89E-04 | 0.212 | 0.944 | 0.480 | 1 | 202,827,300 | LRRN2 | 3'-downstream | 25,624 |
| | rs4951409 | 6.26E-04 | 0.211 | 0.944 | 0.475 | 1 | 202,827,613 | LRRN2 | 3'-downstream | 25,311 |
| | rs7532236 | 6.26E-04 | 0.211 | 0.944 | 0.475 | 1 | 202,828,240 | LRRN2 | 3'-downstream | 24,684 |
| | rs6682208 | 8.70E-04 | 0.206 | 0.944 | 0.478 | 1 | 202,832,806 | LRRN2 | 3'-downstream | 20,118 |
| | rs6594018 | 6.56E-04 | 0.210 | 0.944 | 0.478 | 1 | 202,834,775 | LRRN2 | 3'-downstream | 18,149 |
| | rs11240764 | 6.28E-04 | 0.211 | 0.944 | 0.475 | 1 | 202,835,999 | LRRN2 | 3'-downstream | 16,925 |
| | rs10900601 | 7.37E-05 | 0.244 | 0.845 | 0.227 | 1 | 202,838,673 | LRRN2 | 3'-downstream | 14,251 |
| | rs898387 | 1.24E-05 | 0.268 | 0.696 | 0.235 | 1 | 202,845,996 | LRRN2 | 3''-downstream | 6,928 |
| | rs898386 | 4.22E-05 | 0.253 | 0.727 | 0.229 | 1 | 202,846,320 | LRRN2 | 3'-downstream | 6,604 |
| | rs2045624 | 4.51E-05 | 0.251 | 0.727 | 0.258 | 1 | 202,851,008 | LRRN2 | 3'-downstream | 1,916 |
| | rs7539399 | 3.08E-04 | 0.222 | 0.932 | 0.269 | 1 | 202,856,667 | LRRN2 | intron | 0 |
| | rs884108 | 1.07E-04 | 0.241 | 0.853 | 0.180 | 1 | 202,857,860 | LRRN2 | intron | 0 |
| | rs4951088 | 1.20E-04 | 0.237 | 0.857 | 0.471 | 1 | 202,877,403 | LRRN2 | intron | 0 |
| | rs4951089 | 1.60E-04 | 0.232 | 0.857 | 0.475 | 1 | 202,877,521 | LRRN2 | intron | 0 |
| | rs11240237 | 1.60E-04 | 0.232 | 0.857 | 0.475 | 1 | 202,879,141 | LRRN2 | intro | 0 |
| | rs11240239 | 1.22E-04 | 0.237 | 0.857 | 0.469 | 1 | 202,879,427 | LRRN2 | intron | 0 |
| | rs10900418 | 4.20E-04 | 0.218 | 0.944 | 0.309 | 1 | 202,880,789 | LRRN2 | intron | 0 |
| | rs2772227 | 2.00E-04 | 0.229 | 0.865 | 0.152 | 1 | 202,882,023 | LRRN2 | intron | 0 |
| | rs1329631 | 2.31E-04 | 0.227 | 0.891 | 0.307 | 1 | 202,883,013 | LRRN2 | intron | 0 |
| | rs2815832 | 4.95E-04 | 0.215 | 0.944 | 0.168 | 1 | 202,883,863 | LRRN2 | intron | 0 |
| 1C | rs1028182 | 2.65E-04 | 0.225 | 0.932 | 0.406 | 1 | 205,068,502 | IL19 | intron | 0 |
| | rs4845143 | 9.49E-04 | 0.209 | 0.944 | 0.379 | 1 | 205,069,942 | IL19 | intron | 0 |
| | rs12022129 | 8.37E-04 | 0.206 | 0.944 | 0.394 | 1 | 205,069,997 | IL19 | intron | 0 |
| | rs11119670 | 7.45E-05 | 0.244 | 0.845 | 0.442 | 1 | 205,071,944 | IL19 | intron | 0 |
| | rs2243174 | 7.04E-05 | 0.244 | 0.843 | 0.444 | 1 | 205,078,108 | IL19 | intron | 0 |
| | rs2243188 | 7.11E-05 | 0.244 | 0.843 | 0.442 | 1 | 205,081,095 | IL19 | intron | 0 |
| | rs1798 | 9.12E-04 | 0.205 | 0.944 | 0.195 | 1 | 205,082,748 | IL19 | 3'-UTR | 0 |
| | rs2243193 | 9.77E-05 | 0.240 | 0.853 | 0.453 | 1 | 205,082,848 | IL19 | 3'-UTR | 0 |
| | rs4845144 | 3.50E-04 | 0.222 | 0.932 | 0.458 | 1 | 205,083,271 | TL19 | 3'-downstream | 324 |
| | rs2883036 | 3.99E-04 | −0.221 | 0.944 | 0.361 | 1 | 205,091,385 | TL19 | 3'-downstream | 8,438 |
| 1D | rs780266 | 1.55E-04 | 0.233 | 0.857 | 0.071 | 1 | 231,791,723 | KCNK1 | 5'-upstream | 24,650 |
| | rs1773951 | 1.34E-04 | 0.235 | 0.857 | 0.070 | 1 | 231,802,262 | KCNK1 | 5'-upstream | 14,111 |
| | rs1693219 | 5.89E-05 | 0.247 | 0.823 | 0.289 | 1 | 231,807,892 | KCNK1 | 5'-upstream | 8,481 |
| | rs1693216 | 6.50E-05 | 0.247 | 0.843 | 0.286 | 1 | 231,808,517 | KCNK1 | 5'-upstream | 7,856 |
| | rs780256 | 2.67E-04 | 0.225 | 0.932 | 0.288 | 1 | 231,809,726 | KCNK1 | 5'-upstream | 6,647 |
| | rs636811 | 8.09E-04 | 0.207 | 0.944 | 0.090 | 1 | 231,815,644 | KCNK1 | 5'-upstream | 729 |
| 1E | rs12569163 | 1.78E-06 | 0.292 | 0.394 | 0.118 | 1 | 239,945,170 | WDR64 | intron | 0 |
| | rs7554126 | 4.34E-06 | 0.287 | 0.667 | 0.297 | 1 | 239,953,804 | WDR64 | intron | 0 |
| 3A | rs894013 | 3.19E-05 | 0.261 | 0.711 | 0.472 | 3 | 4,353,142 | SETMAR | 3'-downstream | 19,193 |
| | rs308731 | 8.21E-05 | 0.242 | 0.853 | 0.365 | 3 | 4,354,374 | SETMAR | 3'-downstream | 20,425 |
| 3B | rs1469569 | 7.28E-04 | −0.209 | 0.944 | 0.421 | 3 | 88,158,887 | CGGBP1 | 3'-downstream | 24,903 |
| | rs17549345 | 5.39E-05 | −0.248 | 0.777 | 0.336 | 3 | 88,163,269 | CGGBP1 | 3'-downstream | 20,521 |
| | rs6785333 | 8.00E-04 | −0.209 | 0.944 | 0.414 | 3 | 88,168,323 | CGGBP1 | 3'-downstream | 15,467 |
| | rs9852704 | 9.86E-05 | −0.240 | 0.853 | 0.406 | 3 | 88,171,034 | CGGBP1 | 3'-downstream | 12,756 |
| 4A | rs13127174 | 2.86E-04 | 0.224 | 0.932 | 0.157 | 4 | 16,137,569 | LDB2 | intron | 0 |
| | rs10019178 | 9.42E-04 | −0.204 | 0.944 | 0.424 | 4 | 16,139,177 | LDB2 | intron | 0 |
| | rs10805350 | 3.50E-04 | 0.222 | 0.932 | 0.313 | 4 | 16,140,499 | LDB2 | intron | 0 |
| | rs13138387 | 1.70E-04 | 0.232 | 0.857 | 0.065 | 4 | 16,142,474 | LDB2 | intron | 0 |
| | rs10516301 | 4.97E-05 | 0.250 | 0.750 | 0.072 | 4 | 16,143,251 | LDB2 | intron | 0 |
| | rs16893606 | 7.49E-04 | 0.208 | 0.944 | 0.173 | 4 | 16,146,608 | LDB2 | intron | 0 |
| | rs881790 | 3.46E-04 | 0.221 | 0.932 | 0.195 | 4 | 16,147,055 | LDB2 | intron | 0 |
| | rs13128522 | 2.38E-05 | 0.260 | 0.696 | 0.060 | 4 | 16,149,782 | LDB2 | intron | 0 |
| 4B | rs2078395 | 5.90E-04 | 0.213 | 0.944 | 0.443 | 4 | 181,140,358 | hCG_2025798 | 3'-downstream | 1,081,878 |
| | rs35018391 | 3.23E-05 | 0.255 | 0.711 | 0.049 | 4 | 181,217,870 | hCG_2025798 | 3'-downstream | 1,004,366 |
| | rs41333347 | 3.23E-05 | 0.255 | 0.711 | 0.049 | 4 | 181,227,186 | hCG_2025798 | 3'-downstream | 995,050 |
| | rs7680285 | 3.23E-05 | 0.255 | 0.711 | 0.049 | 4 | 181,228,438 | hCG_2025798 | 3′-downstream | 993,798 |
| | rs2309341 | 9.04E-05 | −0.241 | 0.853 | 0.433 | 4 | 181,590,726 | hCG_2025798 | 3′-downstream | 631,510 |
| 4C | rs11726299 | 5.15E-04 | −0.214 | 0.944 | 0.190 | 4 | 183,556,319 | ODZ3 | intron | 0 |
| | rs727695 | 1.42E-04 | 0.235 | 0.857 | 0.313 | 4 | 183,610,140 | ODZ3 | intron | 0 |
| | rs7676392 | 1.73E-05 | 0.264 | 0.696 | 0.281 | 4 | 183,613,233 | ODZ3 | intron | 0 |
| | rs6830998 | 1.28E-04 | 0.237 | 0.857 | 0.279 | 4 | 183,617,946 | ODZ3 | intron | 0 |
| | rs869239 | 1.60E-05 | 0.265 | 0.696 | 0.495 | 4 | 183,690,033 | ODZ3 | intron | 0 |
| | rs2309691 | 5.36E-05 | 0.248 | 0.777 | 0.491 | 4 | 183,691,063 | ODZ3 | intron | 0 |
| | rs11731599 | 2.98E-05 | 0.257 | 0.711 | 0.489 | 4 | 183,691,126 | ODZ3 | intron | 0 |
| | rs6840083 | 3.01E-04 | 0.223 | 0.932 | 0.421 | 4 | 183,701,671 | ODZ3 | intron | 0 |
| 5A | rs26953 | 1.78E-04 | −0.233 | 0.857 | 0.326 | 5 | 59,850,071 | PART1 | intron | 0 |
| | rs27564 | 5.55E-05 | −0.251 | 0.791 | 0.325 | 5 | 59,873,391 | PART1 | 3′-downstream | 20,403 |
| | rs6863920 | 7.37E-04 | −0.209 | 0.944 | 0.304 | 5 | 59,898,210 | 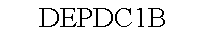 | 3′-downstream | 30,286 |
| | rs6449478 | 3.26E-04 | −0.224 | 0.932 | 0.267 | 5 | 59,909,466 | 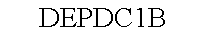 | 3′-downstream | 19,030 |
| | rs2409791 | 1.09E-05 | −0.270 | 0.696 | 0.280 | 5 | 59,920,074 | 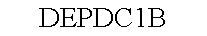 | 3′-downstream | 8,422 |
| | rs4326096 | 3.41E-05 | −0.255 | 0.711 | 0.270 | 5 | 59,926,035 | 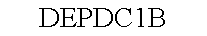 | 3′-downstream | 2,461 |
| | rs206789 | 5.74E-05 | −0.248 | 0.810 | 0.313 | 5 | 59,939,728 | 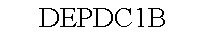 | intron | 0 |
| | rs286158 | 1.34E-04 | −0.235 | 0.857 | 0.309 | 5 | 59,970,010 | 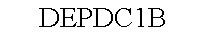 | intron | 0 |
| 5B | rs6884002 | 2.96E-04 | −0.223 | 0.932 | 0.099 | 5 | 154,872,015 | LOC100131033 | 3′-downstream | 18,661 |
| | rs10051752 | 1.99E-04 | −0.229 | 0.865 | 0.085 | 5 | 154,966,485 | LOC100131033 | 3′-downstream | 113,131 |
| | rs7704817 | 7.04E-04 | −0.210 | 0.944 | 0.223 | 5 | 154,968,300 | LOC100131033 | 3′-downstream | 114,946 |
| | rs7379436 | 1.00E-04 | 0.239 | 0.853 | 0.074 | 5 | 155,158,726 | LOC100131033 | 3′-downstream | 305,372 |
| | rs10073856 | 9.49E-05 | 0.241 | 0.853 | 0.071 | 5 | 155,195,298 | LOC100131033 | 3′-downstream | 341,944 |
| | rs6873333 | 3.64E-05 | 0.254 | 0.711 | 0.072 | 5 | 155,195,850 | LOC100131033 | 3′-downstream | 342,496 |
| | rs7378774 | 7.71E-05 | 0.245 | 0.845 | 0.064 | 5 | 155,221,392 | LOC100131033 | 3′-downstream | 368,038 |
| 7A | rs12702213 | 9.29E-05 | 0.240 | 0.853 | 0.058 | 7 | 46,381,080 | LOC730338 | 3′-downstream | 314,296 |
| | rs13239088 | 6.64E-05 | 0.249 | 0.843 | 0.054 | 7 | 46,381,408 | LOC730338 | 3′-downstream | 313,968 |
| | rs12702227 | 9.04E-04 | 0.205 | 0.944 | 0.052 | 7 | 46,519,896 | LOC730338 | 3′-downstream | 175,480 |
| | rs12702228 | 9.04E-04 | 0.205 | 0.944 | 0.052 | 7 | 46,522,406 | LOC730338 | 3′-downstream | 172,970 |
| 7B | rs17598132 | 1.89E-05 | 0.263 | 0.696 | 0.076 | 7 | 96,417,275 | FLJ34048 | 3′-downstream | 53,620 |
| | rs17598306 | 8.60E-06 | 0.273 | 0.696 | 0.079 | 7 | 96,419,747 | FLJ34048 | 3′-downstream | 51,148 |
| | rs2016644 | 4.86E-04 | 0.216 | 0.944 | 0.087 | 7 | 96,433,985 | FLJ34048 | 3′-downstream | 36,910 |
| | rs17657370 | 3.34E-05 | 0.255 | 0.711 | 0.078 | 7 | 96,444,428 | FLJ34048 | 3′-downstream | 26,467 |
| 7C | rs13222164 | 4.97E-04 | 0.215 | 0.944 | 0.150 | 7 | 108,885,600 | LOC100128056 | 5′-downstream | 380,818 |
| | rs12705596 | 1.75E-05 | 0.263 | 0.696 | 0.161 | 7 | 108,893,619 | LOC100128056 | 5′-downstream | 372,799 |
| | rs12705601 | 4.80E-05 | 0.250 | 0.740 | 0.161 | 7 | 108,923,348 | LOC100128056 | 5′-downstream | 343,070 |
| | rs12705602 | 7.91E-05 | 0.243 | 0.845 | 0.166 | 7 | 108,929,460 | LOC100128056 | 5′-downstream | 336,958 |
| | rs13239901 | 5.90E-04 | 0.213 | 0.944 | 0.232 | 7 | 108,935,937 | LOC100128056 | 5′-downstream | 330,481 |
| 7D | rs10240447 | 7.41E-05 | 0.244 | 0.845 | 0.495 | 7 | 157,460,585 | PTPRN2 | intron | 0 |
| | rs4716858 | 6.74E-05 | −0.245 | 0.843 | 0.468 | 7 | 157,461,158 | PTPRN2 | intron | 0 |
| 8A | rs10098626 | 7.78E-04 | 0.208 | 0.944 | 0.442 | 8 | 35,008,456 | LOC100133273 | 3′-downstream | 156,685 |
| | rs10503973 | 8.36E-04 | 0.207 | 0.944 | 0.219 | 8 | 35,018,804 | LOC100133273 | 3′-downstream | 167,033 |
| | rs10503974 | 7.71E-04 | 0.208 | 0.944 | 0.218 | 8 | 35,018,934 | LOC100133273 | 3′-downstream | 167,163 |
| | rs10954975 | 5.56E-04 | 0.213 | 0.944 | 0.217 | 8 | 35,019,032 | LOC100133273 | 3′-downstream | 167,261 |
| | rs11786195 | 4.68E-04 | 0.216 | 0.944 | 0.204 | 8 | 35,020,843 | LOC100133273 | 3′-downstream | 169,072 |
| | rs12678629 | 4.19E-04 | 0.218 | 0.944 | 0.215 | 8 | 35,025,990 | LOC100133273 | 3′-downstream | 174,219 |
| | rs13282927 | 2.71E-04 | 0.226 | 0.932 | 0.212 | 8 | 35,031,277 | LOC100133273 | 3′-downstream | 179,506 |
| | rs1376519 | 1.22E-04 | 0.238 | 0.857 | 0.244 | 8 | 35,045,674 | LOC100133273 | 3′-downstream | 193,903 |
| | rs2981309 | 6.62E-05 | 0.249 | 0.843 | 0.241 | 8 | 35,048,829 | LOC100133273 | 3′-downstream | 197,058 |
| | rs4314649 | 3.10E-05 | 0.257 | 0.711 | 0.242 | 8 | 35,052,669 | LOC100133273 | 3′-downstream | 200,898 |
| | rs2923661 | 3.46E-05 | 0.255 | 0.711 | 0.240 | 8 | 35,061,182 | LOC100133273 | 3′-downstream | 209,411 |
| | rs2981317 | 2.37E-04 | 0.227 | 0.891 | 0.200 | 8 | 35,069,374 | LOC100133273 | 3′-downstream | 217,603 |
| | rs4739296 | 6.64E-04 | 0.210 | 0.944 | 0.215 | 8 | 35,073,349 | LOC100133273 | 3′-downstream | 221,578 |
| 8B | rs7835942 | 8.01E-05 | −0.243 | 0.845 | 0.227 | 8 | 61,960,345 | CHD7 | 3′-downstream | 18,326 |
| | rs4738839 | 8.01E-05 | −0.243 | 0.845 | 0.227 | 8 | 61,960,444 | CHD7 | 3′-downstream | 18,425 |
| 8C | rs1610110 | 9.58E-07 | 0.299 | 0.318 | 0.083 | 8 | 96,185,038 | 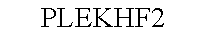 | 5′-downstream | 30,170 |
| | rs1561715 | 9.58E-07 | 0.299 | 0.318 | 0.083 | 8 | 96,189,090 | 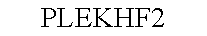 | 5′-downstream | 26,118 |
| | rs1561714 | 2.14E-05 | 0.261 | 0.696 | 0.087 | 8 | 96,189,151 | 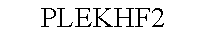 | 5′-downstream | 26,057 |
| | rs4392868 | 8.24E-06 | 0.274 | 0.696 | 0.087 | 8 | 96,189,248 | 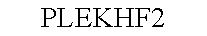 | 5′-downstream | 25,960 |
| | rs700734 | 3.82E-07 | 0.309 | 0.259 | 0.081 | 8 | 96,196,206 | 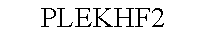 | 5′-downstream | 19,002 |
| | rs212551 | 6.85E-05 | 0.245 | 0.843 | 0.132 | 8 | 96,201,280 | 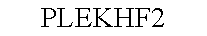 | 5′-downstream | 13,928 |
| 8D | rs11559202 | 6.57E-05 | 0.245 | 0.843 | 0.141 | 8 | 99,075,912 | MATN2 | exon | 0 |
| | rs1559201 | 6.57E-05 | 0.245 | 0.843 | 0.141 | 8 | 99,075,924 | MATN2 | exon | 0 |
| | rs2290467 | 1.48E-04 | 0.234 | 0.857 | 0.108 | 8 | 99,109,330 | MATN2 | intron | 0 |
| | rs2279120 | 2.13E-04 | 0.232 | 0.871 | 0.112 | 8 | 99,115,216 | MATN2 | intron | 0 |
| | rs3088121 | 9.52E-05 | 0.241 | 0.853 | 0.105 | 8 | 99,117,225 | MATN2 | 3′-UTR | 0 |
| 8E | rs4463400 | 2.06E-04 | 0.229 | 0.867 | 0.148 | 8 | 141,289,178 | 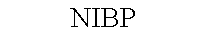 | intron | 0 |
| | rs6578080 | 3.31E-04 | 0.222 | 0.932 | 0.228 | 8 | 141,291,830 | 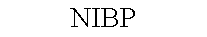 | intron | 0 |
| | rs4397386 | 1.03E-04 | 0.242 | 0.853 | 0.214 | 8 | 141,294,034 | 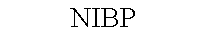 | intron | 0 |
| | rs7387053 | 5.14E-05 | 0.249 | 0.757 | 0.218 | 8 | 141,295,342 | 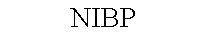 | intron | 0 |
| | rs4487737 | 5.14E-05 | 0.249 | 0.757 | 0.218 | 8 | 141,296,740 | 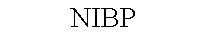 | intron | 0 |
| | rs13275618 | 8.46E-04 | −0.207 | 0.944 | 0.382 | 8 | 141,298,387 | 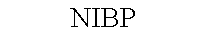 | intron | 0 |
| | rs6578083 | 7.41E-05 | −0.244 | 0.845 | 0.374 | 8 | 141,299,161 | 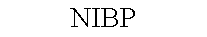 | intron | 0 |
| | rs7814976 | 4.80E-04 | −0.221 | 0.944 | 0.334 | 8 | 141,306,070 | 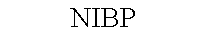 | intron | 0 |
| | rs7841539 | 5.82E-04 | −0.212 | 0.944 | 0.496 | 8 | 141,308,196 | 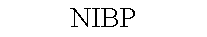 | intron | 0 |
| | rs6578084 | 3.66E-04 | −0.220 | 0.932 | 0.366 | 8 | 141,311,849 | 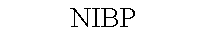 | intron | 0 |
| | rs9324519 | 3.96E-04 | −0.219 | 0.944 | 0.375 | 8 | 141,312,047 | 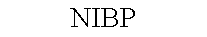 | intron | 0 |
| 9A | rs12553351 | 1.49E-05 | 0.265 | 0.696 | 0.117 | 9 | 6,828,348 | 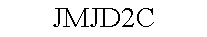 | intron | 0 |
| | rs4742269 | 4.73E-06 | 0.280 | 0.667 | 0.139 | 9 | 6,839,317 | 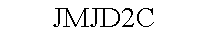 | intron | 0 |
| 9B | rs5023021 | 9.91E-04 | 0.204 | 0.944 | 0.120 | 9 | 11,094,151 | LOC646114 | 3′-downstream | 63,866 |
| | rs109595S4 | 2.33E-04 | 0.227 | 0.891 | 0.076 | 9 | 11,121,371 | LOC646114 | 3′-downstream | 91,086 |
| | rs7866090 | 4.45E-04 | 0.217 | 0.944 | 0.103 | 9 | 11,130,883 | LOC646114 | 3′-downstream | 100,598 |
| | rs10809359 | 2.22E-05 | 0.261 | 0.696 | 0.109 | 9 | 11,135,717 | LOC646114 | 3′-downstream | 105,432 |
| | rs12380191 | 7.47E-05 | 0.244 | 0.845 | 0.101 | 9 | 11,170,739 | LOC646114 | 3′-downstream | 140,454 |
| | rs12376660 | 3.71E-05 | 0.254 | 0.711 | 0.089 | 9 | 11,175,487 | LOC646114 | 3′-downstream | 145,202 |
| | rs12380100 | 1.55E-05 | 0.265 | 0.696 | 0.097 | 9 | 11,177,013 | LOC646114 | 3′-downstream | 146,728 |
| | rs10959622 | 1.02E-04 | 0.239 | 0.853 | 0.099 | 9 | 11,201,722 | LOC646114 | 3′-downstream | 171,437 |
| | rs11788082 | 8.03E-05 | 0.243 | 0.845 | 0.150 | 9 | 11,208,170 | LOC646114 | 3′-downstream | 177,885 |
| | rs10809395 | 2.50E-05 | 0.259 | 0.704 | 0.103 | 9 | 11,223,740 | LOC646114 | 3′-downstream | 193,455 |
| | rs10118338 | 6.89E-05 | 0.245 | 0.843 | 0.139 | 9 | 11,226,474 | LOC646114 | 3′-downstream | 196,189 |
| | rs10124468 | 3.48E-04 | 0.221 | 0.932 | 0.115 | 9 | 11,235,830 | LOC646114 | 3′-downstream | 205,545 |
| 10A | rs11250461 | 1.19E-04 | 0.237 | 0.857 | 0.226 | 10 | 1,394,502 | ADARB2 | intron | 0 |
| | rs11250464 | 5.61E-06 | 0.279 | 0.676 | 0.175 | 10 | 1,396,364 | ADARB2 | intron | 0 |
| | rs4554799 | 9.00E-06 | 0.272 | 0.696 | 0.170 | 10 | 1,397,728 | ADARB2 | intron | 0 |
| | rs2387653 | 6.69E-04 | 0.212 | 0.944 | 0.130 | 10 | 1,397,826 | ADARB2 | intron | 0 |
| | rs4880503 | 4.54E-05 | 0.251 | 0.727 | 0.171 | 10 | 1,426,858 | ADARB2 | intron | 0 |
| | rs11250496 | 1.38E-04 | 0.235 | 0.857 | 0.166 | 10 | 1,437,105 | ADARB2 | intron | 0 |
| | rs10508213 | 1.68E-04 | −0.232 | 0.857 | 0.112 | 10 | 1,601,165 | ADARB2 | intron | 0 |
| 11A | rs2862667 | 4.09E-05 | 0.252 | 0.725 | 0.266 | 11 | 43,099,468 | LOC100128134 | 5′-downstream | 44,525 |
| | rs10838023 | 7.64E-05 | 0.243 | 0.845 | 0.267 | 11 | 43,113,488 | LOC100128134 | 5′-downstream | 58,545 |
| | rs2018445 | 9.12E-04 | 0.211 | 0.944 | 0.268 | 11 | 43,117,351 | LOC100128134 | 5′-downstream | 62,408 |
| 20A | rs10485526 | 8.30E-04 | −0.206 | 0.944 | 0.361 | 20 | 15,383,203 | MACROD2 | intron | 0 |
| | rs6079836 | 3.60E-05 | −0.254 | 0.711 | 0.339 | 20 | 15,383,564 | MACROD2 | intron | 0 |
| | rs6079839 | 3.60E-05 | −0.254 | 0.711 | 0.339 | 20 | 15,384,282 | MACROD2 | intron | 0 |
| 21A | rs2776278 | 2.13E-04 | −0.229 | 0.871 | 0.284 | 21 | 36,264,569 | RPL23AP3 | 3′-downstream | 45,524 |
| | rs2249118 | 2.41E-05 | −0.260 | 0.696 | 0.364 | 21 | 36,277,829 | RPL23AP3 | 3′-downstream | 32,264 |
| | rs2212916 | 2.80E-05 | −0.257 | 0.711 | 0.366 | 21 | 36,281,638 | RPL23AP3 | 3′-downstream | 28,455 |
| | rs2249599 | 5.19E-04 | −0.215 | 0.944 | 0.251 | 21 | 36,281,873 | RPL23AP3 | 3′-downstream | 28,220 |
| | rs2249971 | 1.69E-04 | −0.232 | 0.857 | 0.352 | 21 | 36,284,531 | RPL23AP3 | 3′-downstream | 25,562 |
| | rs7280598 | 6.44E-04 | −0.211 | 0.944 | 0.285 | 21 | 36,295,016 | RPL23AP3 | 3′-downstream | 15,077 |
| | rs2032319 | 7.16E-04 | −0.209 | 0.944 | 0.283 | 21 | 36,295,592 | RPL23AP3 | 3′-downstream | 14,501 |

*R values represent correlation coefficients for the association.
^{†}Genes selected for siRNA screening are highlighted by shading.

“Integrated” SNP, basal expression and radiation AUC analyses: The effect of genetic variation on radiation-induced cytotoxicity may result, in part, from the regulation of gene expression. Post radiation gene expression is known to be influenced by DNA sequence variation (Correa and Cheung (2004) Am. J. Hum. Genet. 75:885-890; and Smirnov et al. (2009) Nature 459:587-591. However, few studies have focused on basal gene expression levels and their possible relationship to radiation response, i.e., on information that might be used to predict response. Therefore, an “integrated analysis” that included data for SNPs, basal expression, and radiation AUC also was performed. Specifically, 175 SNPs were identified with p values<10⁻³ that mapped to the 27 identified “loci” (Table 8), and then data for the 54,000 basal expression array probe sets on the Affymetrix U133 Plus 2.0 platform were used to identify SNPs within those loci that might be associated with basal gene expression, in either a cis or trans fashion.

Specifically, 2,432 SNP-expression associations were observed for the 175 SNPs with p values<10⁻⁴. These 2,432 expression probe sets were correlated with radiation AUC, and probe sets with p values<10⁻³ for association with radiation AUC were identified. A less stringent p value cutoff was selected for this final step to capture as much information as possible, with the understanding that many of the associations would be false-positives. This “integrated analysis,” moving from “loci” to SNPs to expression, identified 50 unique SNPs located in 14 of the 27 loci that were significantly associated with data for 47 probe sets that represented 39 unique annotated genes, i.e., basal expression of these genes was associated with radiation AUC with p<10⁻³ (Table 9). None of the SNPs were in cis-regulatory regions, defined as 5Mb on either side of the gene identified. These 50 unique SNPs mapped to 8 different chromosomes, with at least 2 SNPs on each of those chromosomes. The four most significant “loci” or “SNP peak regions” mapped to chromosomes 1, 4, 5 and 8, respectively, and contained the LRRN2, IL19, KCNK1, LDB2, hCG_{—}2025798, DEPDC1B, LOC100131033 and PLEKHF2 genes (Table 9). The SNPs near PLEKHF2 within the “locus” on chromosome 8 (FIG. 5C) were particularly striking since they were associated with variation in the expression of six annotated genes, and variation in the expression of those genes was, in turn, significantly associated with radiation AUC. The chromosome 1 locus contained the largest number of SNPs (19) that were associated with radiation AUC, with p values that ranged from 10⁻³ to 10⁻⁴. Those 19 SNPs were significantly associated with the expression of 12 annotated genes that were also significantly associated with radiation AUC with p values that ranged from 10⁻³ to 10⁻⁴. Six annotated genes were associated with the locus on chromosome 8 that contained the most significant 6 linked SNPs (p values<10⁻⁴), and those 6 genes were significantly associated with radiation AUC, with p values<10⁻³. The chromosome 4 and 5 loci included 7 and 6 SNPs, respectively, that were associated with radiation AUC with p values that ranged from 10⁻³ to 10⁻⁴, and those SNPs were associated with the expression of 11 and 4 unique annotated genes, respectively, with p values that ranged from 10⁻⁴ to 10⁻⁷. Expression levels for those 15 genes also were associated with radiation AUC, with p values<10⁻³.

TABLES 9A, 9B, and 9C
“Integrated analyses” with the top expression probe sets that were
associated with SNPs within “loci” and with radiation AUC
(SNP vs Expression p-value <10⁻⁴, and Expression vs AUC p-value <10⁻³).

| | SNP | | | | |
|---|---|---|---|---|---|
| 9A | SNP ID | MAF | Chr | Position | Closest gene^{†} |
| 1 | rs13128522 | 0.060 | 4 | 16,149,782 | LDB2 |
| 2 | rs10516301 | 0.072 | 4 | 16,143,251 | LDB2 |
| 3 | rs13128522 | 0.060 | 4 | 16,149,782 | LDB2 |
| 4 | rs1 3138387 | 0.065 | 4 | 16,142,474 | LDB2 |
| 5 | rs13128522 | 0.060 | 4 | 16,149,782 | LDB2 |
| 6 | rs4742269 | 0.139 | 9 | 6,839,317 | 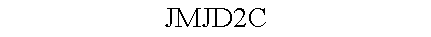 |
| 7 | rs12702213 | 0.058 | 7 | 46,381,080 | LOC730338 |
| 8 | rs13239088 | 0.054 | 7 | 46,381,408 | LOC730338 |
| 9 | rs6873333 | 0.072 | 5 | 155,195,850 | LOC100131033 |
| 10 | rs7378774 | 0.064 | 5 | 155,221,392 | LOC100131033 |
| 11 | rs10073856 | 0.071 | 5 | 155,195,298 | LOC100131033 |
| 12 | rs780266 | 0.071 | 1 | 231,791,723 | KCNK1 |
| 13 | rs1773951 | 0.070 | 1 | 231,802,262 | KCNK1 |
| 14 | rs10516301 | 0.072 | 4 | 16,143,251 | LDB2 |
| 15 | rs10805350 | 0.313 | 4 | 16,140,499 | LDB2 |
| 16 | rs13128522 | 0.060 | 4 | 16,149,782 | LDB2 |
| 17 | rs13138387 | 0.065 | 4 | 16,142,474 | LDB2 |
| 18 | rs898386 | 0.229 | 1 | 202,846,320 | LRRN2 |
| 19 | rs7539399 | 0.269 | 1 | 202,856,667 | LRRN2 |
| 20 | rs898387 | 0.235 | 1 | 202,845,996 | LRRN2 |
| 21 | rs898386 | 0.229 | 1 | 202,846,320 | LRRN2 |
| 22 | rs898387 | 0.235 | 1 | 202,845,996 | LRRN2 |
| 23 | rs1561715 | 0.083 | 8 | 96,189,090 | 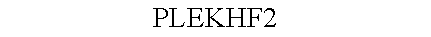 |
| 24 | rs1610110 | 0.083 | 8 | 96,185,038 | 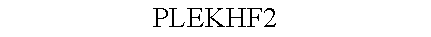 |
| 25 | rs7000734 | 0.081 | 8 | 96,196,206 | 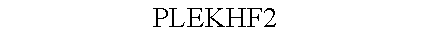 |
| 26 | rs4392868 | 0.087 | 8 | 96,189,248 | 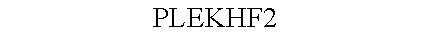 |
| 27 | rs13128522 | 0.060 | 4 | 16,149,782 | LDB2 |
| 28 | rs2883036 | 0.361 | 1 | 205,091,385 | IL19 |
| 29 | rs6873333 | 0.072 | 5 | 155,195,850 | LOC100131033 |
| 30 | rs7379436 | 0.074 | 5 | 155,158,726 | LOC100131033 |
| 31 | rs7378774 | 0.064 | 5 | 155,221,392 | LOC100131033 |
| 32 | rs10073856 | 0.071 | 5 | 155,195,298 | LOC100131033 |
| 33 | rs1561714 | 0.087 | 8 | 96,189,151 | 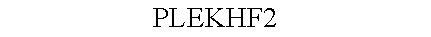 |
| 34 | rs1561715 | 0.083 | 8 | 96,189,090 | 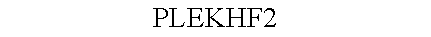 |
| 35 | rs1610110 | 0 083 | 8 | 96,185,038 | 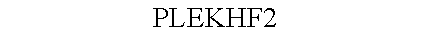 |
| 36 | rs7000734 | 0.081 | 8 | 96,196,206 | 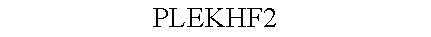 |
| 37 | rs212551 | 0.132 | 8 | 96,201,280 | 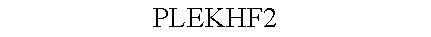 |
| 38 | rs4392868 | 0.087 | 8 | 96,189,248 | 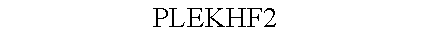 |
| 39 | rs13138387 | 0.065 | 4 | 16,142,474 | LDB2 |
| 40 | rs4951407 | 0.489 | 1 | 202,824,693 | LRRN2 |
| 41 | rs2212916 | 0.366 | 21 | 36,281,638 | RPL23AP3 |
| 42 | rs780256 | 0.288 | 1 | 231,809,726 | KCNK1 |
| 43 | rs1693219 | 0.289 | 1 | 231,807,892 | KCNK1 |
| 44 | rs1693216 | 0.286 | 1 | 231,808,517 | KCNK1 |
| 45 | rs2249971 | 0.352 | 21 | 36,284,531 | RPL23AP3 |
| 46 | rs212551 | 0.132 | 8 | 96,201,280 | 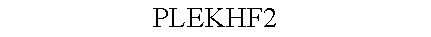 |
| 47 | rs7680285 | 0.049 | 4 | 181,228,438 | hCG_2025798 |
| 48 | rs35018391 | 0.049 | 4 | 181,217,870 | hCG_2025798 |
| 49 | rs41333347 | 0.049 | 4 | 181,227,186 | hCG_2025798 |
| 50 | rs780266 | 0.071 | 1 | 231,791,723 | KCNK1 |
| 51 | rs1773951 | 0.070 | 1 | 231,802,262 | KCNK1 |
| 52 | rs884108 | 0.180 | 1 | 202,857,860 | LRRN2 |
| 53 | rs11240237 | 0.475 | 1 | 202,879,141 | LRRN2 |
| 54 | rs11240239 | 0.469 | 1 | 202,879,427 | LRRN2 |
| 55 | rs4951089 | 0.475 | 1 | 202,877,521 | LRRN2 |
| 56 | rs4951088 | 0.471 | 1 | 202,877,403 | LRRN2 |
| 57 | rs11250496 | 0.166 | 10 | 1,437,105 | ADARB2 |
| 58 | rs212551 | 0.132 | 8 | 96,201,280 | GET 44 |
| 59 | rs13128522 | 0.060 | 4 | 16,149,782 | LDB2 |
| 60 | rs10516301 | 0.072 | 4 | 16,143,251 | LDB2 |
| 61 | rs13128522 | 0.060 | 4 | 16,149,782 | LDB2 |
| 62 | rs13138387 | 0.065 | 4 | 16,142,474 | LDB2 |
| 63 | rs17657370 | 0.078 | 7 | 96,444,428 | FLJ34048 |
| 64 | rs17598132 | 0.076 | 7 | 96,417,275 | FLJ34048 |
| 65 | rs898386 | 0.229 | 1 | 202,846,320 | LRRN2 |
| 66 | rs780266 | 0.071 | 1 | 231,791,723 | KCNK1 |
| 67 | rs1773951 | 0.070 | 1 | 231,802,262 | KCNK1 |
| 68 | rs1050821 | 0.112 | 10 | 1,601,165 | ADARB2 |
| 69 | rs898386 | 0.229 | 1 | 202,846,320 | LRRN2 |
| 70 | rs898387 | 0.235 | 1 | 202,845,996 | LRRN2 |
| 71 | rs1561715 | 0.083 | 8 | 96,189,090 | 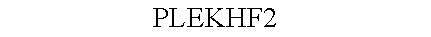 |
| 72 | rs1610110 | 0.083 | 8 | 96,185,038 | 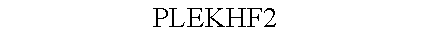 |
| 73 | rs7000734 | 0.081 | 8 | 96,196,206 | 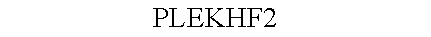 |
| 74 | rs212551 | 0.132 | 8 | 96,201,280 | 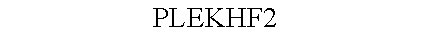 |
| 75 | rs4392868 | 0.087 | 8 | 96,189,248 | 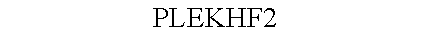 |
| 76 | rs13128522 | 0.060 | 4 | 16,149,782 | LDB2 |
| 77 | rs6873333 | 0.072 | 5 | 155,195,850 | LOC100131033 |
| 78 | rs7379436 | 0.074 | 5 | 155,158,726 | LOC100131033 |
| 79 | rs7378774 | 0.064 | 5 | 155,221,392 | LOC100131033 |
| 80 | rs10073856 | 0.071 | 5 | 155,195,298 | LOC100131033 |
| 81 | rs10516301 | 0.072 | 4 | 16,143,251 | LDB2 |
| 82 | rs13128522 | 0.060 | 4 | 16,149,782 | LDB2 |
| 83 | rs13138387 | 0.065 | 4 | 16,142,474 | LDB2 |
| 84 | rs13138387 | 0.065 | 4 | 16,142,474 | LDB2 |
| 85 | rs780266 | 0.071 | 1 | 231,791,723 | KCNK1 |
| 86 | rs1773951 | 0.070 | 1 | 231,802,262 | KCNK1 |
| 87 | rs1561715 | 0.083 | 8 | 96,189,090 | 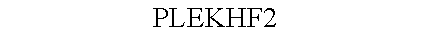 |
| 88 | rs1610110 | 0.083 | 8 | 96,185,03 | 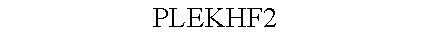 |
| 89 | rs7000734 | 0.081 | 8 | 96,196,206 | 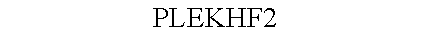 |
| 90 | rs10516301 | 0.072 | 4 | 16,143,251 | LDB2 |
| 91 | rs884108 | 0.180 | 1 | 202,857,860 | LRRN2 |
| 92 | rs10516301 | 0.072 | 4 | 16,143,251 | LDB2 |
| 93 | rs13128522 | 0.060 | 4 | 16,149,782 | LDB2 |
| 94 | rs2243188 | 0.442 | 1 | 205,081,095 | IL19 |
| 95 | rs4845144 | 0.458 | 1 | 205,083,271 | 1L19 |
| 96 | rs2243174 | 0.444 | 1 | 205,078,108 | 1L19 |
| 97 | rs11119670 | 0.442 | 1 | 205,071,944 | IL19 |
| 98 | rs4326096 | 0.270 | 5 | 59,926,035 | 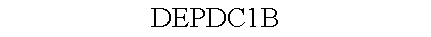 |
| 99 | rs2409791 | 0.280 | 5 | 59,920,074 | 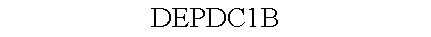 |
| 100 | rs13138387 | 0.065 | 4 | 16,142,474 | LDB2 |
| 101 | rs10959554 | 0.076 | 9 | 11,121,371 | LOC646114 |
| 102 | rs11250464 | 0.175 | 10 | 1,396,364 | ADARB2 |
| 103 | rs2387653 | 0.130 | 10 | 1,397,826 | ADARB2 |
| 104 | rs780266 | 0.071 | 1 | 231,791,723 | KCNK1 |
| 105 | rs1773951 | 0.070 | 1 | 231,802,262 | KCNK1 |
| 106 | rs1561715 | 0.083 | 8 | 96,189,090 | 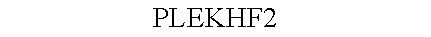 |
| 107 | rs1610110 | 0.083 | 8 | 96,185,038 | 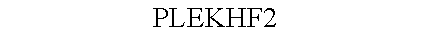 |
| 108 | rs7000734 | 0.081 | 8 | 96,196,206 | 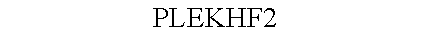 |
| 109 | rs4392868 | 0.087 | 8 | 96,189,248 | 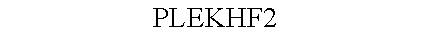 |

| 9B | Probe set | | | | | |
|---|---|---|---|---|---|---|
| ID | Probe | Chr | Gene Symbol | | | |
| 1 | 204004_at | 12 | 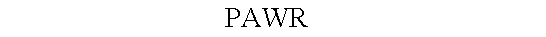 | | | |
| 2 | 207238_s_at | 1 | PTPRC | | | |
| 3 | 207238_s_at | 1 | PTPRC | | | |
| 4 | 207238_s_at | 1 | PTPRC | | | |
| 5 | 204005_s_at | 12 | 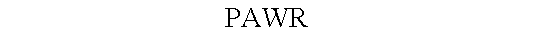 | | | |
| 6 | 214173_x_at | 19 | 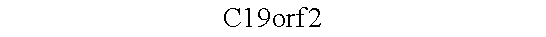 | | | |
| 7 | 227806_at | 16 | C16orf74 | | | |
| 8 | 227806_at | 16 | C16orf74 | | | |
| 9 | 220953_s_at | 5 | 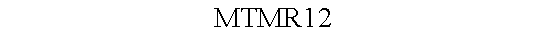 | | | |
| 10 | 220953_s_at | 5 | 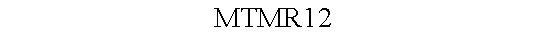 | | | |
| 11 | 220953_s_at | 5 | 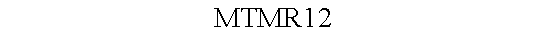 | | | |
| 12 | 1557984_s_at | 12 | RPAP3 | | | |
| 13 | 1557984_s_at | 12 | RPAP3 | | | |
| 14 | 212588_at | 1 | PTPRC | | | |
| 15 | 212588_at | 1 | PTPRC | | | |
| 16 | 212588_at | 1 | PTPRC | | | |
| 17 | 212588_at | 1 | PTPRC | | | |
| 18 | 216685_s_at | 9 | MTAP | | | |
| 19 | 216685_s_at | 9 | MTAP | | | |
| 20 | 216685_s_at | 9 | MTAP | | | |
| 21 | 204652_s_at | 7 | 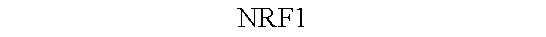 | | | |
| 22 | 204652_s_at | 7 | 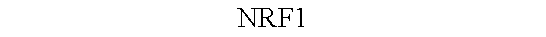 | | | |
| 23 | 205981_s_at | 4 | ING2 | | | |
| 24 | 205981_s_at | 4 | ING2 | | | |
| 25 | 205981_s_at | 4 | ING2 | | | |
| 26 | 205981_s_at | 4 | ING2 | | | |
| 27 | 226231_at | 20 | — | | | |
| 28 | 1566001_at | 16 | — | | | |
| 29 | 201946_s_at | 12 | CCT2 | | | |
| 30 | 201946_s_ at | 12 | CCT2 | | | |
| 31 | 201946_s_ at | 12 | CCT2 | | | |
| 32 | 201946_s_at | 12 | CCT2 | | | |
| 33 | 1555852_at | 6 | — | | | |
| 34 | 1555852_at | 6 | — | | | |
| 35 | 1555852_at | 6 | — | | | |
| 36 | 1555852_at | 6 | — | | | |
| 37 | 1555852_at | 6 | — | | | |
| 38 | 1555852_at | 6 | — | | | |
| 39 | 203524_s_at | 22 | MPST | | | |
| 40 | 203218_at | 5 | MAPK9 | | | |
| 41 | 213498_at | 11 | CREB3L1 | | | |
| 42 | 202180_s_at | 16 | MVP | | | |
| 43 | 202180_s_at | 16 | MVP | | | |
| 44 | 202180_s_at | 16 | MVP | | | |
| 45 | 202180_s_at | 16 | MVP | | | |
| 46 | 202180_s_at | 16 | MVP | | | |
| 47 | 200650_s_at | 1 | 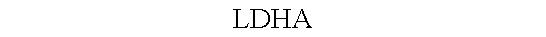 | | | |
| 48 | 200650_s_at | 1 | 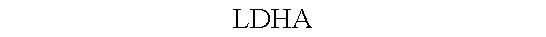 | | | |
| 49 | 200650_s_at | 1 | 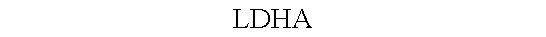 | | | |
| 50 | 207079_s_at | 14 | MED6 | | | |
| 51 | 207079_s_at | 14 | MED6 | | | |
| 52 | 222714_s_at | 8 | LACTB2 | | | |
| 53 | 222714_s_at | 8 | LACTB2 | | | |
| 54 | 222714_s_at | 8 | LACTB2 | | | |
| 55 | 222714_s_at | 8 | LACTB2 | | | |
| 56 | 222714_s_at | 8 | LACTB2 | | | |
| 57 | 224596_at | 9 | SLC44A1 | | | |
| 58 | 1554271_a_at | 1 | 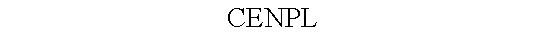 | | | |
| 59 | 219501_at | 13 | ENOX1 | | | |
| 60 | 206935_at | 13 | PCDH8 | | | |
| 61 | 206935_at | 13 | PCDH8 | | | |
| 62 | 206935_at | 13 | PCDH8 | | | |
| 63 | 206935_at | 13 | PCDH8 | | | |
| 64 | 206935_at | 13 | PCDH8 | | | |
| 65 | 229173_at | 2 | KIAA1715 | | | |
| 66 | 222387_s_at | 16 | VPS35 | | | |
| 67 | 222387_s_at | 16 | VPS35 | | | |
| 68 | 203353_s_at | 18 | MBD1 | | | |
| 69 | 204444_at | 10 | KIF11 | | | |
| 70 | 204444_at | 10 | KIF11 | | | |
| 71 | 203856_at | 14 | VRK1 | | | |
| 72 | 203856_at | 14 | VRK1 | | | |
| 73 | 203856_at | 14 | VRK1 | | | |
| 74 | 203856_at | 14 | VRK1 | | | |
| 75 | 203856_at | 14 | VRK1 | | | |
| 76 | 209016_s_at | 12 | KRT7 | | | |
| 77 | 206220_s_at | 13 | RASA3 | | | |
| 78 | 206220_s_at | 13 | RASA3 | | | |
| 79 | 206220_s_at | 13 | RASA3 | | | |
| 80 | 206220_s_at | 13 | RASA3 | | | |
| 81 | 218584_at | 12 | TCTN1 | | | |
| 82 | 218584_at | 12 | TCTN1 | | | |
| 83 | 218584_at | 12 | TCTN1 | | | |
| 84 | 225525_at | 22 | CTA-221G9.4/// | | | |
| | | | LOC100131004 | | | |
| 85 | 1554577_a_at | 23 | PSMD10 | | | |
| 86 | 1554577_a_at | 23 | PSMD1Q | | | |
| 87 | 218634_at | 1 | PHLDA3 | | | |
| 88 | 218634_at | 1 | PHLDA3 | | | |
| 89 | 218634_at | 1 | PHLDA3 | | | |
| 90 | 221698_s_at | 12 | CLEC7A | | | |
| 91 | 243721_at | 18 | — | | | |
| 92 | 212587_s_at | 1 | PTPRC | | | |
| 93 | 212587_s_at | 1 | PTPRC | | | |
| 94 | 235959_at | 1 | — | | | |
| 95 | 235959_at | 1 | — | | | |
| 96 | 235959_at | 1 | — | | | |
| 97 | 235959_at | 1 | — | | | |
| 98 | 219544_at | 13 | 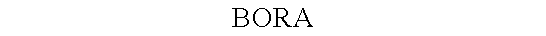 | | | |
| 99 | 219544_at | 13 | 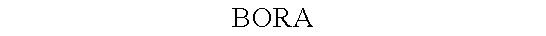 | | | |
| 100 | 212124_at | 10 | ZMIZ1 | | | |
| 101 | 1558794_at | 10 | LOC728190 | | | |
| 102 | 209670_at | 14 | TRAC | | | |
| 103 | 209670_at | 14 | TRAC | | | |
| 104 | 1554493_s_at | 2 | THADA | | | |
| 105 | 1554493_s_at | 2 | THADA | | | |
| 106 | 215750_at | 22 | KIAA1659 | | | |
| 107 | 215750_at | 22 | KIAA1659 | | | |
| 108 | 215750_at | 22 | KIAA1659 | | | |
| 109 | 215750_at | 22 | KIAA1659 | | | |
| 9C | GWAS | | | | | |
| | R value* | P value* | R value | P value | R value | P value |
| | (SNP vs | (SNP vs | (SNP vs | (SNP vs | (EXP vs | (EXP vs |
| ID | Exp) | Exp) | AUC) | AUC) | AUC) | AUC) |
| 1 | −0.251 | 3.53E-05 | 0.260 | 2.38E-05 | −0.266 | 7.46E-06 |
| 2 | −0.275 | 5.16E-06 | 0.250 | 4.97E-05 | −0.264 | 8.81E-06 |
| 3 | −0.288 | 1.74E-06 | 0.260 | 2.38E-05 | −0.264 | 8.81E-06 |
| 4 | −0.277 | 4.31E-06 | 0.232 | 1.70E-04 | −0.264 | 8.81E-06 |
| 5 | −0.276 | 5.00E-06 | 0.260 | 2.38E-05 | −0.261 | 1.10E-05 |
| 6 | −0.238 | 8.37E-05 | 0.280 | 4.73E-06 | −0.254 | 1.83E-05 |
| 7 | −0.250 | 3.48E-05 | 0.240 | 9.29E-05 | −0.251 | 2.45E-05 |
| 8 | −0.245 | 6.52E-05 | 0.249 | 6.64E-05 | −0.251 | 2.45E-05 |
| 9 | −0.242 | 6.14E-05 | 0.254 | 3.64E-05 | −0.240 | 5.40E-05 |
| 10 | −0.254 | 2.96E-05 | 0.245 | 7.71E-05 | −0.240 | 5.40E-05 |
| 11 | −0.247 | 4.40E-05 | 0.241 | 9.49E-05 | −0.240 | 5.40E-05 |
| 12 | −0.252 | 3.13E-05 | 0.233 | 1.55E-04 | −0.239 | 5.94E-05 |
| 13 | −0.252 | 2.91E-05 | 0.235 | 1.34E-04 | −0.239 | 5.94E-05 |
| 14 | −0.308 | 2.86E-07 | 0.250 | 4.97E-05 | −0.233 | 8.78E-05 |
| 15 | −0.253 | 3.14E-05 | 0.222 | 3.50E-04 | −0.233 | 8.78E-05 |
| 16 | −0.333 | 2.65E-08 | 0.260 | 2.38E-05 | −0.233 | 8.78E-05 |
| 17 | −0.294 | 9.98E-07 | 0.232 | 1.70E-04 | −0.233 | 8.78E-05 |
| 18 | −0.257 | 2.14E-05 | 0.253 | 4.22E-05 | −0.233 | 9.19E-05 |
| 19 | −0.242 | 6.39E-05 | 0.222 | 3.08E-04 | −0.233 | 9.19E-05 |
| 20 | −0.259 | 1.75E-05 | 0.268 | 1.24E-05 | −0.233 | 9.19E-05 |
| 21 | −0.278 | 4.32E-06 | 0.253 | 4.22E-05 | −0.230 | 1.13E-04 |
| 22 | −0.266 | 1.04E-05 | 0.268 | 1.24E-05 | −0.230 | 1.13E-04 |
| 23 | −0.248 | 4.09E-05 | 0.299 | 9.58E-07 | −0.229 | 1.20E-04 |
| 24 | −0.248 | 4.09E-05 | 0.299 | 9.58E-07 | −0.229 | 1.20E-04 |
| 25 | −0.239 | 7.62E-05 | 0.309 | 3.82E-07 | −0.229 | 1.20E-04 |
| 26 | −0.248 | 4.08E-05 | 0.274 | 8.24E-06 | −0.229 | 1.20E-04 |
| 27 | −0.271 | 7.48E-06 | 0.260 | 2.38E-05 | −0.227 | 1.36E-04 |
| 28 | −0.260 | 2.07E-05 | −0.221 | 3.99E-04 | 0.224 | 1.70E-04 |
| 29 | −0.284 | 2.35E-06 | 0.254 | 3.64E-05 | −0.224 | 1.72E-04 |
| 30 | −0.246 | 4.56E-05 | 0.239 | 1.00E-04 | −0.224 | 1.72E-04 |
| 31 | −0.283 | 3.10E-06 | 0.245 | 7.71E-05 | −0.224 | 1.72E-04 |
| 32 | −0.290 | 1.49E-06 | 0.241 | 9.49E-05 | −0.224 | 1.72E-04 |
| 33 | −0.295 | 8.57E-07 | 0.261 | 2.14E-05 | −0.223 | 1.79E-04 |
| 34 | −0.322 | 7.04E-08 | 0.299 | 9.58E-07 | −0.223 | 1.79E-04 |
| 35 | −0.322 | 7.04E-08 | 0.299 | 9.58E-07 | −0.223 | 1.79E-04 |
| 36 | −0.302 | 4.57E-07 | 0.309 | 3.82E-07 | −0.223 | 1.79E-04 |
| 37 | −0.239 | 7.95E-05 | 0.245 | 6.85E-05 | −0.223 | 1.79E-04 |
| 38 | −0.312 | 2.01E-07 | 0.274 | 8.24E-06 | −0.223 | 1.79E-04 |
| 39 | −0.252 | 2.93E-05 | 0.232 | 1.70E-04 | −0.223 | 1.81E-04 |
| 40 | −0.239 | 8.10E-05 | 0.206 | 8.84E-04 | −0.221 | 2.10E-04 |
| 41 | −0.237 | 8.99E-05 | −0.257 | 2.80E-05 | 0.219 | 2.40E-04 |
| 42 | 0.261 | 1.54E-05 | 0.225 | 2.67E-04 | 0.219 | 2.47E-04 |
| 43 | 0.247 | 4.47E-05 | 0.247 | 5.89E-05 | 0.219 | 2.47E-04 |
| 44 | 0.253 | 3.15E-05 | 0.247 | 6.50E-05 | 0.219 | 2.47E-04 |
| 45 | −0.241 | 6.89E-05 | −0.232 | 1.69E-04 | 0.219 | 2.47E-04 |
| 46 | 0.258 | 1.87E-05 | 0.245 | 6.85E-05 | 0.219 | 2.47E-04 |
| 47 | −0.242 | 6.36E-05 | 0.255 | 3.23E-05 | −0.216 | 2.89E-04 |
| 48 | −0.242 | 6.36E-05 | 0.255 | 3.23E-05 | −0.216 | 2.89E-04 |
| 49 | −0.242 | 6.36E-05 | 0.255 | 3.23E-05 | −0.216 | 2.89E-04 |
| 50 | −0.246 | 5.00E-05 | 0.233 | 1.55E-04 | −0.213 | 3.60E-04 |
| 51 | −0.242 | 6.23E-05 | 0.235 | 1.34E-04 | −0.213 | 3.60E-04 |
| 52 | −0.240 | 8.32E-05 | 0.241 | 1.07E-04 | −0.211 | 4.07E-04 |
| 53 | −0.242 | 6.33E-05 | 0.232 | 1.60E-04 | −0.211 | 4.07E-04 |
| 54 | −0.238 | 8.54E-05 | 0.237 | 1.22E-04 | −0.211 | 4.07E-04 |
| 55 | −0.252 | 2.94E-05 | 0.232 | 1.60E-04 | −0.211 | 4.07E-04 |
| 56 | −0.242 | 6.12E-05 | 0.237 | 1.20E-04 | −0.211 | 4.07E-04 |
| 57 | −0.254 | 2.65E-05 | 0.235 | 1.38E-04 | −0.211 | 4.13E-04 |
| 58 | −0.239 | 7.84E-05 | 0.245 | 6.85E-05 | −0.211 | 4.14E-04 |
| 59 | 0.246 | 5.11E-05 | 0.260 | 2.38E-05 | 0.211 | 4.18E-04 |
| 60 | 0.276 | 4.76E-06 | 0.250 | 4.97E-05 | 0.209 | 4.55E-04 |
| 61 | 0.271 | 7.23E-06 | 0.260 | 2.38E-05 | 0.209 | 4.55E-04 |
| 62 | 0.245 | 4.92E-05 | 0.232 | 1.70E-04 | 0.209 | 4.55E-04 |
| 63 | 0.239 | 7.84E-05 | 0.255 | 3.34E-05 | 0.209 | 4.55E-04 |
| 64 | 0.245 | 5.33E-05 | 0.263 | 1.89E-05 | 0.209 | 4.55E-04 |
| 65 | −0.245 | 5.33E-05 | 0.253 | 4.22E-05 | −0.208 | 5.00E-04 |
| 66 | −0.243 | 5.96E-05 | 0.233 | 1.55E-04 | −0.208 | 5.03E-04 |
| 67 | −0.242 | 6.21E-05 | 0.235 | 1.34E-04 | −0.208 | 5.03 E-04 |
| 68 | 0.238 | 8.11E-05 | −0.232 | 1.68E-04 | −0.207 | 5.39E-04 |
| 69 | −0.243 | 6.10E-05 | 0.253 | 4.22E-05 | −0.207 | 5.44E-04 |
| 70 | −0.236 | 9.50E-05 | 0.268 | 1.24E-05 | −0.207 | 5.44E-04 |
| 71 | −0.256 | 2.17E-05 | 0.299 | 9.58E-07 | −0.206 | 5.68E-04 |
| 72 | −0.256 | 2.17E-05 | 0.299 | 9.58E-07 | −0.206 | 5.68E-04 |
| 73 | −0.257 | 2.00E-05 | 0.309 | 3.82E-07 | −0.206 | 5.68E-04 |
| 74 | −0.280 | 3.27E-06 | 0.245 | 6.85E-05 | −0.206 | 5.68E-04 |
| 75 | −0.246 | 4.98E-05 | 0.274 | 8.24E-06 | −0.206 | 5.68E-04 |
| 76 | 0.302 | 5.15E-07 | 0.260 | 2.38E-05 | 0.206 | 5.76E-04 |
| 77 | −0.268 | 8.39E-06 | 0.254 | 3.64E-05 | −0.205 | 5.83E-04 |
| 78 | −0.241 | 6.83E-05 | 0.239 | 1.00E-04 | −0.205 | 5.83E-04 |
| 79 | −0.289 | 1.81E-06 | 0.245 | 7.71E-05 | −0.205 | 5.83E-04 |
| 80 | −0.264 | 1.28E-05 | 0.241 | 9.49E-05 | −0.205 | 5.83E-04 |
| 81 | 0.312 | 1.98E-07 | 0.250 | 4.97E-05 | 0.205 | 5.88E-04 |
| 82 | 0.307 | 3.22E-07 | 0.260 | 2.38E-05 | 0.205 | 5.88E-04 |
| 83 | 0.246 | 4.84E-05 | 0.232 | 1.70E-04 | 0.205 | 5.88E-04 |
| 84 | 0.266 | 1.03E-05 | 0.232 | 1.70E-04 | 0.205 | 6.04E-04 |
| 85 | −0.242 | 6.35E-05 | 0.233 | 1.55E-04 | −0.204 | 6.21E-04 |
| 86 | −0.247 | 4.22E-05 | 0.235 | 1.34E-04 | −0.204 | 6.21E-04 |
| 87 | 0.237 | 9.02E-05 | 0.299 | 9.58E-07 | 0.204 | 6.21E-04 |
| 88 | 0.237 | 9.02E-05 | 0.299 | 9.58E-07 | 0.204 | 6.21E-04 |
| 89 | 0.241 | 6.88E-05 | 0.309 | 3.82E-07 | 0.204 | 6.21E-04 |
| 90 | 0.287 | 1.82E-06 | 0.250 | 4.97E-05 | 0.204 | 6.37E-04 |
| 91 | 0.250 | 4.19E-05 | 0.241 | 1.07E-04 | 0.203 | 6.73E-04 |
| 92 | −0.248 | 4.31E-05 | 0.250 | 4.97E-05 | −0.203 | 6.84E-04 |
| 93 | −0.257 | 2.13E-05 | 0.260 | 2.38E-05 | −0.203 | 6.84E-04 |
| 94 | 0.258 | 1.94E-05 | 0.244 | 7.11E-05 | 0.202 | 7.16E-04 |
| 95 | 0.240 | 8.31E-05 | 0.222 | 3.50E-04 | 0.202 | 7.16E-04 |
| 96 | 0.239 | 7.90E-05 | 0.244 | 7.04E-05 | 0.202 | 7.16E-04 |
| 97 | 0.239 | 8.17E-05 | 0.244 | 7.45E-05 | 0.202 | 7.16E-04 |
| 98 | 0.236 | 9.97E-05 | −0.255 | 3.41E-05 | −0.201 | 7.56E-04 |
| 99 | 0.245 | 4.86E-05 | −0.270 | 1.09E-05 | −0.201 | 7.56E-04 |
| 100 | −0.244 | 5.55E-05 | 0.232 | 1.70E-04 | −0.201 | 7.67E-04 |
| 101 | 0.241 | 7.10E-05 | 0.227 | 2.33E-04 | 0.200 | 8.40E-04 |
| 102 | −0.241 | 7.38E-05 | 0.279 | 5.61E-06 | −0.199 | 8.63E-04 |
| 103 | −0.265 | 1.24E-05 | 0.212 | 6.69E-04 | −0.199 | 8.63E-04 |
| 104 | −0.246 | 4.77E-05 | 0.233 | 1.55E-04 | −0.199 | 8.77E-04 |
| 105 | −0.249 | 3.84E-05 | 0.235 | 1.34E-04 | −0.199 | 8.77E-04 |
| 106 | 0.263 | 1.31E-05 | 0.299 | 9.58E-07 | 0.197 | 9.79E-04 |
| 107 | 0.263 | 1.31E-05 | 0.299 | 9.58E-07 | 0.197 | 9.79E-04 |
| 108 | 0.259 | 1.71E-05 | 0.309 | 3.82E-07 | 0.197 | 9.79E-04 |
| 109 | 0.250 | 3.73E-05 | 0.274 | 8.24E-06 | 0.197 | 9.79E-04 |

*R values represent correlation coefficients for each association.
^{†}Genes selected for siRNA screening are highlighted by shading.

Functional validation of candidate genes in tumor cell lines: The initial association experiments were performed with human LCLs. Since non-genetic factors might confound the results of these association studies, and since gene regulation is tissue specific Dimas et al. (2009) Science 325:1246-1250, studies were done with human tumor cell lines. Specifically, two pancreatic cancer cell lines (MIA-PaCa2; p53 mutant and HupT3; p53 mutant), and one cervical cancer cell line (HeLa; p53 wild type (WT)), were used to functionally validate association results obtained with LCLs. These functional experiments involved siRNA knockdown followed by MTS cytotoxicity assays and, subsequently, colony-forming assays. These three cancer cell lines were selected because of their relative sensitivity to radiation after testing with MTS assays.

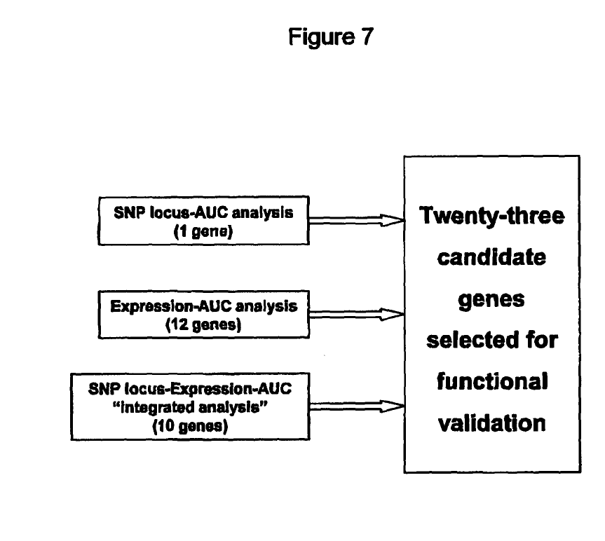

Based on the analysis of the 54,000 basal expression probe sets, 1.3 million SNPs and radiation AUC data, as well as an evaluation of their biological function, 23 candidate genes identified as described herein were selected for siRNA screening with the tumor cell lines. These genes were chosen based on the following criteria: genes with at least one expression array probe set that had a p value<10⁻³ for association with radiation AUC; genes containing SNPs found within a “locus” associated with radiation AUC (p<10⁻⁴); or genes for which expression was associated with both AUC and SNPs (p<10⁻³ for AUC and p<10⁻⁴ for SNPs, i.e., the “integrated analysis”). In addition, the genes had to be expressed in the lymphoblastoid cell lines (expression level>50 after GCRMA normalization) and could not previously have been implicated in radiation sensitivity. Application of this approach resulted in the selection of 10 genes that were identified during the “integrated” analyses; 1 gene was selected based on the SNP-AUC analysis, and 12 genes were selected on the basis of the expression-AUC analysis. This overall selection strategy is depicted graphically in FIG. 7.

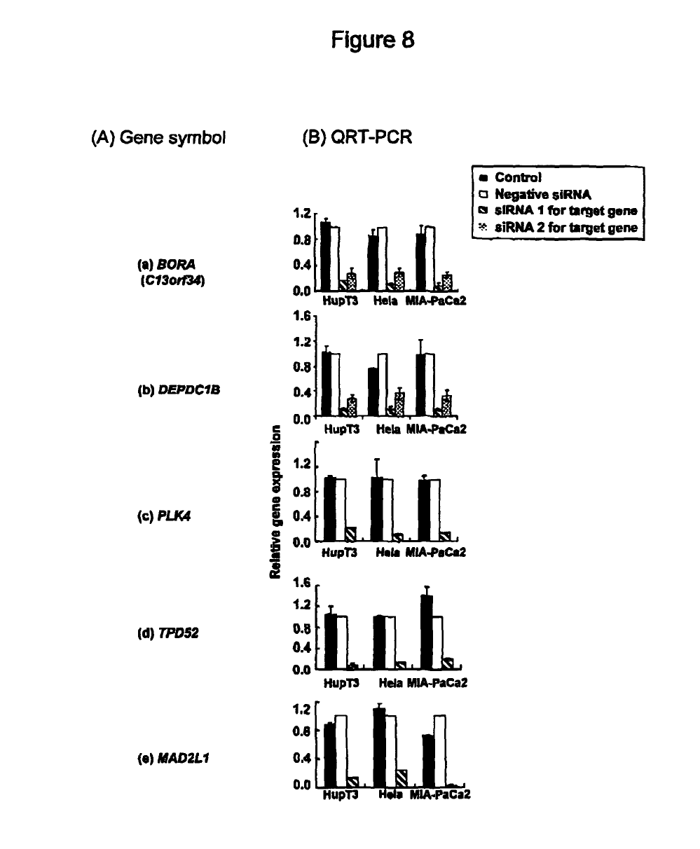

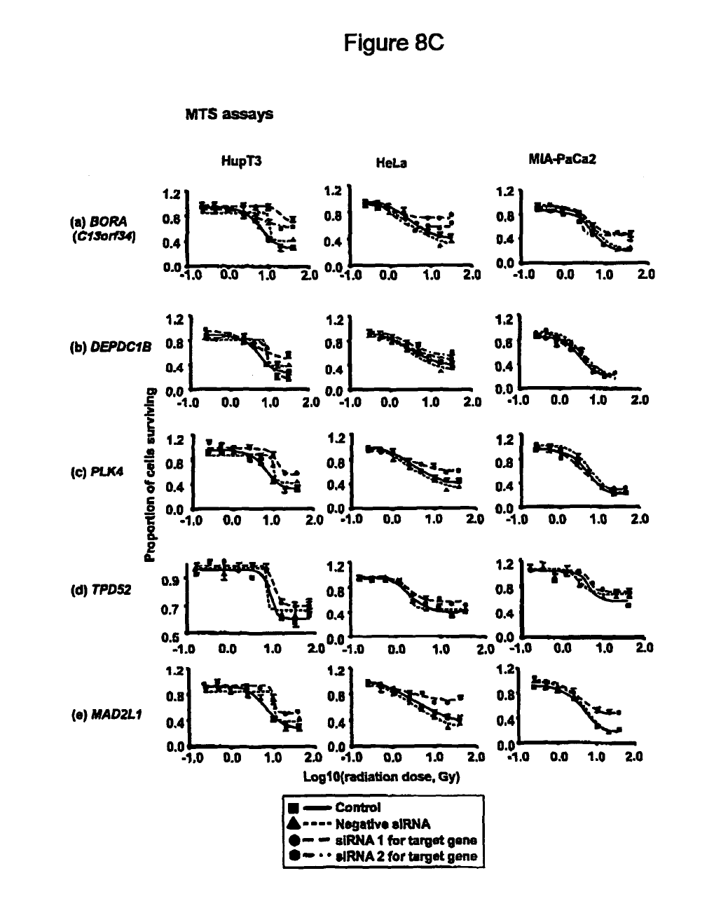

For functional validation, either two unvalidated siRNAs or one validated siRNA (based on QIAGEN data) were used to knockdown each of the 23 candidate genes. If two siRNAs were available, “significance” was defined as a gene with a significant change in apparent AUC for both siRNAs in comparison with a control siRNA. MTS assays were performed with all three tumor cell lines for all 23 of the genes selected for study. Knockdown of 7 genes had a significant effect on radiation sensitivity in one cell line, 3 genes were positive for two cell lines and knockdown of 2 genes significantly altered radiation sensitivity in all three cell lines (Table 10 and FIG. 8). Five genes were then selected for further study for which knockdown with specific siRNAs significantly altered radiation sensitivity in at least 2 cancer cell lines, specifically BORA (C13orf34), MAD2L1, PLK4, TPD52, and TTF1 (Table 10, FIG. 8). DEPDC1B also was included, even though this gene only showed an effect of knockdown on radiation sensitivity in HeLa cells, since two SNPs located 2.5 and 8.4 kb downstream of this gene (rs4326096 and rs2409791) were significantly correlated with the expression of BORA (p=9.97×10⁻⁵ and 4.86×10⁻⁵, respectively), a gene that displayed a functional effect on radiation-induced cytotoxicity in all three cancer cell lines (FIG. 8, Table 10). The two SNPs were highly linked (r²>0.9).

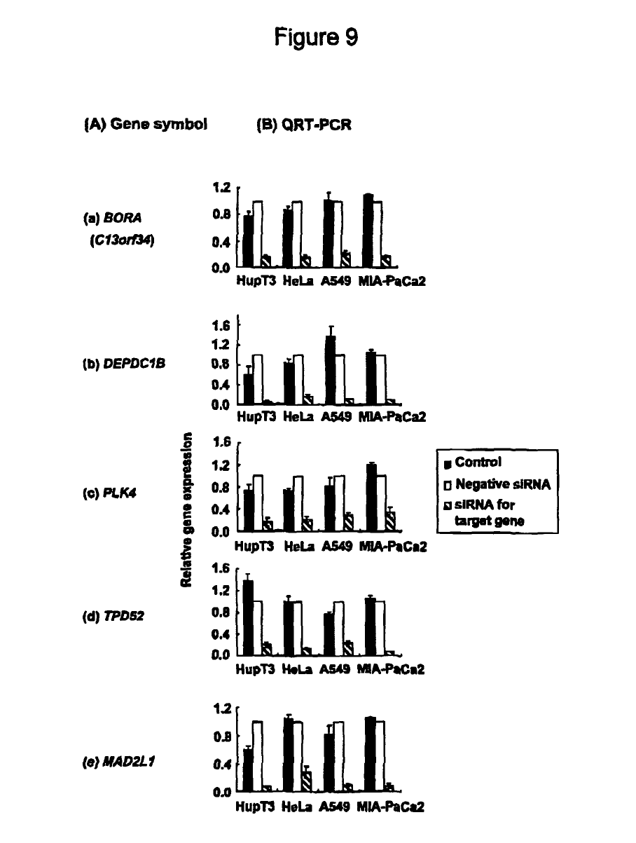

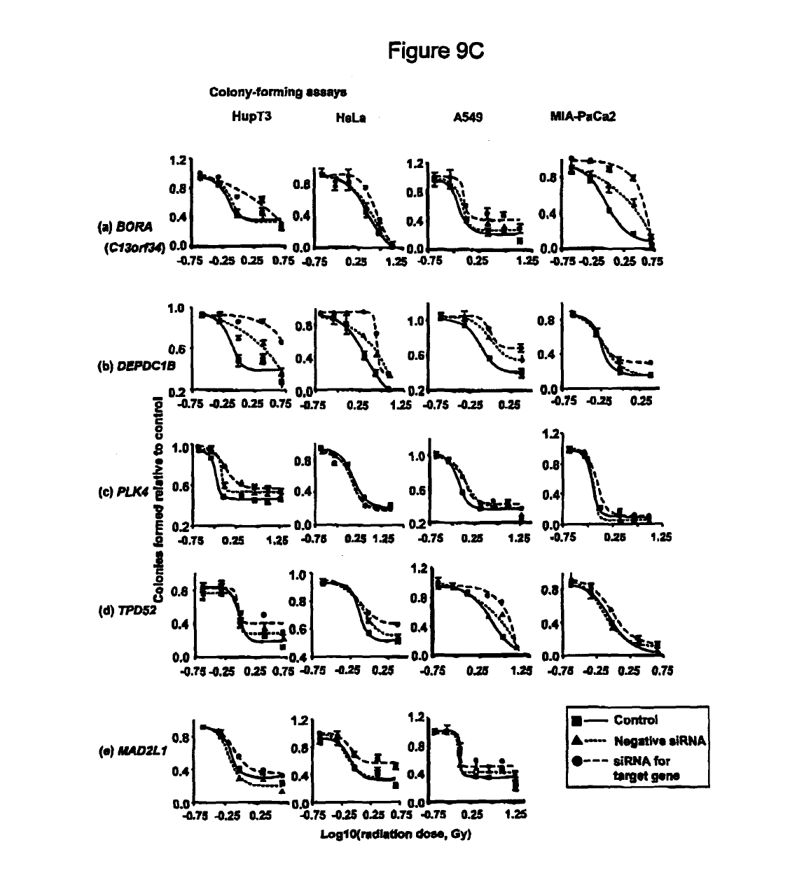

As the next step in the analysis, and to further confirm results obtained with the MTS assay, performed colony-forming assays were performed for these same six genes in the MIA-PaCa2, HupT3 and HeLa cells used to perform the MTS assays. A lung cancer cell line (A459) also was used, since radiation is commonly used to treat lung cancer. As shown graphically in FIG. 9, knockdown of BORA, DEPDC1B and TPD52 desensitized all four cell lines to radiation treatment. MAD2L1 knockdown had a significant impact on radiation effect in HeLa and HupT3, but not in the A549 cell line. However, knockdown of MAD2L1 resulted in lack of colony formation in MIA-PaCa2 cells in the absence of radiation treatment, indicating that this gene might be essential for cell proliferation. Knockdown of PLK4 only desensitized radiation response in HupT3 cells, consistent with the MTS assay results, and TTF1 knockdown did not alter radiation sensitivity in any of the four cell lines.

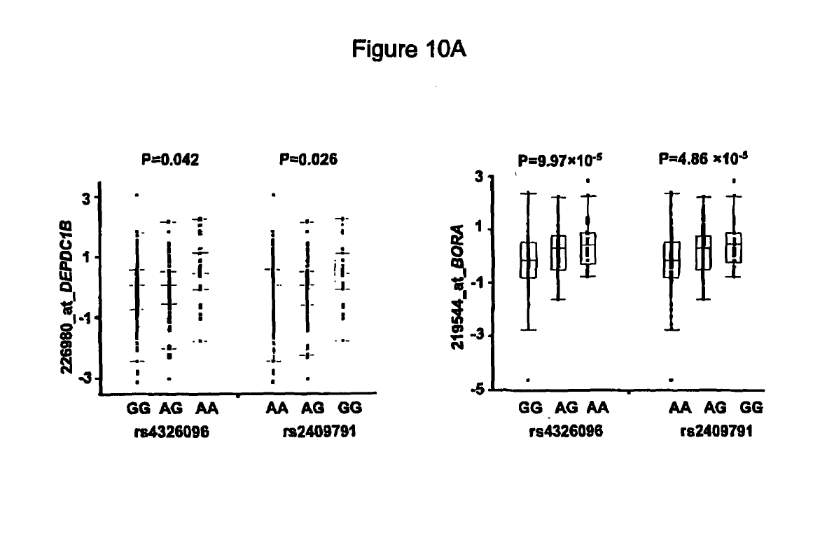

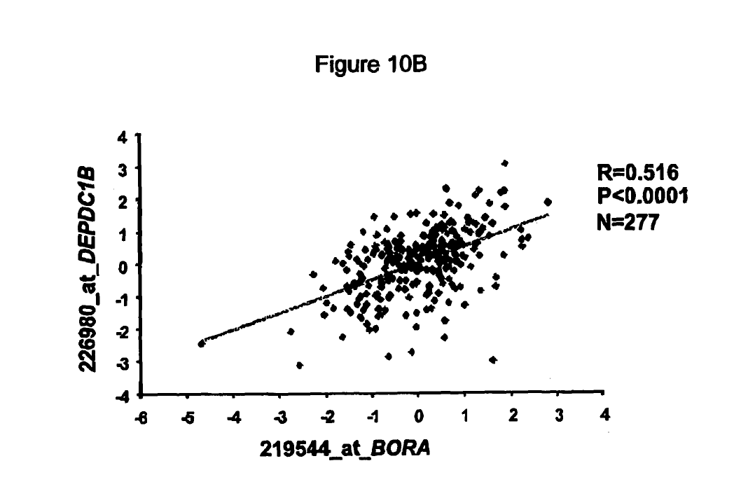

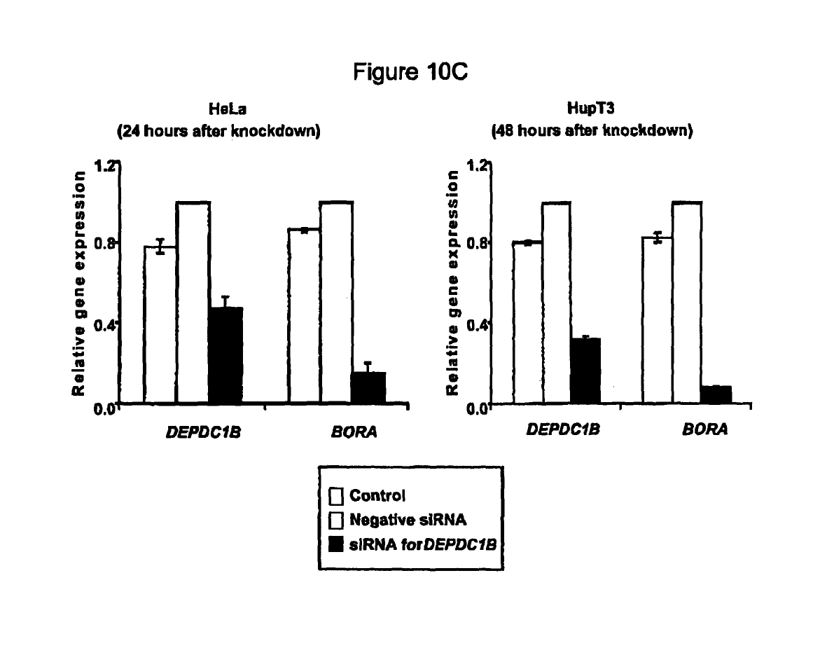

Since the rs4326096 and rs2409791 SNPs were close to DEPDC1B and also were associated with BORA expression, and since BORA knockdown altered radiation sensitivity with both MTS and colony-forming assays, studies were conducted to determine whether the influence of these two SNPs on BORA expression might be mediated through the regulation of DEPDC1B expression. An association analysis was performed for these SNPs using microarray data for DEPDC1B, and the result showed that both SNPs were also highly associated with DEPDC1B gene expression level (FIG. 10A). Furthermore, expression levels for BORA and DEPDC1B were significantly and positively correlated (FIG. 10B, Rₚ=0.516, p<0.0001), a relationship consistent with the results of the siRNA knockdown experiments. Finally to further characterize the relationship between DEPDC1B and BORA, siRNA knockdown of DEPDC1B was performed, followed by determination of BORA expression in HeLa and HupT3 cell lines (FIG. 10C), two cell lines that showed significant effects on radiation sensitivity after knockdown of both genes (FIG. 8). The results for HeLa cells showed that 24 hours after the knockdown of DEPDC1B, BORA expression decreased dramatically, while in HupT3 cells this effect was most prominent 48 hours after DEPDC1B knockdown.

TABLE 10
Candidate genes selected for siRNA screening

| | | Basis for Selection* | | | | | | | | | |
|---|---|---|---|---|---|---|---|---|---|---|---|
| | | Exp. | SNP | | | | | | | | |
| | | vs. | vs. | Integrated | MTS Assays^{†} | | | Colony-forming Assay^{†} | | | |
| ID | Gene Symbol | AUC^{¶} | AUC^{§} | Analysis^{‡} | HupT3 | HeLa | MIAPaCa2 | HupT3 | HeLa | A549 | MIAPaCa2 |
| 1 | BORA (C13orf34) | Yes | — | Yes | Yes | Yes | Yes | Yes | Yes | Yes | Yes |
| 2 | C19orf2 | Yes | — | Yes | — | Yes | — | NP | NP | NP | NP |
| 3 | CENPL | Yes | — | Yes | — | — | Yes | NP | NP | NP | NP |
| 4 | CTNNAL1 | Yes | — | — | — | — | — | NP | NP | NP | NP |
| 5 | DEPDC1B | — | Yes | Yes | — | Yes | — | Yes | Yes | Yes | Yes |
| 6 | FAM62B | Yes | — | — | — | Yes | — | NP | NP | NP | NP |
| 7 | JMJD2C | — | Yes | Yes | — | — | — | NP | NP | NP | NP |
| 8 | KBTBD2 | Yes | — | — | — | — | — | NP | NP | NP | NP |
| 9 | LDHA | Yes | — | Yes | — | — | — | NP | NP | NP | NP |
| 10 | MAD2L1 | Yes | — | — | Yes | Yes | Yes | Yes | Yes | — | NC |
| 11 | MTMR12 | Yes | — | Yes | — | — | — | NP | NP | NP | NP |
| 12 | NIBP (TRAPPC9) | — | Yes | — | — | — | — | NP | NP | NP | NP |
| 13 | NRF1 | Yes | — | Yes | Yes | — | — | NP | NP | NP | NP |
| 14 | PAWR | Yes | — | Yes | — | — | — | NP | NP | NP | NP |
| 15 | PLK4 | Yes | — | — | Yes | Yes | — | Yes | — | — | — |
| 16 | SR140 | Yes | — | — | — | — | — | NP | NP | NP | NP |
| 17 | STS | Yes | — | — | — | — | — | NP | NP | NP | NP |
| 18 | TDP1 | Yes | — | — | — | — | — | NP | NP | NP | NP |
| 19 | TMEM48 | Yes | — | — | — | Yes | — | NP | NP | NP | NP |
| 20 | TPD52 | Yes | — | — | Yes | Yes | — | Yes | Yes | Yes | Yes |
| 21 | TTF1 | Yes | — | — | Yes | Yes | — | — | — | — | — |
| 22 | UBE2D2 | Yes | — | — | — | Yes | — | NP | NP | NP | NP |
| 23 | PLEKHF2 | — | Yes | Yes | — | — | — | NP | NP | NP | NP |

*“Yes” indicates individual candidate genes with the p value listed
^{†}“Yes” indicates knockdown of the gene altered radiation cytotoxicity (AUC values) when compared with control siRNA
^{¶}P < 10⁻³; ^{§}P < 10⁻⁴; ^{‡}SNP vs Exp P < 10⁻⁴ and Exp vs AUC P < 10⁻³
“NP” = not performed; “NC” = no colonies formed for that cell line after knockdown with a specific siRNA

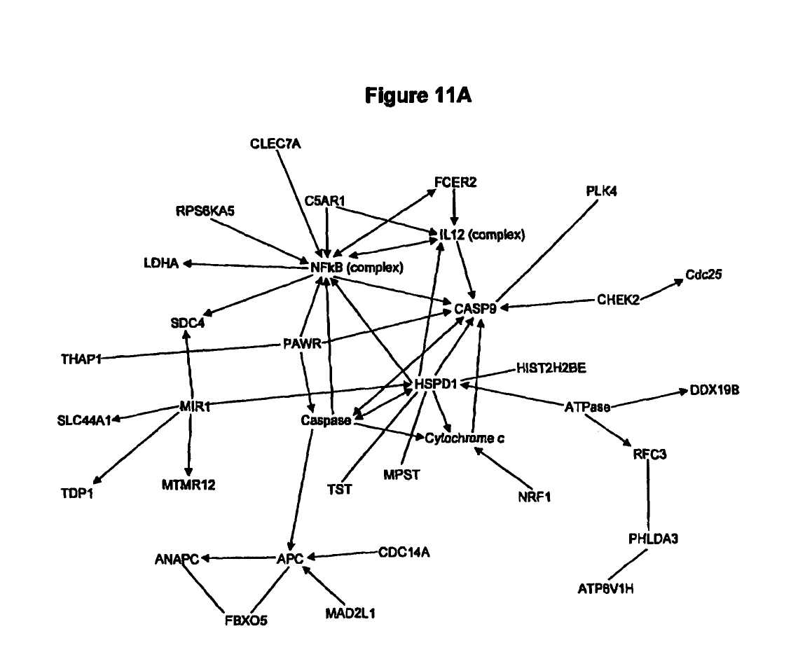

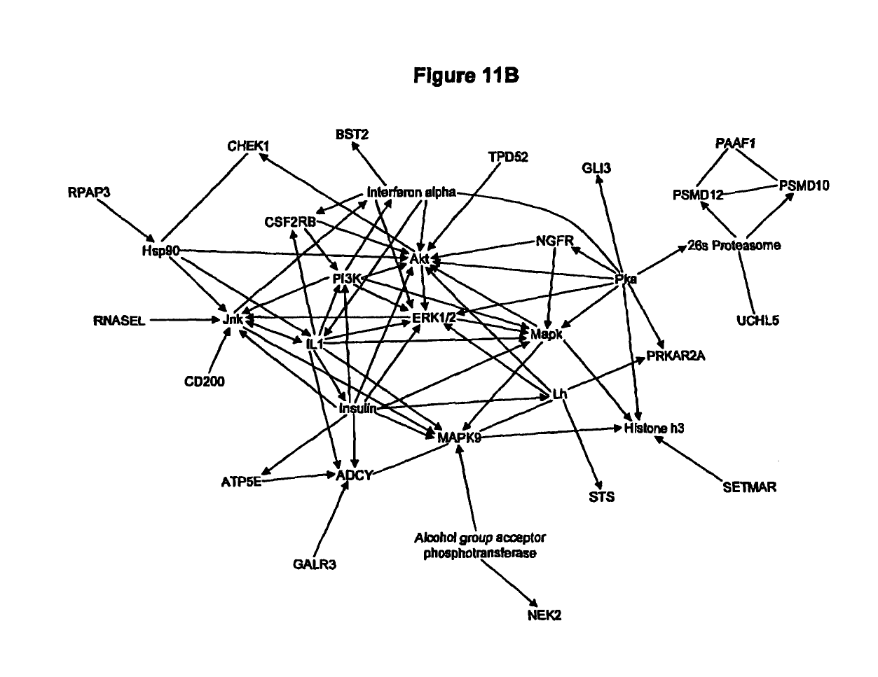

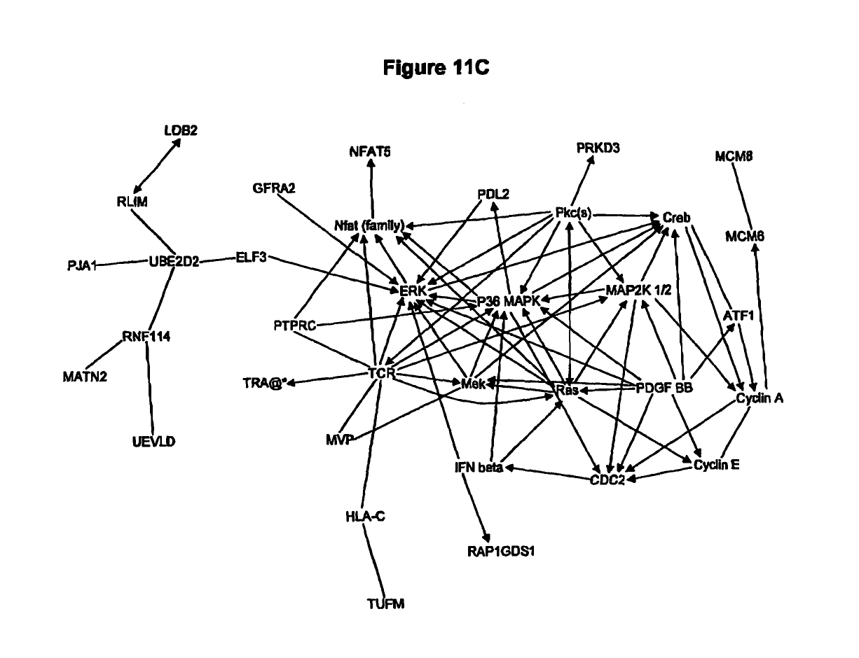

The analyses of SNP, basal gene expression, and radiation cytotoxicity (AUC) data yielded a total of 240 candidate genes, including 211 identified as a result of either expression vs AUC associations (p<10⁻³) or an “integrated analysis” that included SNP expression and radiation AUC data. In addition, 29 genes also were identified based on the association of SNPs with AUC (p<10⁻⁴). When Ingenuity Pathway analysis was performed for these 240 genes, the top three networks all involved “cell death” and centered around NFκB, PI3K/Akt and p38MAPK/ERK as “network hubs” (FIGS. 11A-11C). Many candidate genes identified during these studies have been reported to have altered levels of expression in response to radiation exposure in the NCI-60 cell lines or in lymphoblastoid cell lines, especially p53 dependent genes such as CDC2, PHLDA3 and PTPRC (Amundson et al. (1999) Oncogene 18:3666-3672; Amundson et al. (2005) Oncogene 24:4572-4579; Amundson et al. (2008) Cancer Res. 68:415-424; Amundson et al. (2003) Mol. Cancer Res. 1:445-452; and Jen and Cheung (2005) Cancer Res. 65:7666-7673). In addition, genes such as MEF2B, NRF1, PHPTJ, ZMAT3, CHEKJ, and GALR3 that are up- or down-regulated by ionizing radiation exposure, also were found to be associated with radiation AUC (Amundson et al. (2004) Cancer Res. 64:6368-6371; Dressman et al. (2007) PLoS Med. 4:e106; Jen and Cheung, supra; Paul and Amundson (2008) Int. J. Radiat. Oncol. Biol. Phys. 71:1236-1244; Rzeszowska-Wolny et al. (2009) DNA Repair (Amst) 8:732-738; and Westbury et al. (2009) J. Pathol. 219:131-140. The correlation between basal gene expression and radiation cytotoxicity was of greater interest, however, because basal gene expression may eventually be more useful to help predict radiation sensitivity and resistance, while change in gene expression in response to radiation might be more closely related to downstream signaling events.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An in vitro method for predicting the effectiveness of radiation therapy in a subject, said method comprising:
(a) providing a biological sample from the subject;
(b) assaying the biological sample to determine whether it comprises a radiation therapy signature; and
(c) classifying the subject as having a greater likelihood of responding to radiation therapy if the signature is present in the biological sample, and classifying the subject as having a lesser likelihood of responding to radiation therapy if the signature is not present in the biological sample.

2. The method of claim 1, further comprising communicating to a medical professional information regarding whether or not the signature is present in the biological sample.

3. The method of claim 2, further comprising communicating to a medical professional information indicating that the presence of the signature correlates with effectiveness of radiation therapy.

4. The method of claim 1, wherein said radiation therapy signature comprises one or more markers selected from the group consisting of rs7000734, rs16885294, rs1561715, rs1610110, rs2859631, rs12569163, rs16889440, rs7554126, rs4742269, rs7591064, rs11250464, rs1853665, rs4392868, rs17598306, rs1471356, and rs4554799.

5. The method of claim 1, wherein said radiation therapy signature comprises one or more markers selected from the group consisting of rs4392868, rs297550, rs1946944, rs12548426, rs1610110, rs1561715, rs1561714, rs4734298, rs7000734, and rs212551.

6. The method of claim 1, wherein said radiation therapy signature comprises one or more markers selected from the group consisting of rs1610110, rs1561715, rs1561714, rs4392868, rs7000734, and rs212551.

7. The method of claim 1, wherein said radiation therapy signature comprises one or more markers selected from the group consisting of rs6863920, rs6449478, rs2409791, rs4326096, rs206789, and rs286158.

8. The method of claim 1, wherein said radiation therapy signature comprises one or more markers selected from the group consisting of rs4463400, rs6578080, rs4397386, rs7387053, rs4487737, rs13275618, rs6578083, rs7814976, rs7841539, rs6578084, and rs9324519.

9. The method of claim 1, wherein said radiation therapy signature comprises one or more markers selected from the group consisting of rs12553351 and rs4742269.

10. The method of claim 1, wherein said radiation therapy signature comprises rs1610110.

11. The method of claim 1, wherein said radiation therapy signature comprises rs1561715.

12. The method of claim 1, wherein said radiation therapy signature comprises rs7000734.

13. An in vitro method for determining a dose of radiation for administration to a subject, said method comprising:
(a) providing a biological sample from the subject;
(b) assaying the biological sample to determine whether it comprises a radiation therapy signature; and
(c) determining that the dose is lower if the signature is present in the biological sample than if the signature is not present in the biological sample, and determining that the dose is higher if the signature is not present in the biological sample than if the signature is present in the biological sample.

14. The method of claim 13, further comprising communicating to a medical professional information regarding whether or not the signature is present in the biological sample.

15. The method of claim 14, further comprising communicating to a medical professional information indicating that the presence of the signature correlates with a lower dose of radiation therapy.

16. The method of claim 13, wherein said radiation therapy signature comprises one or more markers selected from the group consisting of rs7000734, rs16885294, rs1561715, rs1610110, rs2859631, rs12569163, rs16889440, rs7554126, rs4742269, rs7591064, rs11250464, rs1853665, rs4392868, rs17598306, rs1471356, and rs4554799.

17. The method of claim 13, wherein said radiation therapy signature comprises one or more markers selected from the group consisting of rs4392868, rs297550, rs1946944, rs12548426, rs1610110, rs1561715, rs1561714, rs4734298, rs7000734, and rs212551.

18. The method of claim 13, wherein said radiation therapy signature comprises one or more markers selected from the group consisting of rs1610110, rs1561715, rs1561714, rs4392868, rs7000734, and rs212551.

19. The method of claim 13, wherein said radiation therapy signature comprises one or more markers selected from the group consisting of rs6863920, rs6449478, rs2409791, rs4326096, rs206789, and rs286158.

20. The method of claim 13, wherein said radiation therapy signature comprises one or more markers selected from the group consisting of rs4463400, rs6578080, rs4397386, rs7387053, rs4487737, rs13275618, rs6578083, rs7814976, rs7841539, rs6578084, and rs9324519.

21. The method of claim 13, wherein said radiation therapy signature comprises one or more markers selected from the group consisting of rs12553351 and rs4742269.

22. The method of claim 13, wherein said radiation therapy signature comprises rs1610110.

23. The method of claim 13, wherein said radiation therapy signature comprises rs1561715.

24. The method of claim 13, wherein said radiation therapy signature comprises rs7000734.
